(19) United States
(12) Patent Application Publication
(10) Pub. No.: US 2025/0177383 A1
(43) Pub. Date: Jun. 5, 2025

(54) DI-CYCLOPROPYL BASED IL-17A MODULATORS AND USES THEREOF

(71) Applicant: DICE ALPHA, INC., Indianapolis, IN (US)

(72) Inventors: Timothy J. CHURCH, South San Francisco, CA (US); Paul R. FATHEREE, San Francisco, CA (US); Martin S. LINSELL, San Mateo, CA (US); Margot G. PAULICK, San Carlos, CA (US); Maureen Kay REILLY, Emerald Hills, CA (US)

(21) Appl. No.: 18/841,320

(22) PCT Filed: Feb. 23, 2023
(86) PCT No.: PCT/US2023/013710

§ 371 Date: Aug. 23, 2024

Related U.S. Application Data
(60) Provisional application No. 63/314,257, filed on Feb. 25, 2022.

(51) Int. Cl. A61K 31/496; C07D 241/04; C07D 401/12; C07D 403/12; C07D 413/12; C07D 413/14; C07D 487/04; C07D 487/08; C07D 487/10

(52) U.S. Cl. CPC A61K 31/496; C07D 241/04; C07D 401/12; C07D 403/12; C07D 413/12; C07D 413/14; C07D 487/04; C07D 487/08; C07D 487/10

(57) ABSTRACT

The disclosure herein provides compounds and pharmaceutical compositions of Formula (I) and Formula (I-A) for the modulation of IL-17A. Compounds and pharmaceutical compositions of Formula (I) and Formula (I-A) are useful for the treatment of inflammatory conditions, such as psoriasis.

### CROSS-REFERENCE

This application claims the benefit of priority of U.S. Provisional Patent Application No. 63/314,257 filed on Feb. 25, 2022, which is incorporated herein by reference in its entirety.

### BACKGROUND OF THE INVENTION

The IL-17 family consists of six cytokines (IL-17A through IL-17F). Interleukin-17A (IL-17A), is an established pro-inflammatory cytokine, which is involved in the induction of IL-6, IL-8, G-CSF, TNF-α, IL-1β, PGE2, and IFN-γ, as well as numerous chemokines and other effectors. IL-17A can form homodimers or heterodimers with its family member, IL-17F and can bind to both IL-17 receptors, IL-17 RA and IL-17 RC, in order to mediate signaling. IL-17A is a major pathological cytokine expressed by Th17 cells, which are involved in the pathology of inflammation and autoimmunity, and also CD8+ T cells, γδ cells, NK cells, NKT cells, macrophages and dendritic cells. Additionally, IL-17A and Th17 are necessary for defense against various microbes despite their involvement in inflammation and autoimmune disorders. Further, IL-17A can act in cooperation with other inflammatory cytokines such as TNF-α, IFN-γ, and IL-1β to mediate pro-inflammatory effects.

To date, there are a few biologics (Secukinumab and Ixekizumab) that have been approved to modulate IL-17A for the treatment of inflammatory diseases, such as psoriasis, ankylosing spondylitis, and psoriatic arthritis. These treatments require injection to a patient as they are not readily absorbed by the gut when orally ingested. Further, these approved biologic treatments have a high cost of entry for patients, limiting the availability to the patient population in need thereof. There are a few small molecule modulators of IL-17A that have been approved for oral administration. However, while these have the convenience of oral administration and a lower cost of entry for patients, they lack the efficacy of approved biologics. Therefore, there exists a need for the development of potent small molecule IL-17A modulators for the treatment of inflammatory diseases and other associated disorders.

### SUMMARY OF THE INVENTION

In certain aspects the present disclosure provides a compound represented by the structure of Formula (I):

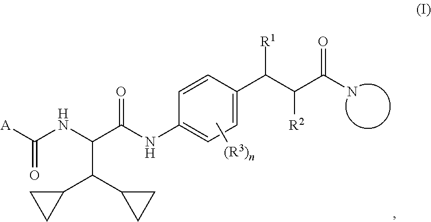

or a pharmaceutically acceptable salt thereof, wherein:
A is selected from a C₃₋₆ carbocycle and 5- to 6-membered heterocycle, any one of which is optionally substituted with one or more substituents independently selected from (a), (b), and (c):
   (a) halogen, —OR¹¹, —N(R¹¹)₂, —C(O)R¹¹, —C(O)OR¹¹, —NO₂, and —CN;
   (b) C₁₋₆ alkyl optionally substituted with one or more substituents independently selected from: halogen, —OR¹, —N(R¹¹)₂, —C(O)R¹¹, —C(O)OR¹¹, —NO₂, —CN, C₃₋₆ carbocycle and 3- to 6-membered heterocycle, wherein each C₃₋₆ carbocycle and 3- to 6-membered heterocycle is optionally substituted with one or more substituents independently selected from halogen, —OR¹¹, —N(R¹¹)₂, —C(O)R¹¹, —C(O)OR¹¹, —NO₂, and —CN; and
   (c) C₃₋₆ carbocycle optionally substituted with one or more substituents independently selected from halogen, C₁₋₄ alkyl, C₁₋₄ haloalkyl, —OR¹¹, —N(R¹¹)₂, —C(O)R¹¹, —C(O)OR¹¹, —NO₂, and —CN;
R¹ is selected from hydrogen, halogen, —OR¹², —N(R¹²)₂, —C(O)R¹², —C(O)OR¹², —NO₂, —CN, and C₁₋₆ alkyl optionally substituted with one or more substituents independently selected from halogen, —OR¹², —N(R¹²)₂, —C(O)R¹², —C(O)OR¹², —NO₂, and —CN;
R² is selected from —N(R^{A})C(O)(R^{B}) and —N(R^{C})C(O)N(R^{D})(R^{E});

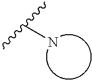 [SMILES: *N]

is selected from a 4- to 12-membered heterocycle optionally substituted with one or more substituents independently selected from:
   halogen, —OR¹³, —N(R¹³)₂, —C(O)R¹³, —C(O)OR¹³, —NO₂, —CN, and C₁₋₆ alkyl optionally substituted with one or more substituents independently selected from halogen, —OR¹³, —N(R¹³)₂, —C(O)R¹³, —C(O)OR¹³, —NO₂, and —CN;
R³ is selected from halogen, —OR¹⁴, —N(R¹⁴)₂, —C(O)R¹⁴, —C(O)OR¹⁴, —NO₂, —CN, C₁₋₆ alkyl and C₃₋₆ carbocycle, wherein each C₁₋₆ alkyl and C₃₋₆ carbocycle of which is optionally substituted with one or more substituents independently selected from halogen, —OR¹⁴, —N(R¹⁴)₂, —C(O)R¹⁴, —C(O)OR¹⁴, —NO₂, and —CN;
R^{A} is selected from hydrogen and C₁₋₆ alkyl optionally substituted with one or more substituents independently selected from halogen, —OR¹⁵, —N(R¹⁵)₂, —C(O)R¹⁵, —C(O)OR¹⁵, —NO₂, and —N;
R^{B} is selected from:
C₁₋₆ alkyl optionally substituted with one or more substituents independently selected from:
   halogen, —OR¹⁶, —N(R¹⁶)₂, —C(O)R¹⁶, —C(O)OR¹⁶, —N(R¹⁶)C(O)OR¹⁶, —NO₂, —CN;
   C₃₋₆ carbocycle and 5- to 6-membered heterocycle, any of which is optionally substituted with one or more substituents independently selected from halogen, —OR¹⁶, —N(R¹⁶)₂, —C(O)R¹⁶, —C(O)OR¹⁶, —NO₂, and —CN; and
C₃₋₆ carbocycle or 4- to 6-membered heterocycle, each of which is optionally substituted with one or more substituents independently selected from:
   halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN;
R^{C} is selected from hydrogen and C₁₋₆ alkyl optionally substituted with one or more substituents independently selected from: halogen, —OR¹⁷, —N(R¹⁷)₂, —C(O)R¹⁷, —C(O)OR¹⁷, —NO₂, and —CN;
R^{D} is selected from C₁₋₆ alkyl and C₃₋₆ carbocycle, any of which is optionally substituted with one or more substituents independently selected from: halogen, —OR¹⁸, —N(R¹⁸)₂, —C(O)R¹¹, —C(O)OR¹⁸, —NO₂, and —CN;
R^{E} is selected from hydrogen, C₁₋₆ alkyl and C₃₋₆carbocycle, any of which is optionally substituted with one or more substituents independently selected from: halogen, —OR¹⁹, —N(R¹⁹)₂, —C(O)R¹⁹, —C(O)OR¹⁹, —NO₂, and —CN;
R¹¹, R¹², R¹³, R¹⁴, R¹⁵, R¹⁶, R¹⁷, R¹⁸, and R¹⁹ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, =O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, =O, and —CN; and
n is selected from 0, 1, 2, 3, and 4.

In certain embodiments, the present disclosure provides a pharmaceutical composition comprising a compound or salt of Formula (I), and a pharmaceutically acceptable excipient.

In certain aspects the present disclosure provides a method of modulating IL-17A in a subject in need thereof, comprising administering to the subject a compound or salt of Formula (I) or a pharmaceutical composition thereof.

In certain aspects the present disclosure provides a method of treating an inflammatory disease or condition comprising administering to a subject in need thereof a compound or salt of Formula (I) or a pharmaceutical composition thereof. In some embodiments, the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain monovalent radical consisting solely of carbon and hydrogen atoms, containing no unsaturation, and preferably having from one to twelve carbon atoms (i.e., C₁-C₁₂ alkyl). The alkyl is attached to the remainder of the molecule through a single bond. In certain embodiments, an alkyl comprises one to twelve carbon atoms (i.e., C₁-C₁₂ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). For example, the alkyl group may be attached to the rest of the molecule by a single bind, such as, methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl), and the like.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein. In certain embodiments, an alkylene comprises one to ten carbon atoms (i.e., C₁-C₁₀ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

The term “C_{x-y}” when used in conjunction with a chemical moiety, such as alkyl, alkenyl, or alkynyl is meant to include groups that contain from x to y carbons in the chain. For example, the term “C₁₋₆ alkyl” refers to substituted or unsubstituted saturated hydrocarbon groups, including straight-chain alkyl and branched-chain alkyl groups that contain from 1 to 6 carbons. The term —C_{x-y} alkylene- refers to a substituted or unsubstituted alkylene chain with from x to y carbons in the alkylene chain. For example, —C₁₋₆ alkylene- may be selected from methylene, ethylene, propylene, butylene, pentylene, and hexylene, any one of which is optionally substituted.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C_{x-y} alkenylene- refers to a substituted or unsubstituted alkenylene chain with from x to y carbons in the alkenylene chain. For example, —C₂₋₆ alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkynylene chain. For example, —C₂₋₆ alkynylene- may be selected from ethynylene, propynylene, butynylene, pentynylene, and hexynylene, any one of which is optionally substituted. An alkynylene chain may have one triple bond or more than one triple bond in the alkynylene chain.

The term “carbocycle” as used herein refers to a saturated, unsaturated or aromatic ring in which each atom of the ring is carbon. Carbocycle include 3- to 10-membered monocyclic rings and 6- to 12-membered bicyclic rings. Each ring of a bicyclic carbocycle may be selected from saturated, unsaturated, and aromatic rings. Bicyclic carbocycles may be fused, bridged or spiro-ring systems. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein.

“Cycloalkyl” refers to a stable fully saturated monocyclic or polycyclic hydrocarbon radical consisting solely of carbon and hydrogen atoms, which includes fused or bridged ring systems, and preferably having from three to twelve carbon atoms (i.e., C₃₋₁₂ cycloalkyl). In certain embodiments, a cycloalkyl comprises three to ten carbon atoms (i.e., C₃₋₁₀ cycloalkyl). In other embodiments, a cycloalkyl comprises five to seven carbon atoms (i.e., C₅₋₇ cycloalkyl). The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Cycloalkyl may be optionally substituted by one or more substituents such as those substituents described herein.

“Cycloalkenyl” refers to a stable unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond (i.e., C₃₋₁₂ cycloalkenyl). In certain embodiments, a cycloalkenyl comprises three to ten carbon atoms (i.e., C₃₋₁₀ cycloalkenyl). In other embodiments, a cycloalkenyl comprises five to seven carbon atoms (i.e., C₅₋₇ cycloalkenyl). The cycloalkenyl may be attached to the rest of the molecule by a single bond. Examples of monocyclic cycloalkenyls include, e.g., cyclopentenyl, cyclohexenyl, cycloheptenyl, and cyclooctenyl. Cycloalkenyl may be optionally substituted by one or more substituents such as those substituents described herein.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Aryl may be optionally substituted by one or more substituents such as those substituents described herein.

A “C_{x-y} carbocycle” is meant to include groups that contain from x to y carbons in a ring. For example, the term “C₃₋₆ carbocycle” can be a saturated, unsaturated or aromatic ring system that contains from 3 to 6 carbon atoms-any of which is optionally substituted as provided herein.

The term “heterocycle” as used herein refers to a saturated, unsaturated, non-aromatic or aromatic ring comprising one or more heteroatoms. Exemplary heteroatoms include N, O, Si, P, B, and S atoms. Heterocycles include 3- to 10-membered monocyclic rings and 6- to 12-membered bicyclic rings. Each ring of a bicyclic heterocycle may be selected from saturated, unsaturated, and aromatic rings. In some embodiments, the heterocycle comprises at least one heteroatom selected from oxygen, nitrogen, sulfur, or any combination thereof. In some embodiments, the heterocycle comprises at least one heteroatom selected from oxygen, nitrogen, or any combination thereof. In some embodiments, the heterocycle comprises at least one heteroatom selected from oxygen, sulfur, or any combination thereof. In some embodiments, the heterocycle comprises at least one heteroatom selected from nitrogen, sulfur, or any combination thereof. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems. In an exemplary embodiment, a heterocycle, e.g., pyridyl, may be fused to a saturated or unsaturated ring, e.g., cyclohexane, cyclopentane, or cyclohexene. Heterocycle may be optionally substituted by one or more substituents such as those substituents described herein.

“Heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. In some embodiments, the heterocycloalkyl comprises at least one heteroatom selected from oxygen, nitrogen, sulfur, or any combination thereof. In some embodiments, the heterocycloalkyl comprises at least one heteroatom selected from oxygen, nitrogen, or any combination thereof. In some embodiments, the heterocycloalkyl comprises at least one heteroatom selected from oxygen, sulfur, or any combination thereof. In some embodiments, the heterocycloalkyl comprises at least one heteroatom selected from nitrogen, sulfur, or any combination thereof. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Heterocycloalkyl may be optionally substituted by one or more substituents such as those substituents described herein.

The term “heteroaryl” refers to a radical derived from a 3- to 12-membered aromatic ring radical that comprises one to eleven carbon atoms and at least one heteroatom wherein each heteroatom may be selected from N, O, and S. In some embodiments, the heteroaryl comprises at least one heteroatom selected from oxygen, nitrogen, sulfur, or any combination thereof. In some embodiments, the heteroaryl comprises at least one heteroatom selected from oxygen, nitrogen, or any combination thereof. In some embodiments, the heteroaryl comprises at least one heteroatom selected from oxygen, sulfur, or any combination thereof. In some embodiments, the heteroaryl comprises at least one heteroatom selected from nitrogen, sulfur, or any combination thereof. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Heteroaryl includes aromatic single ring structures, preferably 5- to 6-membered rings, whose ring structures include at least one heteroatom, preferably one to four heteroatoms, more preferably one or two heteroatoms. Heteroaryl groups include, for example, pyrrole, furan, thiophene, imidazole, oxazole, thiazole, pyrazole, pyridine, pyrazine, pyridazine, and pyrimidine, and the like. Heteroaryl may be optionally substituted by one or more substituents such as those substituents described herein. Heteroaryl also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl may be optionally substituted by one or more substituents such as those substituents described herein.

An “X-membered heterocycle” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

“Alkoxy” refers to a radical bonded through an oxygen atom of the formula —O-alkyl, where alkyl is an alkyl chain as defined above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, and I). When an alkyl group is substituted with more than one halogen radicals, each halogen may be independently selected for example, 1-chloro,2-fluoroethane.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazine (═N—NH₂), —R^{b}—OR^{a}, —R—OC(O)—R^{a}, —R^{b}—OC(O)—OR^{a}, —R^{b}—OC(O)—N(R^{a})₂, —R^{b}—N(R^{a})₂, —R—C(O)R^{a}, —R^{b}—C(O)OR^{a}, —R—C(O)N(R^{a})₂, —R—O—R^{C}—C(O)N(R^{a})₂, —R—N(R^{a})C(O)O R^{a}, —R—N(R^{a})C(O)R^{a}, —R—N(R^{a})S(O)ₜR^{a} (where t is 1 or 2), —R—S(O)ₜR^{a} (where t is 1 or 2), —R—S(O)ₜOR^{a} (where t is 1 or 2), and —R^{b}—S(O)N(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo(═N—OH), hydrazine (═N—NH₂), —R—OR^{a}, —R^{b}—O—C(O)—R^{a}, —R—OC(O)—OR^{a}, —R—OC(O)—N(R^{a})₂, —R—N(R^{a})₂, —R—C(O)R^{a}, —R—C(O)OR^{a}, —R—C(O)N(R^{a})₂, —R^{b}—O—R^{C}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R—S(O)ₜR^{a} (where t is 1 or 2), —R—S(O)ₜOR^{a} (where t is 1 or 2) and —R—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine(═N—NH₂), —R—OR^{a}, —R—OC(O)—R^{a}, —R—OC(O)—OR^{a}, —R—OC(O)—N(R^{a})₂, —R—N(R^{a})₂, —R^{b}—C(O)R^{a}, —R—C(O)OR^{a}, —R—C(O)N(R^{a})₂, —R—O—R^{C}—C(O)N(R^{a})₂, —R—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R—S(O)ₜN(R^{a})₂ (where t is 1 or 2); and wherein each R^{b} is independently selected from a direct bond or a straight or branched alkylene, alkenylene, or alkynylene chain, and each R^{C} is a straight or branched alkylene, alkenylene or alkynylene chain. It will be understood by those skilled in the art that substituents can themselves be substituted, if appropriate.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described herein, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms “administer”, “administered”, “administers” and “administering” a compound should be understood to mean providing a compound of the invention or a salt of a compound of the invention to the individual in need.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “inhibit”, “selective inhibition” or “selectively inhibit” refers to an agent's (chemical or biological) ability to preferentially reduce the target signaling activity as compared to off-target signaling activity, via direct or indirect interaction with the target.

A “therapeutic effect,” as used herein, encompasses a therapeutic benefit and/or a prophylactic benefit as described above. A prophylactic effect includes delaying or eliminating the appearance of a disease or condition, onset of symptoms of a disease or condition, slowing, halting, or reversing the progression of a disease or condition, or any combination thereof.

### Compounds

In some aspects, the present disclosure provides a compound represented by the structure of Formula (I):

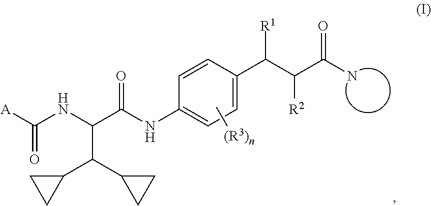

or a pharmaceutically acceptable salt thereof, wherein:
A is selected from a C₃₋₆ carbocycle and 5- to 6-membered heterocycle, any one of which is optionally substituted with one or more substituents independently selected from (a), (b), and (c):
   (a) halogen, —OR¹¹, —N(R¹¹)₂, —C(O)R¹¹, —C(O)OR¹¹, —NO₂, and —CN;
   (b) C₁₋₆ alkyl optionally substituted with one or more substituents independently selected from: halogen, —OR¹¹, —N(R¹¹)₂, —C(O)R¹¹, —C(O)OR¹¹, —NO₂, —CN, C₃₋₆ carbocycle and 3- to 6-membered heterocycle, wherein each C₃₋₆ carbocycle and 3- to 6-membered heterocycle is optionally substituted with one or more substituents independently selected from halogen, —OR¹¹, —N(R¹¹)₂—C(O)R¹¹, —C(O)OR¹¹, —NO₂, and —CN; and
   (c) C₃₋₆ carbocycle optionally substituted with one or more substituents independently selected from halogen, C₁₋₄ alkyl, C₁₋₄ haloalkyl, —OR¹¹, —N(R¹¹)₂, —C(O)R¹¹, —C(O)OR¹, —NO₂, and —CN;
R¹ is selected from hydrogen, halogen, —OR¹², —N(R¹²)₂, —C(O)R¹², —C(O)OR¹², —NO₂, —CN, and C₁₋₆ alkyl optionally substituted with one or more substituents independently selected from halogen, —OR¹², —N(R¹²)₂, —C(O)R¹², —C(O)OR¹², —NO₂, and —CN;
R² is selected from —N(R^{A})C(O)(R^{B}) and —N(R^{C})C(O)N(R^{D})(R^{E});

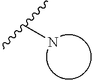 [SMILES: *N]

is selected from a 4- to 12-membered heterocycle optionally substituted with one or more substituents independently selected from:
   halogen, —OR¹³, —N(R¹³)₂, —C(O)R¹³, —C(O)OR¹³, —NO₂, —CN, and C₁₋₆ alkyl optionally substituted with one or more substituents independently selected from halogen, —OR¹³, —N(R¹³)₂, —C(O)R¹³, —C(O)OR¹³, —NO₂, and —CN;
R³ is selected from halogen, —OR¹⁴, —N(R¹⁴)₂, —C(O)R¹⁴, —C(O)OR¹⁴, —NO₂, —CN, C₁₋₆ alkyl and C₃₋₆ carbocycle, wherein each C₁₋₆ alkyl and C₃₋₆ carbocycle of which is optionally substituted with one or more substituents independently selected from halogen, —OR¹⁴, —N(R¹⁴)₂, —C(O)R¹⁴, —C(O)OR¹⁴, —NO₂, and —CN;
R^{A} is selected from hydrogen and C₁₋₆ alkyl optionally substituted with one or more substituents independently selected from halogen, —OR¹⁵, —N(R¹⁵)₂, —C(O)R¹⁵, —C(O)OR¹⁵, —NO₂, and —CN;
R^{B} is selected from:
C₁₋₆ alkyl optionally substituted with one or more substituents independently selected from:
   halogen, —OR¹⁶, —N(R¹⁶)₂, —C(O)R¹⁶, —C(O)OR¹⁶, —N(R¹⁶)C(O)OR¹⁶, —NO₂, —CN;
   C₃₋₆ carbocycle and 5- to 6-membered heterocycle, any of which is optionally substituted with one or more substituents independently selected from halogen, —OR¹⁶, —N(R¹⁶)₂, —C(O)R¹⁶, —C(O)OR¹⁶, —NO₂, and —CN; and
C₃₋₆ carbocycle or 4- to 6-membered heterocycle, each of which is optionally substituted with one or more substituents independently selected from:
   halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN;
R^{C} is selected from hydrogen and C₁₋₆ alkyl optionally substituted with one or more substituents independently selected from: halogen, —OR¹⁷, —N(R¹⁷)₂, —C(O)R¹⁷, —C(O)OR¹⁷, —NO₂, and —CN;
R^{D} is selected from C₁₋₆ alkyl and C₃₋₆ carbocycle, any of which is optionally substituted with one or more substituents independently selected from: halogen, —OR¹⁸, —N(R¹⁸)₂, —C(O)R¹⁸, —C(O)OR¹⁸, —NO₂, and —CN;
R^{E} is selected from hydrogen, C₁₋₆ alkyl and C₃₋₆carbocycle, any of which is optionally substituted with one or more substituents independently selected from: halogen, —OR¹⁹, —N(R¹⁹)₂, —C(O)R¹⁹, —C(O)OR¹⁹, —NO₂, and —CN;
R¹¹, R¹², R¹³, R¹⁴, R¹⁵, R¹⁶, R¹⁷, R¹⁸, and R¹⁹ are each independently selected at each occurrence from:
   hydrogen;
   C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
   C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN; and
n is selected from 0, 1, 2, 3, and 4.

In some embodiments, for the compound or salt of Formula (I), n is selected from 0, 1, 2, and 3. In some embodiments, n is selected from 0, 1, and 2. In some embodiments, n is selected from 0 and 1. In some embodiments, n is selected from 1, 2, 3, and 4. In some embodiments, n is selected from 2, 3, and 4. In some embodiments, n is selected from 3 and 4. In some embodiments, n is 0. In some embodiments, n is 1. In some embodiments, n is 2. In some embodiments, n is 3. In some embodiments, n is 4.

In some embodiments, for the compound or salt of Formula (I), each R³ is independently selected from halogen, —OR¹⁴, —N(R¹⁴)₂, —C(O)R¹⁴, —C(O)OR¹⁴, —NO₂, and —CN. In some embodiments, each R³ is independently selected from halogen, —OR¹⁴, —NO₂, and —CN. In some embodiments, each R³ is independently selected from chlorine, fluorine, bromine, and —OR¹⁴. In some embodiments, each R³ is independently selected from chlorine, fluorine, and bromine. In some embodiments, each R³ is independently selected from chlorine and fluorine. In some embodiments, each R³ is fluorine.

In some embodiments, for the compound or salt of Formula (I), each R³ is selected from C₁₋₆ alkyl and C₃₋₆ carbocycle, wherein each C₁₋₆ alkyl and C₃₋₆ carbocycle of which is optionally substituted with one or more substituents independently selected from halogen, —OR¹⁴, —N(R¹⁴)₂, —C(O)R¹⁴, —C(O)OR¹⁴, —NO₂, and —CN. In some embodiments, each R³ is selected from methyl, ethyl, propyl, isopropyl, cyclopropyl, cyclobutyl, cyclopentyl, cyclohexyl, and phenyl, each of which is optionally substituted with one or more substituents independently selected from halogen, —OR¹⁴, —N(R¹⁴)₂, —C(O)R¹⁴, —C(O)OR¹⁴, —NO₂, and —CN.

In some embodiments, for the compound or salt of Formula (I), each R³ is independently selected from halogen, —OR¹⁴, —N(R¹⁴)₂, —CN, C₁₋₃ alkyl, C₁₋₃ haloalkyl, and optionally substituted C₃₋₆ saturated carbocycle. In some embodiments, each R³ is independently selected from halogen, —OR¹⁴, —N(R¹⁴)₂, —CN, methyl, ethyl, propyl, isopropyl, cyclopropyl, cyclobutyl, cyclopentyl, and cyclohexyl. In some embodiments, each R³ is independently selected from halogen, —OR¹⁴, —N(R¹⁴)₂, —CN, C₁₋₃ alkyl and C₁₋₃ haloalkyl. In some embodiments, each R³ is independently selected from fluorine, chlorine, —OR¹⁴, —N(R¹⁴)₂, —CN, C₁₋₃ alkyl and C₁₋₃ haloalkyl. In some embodiments, each R³ is independently selected from fluorine, chlorine, —OR¹⁴, —N(R¹⁴)₂, and —CN. In some embodiments, each R³ is independently selected from fluorine and chlorine. In some embodiments, each R³ is selected from fluorine

In some embodiments, for the compound or salt of Formula (I), n is selected from 1, 2 and 3; and each R³ is independently selected from halogen, —OR¹⁴, —N(R¹⁴)₂, —CN, C₁₋₃ alkyl and C₁₋₃ haloalkyl. In some embodiments, n is selected from 1, 2 and 3; and each R³ is independently selected from fluorine, chlorine, —OR¹⁴, —N(R¹⁴)₂, —CN, C₁₋₃ alkyl and C₁₋₃ haloalkyl. In some embodiments, n is selected from 1, 2 and 3; and each R³ is selected from fluorine.

In some embodiments, for the compound or salt of Formula (I), n is 1; and R³ is independently selected from halogen, —OR¹⁴, —N(R¹⁴)₂, —CN, C₁₋₃ alkyl and C₁₋₃ haloalkyl. In some embodiments, n is 1; and R³ is independently selected from fluorine, chlorine, —OR¹⁴, —N(R¹⁴)₂, —CN, C₁₋₃ alkyl and C₁₋₃ haloalkyl. In some embodiments, n is 1; and R³ is selected from fluorine.

In some embodiments, for the compound or salt of Formula (I) or (I-A), R¹ is selected from hydrogen, halogen, —OR¹², —N(R¹²)₂, —C(O)R¹², —C(O)OR¹², —NO₂, and —CN. In some embodiments, R¹ is selected from halogen, —OR¹², —N(R¹²)₂, —C(O)R¹², —C(O)OR¹², —NO₂, and —CN. In some embodiments, R¹ is selected from C₁₋₆ alkyl optionally substituted with one or more substituents independently selected from halogen, —OR¹², —N(R¹²)₂, —C(O)R¹², —C(O)OR¹², —NO₂, and —CN. In some embodiments, R¹ is selected from hydrogen, halogen, —OR¹², —N(R¹²)₂, —CN, C₁₋₃ alkyl and C₁₋₃ haloalkyl. In some embodiments, R¹ is selected from halogen, —OR¹², —N(R¹²)₂, —CN, C₁₋₃ alkyl and C₁₋₃ haloalkyl. In some embodiments, R¹ is selected from methyl, ethyl, propyl, and isopropyl, any of which is optionally substituted with halogen, —OR¹², —N(R¹²)₂, and —CN. In some embodiments, R¹ is selected from methyl, ethyl, propyl, isopropyl, and trifluoromethyl. In some embodiments, R¹ is selected from methyl, ethyl, propyl, and isopropyl. In some embodiments, R¹ is selected from selected from methyl and trifluoromethyl. In some embodiments, R¹ is selected from selected from methyl. In some embodiments, R¹ is selected from selected from trifluoromethyl.

In some embodiments, for the compound or salt of Formula (I) or (I-A), A is selected from a saturated C₃₋₆ carbocycle and a 5-membered heteroaryl, any of which is optionally substituted with one or more substituents independently selected from: from (a), (b), and (c):
(a) halogen, —OR¹¹, —N(R¹¹)₂, —C(O)R¹¹, —C(O)OR¹¹, —NO₂, and —CN;
(b) C₁₋₃ alkyl optionally substituted with one or more substituents independently selected from halogen, —OR¹¹, —N(R¹¹)₂, —C(O)R¹¹, —C(O)OR¹, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹¹, —N(R¹¹)₂, —C(O)R¹¹, —C(O)OR¹¹, —NO₂, and —CN; and
(c) C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is selected from cyclopropyl, cyclobutyl, cyclopentyl, cyclohexyl, phenyl, pyrazole, imidazole, oxazole, isoxazole, oxadiazole, triazole, and tetrazole, any of which is optionally substituted with one or more substituents independently selected from: from (a), (b), and (c):
(a) halogen, —OR¹¹, —N(R¹¹)₂, —C(O)R¹¹, —C(O)OR¹¹, —NO₂, and —CN;
(b) C₁₋₃ alkyl optionally substituted with one or more substituents independently selected from halogen, —OR¹¹, —N(R¹¹)₂, —C(O)R¹¹, —C(O)OR¹, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹¹, —N(R¹¹)₂, —C(O)R¹¹, —C(O)OR¹¹, —NO₂, and —CN; and
(c) C₃₋₆ carbocycle each of which is optionally substituted with one or more substituents selected from: halogen, —OR¹, —N(R¹¹)₂, —C(O)R¹¹, —C(O)OR¹¹, —NO₂, and —CN.

In some embodiments, for the compound or salt of Formula (I) or (I-A), A is selected from cyclopropyl, cyclobutyl, cyclopentyl, cyclohexyl, and phenyl, any of which is optionally substituted with one or more substituents independently selected from: from (a), (b), and (c):
(a) halogen, —OR¹¹, —N(R¹¹)₂, —C(O)R¹¹, —C(O)OR¹¹, —NO₂, and —CN;
(b) C₁₋₃ alkyl optionally substituted with one or more substituents independently selected from halogen, —OR¹¹, —N(R¹¹)₂, —C(O)R¹¹, —C(O)OR¹, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹¹, —N(R¹¹)₂, —C(O)R¹¹, —C(O)OR¹¹, —NO₂, and —CN; and
(c) C₃₋₆ carbocycle each of which is optionally substituted with one or more substituents selected from: halogen, —OR¹, —N(R¹¹)₂, —C(O)R¹¹, —C(O)OR¹¹, —NO₂, and —CN.

In some embodiments, for the compound or salt of Formula (I) or (I-A), A is selected from pyrazole, imidazole, oxazole, isoxazole, oxadiazole, triazole, and tetrazole, any of which is optionally substituted with one or more substituents independently selected from: from (a), (b), and (c):
(a) halogen, —OR¹¹, —N(R¹¹)₂, —C(O)R¹¹, —C(O)OR¹¹, —NO₂, and —CN;
(b) C₁₋₃ alkyl optionally substituted with one or more substituents independently selected from halogen, —OR¹¹, —N(R¹¹)₂, —C(O)R¹¹, —C(O)OR¹, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹¹, —N(R¹¹)₂, —C(O)R¹¹, —C(O)OR¹¹, —NO₂, and —CN; and
(c) C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is selected from cyclopropyl, cyclobutyl, cyclopentyl, cyclohexyl, phenyl, pyrazole, imidazole, oxazole, isoxazole, oxadiazole, triazole, and tetrazole, any of which is optionally substituted with one or more substituents independently selected from halogen, —OR¹¹, —N(R¹¹)₂, —C(O)R¹¹, —C(O)OR¹, —NO₂, and —CN. In some embodiments, A is selected from cyclopropyl, cyclobutyl, cyclopentyl, cyclohexyl, and phenyl, any of which is optionally substituted with one or more substituents independently selected from halogen, —OR¹¹, —N(R¹¹)₂, —C(O)R¹¹, —C(O)OR¹¹, —NO₂, and —CN. In some embodiments, A is selected from pyrazole, imidazole, oxazole, isoxazole, oxadiazole, triazole, and tetrazole, any of which is optionally substituted with one or more substituents independently selected from halogen, —OR¹¹, —N(R¹¹)₂, —C(O)R¹¹, —C(O)OR¹¹, —NO₂, and —CN.

In some embodiments, for the compound or salt of Formula (I) or (I-A), A is selected from cyclopropyl, cyclobutyl, cyclopentyl, cyclohexyl, phenyl, pyrazole, imidazole, oxazole, isoxazole, oxadiazole, triazole, and tetrazole, any of which is optionally substituted with one or more substituents independently selected from C₁₋₃ alkyl optionally substituted with one or more substituents independently selected from halogen, —OR¹, —N(R¹¹)₂, —C(O)R¹¹, —C(O)OR¹¹, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹, —N(R¹¹)₂, —C(O)R¹¹, —C(O)OR¹¹, —NO₂, and —CN. In some embodiments, A is selected from cyclopropyl, cyclobutyl, cyclopentyl, cyclohexyl, and phenyl, any of which is optionally substituted with one or more substituents independently selected from C₁₋₃ alkyl optionally substituted with one or more substituents independently selected from halogen, —OR¹¹, —N(R¹¹)₂, —C(O)R¹¹, —C(O)OR¹¹, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹¹, —N(R¹¹)₂, —C(O)R¹¹, —C(O)OR¹¹, —NO₂, and —CN. In some embodiments, A is selected from pyrazole, imidazole, oxazole, isoxazole, oxadiazole, triazole, and tetrazole, any of which is optionally substituted with one or more substituents independently selected from C₁₋₃ alkyl optionally substituted with one or more substituents independently selected from halogen, —OR¹, —N(R¹¹)₂, —C(O)R¹¹, —C(O)OR¹¹, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is selected from cyclopropyl, cyclobutyl, cyclopentyl, cyclohexyl, phenyl, pyrazole, imidazole, oxazole, isoxazole, oxadiazole, triazole, and tetrazole, any of which is optionally substituted with one or more substituents independently selected from C₃₋₆ carbocycle each of which is optionally substituted with one or more substituents selected from: halogen, —OR¹, —N(R¹¹)₂, —C(O)R¹¹, —C(O)OR¹¹, —NO₂, and —CN. In some embodiments, A is selected from cyclopropyl, cyclobutyl, cyclopentyl, cyclohexyl, and phenyl, any of which is optionally substituted with one or more substituents independently selected from C₃₋₆ carbocycle each of which is optionally substituted with one or more substituents selected from: halogen, —OR¹, —N(R¹¹)₂, —C(O)R¹¹, —C(O)OR¹¹, —NO₂, and —CN. In some embodiments, A is selected from pyrazole, imidazole, oxazole, isoxazole, oxadiazole, triazole, and tetrazole, any of which is optionally substituted with one or more substituents independently selected from C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is a saturated C₃₋₆ carbocycle optionally substituted with one or more substituents independently selected from halogen, —OR¹, —N(R¹¹)₂, —CN, C₁₋₃ alkyl, and C₁₋₃ haloalkyl. In some embodiments, A is a C₃₋₆ cycloalkyl selected from cyclopropyl, cyclobutyl, cyclopentyl, and cyclohexyl, any of which is optionally substituted with one or more substituents independently selected from halogen, —OR¹¹, —N(R¹¹)₂, —CN, C₁₋₃ alkyl, and C₁₋₃ haloalkyl. In some embodiments, A is a saturated C₃₋₆ carbocycle selected from cyclopropyl, cyclobutyl, cyclopentyl, and cyclohexyl, any of which is optionally substituted with one or more substituents independently selected from halogen, —OR¹¹, —N(R¹¹)₂, —CN, C₁₋₃ alkyl, and C₁₋₃ haloalkyl. In some embodiments, A is selected from cyclopropyl, cyclobutyl, cyclopentyl, and cyclohexyl, any of which is optionally substituted with one or more substituents independently selected from halogen, —OR¹¹, —N(R¹¹)₂, —CN, C₁₋₃ alkyl, and C₁₋₃ haloalkyl. In some embodiments, A is selected from cyclopropyl, cyclobutyl, cyclopentyl, and cyclohexyl, any of which is optionally substituted with one or more substituents independently selected from halogen, —OR¹¹, C₁₋₃ alkyl, and C₁₋₃ haloalkyl. In some embodiments, A is selected from cyclopropyl, cyclobutyl, cyclopentyl, and cyclohexyl, any of which is optionally substituted with one or more substituents independently selected from flourine, chlorine, bromine, C₁₋₃ alkyl, and C₁₋₃ haloalkyl. In some embodiments, A is selected from cyclopropyl, cyclobutyl, cyclopentyl, and cyclohexyl, any of which is optionally substituted with one or more substituents independently selected from flourine, chlorine, and bromine.

In some embodiments, for the compound or salt of Formula (I) or (I-A), A is an optionally substituted C₃₋₆ carbocycle. In some embodiments, A is selected from C₃₋₅ carbocycle, C₃₋₄ carbocycle, C₄₋₆ carbocycle, and C₅₋₆ carbocycle, any of which is optionally substituted. In some embodiments, A is selected from C₃ carbocycle, C₄ carbocycle, C₅ carbocycle, and C₆ carbocycle, any of which is optionally substituted. In some embodiments, A is an optionally substituted C₃₋₆ saturated carbocycle. is an optionally substituted unsaturated C₃₋₆ carbocycle. In some embodiments, A is a C₃₋₆ carbocycle selected from cyclopropyl, cyclobutyl, cyclopentyl, cyclohexyl, and phenyl, any of which is optionally substituted. In some embodiments, A is a C₃₋₆ saturated carbocycle selected from cyclopropyl, cyclobutyl, cyclopentyl, and cyclohexyl, any of which is optionally substituted.

In some embodiments, for the compound or salt of Formula (I) or (I-A), A is a saturated C₃₋₆ carbocycle, any one of which is optionally substituted with one or more substituents independently selected from halogen, —OR¹¹, —N(R¹¹)₂, —CN, C₁₋₃ alkyl, and C₁₋₃ haloalkyl. In some embodiments, A is a saturated C₃₋₆ carbocycle selected from cyclopropyl, cyclobutyl, cyclopentyl, and cyclohexyl, any one of which is optionally substituted with one or more substituents independently selected from halogen, —OR¹¹, —N(R¹¹)₂, —CN, C₁₋₃ alkyl, and C₁₋₃ haloalkyl. In some embodiments, A is a saturated C₃₋₆ carbocycle selected from cyclopropyl, cyclobutyl, cyclopentyl, and cyclohexyl, any one of which is optionally substituted with one or more substituents independently selected from flourine, chlorine, bromine, —OR¹¹, —N(R¹¹)₂, —CN, C₁₋₃ alkyl, and C₁₋₃ haloalkyl. In some embodiments, A is a saturated C₃₋₆ carbocycle selected from cyclopropyl, cyclobutyl, cyclopentyl, and cyclohexyl, any one of which is optionally substituted with one or more substituents independently selected from flourine, chlorine, bromine, —OR¹¹, C₁₋₃ alkyl, and C₁₋₃ haloalkyl. In some embodiments, A is cyclopropyl optionally substituted with halogen, C₁₋₃ alkyl, and C₁₋₃ haloalkyl. In some embodiments, A is cyclopropyl optionally substituted with flourine, chlorine, bromine, C₁₋₃ alkyl, and C₁₋₃ haloalkyl. In some embodiments, A is cyclopropyl optionally substituted with flourine, chlorine, and bromine. In some embodiments, A is

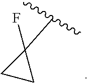 [SMILES: *C1(F)CC1]

In some embodiments, for the compound or salt of Formula (I) or (I-A), A is a 5- to 6-membered heterocycle comprising at least one heteroatom selected from nitrogen, oxygen, sulfur, and a combination thereof. In some embodiments, A is a 5- to 6-membered heterocycle comprising at least one heteroatom selected from nitrogen, oxygen, and a combination thereof. In some embodiments, A is a 5- to 6-membered heterocycle comprising at least one heteroatom selected from nitrogen, sulfur, and a combination thereof. In some embodiments, A is a 5- to 6-membered heterocycle comprising at least one heteroatom selected from oxygen, sulfur, and a combination thereof. In some embodiments, A is a 5- to 6-membered heterocycle comprising at least one nitrogen heteroatom. In some embodiments, A is a 5- to 6-membered heterocycle comprising at least one oxygen heteroatom. In some embodiments, A is a 5- to 6-membered heterocycle comprising at least one sulfur heteroatom. In some embodiments, A is a 5- to 6-membered saturated heterocycle. In some embodiments, A is a 5- to 6-membered unsaturated heterocycle. In some embodiments, A is a 5- to 6-membered heteroaryl.

In some embodiments, for the compound or salt of Formula (I) or (I-A), A is a 5- to 6-membered heteroaryl comprising at least one heteroatom selected from nitrogen, oxygen, sulfur, and a combination thereof. In some embodiments, A is a 5- to 6-membered heteroaryl comprising at least one heteroatom selected from nitrogen, oxygen, and a combination thereof. In some embodiments, A is a 5- to 6-membered heteroaryl comprising at least one heteroatom selected from nitrogen, sulfur, and a combination thereof. In some embodiments, A is a 5- to 6-membered heteroaryl comprising at least one heteroatom selected from oxygen, sulfur, and a combination thereof. In some embodiments, A is a 5- to 6-membered heteroaryl comprising at least one nitrogen heteroatom. In some embodiments, A is a 5- to 6-membered heteroaryl comprising at least one oxygen heteroatom. In some embodiments, A is a 5- to 6-membered heteroaryl comprising at least one sulfur heteroatom.

In some embodiments, for the compound or salt of Formula (I) or (I-A), A is a 5- to 6-membered heteroaryl optionally substituted with one or more substituents independently selected from:
C₁₋₆ alkyl optionally substituted with one or more substituents independently selected from: halogen, —OR¹¹, —N(R¹¹)₂, —C(O)R¹¹, —C(O)OR¹¹, —NO₂, —CN; and 5- to 6-membered heterocycle each of which is optionally substituted with one or more substituents selected from: halogen, —OR¹¹, —N(R¹¹)₂, —C(O)R¹¹, —C(O)OR¹¹, —NO₂, and —CN; and
C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is pyrazole, imidazole, triazole, tetrazole, thiophene, oxazole, isoxazole, thiazole, isothiazole, oxadiazole, thiadizaole, pyridine, pyridazine, pyrimidine, pyrazine, and triazine, any of which is optionally substituted with one or more substituents independently selected from:
C₁₋₆ alkyl optionally substituted with one or more substituents independently selected from: halogen, —OR¹¹, —N(R¹¹)₂, —C(O)R¹, —C(O)OR¹¹, —NO₂, —CN; and 5- to 6-membered heterocycle each of which is optionally substituted with one or more substituents selected from: halogen, —OR¹¹, —N(R¹¹)₂, —C(O)R¹¹, —C(O)OR¹¹, —NO₂, and —CN; and
C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is a 6-membered heteroaryl optionally substituted with one or more substituents independently selected from:
C₁₋₆ alkyl optionally substituted with one or more substituents independently selected from: halogen, —OR¹¹, —N(R¹¹)₂, —C(O)R¹¹, —C(O)OR¹¹, —NO₂, —CN; and 5- to 6-membered heterocycle each of which is optionally substituted with one or more substituents selected from: halogen, —OR¹¹, —N(R¹¹)₂, —C(O)R¹¹, —C(O)OR¹¹, —NO₂, and —CN; and
C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is pyridine, pyridazine, pyrimidine, pyrazine, and triazine, any of which is optionally substituted with one or more substituents independently selected from:
C₁₋₆ alkyl optionally substituted with one or more substituents independently selected from: halogen, —OR¹¹, —N(R¹¹)₂, —C(O)R¹¹, —C(O)OR¹¹, —NO₂, —CN; and 5- to 6-membered heterocycle each of which is optionally substituted with one or more substituents selected from: halogen, —OR¹¹, —N(R¹¹)₂, —C(O)R¹¹, —C(O)OR¹¹, —NO₂, and —CN; and
C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is a 5-membered heteroaryl optionally substituted with one or more substituents independently selected from:
C₁₋₆ alkyl optionally substituted with one or more substituents independently selected from: halogen, —OR¹¹, —N(R¹¹)₂, —C(O)R¹¹, —C(O)OR¹¹, —NO₂, —CN; and 5- to 6-membered heterocycle each of which is optionally substituted with one or more substituents selected from: halogen, —OR¹¹, —N(R¹¹)₂, —C(O)R¹¹, —C(O)OR¹¹, —NO₂, and —CN; and
C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is pyrazole, imidazole, oxazole, isoxazole, oxadiazole, triazole, tetrazole, any one of which is optionally substituted with one or more substituents independently selected from:
C₁₋₆ alkyl optionally substituted with one or more substituents independently selected from: halogen, —OR¹¹, —N(R¹¹)₂, —C(O)R¹¹, —C(O)OR¹¹, —NO₂, —CN; and 5- to 6-membered heterocycle each of which is optionally substituted with one or more substituents selected from: halogen, —OR¹¹, —N(R¹¹)₂, —C(O)R¹¹, —C(O)OR¹¹, —NO₂, and —CN; and
C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is pyrazole, and oxadiazole, each one of which is optionally substituted with one or more substituents independently selected from:
C₁₋₆ alkyl optionally substituted with one or more substituents independently selected from: halogen, —OR¹¹, —N(R¹¹)₂, —C(O)R¹¹, —C(O)OR¹¹, —NO₂, —CN; and 5- to 6-membered heterocycle each of which is optionally substituted with one or more substituents selected from: halogen, —OR¹, —N(R¹¹)₂, —C(O)R¹¹, —C(O)OR¹¹, —NO₂, and —CN; and
C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

In some embodiments, for the compound or salt of Formula (I) or (I-A), A is selected from pyrazole and oxadiazole, each of which is optionally substituted with one or more substituents independently selected from C₁₋₃ alkyl, C₁₋₃ haloalkyl, C₁₋₃ alkyl-OR¹¹, and C₁₋₃ alkyl substituted with 5- to 6-membered saturated heterocycle.

In some embodiments, for the compound or salt of Formula (I) or (I-A), A is selected from pyrazole and oxadiazole, each of which is optionally substituted with methyl, ethyl, propyl, and isopropyl.

In some embodiments, for the compound or salt of Formula (I) or (I-A), A is selected from:

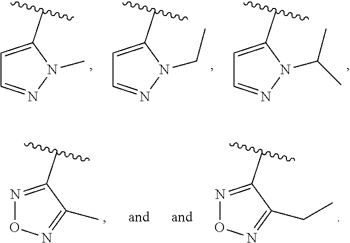

In some embodiments, A is selected from

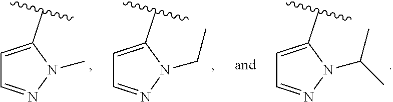

In some embodiments, A is

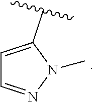 [SMILES: *c1ccnn1C]

In some embodiments, A is

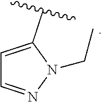 [SMILES: *c1ccnn1CC]

In some embodiments, A is

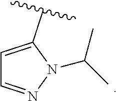 [SMILES: *c1ccnn1C(C)C]

In some embodiments, A is selected from

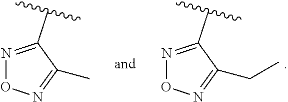

In some embodiments, A is

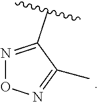 [SMILES: *c1nonc1C]

In some embodiments, A is

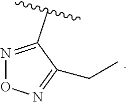 [SMILES: *c1nonc1CC]

In some embodiments, for the compound or salt of Formula (I) or (I-A), A is selected from pyrazole and oxadiazole, each of which is optionally substituted with C₁₋₃ haloalkyl, and C₁₋₃ alkyl-OR¹¹. In some embodiments, A is selected from:

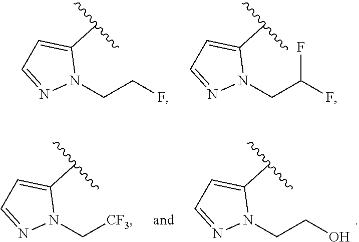

In some embodiments, A is selected from

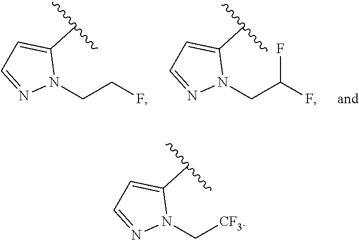

In some embodiments, A is

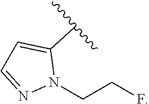 [SMILES: *c1ccnn1CCF]

In some embodiments, A is

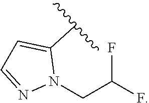 [SMILES: *c1ccnn1CC(F)F]

In some embodiments, A is

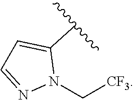 [SMILES: *c1ccnn1CC(F)(F)F]

In some embodiments, A is

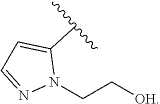 [SMILES: *c1ccnn1CCO]

In some embodiments, for the compound or salt of Formula (I) or (I-A), A is selected from pyrazole and oxadiazole, each of which is optionally substituted with C₁₋₃ alkyl substituted with 5- to 6-membered saturated heterocycle. In some embodiments, A is selected from pyrazole and oxadiazole, each of which is optionally substituted with C₁₋₃ alkyl substituted with pyrrolidine, pyrazolidine, imidazolidine, tetrahydrofuran, tetrahydrothiophene, oxathiolane, piperidine, piperazine, tetrahydropyran, thiane, dithiane, morpholine, and thiomorpholine. In some embodiments, A is

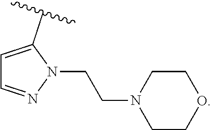 [SMILES: *c1ccnn1CCN1CCOCC1]

In some embodiments, for the compound or salt of Formula (I) or (I-A), A is selected from pyrazole and oxadiazole, each of which is optionally substituted with one or more substituents independently selected from saturated C₃₋₆ carbocycle optionally substituted with one or more substituents selected from: halogen, —OR¹¹, —N(R¹¹)₂, —C(O)R¹¹, —C(O)OR¹¹, —NO₂, and —CN. In some embodiments, A is selected from pyrazole and oxadiazole, each of which is optionally substituted by cyclopropyl, wherein the cyclopropyl is optionally substituted with one or more fluorine or chlorine atoms. In some embodiments, A is selected from:

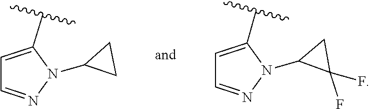 [SMILES: *c1ccnn1C1CC1 ; *c1ccnn1C1CC1(F)F]

In some embodiments, A is

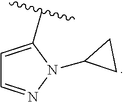 [SMILES: *c1ccnn1C1CC1]

In some embodiments, A is

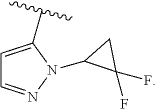 [SMILES: *c1ccnn1C1CC1(F)F]

In some embodiments, for the compound or salt of Formula (I) or (I-A), R² is selected from —N(R^{C})C(O)N(R^{D})(R^{E}). In some embodiments, R² is selected from —N(R^{C})C(O)N(R^{D})(R^{E}), wherein:
R^{C} is selected from hydrogen;
R^{D} is selected from C₁₋₆ alkyl and saturated C₃₋₆ carbocycle, any of which is optionally substituted with one or more substituents independently selected from: halogen, —OR¹⁸, —N(R¹⁸)₂, —C(O)R¹⁸, —C(O)OR¹⁸, —NO₂, and —CN; and
R^{E} is selected from C₁₋₆ alkyl and saturated C₃₋₆carbocycle, any of which is optionally substituted with one or more substituents independently selected from: halogen, —OR¹⁹, —N(R¹⁹)₂, —C(O)R¹⁹, —C(O)OR¹⁹, —NO₂, and —CN.

In some embodiments, for the compound or salt of Formula (I) or (I-A), R² is selected from —N(R^{C})C(O)N(R^{D})(R^{E}). In some embodiments, R² is selected from —N(R^{C})C(O)N(R^{D})(R^{E}), wherein:
R^{C} is selected from hydrogen;
R^{D} is selected from C₁₋₆ alkyl, cyclopropyl, cyclobutyl, cyclopentyl, and cyclohexyl, any of which is optionally substituted with one or more substituents independently selected from: halogen, —OR¹⁸, —N(R¹⁸)₂, —C(O)R¹⁸, —C(O)OR¹⁸, —NO₂, and —CN; and
R^{E} is selected from C₁₋₆ alkyl cyclopropyl, cyclobutyl, cyclopentyl, and cyclohexyl, any of which is optionally substituted with one or more substituents independently selected from: halogen, —OR¹⁹, —N(R¹⁹)₂, —C(O)R¹⁹, —C(O)OR¹⁹, —NO₂, and —CN.

In some embodiments, for the compound or salt of Formula (I) or (I-A), R^{D} is selected from C₁₋₃ alkyl and cyclopropyl, any of which is optionally substituted with one or more substituents independently selected from: halogen, —OR¹⁸, —N(R¹⁸)₂, —NO₂, and —CN; and R^{E} is selected from C₁₋₃ alkyl. In some embodiments, R^{E} is selected from C₁₋₃ alkyl and cyclopropyl, any of which is optionally substituted with one or more substituents independently selected from: halogen, —OR¹⁹, —N(R¹⁹)₂, —NO₂, and —CN; and R^{D} is selected from C₁₋₃ alkyl. In some embodiments, R² is selected from:

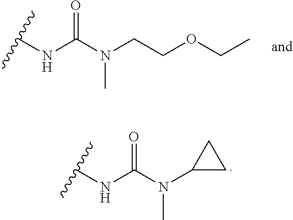

In some embodiments, R² is

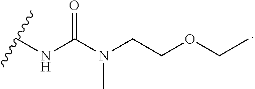 [SMILES: *NC(=O)N(C)CCOCC]

In some embodiments, R² is

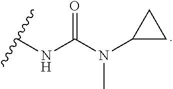 [SMILES: *NC(=O)N(C)C1CC1]

In some embodiments, for the compound or salt of Formula (I) or (I-A), R² is selected from —N(R^{A})C(O)(R^{B}); and R^{A} is selected from C₁₋₆ alkyl optionally substituted with one or more substituents independently selected from halogen, —OR¹⁵, —N(R¹⁵)₂, —C(O)R¹⁵, —C(O)OR¹⁵, —NO₂, and —CN. In some embodiments, R² is selected from —N(R^{A})C(O)(R^{B}); and R^{A} is selected from C₁₋₆ alkyl optionally substituted with one or more substituents independently selected from halogen, —OR¹⁵, and —N(R¹⁵)₂. In some embodiments, R² is selected from —N(R^{A})C(O)(R^{B}); and R^{A} is selected from C₁₋₆ alkyl optionally substituted with one or more substituents independently selected from —C(O)R¹⁵, —C(O)OR¹⁵, —NO₂, and —CN. In some embodiments, R² is selected from —N(R^{A})C(O)(R^{B}); and R^{A} is hydrogen.

In some embodiments, for the compound or salt of Formula (I) or (I-A), R² is selected from —N(R^{A})C(O)(R^{B}); and R^{B} is selected from C₁₋₆ alkyl optionally substituted with one or more substituents independently selected from: halogen, —OR¹⁶, —N(R¹⁶)₂, —C(O)R¹⁶, —C(O)OR¹⁶, —N(R¹⁶)C(O)OR¹⁶, —NO₂, and —CN. In some embodiments, R² is selected from —N(R^{A})C(O)(R^{B}); and R^{B} is selected from C₁₋₆ alkyl optionally substituted with one or more substituents independently selected from C₃₋₆ carbocycle and 5- to 6-membered heterocycle, any of which is optionally substituted with one or more substituents independently selected from halogen, —OR¹⁶, —N(R¹⁶)₂, —C(O)R¹⁶, —C(O)OR¹⁶, —NO₂, and —CN. In some embodiments, R² is selected from —N(R^{A})C(O)(R^{B}); and R^{B} is selected from C₃₋₆ carbocycle or 4- to 6-membered heterocycle, each of which is optionally substituted with one or more substituents independently selected from: halogen, —OR¹³, —N(R¹⁶)₂, —C(O)R¹⁶, —C(O)OR¹⁶, —NO₂, and —CN. In some embodiments, R² is selected from —N(R^{A})C(O)(R^{B} and R^{B} is selected from C₃₋₆ carbocycle or 4- to 6-membered heterocycle, each of which is optionally substituted with one or more substituents independently selected from: C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN.

In some embodiments, for the compound or salt of Formula (I) or (I-A), R² is selected from —N(R^{A})C(O)(R^{B}); R^{A} is hydrogen; and R^{B} is selected from C₁₋₆ alkyl optionally substituted with one or more substituents independently selected from: halogen, —OR¹⁶, —N(R¹⁶)₂, —C(O)R¹⁶, —C(O)OR¹⁶, —N(R¹⁶)C(O)OR¹⁶, —NO₂, and —CN. In some embodiments, R² is selected from —N(R^{A})C(O)(R^{B}); R^{A} is hydrogen; and R^{B} is selected from C₁₋₆ alkyl optionally substituted with one or more substituents independently selected from C₃₋₆ carbocycle and 5- to 6-membered heterocycle, any of which is optionally substituted with one or more substituents independently selected from halogen, —OR¹⁶, —N(R¹⁶)₂, —C(O)R¹⁶, —C(O)OR¹⁶, —NO₂, and —CN. In some embodiments, R² is selected from —N(RA)C(O)(R^{B}); R^{A} is hydrogen; and R^{B} is selected from C₃₋₆ carbocycle or 4- to 6-membered heterocycle, each of which is optionally substituted with one or more substituents independently selected from: halogen, —OR¹³, —N(R¹⁶)₂, —C(O)R¹⁶, —C(O)OR¹⁶, —NO₂, and —CN. In some embodiments, R² is selected from —N(R^{A})C(O)(R^{B}); and R^{A} is hydrogen; R^{B} is selected from C₃₋₆ carbocycle or 4- to 6-membered heterocycle, each of which is optionally substituted with one or more substituents independently selected from: C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN.

In some embodiments, for the compound or salt of Formula (I) or (I-A), R² is selected from —N(R^{A})C(O)(R^{B}); R^{A} is C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, —CN; and R^{B} is selected from C₁₋₆ alkyl optionally substituted with one or more substituents independently selected from: halogen, —OR¹⁶, —N(R¹⁶)₂, —C(O)R¹⁶, —C(O)OR¹⁶, —N(R¹⁶)C(O)OR¹⁶, —NO₂, and —CN. In some embodiments, R² is selected from —N(R^{A})C(O)(R^{B}); R^{A} is C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, —CN; and R^{B} is selected from C₁₋₆ alkyl optionally substituted with one or more substituents independently selected from C₃₋₆ carbocycle and 5- to 6-membered heterocycle, any of which is optionally substituted with one or more substituents independently selected from halogen, —OR¹⁶, —N(R¹⁶)₂, —C(O)R¹⁶, —C(O)OR¹⁶, —NO₂, and —CN. In some embodiments, R² is selected from —N(R^{A})C(O)(R^{B}); R^{A} is C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, —CN; and R^{B} is selected from C₃₋₆ carbocycle or 4- to 6-membered heterocycle, each of which is optionally substituted with one or more substituents independently selected from: halogen, —OR¹³, —N(R¹⁶)₂, —C(O)R¹⁶, —C(O)OR¹⁶, —NO₂, and —CN. In some embodiments, R² is selected from —N(R^{A})C(O)(R^{B}); and R^{A} is C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, —CN; R^{B} is selected from C₃₋₆ carbocycle or 4- to 6-membered heterocycle, each of which is optionally substituted with one or more substituents independently selected from: C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN.

In some embodiments, for the compound or salt of Formula (I) or (I-A), R² is —N(R^{A})C(O)(R^{B}), wherein:
R^{A} is selected from hydrogen, C₁₋₆ alkyl, and C₁₋₆ haloalkyl; and
R^{B} is selected from:
   C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, —CN; C₃₋₆ carbocycle and 5- to 6-membered heterocycle, any of which is optionally substituted with one or more substituents independently selected from: halogen, —OR¹⁶, —N(R¹⁶)₂, —C(O)R¹⁶, —C(O)OR¹⁶, —NO₂, and —CN; and
   4- to 6-membered heterocycle optionally substituted with one or more substituents independently selected from: halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from: halogen, —OR¹⁶, —N(R¹⁶)₂, —C(O)R¹⁶, —C(O)OR¹⁶, —NO₂, and —CN.

In some embodiments, for the compound or salt of Formula (I) or (I-A), R² is —N(R^{A})C(O)(R^{B}), wherein:
R^{A} is selected from hydrogen C₁₋₆ alkyl, and C₁₋₆ haloalkyl; and
R^{B} is selected from: C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, and —CN.

In some embodiments, for the compound or salt of Formula (I) or (I-A), R² is —N(R^{A})C(O)(R^{B}), wherein:
R^{A} is selected from hydrogen C₁₋₆ alkyl, and C₁₋₆ haloalkyl; and
R^{B} is selected from: C₁₋₆ alkyl optionally substituted with one or more substituents independently selected from C₃₋₆ carbocycle and 5- to 6-membered heterocycle, any of which is optionally substituted with one or more substituents independently selected from: halogen, —OR¹⁶, —N(R¹⁶)₂, —C(O)R¹⁶, —C(O)OR¹⁶, —NO₂, and —CN.

In some embodiments, for the compound or salt of Formula (I) or (I-A), R^{A} is hydrogen; and R^{B} is selected from: C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹⁶, —N(R¹⁶)₂, —C(O)R¹⁶, —C(O)OR¹⁶, —NO₂, and —CN.

In some embodiments, for the compound or salt of Formula (I) or (I-A), R^{A} is hydrogen; and R^{B} is selected from: C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, and —CN. In some embodiments, R^{A} is hydrogen; and R^{B} is selected from: methyl, ethyl, propyl, and isobutyl, any of which is optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, and —CN.

In some embodiments, for the compound or salt of Formula (I) or (I-A), R^{B} is selected from C₁₋₆ alkyl and C₁₋₆ haloalkyl. In some embodiments, R^{A} is hydrogen; and R^{B} is selected from C₁₋₆ alkyl and C₁₋₆ haloalkyl. In some embodiments, R^{A} is hydrogen; and R^{B} is selected from C₁₋₆ alkyl optionally substituted with one or more substituents independently selected from chlorine, fluorine, and bromine. In some embodiments, R^{A} is hydrogen; and R^{B} is selected from C₁₋₆ alkyl optionally substituted with one or more fluorine atoms. In some embodiments, R² is selected from:

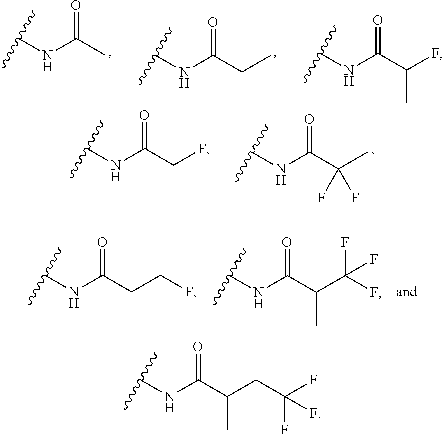

In some embodiments, R² is selected from:

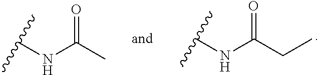

In some embodiments, R² is selected from:

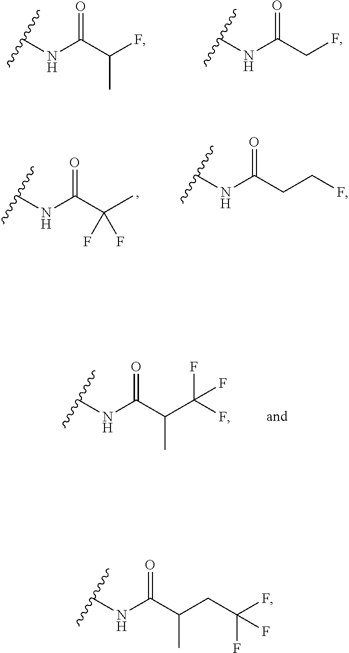

In some embodiments, for the compound or salt of Formula (I) or (I-A), R^{B} is selected from: C₁₋₆ alkyl optionally substituted with one or more substituents independently selected from halogen, —OR¹⁶ and N(R¹⁶)C(O)OR¹⁶. In some embodiments, R^{A} is hydrogen; and R^{B} is selected from: C₁₋₆ alkyl optionally substituted with one or more substituents independently selected from halogen, —OR¹⁶ and N(R¹⁶)C(O)OR¹⁶. In some embodiments, R^{A} is hydrogen; and R^{B} is selected from: C₁₋₆ alkyl optionally substituted with one or more substituents independently selected from chlorine, fluorine, bromine, —OR¹⁶ and —N(R¹⁶)C(O)OR¹⁶. In some embodiments, R^{A} is hydrogen; and R^{B} is selected from: C₁₋₆ alkyl optionally substituted with one or more substituents independently selected from chlorine, fluorine, bromine, and —N(R¹⁶)C(O)OR¹⁶. In some embodiments, R^{A} is hydrogen; and R^{B} is selected from: C₁₋₆ alkyl optionally substituted with one or more substituents independently selected from —OR¹⁶ and —N(R¹⁶)C(O)OR¹⁶. In some embodiments, R^{A} is hydrogen; and R^{B} is selected from: C₁₋₆ alkyl optionally substituted with one or more substituents independently selected from chlorine, fluorine, bromine, and —OR¹⁶.

In some embodiments, for the compound or salt of Formula (I) or (I-A), R² is selected from:

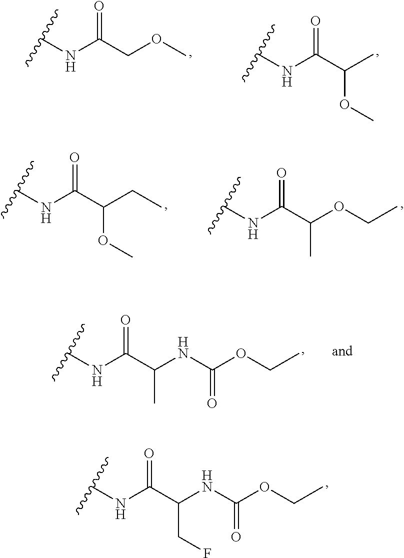

In some embodiments, R² is selected from:

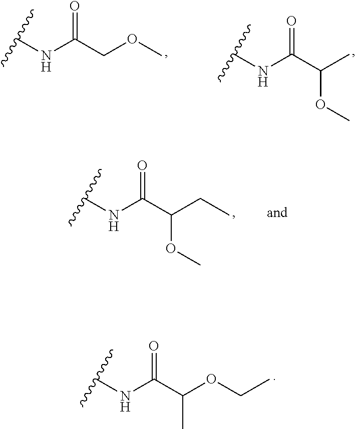

In some embodiments, R² is

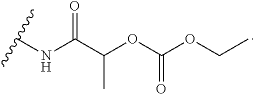 [SMILES: *NC(=O)C(C)OC(=O)OCC]

In some embodiments, R² is

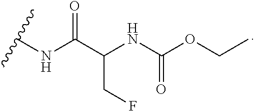 [SMILES: *NC(=O)C(CF)NC(=O)OCC]

In some embodiments, for the compound or salt of Formula (I) or (I-A), R^{B} is C₁₋₆ alkyl optionally substituted with —OR¹⁶. In some embodiments, R^{A} is hydrogen; and R^{B} is C₁₋₆ alkyl optionally substituted with —OR¹⁶. In some embodiments, R^{A} is hydrogen; and R^{B} is C₁₋₆ alkyl optionally substituted with —OR¹⁶, wherein R¹⁶ is C₁₋₆ alkyl substituted with —O—C₁₋₆ alkyl. In some embodiments, R² is

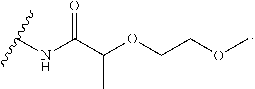 [SMILES: *NC(=O)C(C)OCCOC]

In some embodiments, for the compound or salt of Formula (I) or (I-A), R^{B} is selected from: C₁₋₆ alkyl optionally substituted with one or more substituents independently selected from C₃₋₆ carbocycle and 5- to 6-membered heterocycle, any one of which is optionally substituted with one or more substituents independently selected from halogen, —OR¹⁶, —N(R¹⁶)₂, —NO₂, and —CN. In some embodiments, R^{A} is hydrogen; and R^{B} is selected from: C₁₋₆ alkyl optionally substituted with one or more substituents independently selected from C₃₋₆ carbocycle and 5- to 6-membered heterocycle, any one of which is optionally substituted with one or more substituents independently selected from halogen, —OR¹⁶, —N(R¹⁶)₂, —NO₂, and —CN.

In some embodiments, for the compound or salt of Formula (I) or (I-A), R^{A} is hydrogen; and R^{B} is selected from: C₁₋₆ alkyl optionally substituted with one or more substituents independently selected from optionally substituted C₃₋₆ carbocycle. In some embodiments, R^{A} is hydrogen; and R^{B} is selected from: C₁₋₆ alkyl optionally substituted with one or more substituents independently selected from cyclopropyl, cyclobutyl, cyclopentyl, and cyclohexyl. In some embodiments, R^{A} is hydrogen; and R^{B} is selected from: C₁₋₆ alkyl optionally substituted with one or more cyclopropyl substituents. In some embodiments, R² is selected from

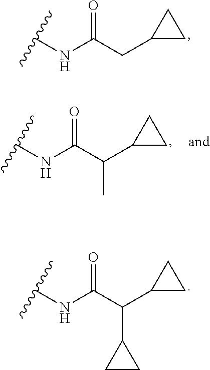

In some embodiments, R² is

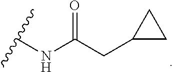 [SMILES: *NC(=O)CC1CC1]

In some embodiments, R² is

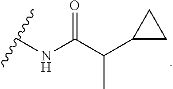 [SMILES: *NC(=O)C(C)C1CC1]

In some embodiments, R² is

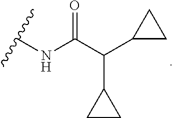 [SMILES: *NC(=O)C(C1CC1)C1CC1]

In some embodiments, for the compound or salt of Formula (I) or (I-A), R^{B} is selected from: C₁₋₆ alkyl optionally substituted with one or more substituents independently selected from 5-membered heterocycle optionally substituted with one or more substituents independently selected from halogen, —OR¹⁶, —N(R¹⁶)₂, —NO₂, and —CN. In some embodiments, R^{A} is hydrogen; and R^{B} is selected from: C₁₋₆ alkyl optionally substituted with one or more substituents independently selected from 5-membered heterocycle optionally substituted with one or more substituents independently selected from halogen, —OR¹⁶, —N(R¹⁶)₂, —NO₂, and —CN.

In some embodiments, for the compound or salt of Formula (I) or (I-A), R^{A} is hydrogen; and R^{B} is selected from: C₁₋₆ alkyl optionally substituted with one or more pyrazole, imidazole, triazole, tetrazole, thiophene, oxazole, isoxazole, thiazole, isothiazole, oxadiazole, and thiadizaole, any of which is optionally substituted with one or more substituents independently selected from halogen, —OR¹⁶, —N(R¹⁶)₂, —NO₂, and —CN. In some embodiments, R^{A} is hydrogen; and R^{B} is selected from: C₁₋₆ alkyl optionally substituted with one or more pyrazolyl, imidazolyl, triazolyl, tetrazolyl, thiopheneyl, oxazolyl, isoxazolyl, thiazolyl, isothiazolyl, oxadiazolyl, and thiadizaolyl, any of which is optionally substituted with one or more substituents independently selected from halogen, —OR¹⁶, —N(R¹⁶)₂, —NO₂, and —CN. In some embodiments, R² is

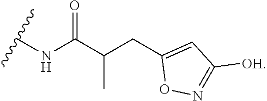 [SMILES: *NC(=O)C(C)Cc1cc(O)no1]

In some embodiments, for the compound or salt of Formula (I) or (I-A), R^{B} is selected from: C₁₋₆ alkyl optionally substituted with —N(R¹⁶)₂; and R¹⁶ is selected at each occurrence from hydrogen, C₁₋₃ alkyl, and 3- to 6-membered heterocycle, wherein each C₁₋₃ alkyl and 3- to 6-membered heterocycle is optionally substituted. In some embodiments, R^{A} is hydrogen; and R^{B} is selected from: C₁₋₆ alkyl optionally substituted with —N(R¹⁶)₂; and R¹⁶ is selected at each occurrence from hydrogen, C₁₋₃ alkyl, and 3- to 6-membered heterocycle, wherein each C₁₋₃ alkyl and 3- to 6-membered heterocycle is optionally substituted. In some embodiments, R² is selected from:

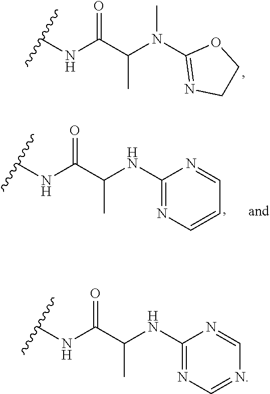 [SMILES: *NC(=O)C(C)N(C)C1=NCCO1 ; *NC(=O)C(C)Nc1ncccn1 ; *NC(=O)C(C)Nc1ncncn1]

In some embodiments, R² is

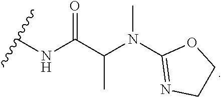 [SMILES: *NC(=O)C(C)N(C)C1=NCCO1]

In some embodiments, R² is

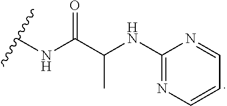 [SMILES: *NC(=O)C(C)Nc1ncccn1]

In some embodiments, R²

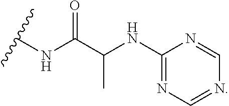 [SMILES: *NC(=O)C(C)Nc1ncncn1]

In some embodiments, for the compound or salt of Formula (I) or (I-A), R^{B} is selected from a 4- to 6-membered heterocycle optionally substituted with one or more substituents independently selected from halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN. In some embodiments, R^{B} is selected from a 4- to 6-membered saturated heterocycle, any one of which is optionally substituted with one or more substituents independently selected from halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN. In some embodiments, R^{B} is selected from azetidine and pyrrolidine, each of which is optionally substituted with halogen, —OR¹³, —C(O)R¹⁶, —C(O)OR¹⁶.

In some embodiments, for the compound or salt of Formula (I) or (I-A), R^{A} is hydrogen; and R^{B} is selected from a 4- to 6-membered heterocycle optionally substituted with one or more substituents independently selected from halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN. In some embodiments, R^{A} is hydrogen; and R^{B} is selected from a 4- to 6-membered saturated heterocycle, any one of which is optionally substituted with one or more substituents independently selected from halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN. In some embodiments, R^{A} is hydrogen; and R^{B} is selected from azetidine and pyrrolidine, each of which is optionally substituted with halogen, —OR¹³, —C(O)R¹⁶, —C(O)OR¹⁶.

In some embodiments, for the compound or salt of Formula (I) or (I-A), R² is selected from:

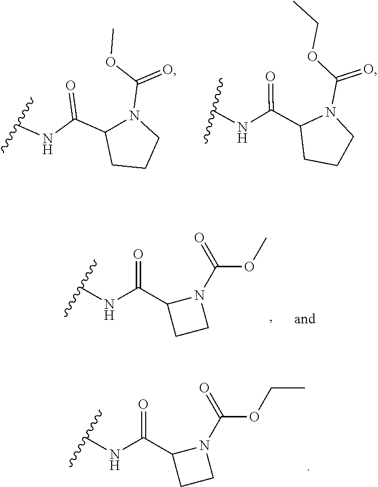 [SMILES: *NC(=O)C1CCCN1C(=O)OC ; *NC(=O)C1CCCN1C(=O)OCC ; *NC(=O)C1CCN1C(=O)OC ; *NC(=O)C1CCN1C(=O)OCC]

In some embodiments, R² is selected from:

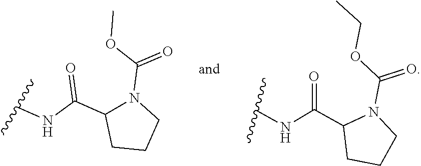 [SMILES: *NC(=O)C1CCCN1C(=O)OC ; *NC(=O)C1CCCN1C(=O)OCC]

In some embodiments, R² is selected from:

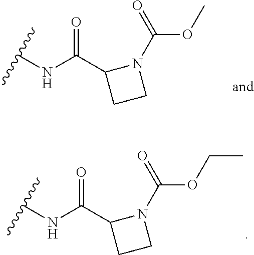 [SMILES: *NC(=O)C1CCN1C(=O)OC ; *NC(=O)C1CCN1C(=O)OCC]

In some embodiments, for the compound or salt of Formula (I) or (I-A),

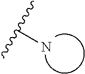 [SMILES: *N]

is selected from a 4- to 6-membered monocyclic heterocycle and 5- to 10-membered bicyclic fused heterocycle each of which is optionally substituted with one or more substituents independently selected from: halogen, —OR¹³, —N(R¹³)₂, —NO₂, —CN, and C₁₋₆ alkyl optionally substituted with one or more substituents independently selected from halogen, —OR³, —N(R¹³)₂, —NO₂, and —CN.

In some embodiments, for the compound or salt of Formula (I) or (I-A),

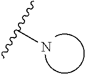 [SMILES: *N]

is selected from 4- to 6-membered monocyclic heterocycle, any of which is optionally substituted with one or more substituents independently selected from halogen, —OR¹³, —N(R¹³)₂, and C₁₋₆ alkyl optionally substituted with one or more substituents independently selected from: halogen, and —OR¹³. In some embodiments,

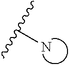 [SMILES: *N]

is selected from azetidine, pyrrolidine, pyrazolidine, imidazolidine, piperidine, morpholine, and piperazine, each of which is optionally substituted with one or more substituents independently selected from halogen, —OR¹³, —N(R¹³)₂, and C₁₋₆ alkyl optionally substituted with one or more substituents independently selected from: halogen, and —OR¹³. In some embodiments,

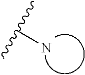 [SMILES: *N]

is selected from pyrrolidine, piperidine, morpholine, and piperazine, each of which is optionally substituted with one or more substituents selected from —OR¹³, —N(R¹³)₂, and C₁₋₃ alkyl. In some embodiments,

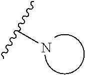 [SMILES: *N]

is selected from:

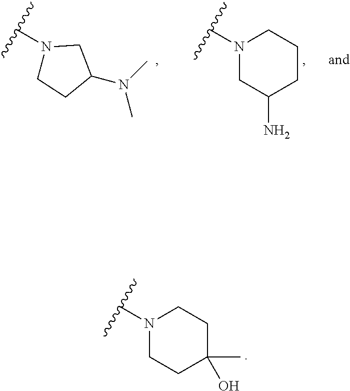

In some embodiments,

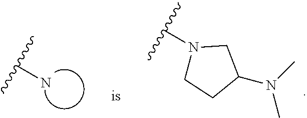

In some embodiments

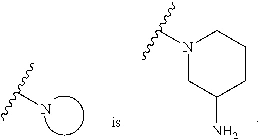

In some embodiments,

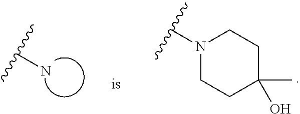

In some embodiments, for the compound or salt of Formula (I) or (I-A),

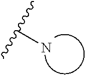 [SMILES: *N]

is selected from pyrrolidine, piperidine, morpholine, and piperazine, each of which is optionally substituted with one or more substituents selected from C₁₋₃ alkyl optionally substituted with one or more substituents independently selected from: halogen and —OR¹³. In some embodiments,

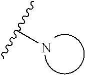 [SMILES: *N]

is selected from pyrrolidine, piperidine, morpholine, and piperazine, each of which is optionally substituted with one or more substituents selected from C₁₋₃ alkyl optionally substituted with one or more substituents independently selected from: chlorine, fluorine, bromine, and —OR¹³. In some embodiments,

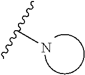 [SMILES: *N]

is selected from:

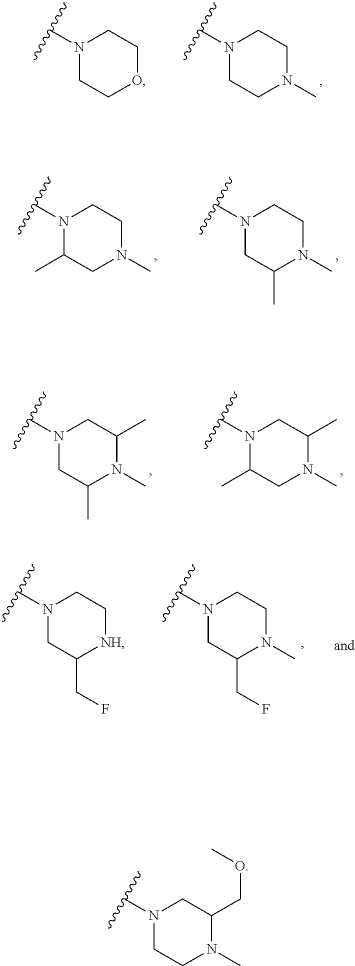

In some embodiments,

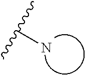 [SMILES: *N]

is selected from:

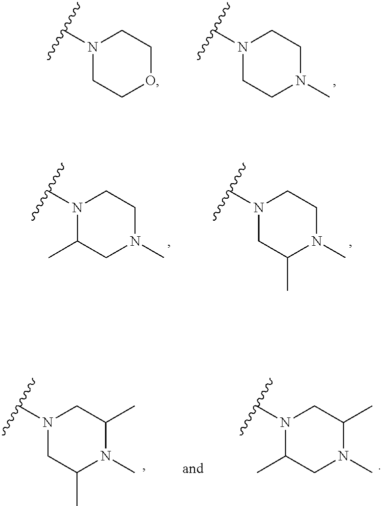

In some embodiments,

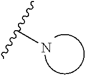 [SMILES: *N]

is selected from:

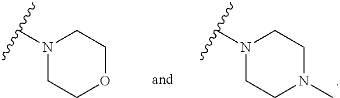

In some embodiments,

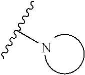 [SMILES: *N]

is selected from:

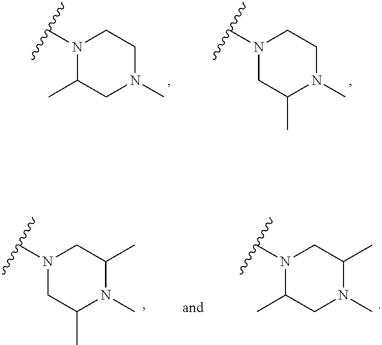

In some embodiments,

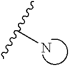 [SMILES: *N]

is selected from:

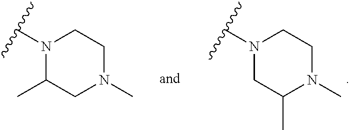

In some embodiments,

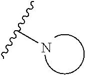 [SMILES: *N]

is selected from:

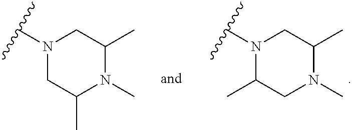

In some embodiments,

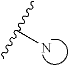 [SMILES: *N]

is selected from:

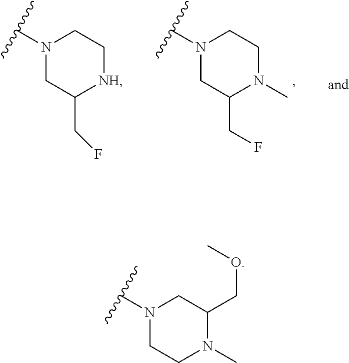

In some embodiments,

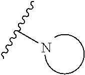 [SMILES: *N]

is selected from:

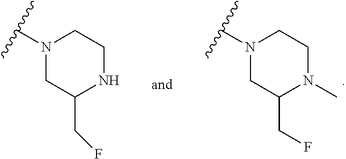

In some embodiments,

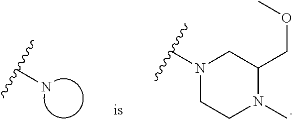

In some embodiments, for the compound or salt of Formula (I) or (I-A),

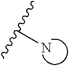 [SMILES: *N]

is selected from 7- to 10-membered bicyclic heterocycle, any one of which is optionally substituted with one or more substituents independently selected from C₁₋₆ alkyl optionally substituted with halogen, —OR¹³, —N(R¹³)₂, —NO₂, and —CN. In some embodiments,

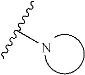 [SMILES: *N]

is selected from 7- to 10-membered bicyclic heterocycle, any one of which is optionally substituted with C₁₋₃ alkyl. In some embodiments,

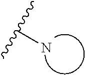 [SMILES: *N]

is selected from 7- to 10-membered bicyclic heterocycle, any one of which is optionally substituted with methyl, ethyl, propyl, and isopropyl. In some embodiments,

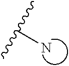 [SMILES: *N]

is selected from:

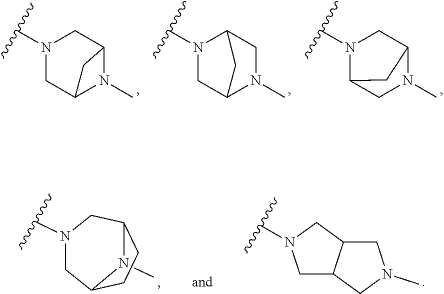

In some embodiments,

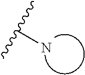 [SMILES: *N]

is selected from:

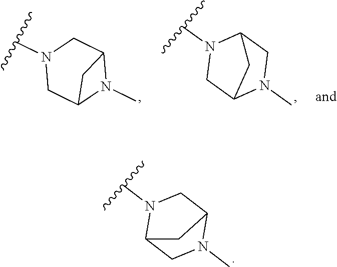

In some embodiments,

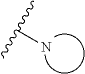 [SMILES: *N]

is selected from:

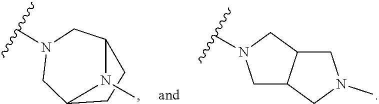

In some embodiments, for the compound or salt of Formula (I) or (I-A),

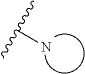 [SMILES: *N]

is selected from 5- to 8-membered spirocyclic heterocycle, any one of which is optionally substituted with one or more substituents independently selected from C₁₋₆ alkyl optionally substituted with halogen, —OR¹³, —N(R¹³)₂, —NO₂, and —CN. In some embodiments,

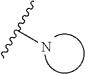 [SMILES: *N]

is selected from 5- to 8-membered spirocyclic heterocycle, any one of which is optionally substituted with one or more substituents independently selected from methyl, ethyl, propyl, and isopropyl, any of which is optionally substituted with halogen, —OR¹³, —N(R¹³)₂, —NO₂, and —CN. In some embodiments,

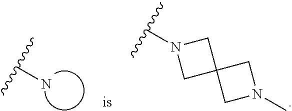

In some embodiments, for the compound or salt of Formula (I), Formula (I) is represented by Formula (I-A):

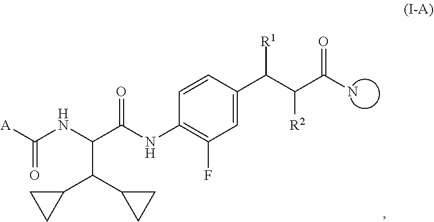

wherein each substituent is defined as in Formula (I).

In some embodiments, Formula (I) or Formula (I-A) is selected from:

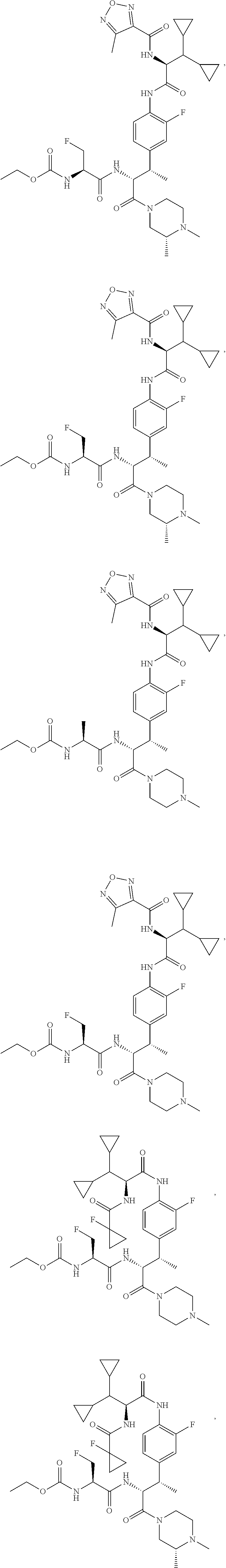

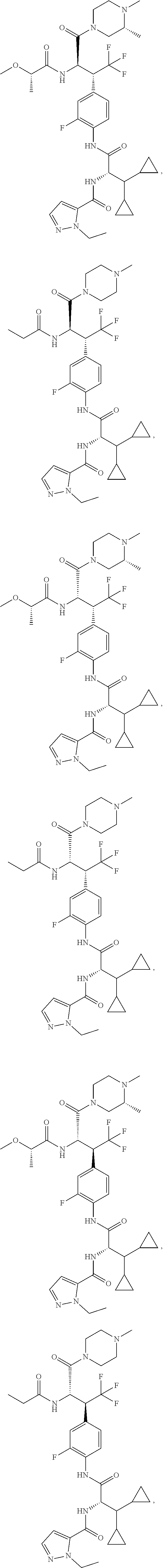

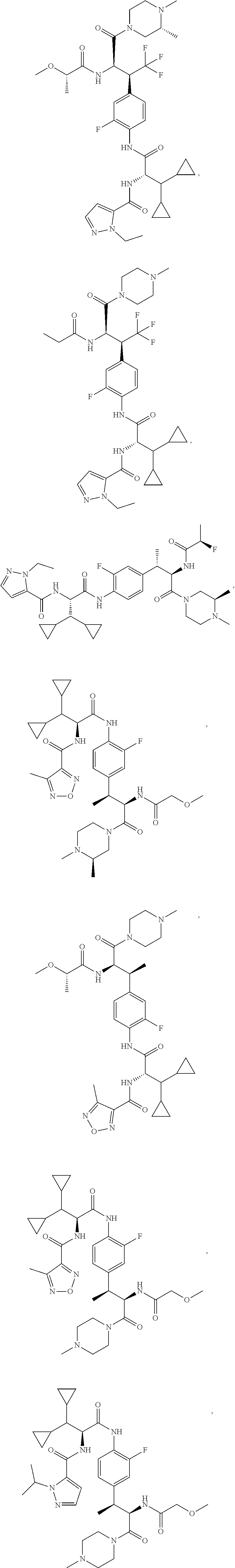

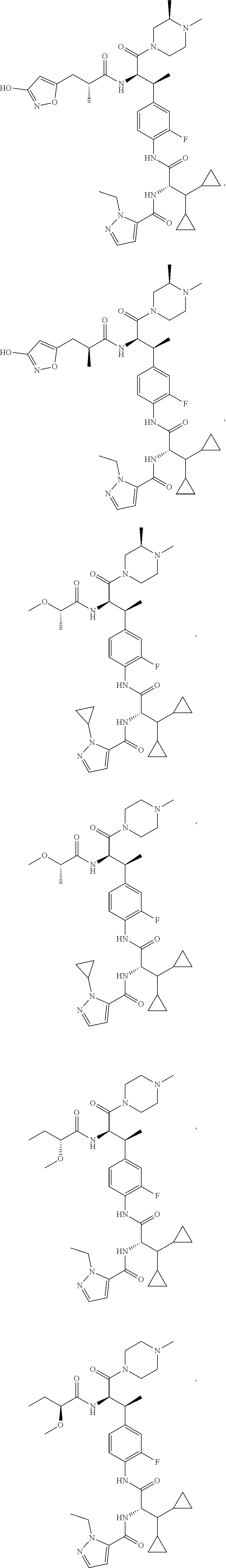

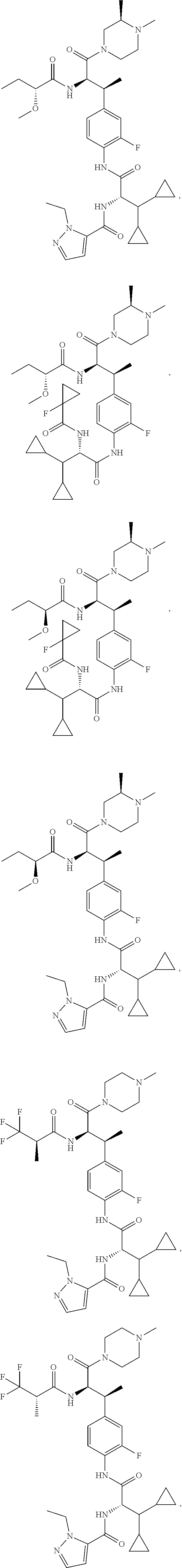

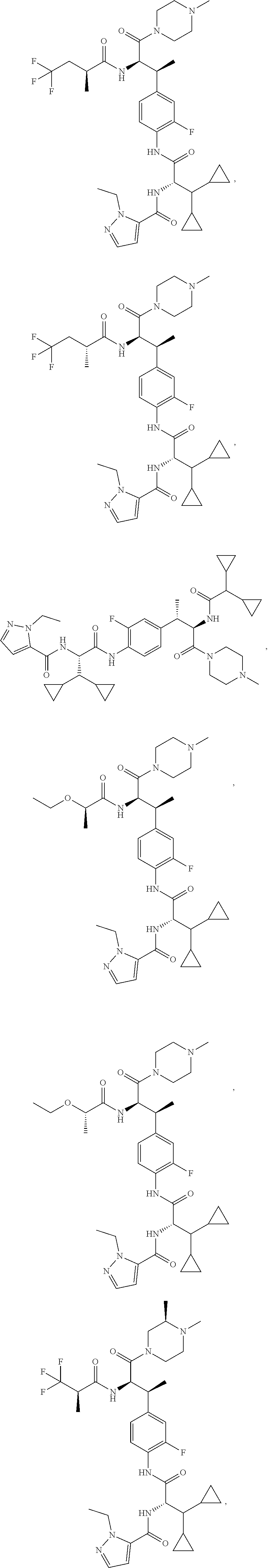

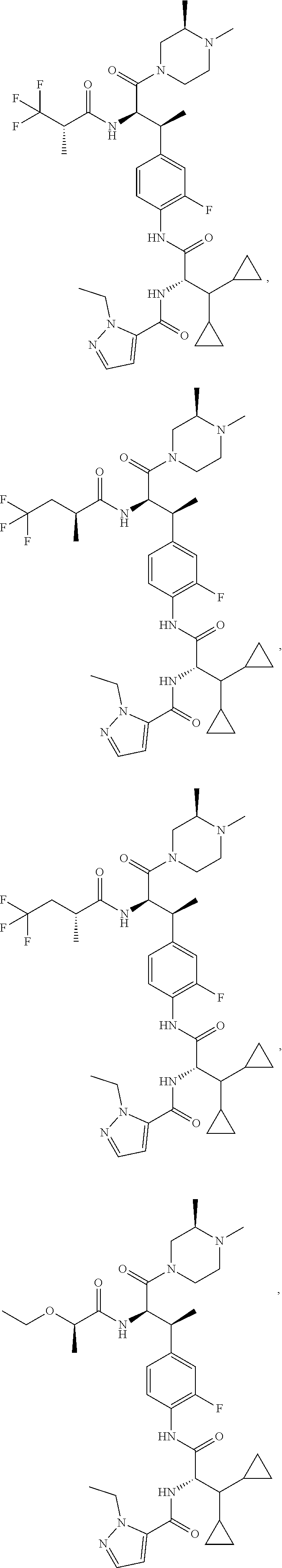 [SMILES: CCn1nccc1C(=O)N[C@H](C(=O)Nc1ccc([C@H](C)[C@@H](NC(=O)[C@H](C)C(F)(F)F)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)[C@@H](C)CC(F)(F)F)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)[C@H](C)CC(F)(F)F)C(=O)N2CCN(C)[C@H](C)C2)cc1F)C(C1CC1)C1CC1 ; CCO[C@H](C)C(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1]

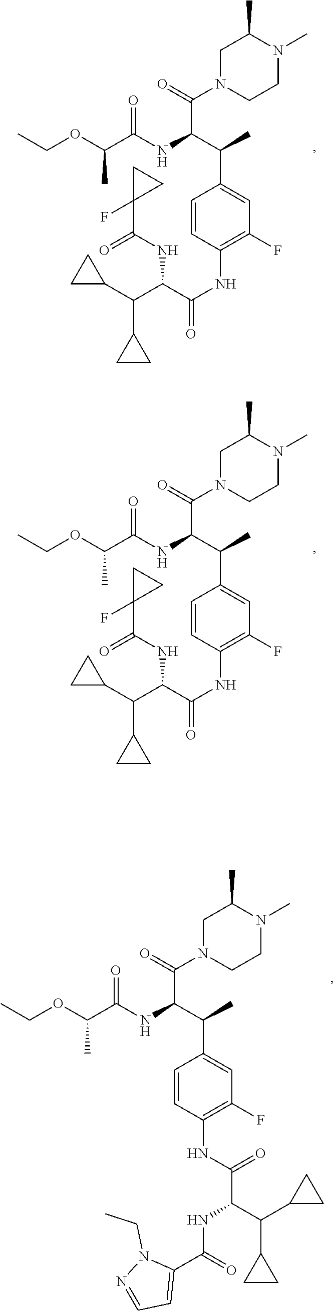

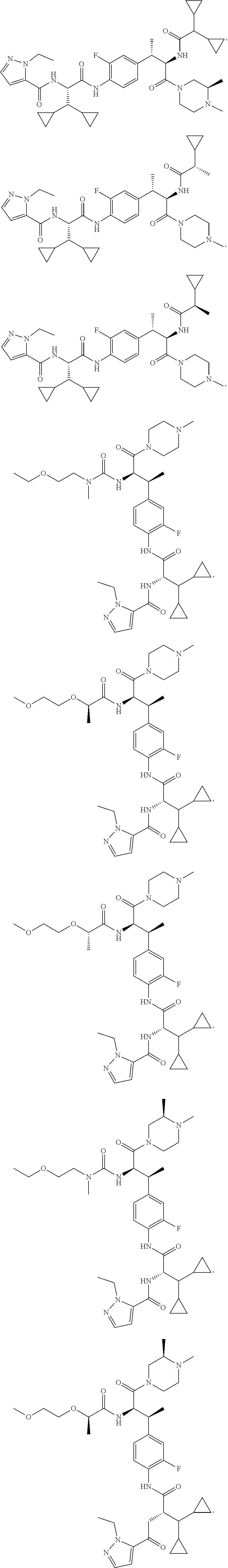

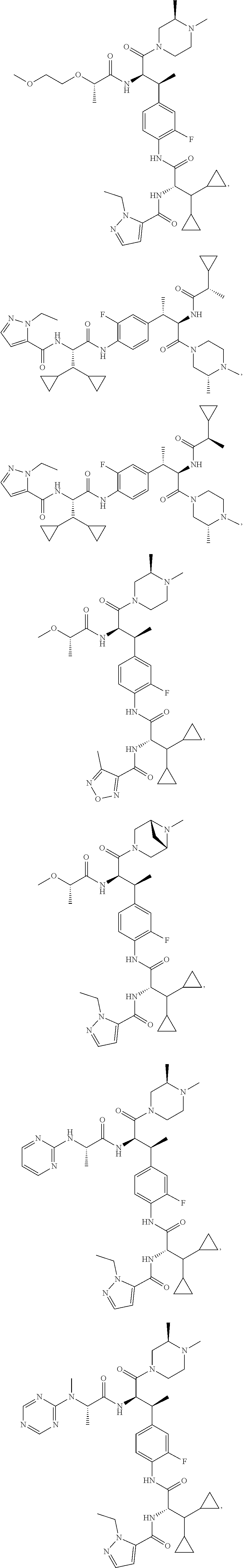

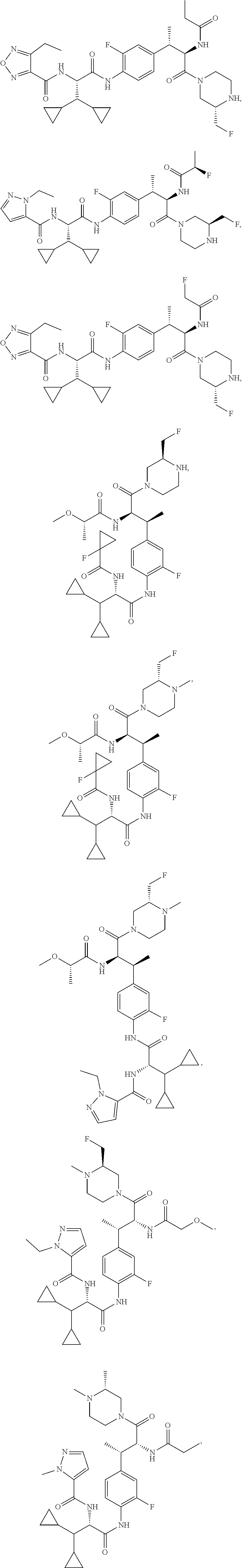

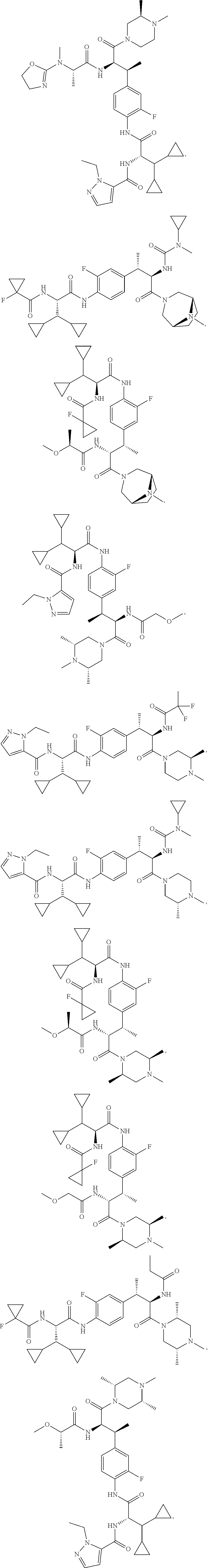

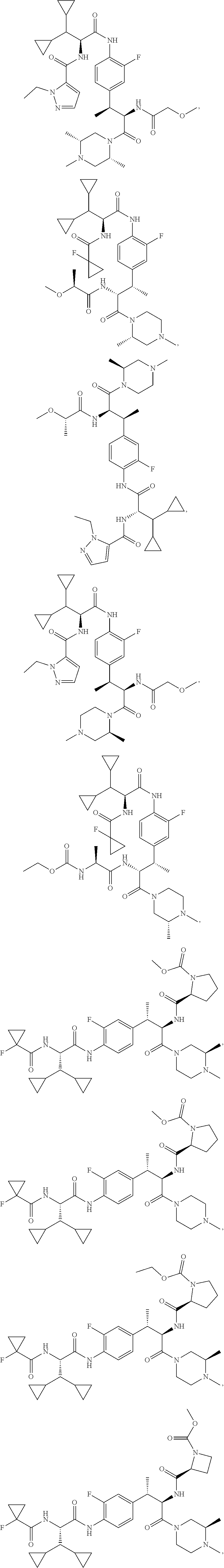

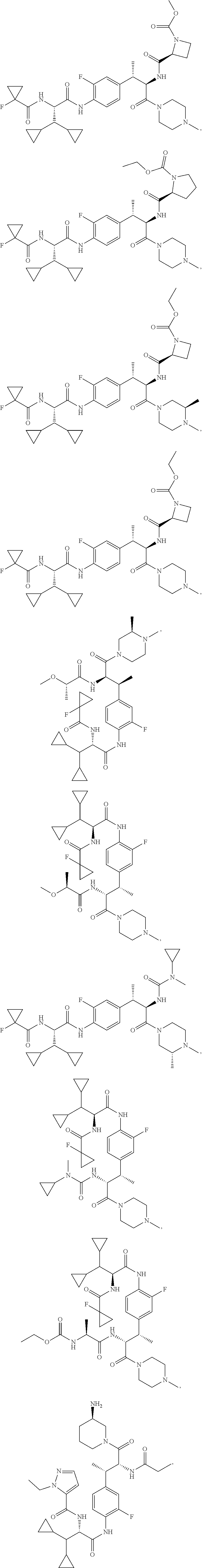

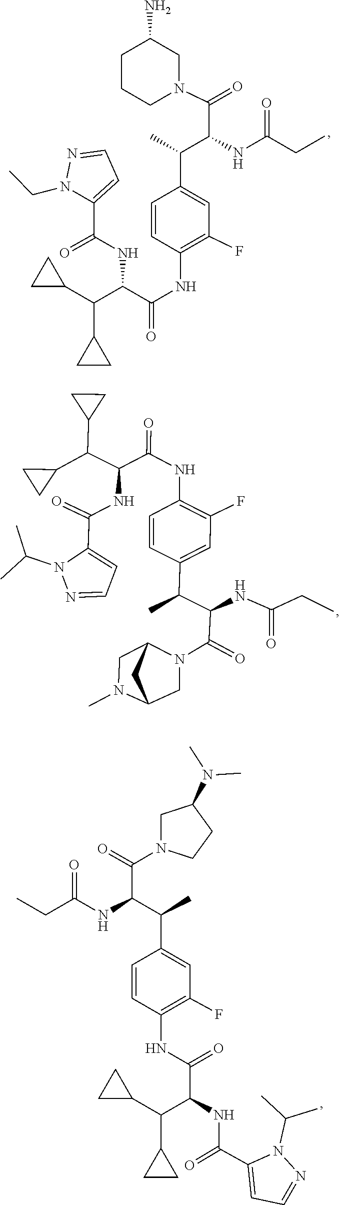

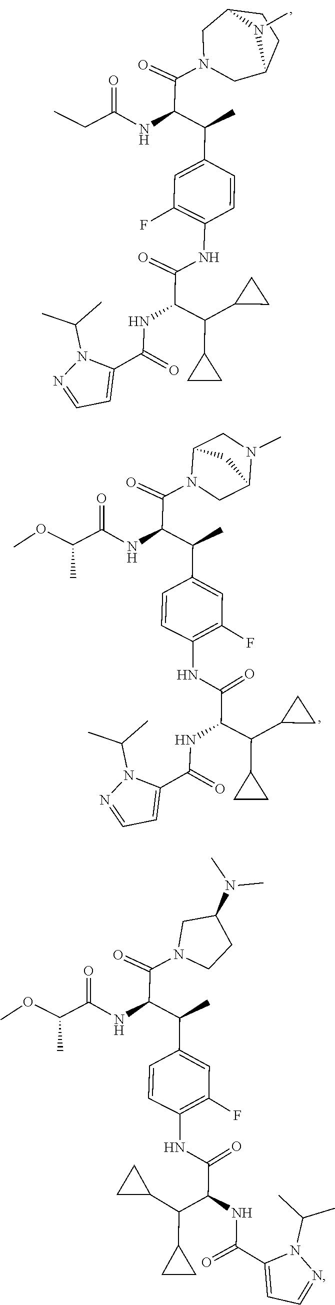

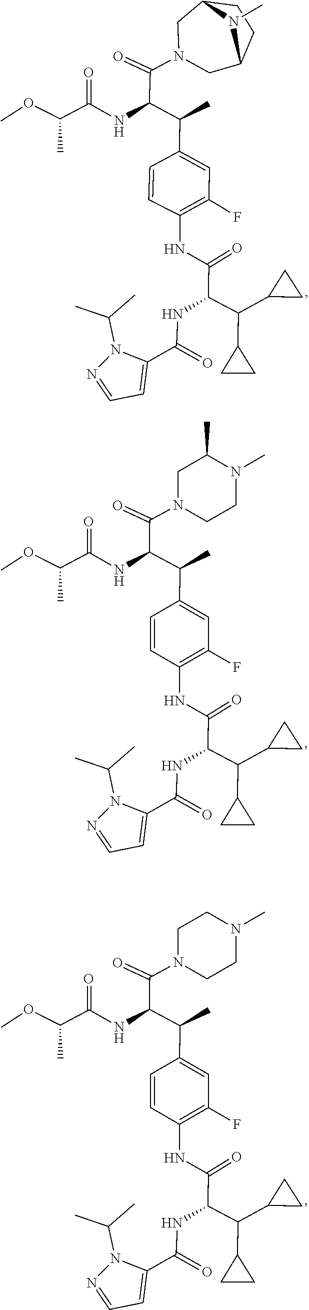 [SMILES: CO[C@@H](C)C(=O)N[C@@H](C(=O)N1C[C@H]2CC[C@@H](C1)N2C)[C@@H](C)c1ccc(NC(=O)[C@@H](NC(=O)c2ccnn2C(C)C)C(C2CC2)C2CC2)c(F)c1 ; CO[C@@H](C)C(=O)N[C@@H](C(=O)N1CCN(C)[C@H](C)C1)[C@@H](C)c1ccc(NC(=O)[C@@H](NC(=O)c2ccnn2C(C)C)C(C2CC2)C2CC2)c(F)c1 ; CO[C@@H](C)C(=O)N[C@@H](C(=O)N1CCN(C)CC1)[C@@H](C)c1ccc(NC(=O)[C@@H](NC(=O)c2ccnn2C(C)C)C(C2CC2)C2CC2)c(F)c1]

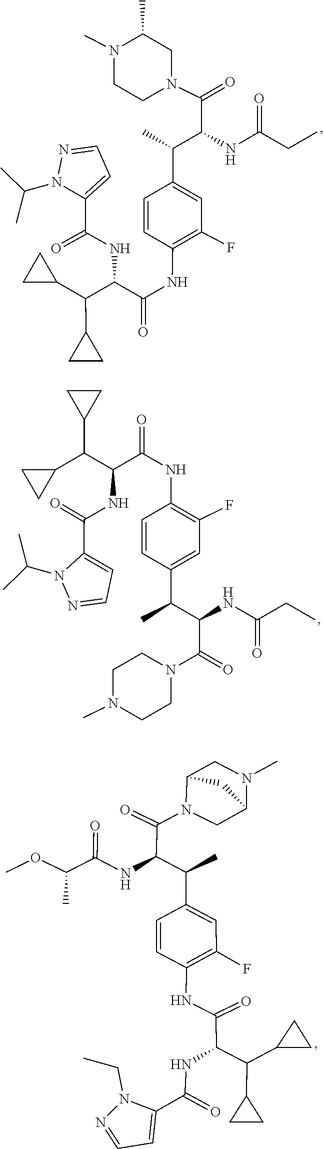

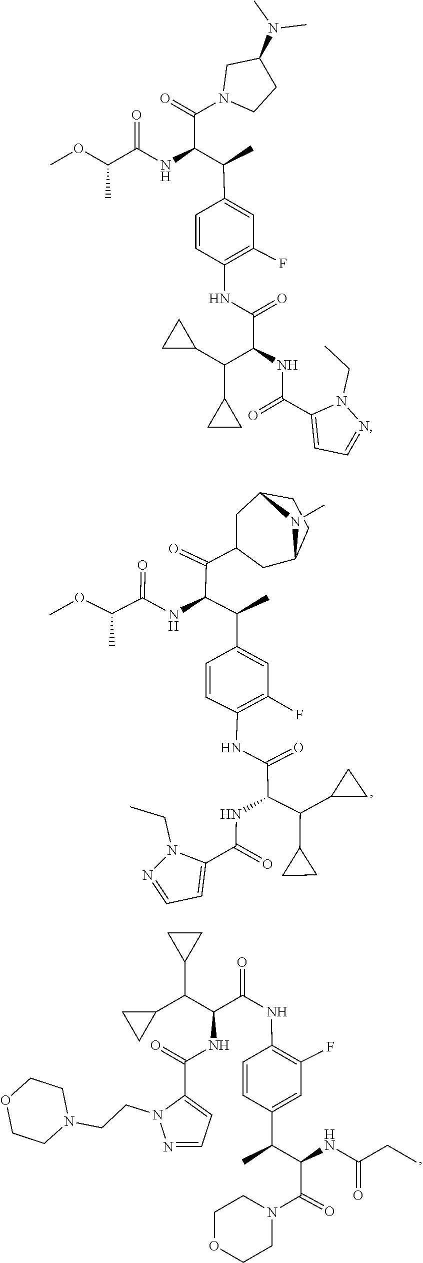

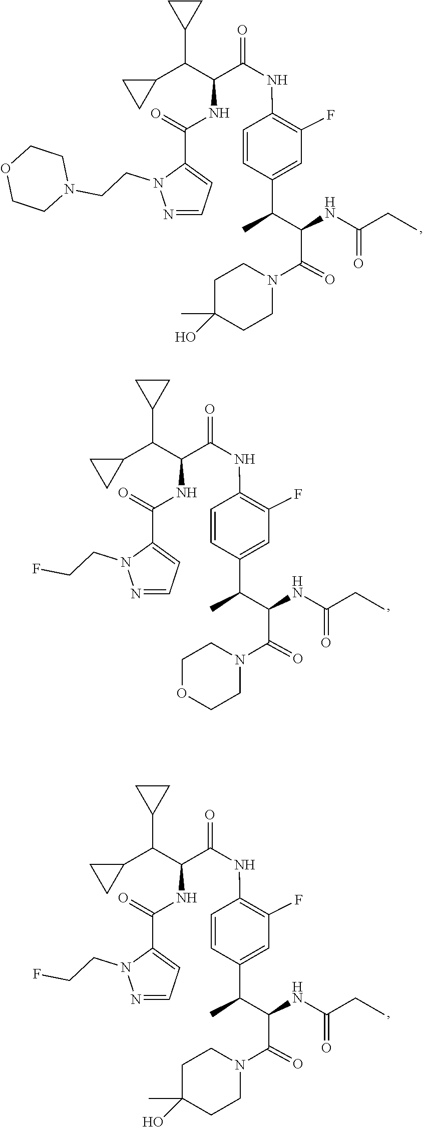 [SMILES: CCC(=O)N[C@@H](C(=O)N1CCC(C)(O)CC1)[C@@H](C)c1ccc(NC(=O)[C@@H](NC(=O)c2ccnn2CCN2CCOCC2)C(C2CC2)C2CC2)c(F)c1 ; CCC(=O)N[C@@H](C(=O)N1CCOCC1)[C@@H](C)c1ccc(NC(=O)[C@@H](NC(=O)c2ccnn2CCF)C(C2CC2)C2CC2)c(F)c1 ; CCC(=O)N[C@@H](C(=O)N1CCC(C)(O)CC1)[C@@H](C)c1ccc(NC(=O)[C@@H](NC(=O)c2ccnn2CCF)C(C2CC2)C2CC2)c(F)c1]

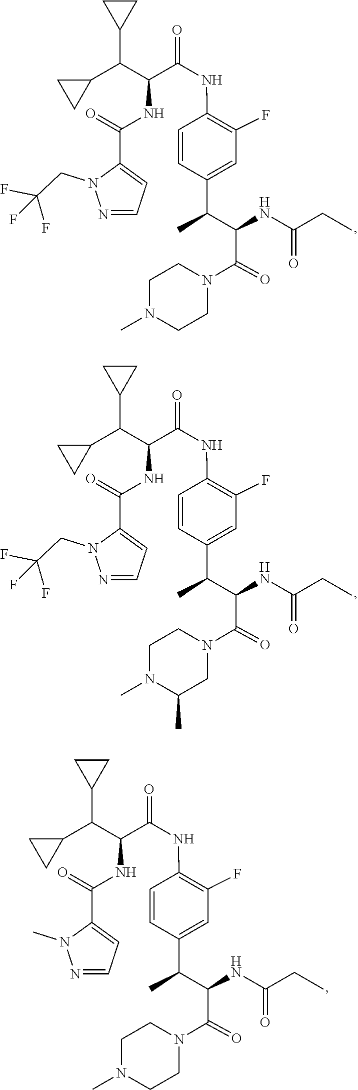

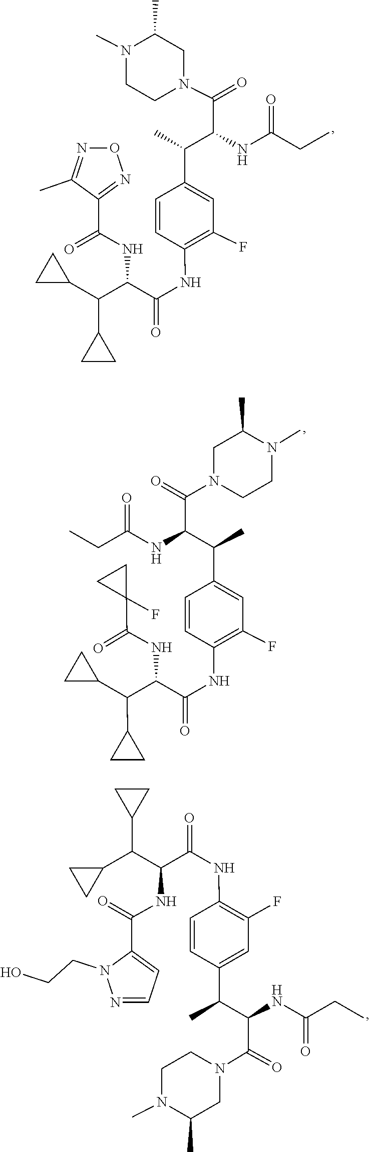

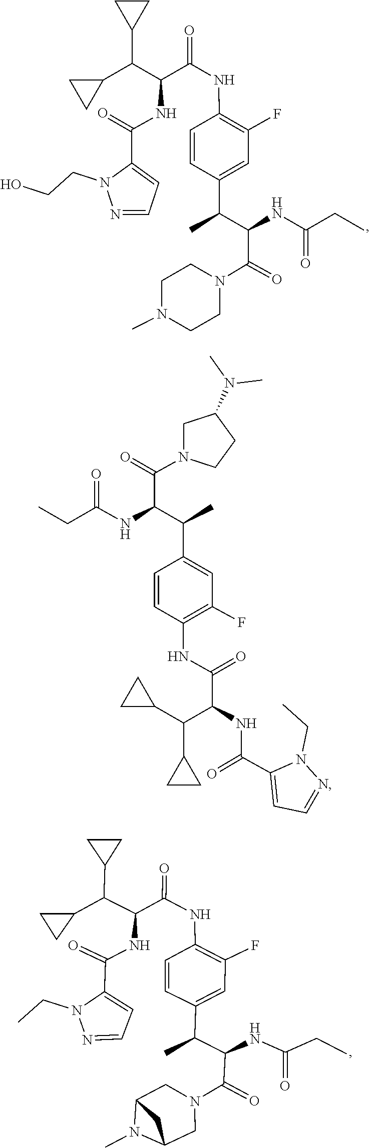

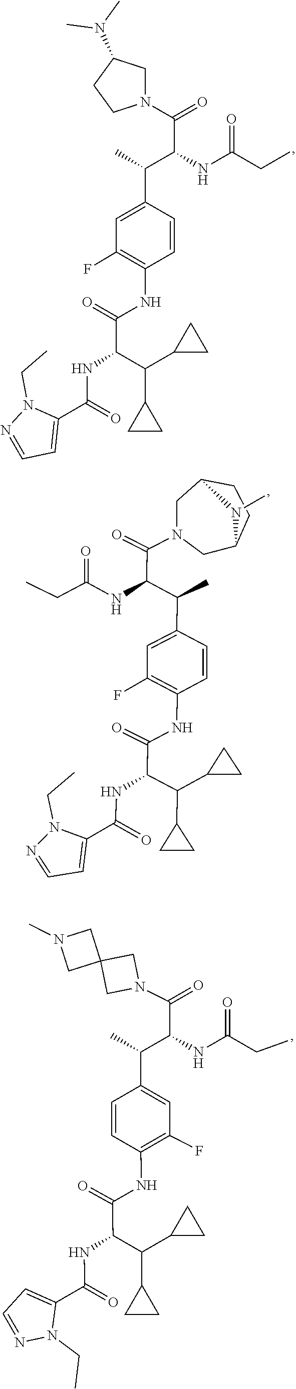

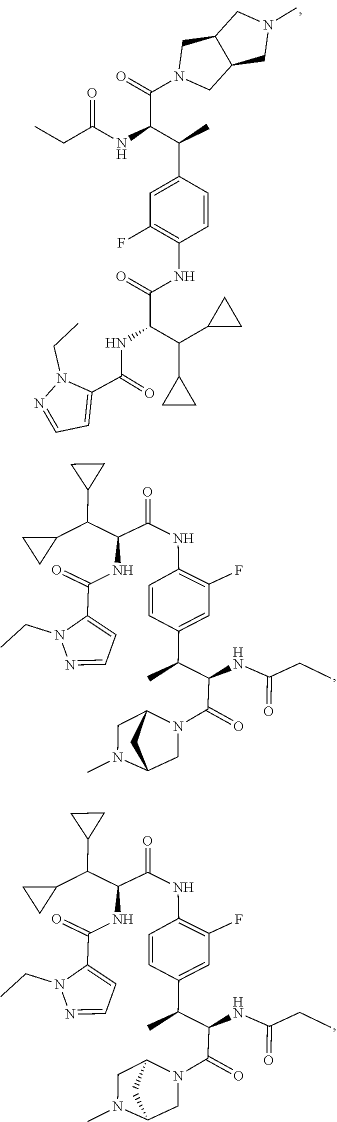

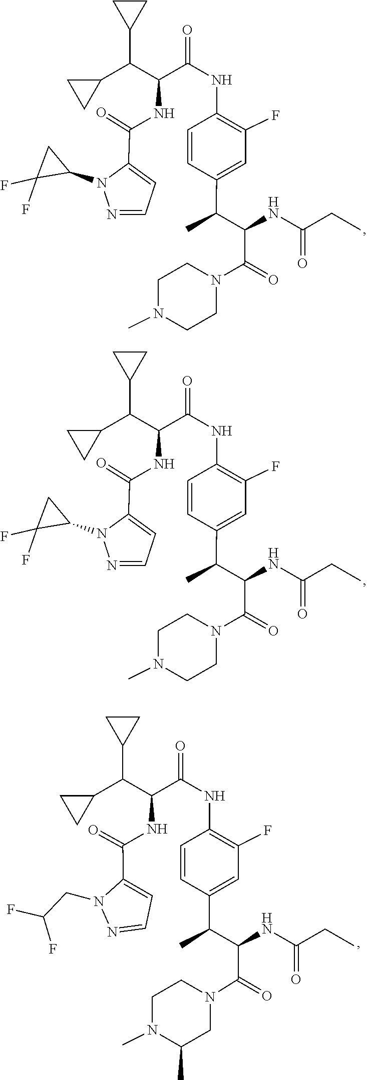

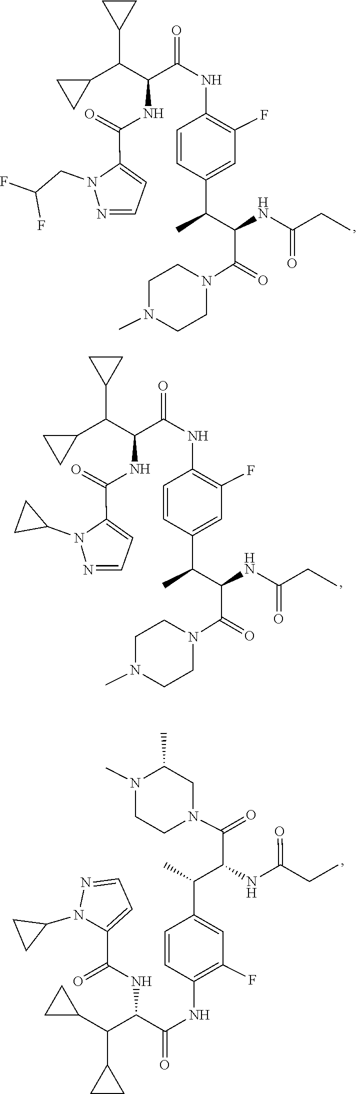

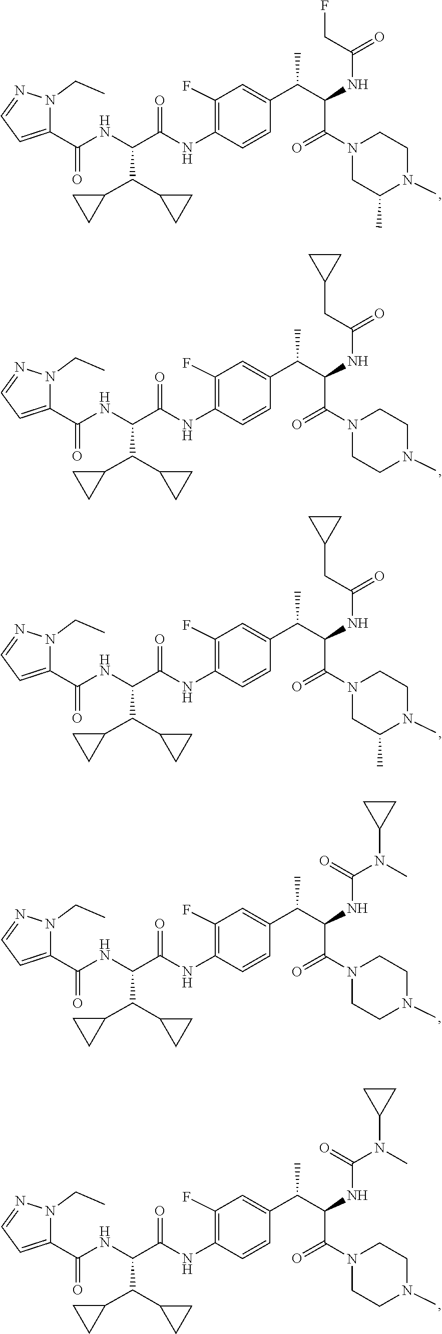

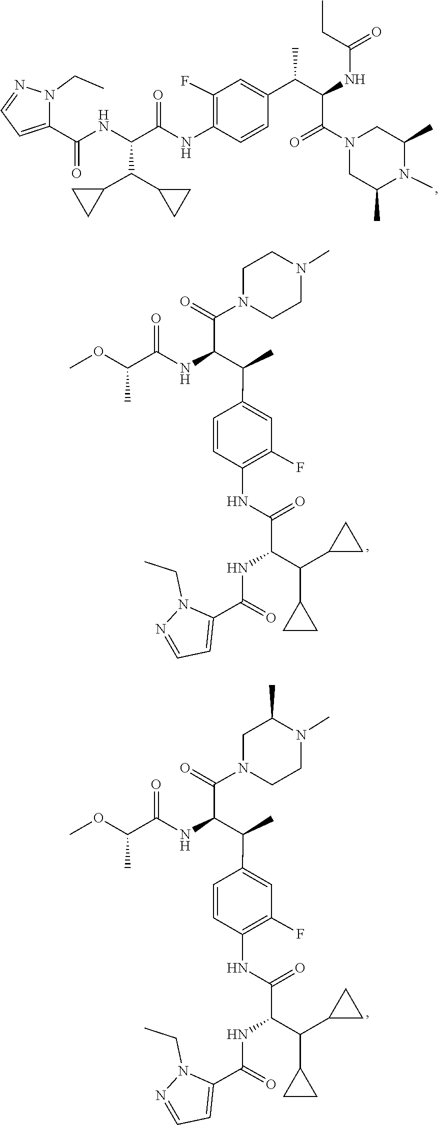

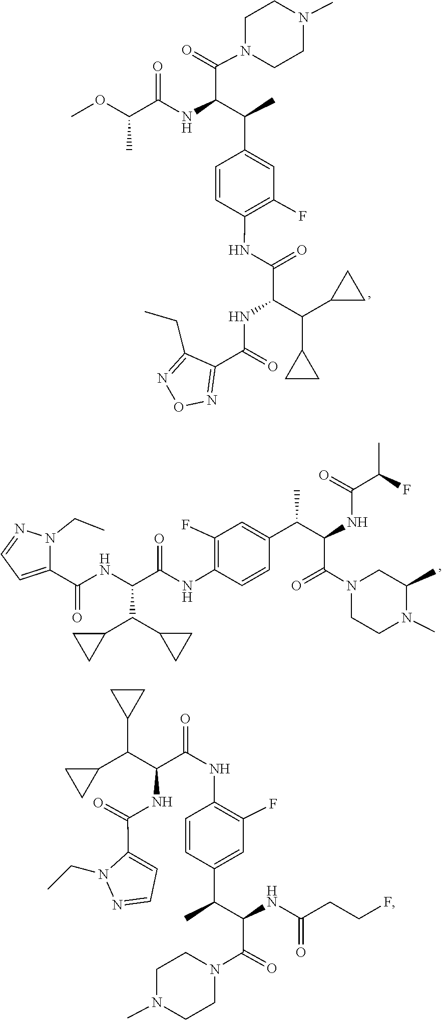

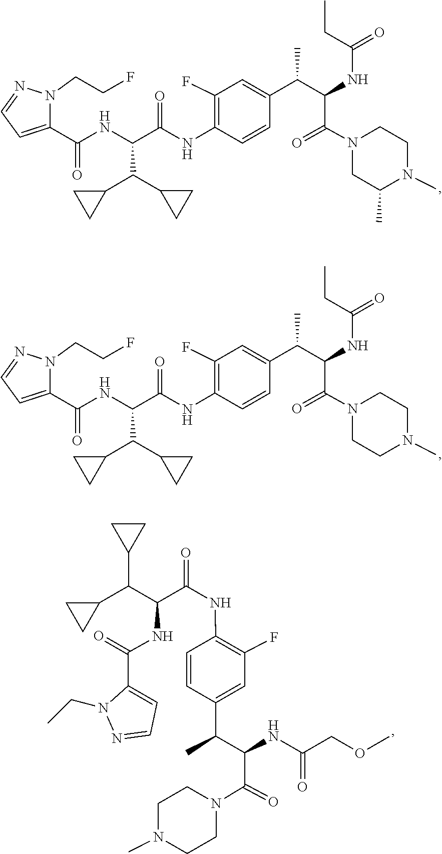

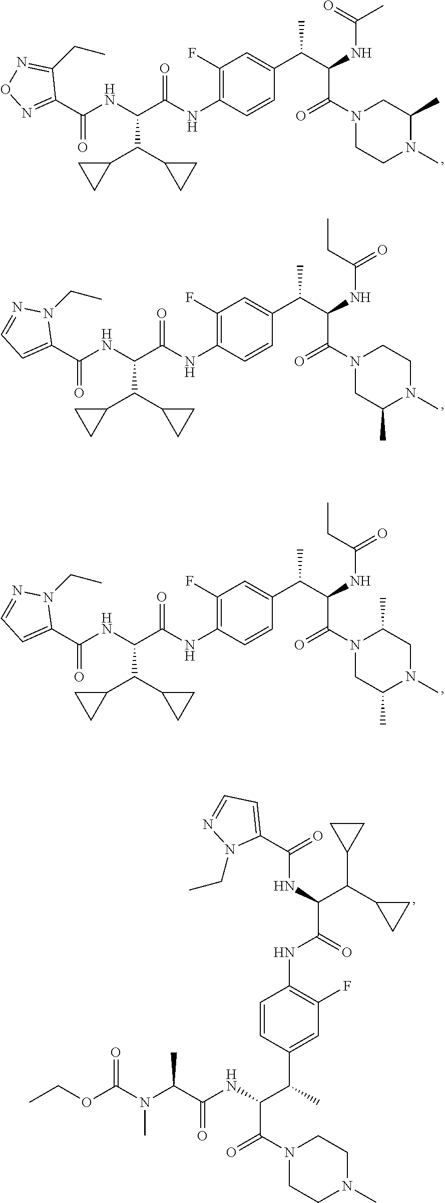

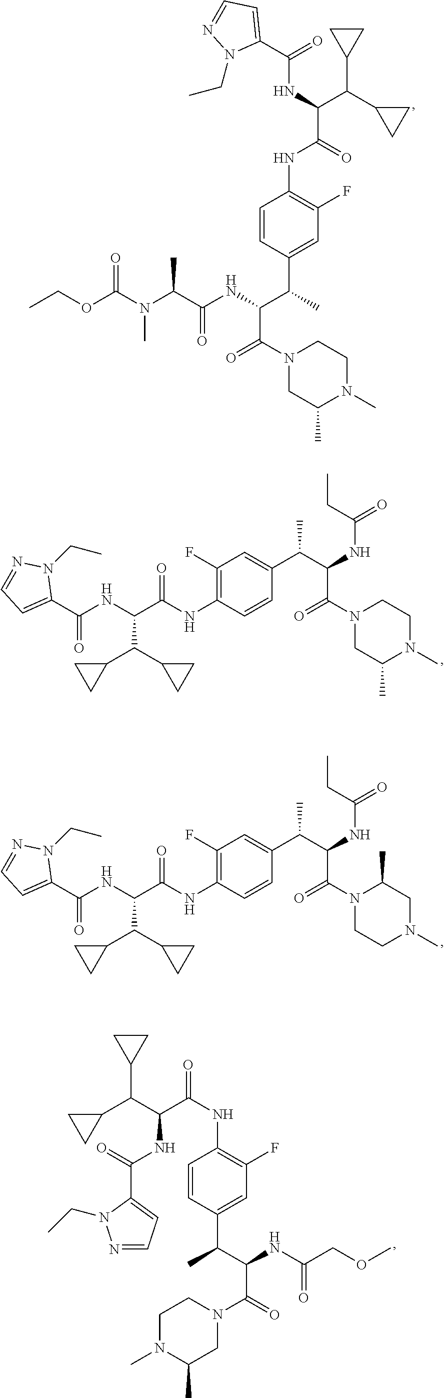

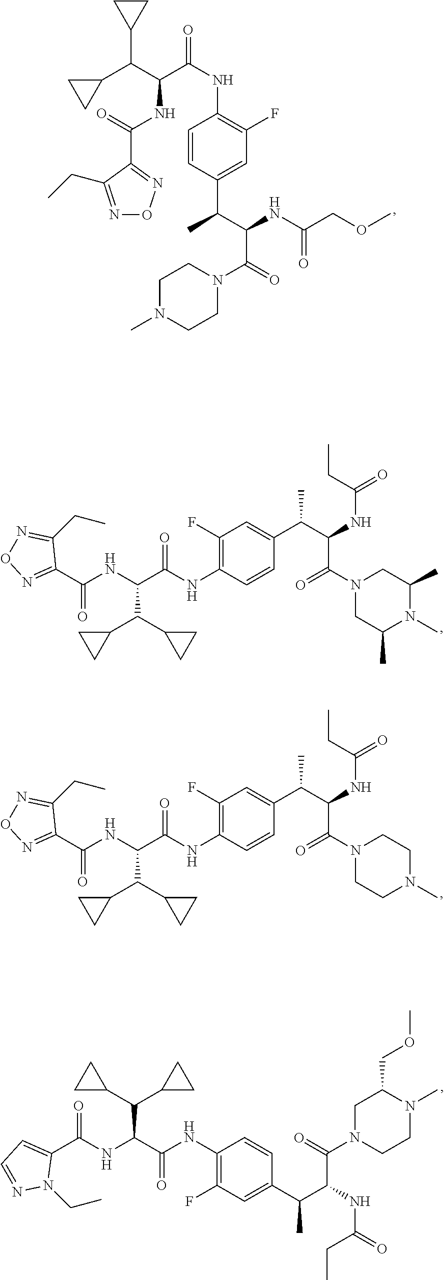 [SMILES: CCc1nonc1C(=O)N[C@H](C(=O)Nc1ccc([C@H](C)[C@@H](NC(=O)COC)C(=O)N2CCN(C)CC2)cc1F)C(C1CC1)C1CC1 ; CCC(=O)N[C@@H](C(=O)N1C[C@@H](C)N(C)[C@@H](C)C1)[C@@H](C)c1ccc(NC(=O)[C@@H](NC(=O)c2nonc2CC)C(C2CC2)C2CC2)c(F)c1 ; CCC(=O)N[C@@H](C(=O)N1CCN(C)CC1)[C@@H](C)c1ccc(NC(=O)[C@@H](NC(=O)c2nonc2CC)C(C2CC2)C2CC2)c(F)c1 ; CCC(=O)N[C@@H](C(=O)N1CCN(C)[C@@H](COC)C1)[C@@H](C)c1ccc(NC(=O)[C@@H](NC(=O)c2ccnn2CC)C(C2CC2)C2CC2)c(F)c1]

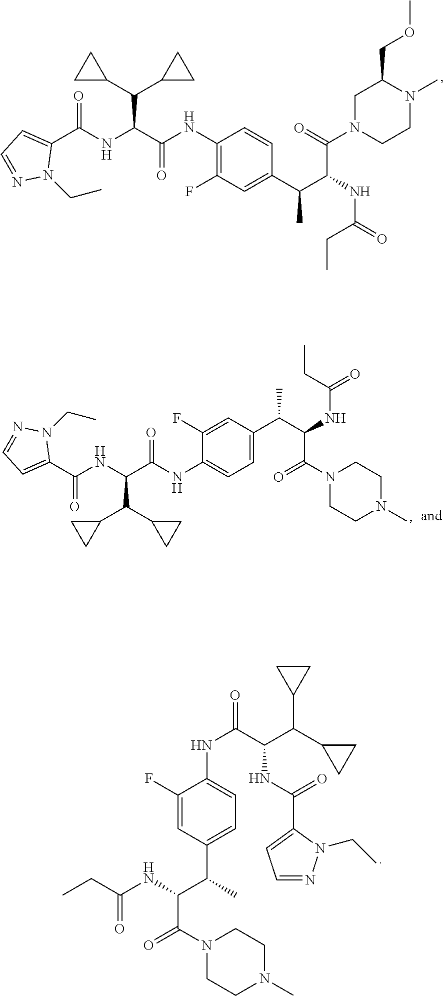

In certain aspects, the present disclosure provides a compound or salt represented by the structure of Formula (I) wherein:
A is a 5-membered heteroaryl selected from pyrazole, imidazole, oxazole, isoxazole, oxadiazole, triazole, and tetrazole, any of which is optionally substituted with one or more substituents independently selected from halogen, —OR¹¹, —N(R¹¹)₂, —C(O)R¹¹, —C(O)OR¹¹, —NO₂, —CN, and C₁₋₆ alkyl optionally substituted with one or more substituents independently selected from halogen, —OR¹¹, —N(R¹¹)₂, —C(O)R¹¹, —C(O)OR¹, —NO₂, and —CN, for example, A is pyrazole optionally substituted with one or more halogen, C₁₋₆ alkyl or C₁₋₆ haloalkyl;
R¹ is selected from hydrogen, halogen, —OR¹², —N(R¹²)₂, —NO₂, —CN, C₁₋₆ alkyl, and C₁₋₆ haloalkyl, for example, R¹ is C₁₋₆ alkyl or C₁₋₆ haloalkyl;
R² is —N(R^{A})C(O)(R^{B});
R^{A} is selected from hydrogen, C₁₋₆ alkyl, and C₁₋₆ haloalkyl, for example, R^{A} is hydrogen;
R^{B} is selected from C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, -and CN, for example, R^{B} is C₁₋₆ alkyl optionally substituted with one or more halogen, —OR¹⁶ or —C(O)OR¹⁶;

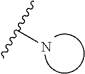 [SMILES: *N]

is selected from is selected from pyrrolidine, piperidine, morpholine, and piperazine, each of which is optionally substituted with one or more substituents selected from C₁₋₃ alkyl optionally substituted with one or more substituents independently selected from: halogen and —OR¹³, for example,

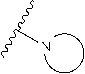 [SMILES: *N]

is piperazine optionally substituted with one or more C₁₋₃ alkyl, C₁₋₃ haloalkyl, or C₁₋₃ alkyl-OR¹³;
n is selected from 0, 1 and 2, for example, n is 1;
R³ is selected from halogen, C₁₋₃ alkyl, and C₁₋₃ haloalkyl, for example, R³ is halogen; and
R¹¹, R¹², R¹³, and R¹⁶ are each independently selected at each occurrence from hydrogen and C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN, for example R¹¹, R¹², R¹³, and R¹⁶ are each independently hydrogen, C₁₋₃ alkyl, or C₁₋₃ haloalkyl.

In certain aspects, the present disclosure provides a compound or salt represented by the structure of Formula (I) wherein:
A is a saturated C₃₋₆ carbocycle selected from cyclopropyl, cyclobutyl, cyclopentyl, and cyclohexyl, any one of which is optionally substituted with one or more substituents independently selected from halogen, —OR¹¹, —N(R¹¹)₂, —CN, C₁₋₆ alkyl, and C₁₋₆ haloalkyl, for example, A is cyclopropyl optionally substituted with one or more halogen, C₁₋₆ alkyl or C₁₋₆ haloalkyl;
R¹ is selected from hydrogen, halogen, —OR¹², —N(R¹²)₂, —NO₂, —CN, C₁₋₆ alkyl, and C₁₋₆ haloalkyl, for example, R¹ is C₁₋₆ alkyl or C₁₋₆ haloalkyl;
R² is —N(R^{A})C(O)(R^{B});
R^{A} is selected from hydrogen, C₁₋₆ alkyl, and C₁₋₆ haloalkyl, for example, R^{A} is hydrogen;
R^{B} is selected from C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, -and CN, for example, R^{B} is C₁₋₆ alkyl optionally substituted with one or more halogen, —OR¹⁶ or —C(O)OR¹⁶;

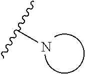 [SMILES: *N]

is selected from is selected from pyrrolidine, piperidine, morpholine, and piperazine, each of which is optionally substituted with one or more substituents selected from C₁₋₃ alkyl optionally substituted with one or more substituents independently selected from: halogen and —OR¹³, for example,

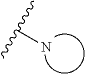 [SMILES: *N]

is piperazine optionally substituted with one or more C₁₋₃ alkyl, C₁₋₃ haloalkyl, or C₁₋₃ alkyl-OR¹³;
n is selected from 0, 1 and 2, for example, n is 1;
R³ is selected from halogen, C₁₋₃ alkyl, and C₁₋₃ haloalkyl, for example, R³ is halogen; and
R¹¹, R¹², R¹³, and R¹⁶ are each independently selected at each occurrence from hydrogen and C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN, for example R¹¹, R¹², R¹, and R¹⁶ are each independently hydrogen, C₁₋₃ alkyl, or C₁₋₃ haloalkyl.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z—, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R—S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

When stereochemistry is not specified in a chemical structure, molecules with stereocenters described herein include isomers, such as enantiomers and diastereomers, mixtures of enantiomers, including racemates, mixtures of diastereomers, and other mixtures thereof, to the extent they can be made by one of ordinary skill in the art by routine experimentation. In certain embodiments, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration.

In certain embodiments, compositions of the disclosure may comprise two or more enantiomers or diatereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

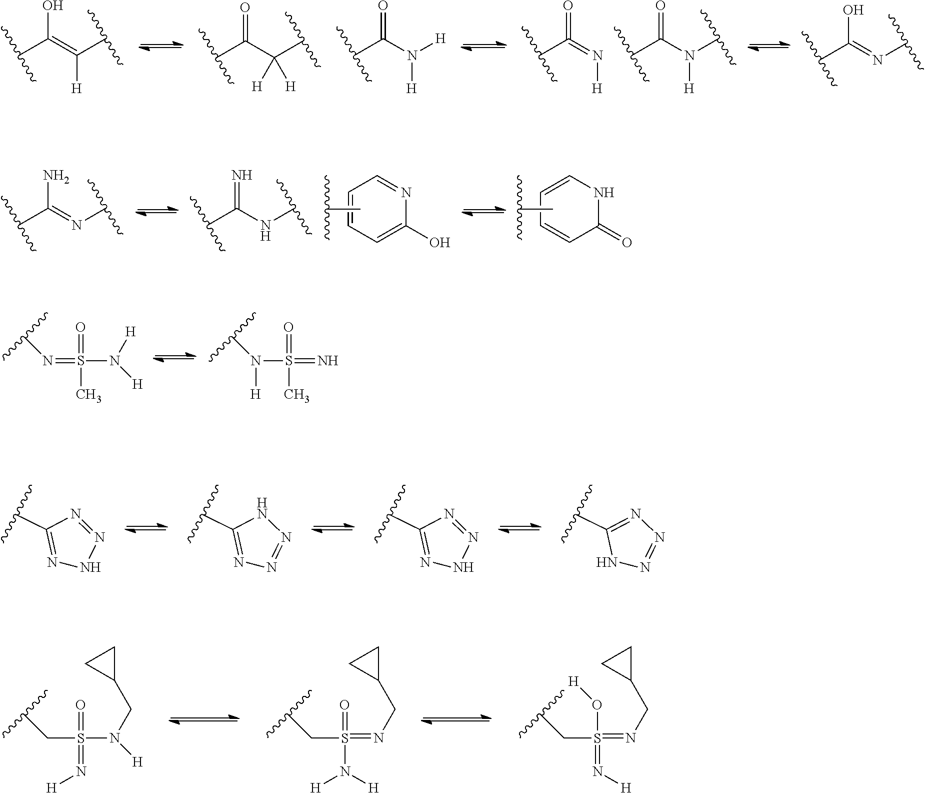

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (I). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formula (I), and at least one pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

A compound or salt of any one of Formula (I), may be formulated in any suitable pharmaceutical formulation. A pharmaceutical formulation of the present disclosure typically contains an active ingredient (e.g., compound or salt of any one of Formula (I)), and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

Pharmaceutical compositions may also be prepared from a compound or salt of any one of Formula (I), and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

### Methods of Treatment

In certain embodiments, a compound or salt of Formula (I), or a salt thereof, can be used to treat or prevent a disease or condition that is mediated directly or indirectly by IL-17A. Such diseases include inflammatory diseases and conditions, proliferative diseases (e.g., cancer), autoimmune diseases and other disease described herein. The methods generally involve administering therapeutically effective amounts of compounds disclosed herein or a pharmaceutical composition thereof to the subject.

Increased levels of IL-17A have been associated with several conditions including airway inflammation, rheumatoid arthritis (RA), osteoarthritis, bone erosion, intraperitoneal abscesses and adhesions, inflammatory bowel disorder (IBD), allograft rejection, psoriasis, psoriatic arthritis, ankylosing spondylitis, certain types of cancer, angiogenesis, atherosclerosis and multiple sclerosis (MS). Both IL-17A and IL-17R are upregulated in the synovial tissue of RA patients. IL-17A exerts its role in pathogenesis of RA through IL-1-β and TNF-α dependent and independent pathways. IL-17A stimulates secretion of other cytokines and chemokines, e.g., TNF-α, IL-1β, IL-6, IL-8 and Gro-α. IL-17A directly contributes to disease progression in RA. Injection of IL-17A into the mouse knee promotes joint destruction independently of IL-1 β activity (Ann Rheum Dis 2000, 59:529-32). Anti-IL-1P antibody has no effect on IL-17A induced inflammation and joint damage (J Immunol 2001, 167:1004-1013). In an SCW-induced murine arthritis model, IL-17A induced inflammatory cell infiltration and proteoglycan depletion in wild-type and IL-1P knockout and TNF-α knockout mice. IL-17A knockout mice are phenotypically normal in the absence of antigenic challenge but have markedly reduced arthritis following type II collagen immunization (J Immunol 2003, 171:6173-6177). Increased levels of IL-17A-secreting cells have also been observed in the facet joints of patients suffering from ankylosing spondylitis (H Appel et al., Arthritis Res Therap 2011, 13:R95).

In certain aspects, the disclosure provides methods of modulating IL-17A in a subject in need thereof, comprising administering to said subject a compound or salt of Formula (I). In certain embodiments, a compound or salt of Formula (I) inhibits the activity of IL-17A in a subject in need thereof.

In certain embodiments, a compound or salt of Formula (I) is used to treat or prevent an inflammatory disease or condition. In certain embodiments, a compound or salt of Formula (I) is administered to a subject in need thereof to treat an inflammatory disease or condition, e.g., psoriasis.

In certain embodiments, a compound or salt of Formula (I) is used to treat or prevent an inflammatory disease or condition is selected from, plaque psoriasis, guttate psoriasis, inverse psoriasis, pustular psoriasis, erythrodermic psoriasis, aspsoriatic arthritis, ankyslosing spondylitis, hidradenitis suppurutiva, rheumatoid arthritis, Palmoplantar Psoriasis, Spondyloarthritis, and Non-infectious Uveitis. In certain embodiments, a compound or salt of Formula (I) is used to treat or prevent psoriasis.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-8 show the general procedure for the preparation of the claimed IL-17A modulators. Examples 9-10 illustrate bioassay procedures for IL-17A/A and IL-17A/F inhibition. Example 11 illustrates comparative data of selected claimed IL-17A modulators against reference compounds.

Table 1 shows, spectroscopic data, bioassay inhibition, and synthetic procedures for the claimed IL-17A modulators. Table 2 shows selected claimed IL-17A modulators and selected comparative compounds. Table 2.6 shows microsomal stability data for selected claimed IL-17A modulators and selected comparative compounds. Table 2.6 shows Rat pharmacokinetic data for selected claimed IL-17A modulators and selected comparative compounds.

### Example 1—Synthesis of Intermediates A and B

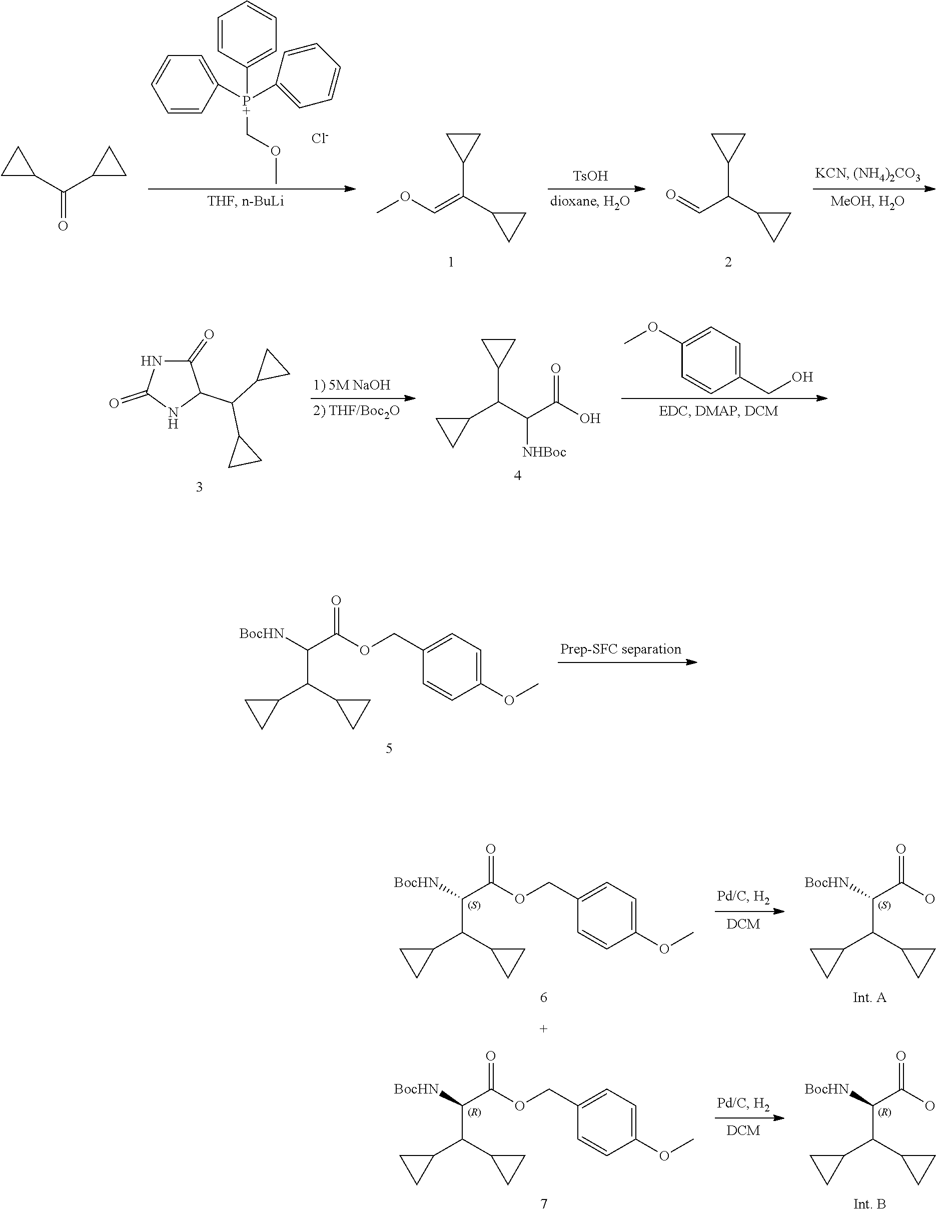

Step 1: To a solution of (Methoxymethyl)triphenylphosphonium chloride (140.04 g, 408.52 mmol, 1.5 eq) in THF (1000 mL) was added dropwise n-BuLi (2.5 M, 163.41 mL, 1.5 eq) at 5° C. under N₂. After addition, the mixture was stirred at 5° C. for 1 hr, and then dicyclopropylmethanone (30 g, 272.35 mmol, 30.71 mL, 1 eq) was added dropwise. The resulting mixture was stirred at 60° C. for 12 hrs. To the reaction was added H₂O (200 mL) and extracted with ethyl acetate (500 mL*2). The combined organic layers were dried over Na₂SO₄, filtered and concentrated. The residue was purified by column chromatography (SiO₂, Petroleum ether: Ethyl acetate=100:1 to 50:1). Compound 1 (30 g, 217.07 mmol, 79.70% yield) was obtained as yellow oil. ¹H NMR: (400 MHz, CDCl₃) δ 5.87 (s, 1H), 3.58 (s, 3H), 1.84-1.78 (m, 1H), 0.85-0.73 (m, 1H), 0.73-0.72 (m, 2H), 0.61-0.57 (m, 2H), 0.47-0.45 (m, 2H), 0.25-0.23 (m, 2H).

Step 2: To a mixture of Compound 1 (30 g, 217.07 mmol, 1 eq) in dioxane (500 mL) and H₂O (50 mL) was added TsOH (149.52 g, 868.27 mmol, 4 eq). The mixture was stirred at 110° C. for 3h. To the reaction was added sat. NaHCO₃ (1000 mL) and extracted with ethyl acetate (1000 mL*2). The combined organic layers were dried over Na₂SO₄, filtered and concentrated. The crude product was distilled in vacuum (120° C., 0.1 Mpa pressure). Compound 2 (21 g, 169.11 mmol, 77.91% yield) was obtained as colorless oil. ¹H NMR: (400 MHz, CDCl₃) δ 9.76-9.72 (m, 1H), 1.04-0.96 (m, 1H), 0.87-0.85 (m, 2H), 0.58-0.53 (m, 4H), 0.26-0.24 (m, 4H).

Step 3: To a mixture of Compound 2 (7 g, 56.37 mmol, 1 eq) in MeOH (35 mL) and H₂O (35 mL) was added KCN (5.51 g, 84.55 mmol, 3.62 mL, 1.5 eq) and (NH₄)₂CO₃ (16.25 g, 169.11 mmol, 18.05 mL, 3 eq). The mixture was stirred at 60° C. for 12 hrs. To the reaction was added sat NaHCO₃ (200 mL) and extracted with ethyl acetate (200 mL*2). The combined organic layers were dried over Na₂SO₄, filtered and concentrated. The residue was purified by column chromatography (SiO₂, Petroleum ether: Ethyl acetate=10:1 to 1:1). Compound 3 (6 g, 30.89 mmol, 54.80% yield, 100% purity) was obtained as white solid. LC-MS: rt=0.87 min; m/z=195.1 for [M+H]⁺. ¹H NMR: (400 MHz, CDCl₃) δ 8.52 (s, 1H), 6.51 (s, 1H), 4.26 (s, 1H), 0.87-0.79 (m, 3H), 0.75-0.72 (m, 1H), 0.52-0.50 (m, 2H), 0.50-0.49 (m, 2H), 0.24-0.21 (m, 3H).

Step 4: To a mixture of Compound 3 (10 g, 51.49 mmol, 100% purity, 1 eq) in H₂O (200 mL) was added NaOH (5 M, 102.97 mL, 10 eq). The mixture was stirred at 100° C. for 12 hrs. Then the mixture was cooled at 20° C., then the mixture was adjusted the pH to 7˜8 with 5 M HCl (1 mL). Boc₂O (16.85 g, 77.23 mmol, 17.74 mL, 1.5 eq) and THF (100 mL) was added dropwise slowly to the above mixture. The mixture was stirred at 25° C. for 4 hrs. To the reaction was added H₂O (100 mL) and extracted with ethyl acetate (100 mL*2). The combined organic layers were dried over Na₂SO₄, filtered and concentrated. The residue was purified by column chromatography (SiO₂, Petroleum ether: Ethyl acetate=10:1 to 1:1). Compound 4 (11 g, 38.02 mmol, 73.85% yield, 93.1% purity) was obtained as yellow oil. LC-MS: rt=0.93 min; m/z=292.0 for [M+Na]⁺. ¹H NMR: (400 MHz, CDCl₃) δ 10.03 (s, 1H), 6.04-5.27 (m, 1H), 4.56-4.37 (m, 1H), 1.45 (s, 9H), 0.78-0.52 (m, 3H), 0.27-0.25 (m, 4H), 0.23-0.20 (m, 4H).

Step 5: A mixture of Compound 4 (2.0 g, 6.92 mmol, 93.1% purity, 1 eq), 4-methoxybenzyl alcohol (1.15 g, 8.30 mmol, 1.03 mL, 1.2 eq), DCC (2.14 g, 10.38 mmol, 2.10 mL, 1.5 eq) and DMAP (845.49 mg, 6.92 mmol, 1 eq) in DCM (50 mL) was stirred at 20° C. for 16 hr. To the reaction was added H₂O (100 mL) and extracted with DCM (100 mL*2). The combined organic layers were dried over Na₂SO₄, filtered and concentrated. The residue was purified by prep-HPLC (column: Phenomenex luna C18 (250*70 mm, 10 um); mobile phase: [water (0.1% TFA)-ACN]; B %: 58%-86%, 21 min). Compound 5 (2 g, 5.07 mmol, 73.31% yield, 98.8% purity) was obtained as white solid. LC-MS: rt=1.10 min; m/z=412.2 for [M+Na]⁺. ¹H NMR: (400 MHz, CDCl₃) δ 7.30-7.27 (m, 2H), 6.90-6.87 (m, 2H), 5.26 (d, J=9.2 Hz, 1H), 5.09 (s, 2H), 4.55-4.36 (m, 1H), 3.82 (s, 3H), 1.45 (s, 9H), 0.73-0.63 (m, 3H), 0.48-0.47 (m, 3H), 0.19-0.18 (m, 1H), 0.17-0.15 (m, 3H), 0.14-0.01 (m, 1H).

Step 6: The residue of compound 5 was purified by Prep-SFC (column: DAICEL CHIRALPAK AY-H (250 mm*30 mm, 10 um); mobile phase: [0.1% NH₃H₂O ETOH]; B %: 70%-70%, 10.5; 160 min). Compound 6 (0.9 g, 2.30 mmol, 45.36% yield, 99.6% purity) was obtained as yellow oil. LC-MS: rt=1.09 min; m/z=412.2 for [M+Na]⁺. ¹H NMR: (400 MHz, CDCl₃) δ 7.30-7.27 (m, 2H), 6.90-6.87 (m, 2H), 5.26 (d, J=8.8 Hz, 1H), 5.09 (s, 2H), 4.55-4.36 (m, 1H), 3.81 (s, 3H), 1.45 (s, 9H), 0.73-0.63 (m, 3H), 0.48-0.47 (m, 3H), 0.19-0.18 (m, 1H), 0.17-0.15 (m, 3H), 0.14-0.01 (m, 1H). Compound 7 (0.9 g, 2.30 mmol, 45.36% yield, 99.7% purity) was obtained as yellow oil. LC-MS: rt=1.09 min; m/z=412.2 for [M+Na]⁺. ¹H NMR: (400 MHz, CDCl₃) δ 7.30-7.27 (m, 2H), 6.90-6.87 (m, 2H), 5.26 (d, J=9.2 Hz, 1H), 5.09 (s, 2H), 4.55-4.36 (m, 1H), 3.82 (s, 3H), 1.45 (s, 9H), 0.73-0.63 (m, 3H), 0.48-0.47 (m, 3H), 0.19-0.18 (m, 1H), 0.17-0.15 (m, 3H), 0.14-0.01 (m, 1H).

Step 7a: To a solution of Compound 6 (250 mg, 641 umol, 1.00 eq) in DCM (2.00 mL) was added Pd/C (100 mg, 10.0% purity) under N₂. The suspension was degassed under vacuum and purged with H₂ for several times. The mixture was stirred under H₂ (15 Psi) at 25° C. for 24 hrs. The reaction mixture was filtered, and the filter was concentrated. Intermediate A (0.170 g, 631 umol, 98.3% yield) was obtained as colorless oil. LC-MS: rt=0.85 min; m/z=292.2 for [M+Na]⁺.

X-ray crystallographic analysis of Intermediate A confirmed the following absolute stereochemistry:

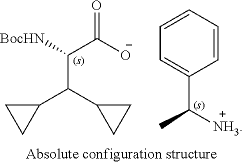

Step 7b: To a solution of Compound 7 (250 mg, 641 umol, 1.00 eq) in DCM (2.00 mL) was added Pd/C (100 mg, 10.0% purity) under N₂. The suspension was degassed under vacuum and purged with H₂ for several times. The mixture was stirred under H₂ (15 Psi) at 25° C. for 24 hrs. The reaction mixture was filtered, and the filter was concentrated. Intermediate B (0.170 g, 631 umol, 98.3% yield) was obtained as colorless oil, which was used without further characterization.

### Example 2. General Scheme—Synthesis of Intermediate C

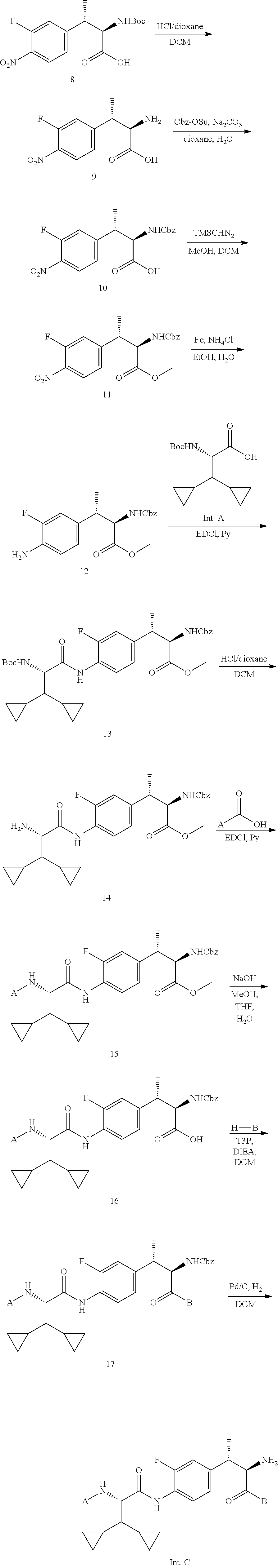

Step 1: To a solution of Compound 8 (25.0 g, 73.0 mmol, 1.00 eq) in DCM (200 mL) was added HCl/dioxane (4 M, 182 mL, 10.0 eq) at 0° C. The mixture was stirred at 25° C. for 3 hrs. The reaction mixture was concentrated to give a product. Compound 9 (17.0 g, 61.0 mmol, 83.5% yield, HCl) was obtained as a yellow solid. LC-MS: rt=0.16 min; m/z=243.2 for [M+H]⁺. Procedures for the synthesis of Compound 8 can be found in U.S. application Ser. No. 17/118,947 and U.S. application Ser. No. 16/783,268.

Step 2: To a solution of compound 9 (17.0 g, 61.0 mmol, 1.00 eq, HCl) in dioxane (170 mL) was added a solution of Na₂CO₃ (19.4 g, 183 mmol, 3.00 eq) in H₂O (170 mL) and Cbz-OSu (30.4 g, 122 mmol, 2.00 eq) at 0° C. That mixture was added at 25° C. for 12 hrs. The reaction mixture was quenched by addition 1 M aq HCl 100 mL at 0° C. to pH=4, and then extracted with ethyl acetate 400 mL (200 mL*2). The combined organic layers were brine, dried over Na₂SO₄, filtered and concentrated under reduced pressure to give a residue. Compound 10 (22.0 g, 58.4 mmol, 95.8% yield) was obtained as a yellow oil. LC-MS: rt=0.84 min; m/z=375.1 for [M−H]—.

Step 3: To a solution of compound 10 (22.0 g, 58.4 mmol, 1.00 eq) in MeOH (22.0 mL), DCM (220 mL) was added TMSCHN₂ (2 M, 43.8 mL, 1.50 eq) at 0° C., the mixture was stirred at 25° C. for 12 hrs. The reaction mixture was quenched by addition 10% AcOH 50.0 mL at 0° C., and then diluted with H₂O 200 mL and extracted with DCM 400 mL (200 mL*2). The combined organic layers were dried over Na₂SO₄, filtered and concentrated under reduced pressure to give a residue. The residue was purified by column chromatography (SiO₂, Petroleum ether: Ethyl acetate=100:1 to 10:1) Compound 11 (21.0 g, 53.8 mmol, 92.0% yield) was obtained as a yellow oil. LC-MS: rt=0.92 min; m/z=413.1 for [M+Na]⁺.

Step 4: To a solution of compound 11 (21.0 g, 53.8 mmol, 1.00 eq) in EtOH (300 mL) was added a solution of a solution of NH₄Cl (14.3 g, 268 mmol, 5.00 eq) in H₂O (100 mL), Fe (15.0 g, 268 mmol, 5.00 eq). The mixture was stirred at 80° C. for 1 hr. The reaction mixture was filtered and concentrated under reduced pressure to remove EtOH, and then diluted with H₂O (100 mL) and extracted with DCM (200 mL*2). The combined organic layers were washed with brine (200 mL*2), dried over Na₂SO₄, filtered and concentrated under reduced pressure to give a residue. Compound 12 (19.0 g, 52.7 mmol, 98.0% yield) was obtained as a white solid. LC-MS: rt=0.96 min; m/z=361.3 for [M+H]⁺.

Step 5: To a solution of compound 12 (2.50 g, 6.94 mmol, 1.00 eq) and Intermediate A (2.39 g, 8.88 mmol, 1.28 eq) in pyridine (20.0 mL) was added EDCl (3.99 g, 20.8 mmol, 3.00 eq). The mixture was stirred at 25° C. for 2 hrs. The reaction mixture was diluted with H₂O (50.0 mL) and extracted with DCM (50.0 mL*3). The combined organic layers were washed with Na₂SO₄ (50.0 mL*3), dried over Na₂SO₄, filtered and concentrated under reduced pressure to give a residue. The crude product was triturated with MTBE (50.0 ml) at 25° C. for 2 hrs. The residue was purified by Prep-HPLC (basic condition, column: Waters Xbridge 150*25 mm*5 um; mobile phase: [water (0.05% ammonia hydroxide v/v)-ACN]; B %: 53%-83%, 10 min). Compound 13 (4.20 g, 6.87 mmol, 98.9% yield) was obtained as a white solid. LC-MS: rt=1.14 min; m/z=512.4 for [M+H-boc]⁺.

Step 6: To a solution of compound 13 (3.00 g, 4.90 mmol, 1.00 eq) in DCM (20.0 mL) was added HCl/dioxane (4 M, 12.2 mL, 10.0 eq) at 0° C. The mixture was stirred at 25° C. for 1 hr. The reaction mixture was concentrated under reduced pressure to give a residue. Compound 14 (2.60 g, 4.74 mmol, 96.7% yield, HCl) was obtained as a white solid. LC-MS: rt=1.09 min; m/z=512.4 for [M+H]⁺.

Step 7: To a solution of compound 14 (2.10 g, 3.83 mmol, 1.00 eq, HCl) and 1-ethyl-1H-pyrazole-5-carboxylic acid (“A-COOH”,

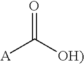

(805 mg, 5.75 mmol, 1.50 eq) in pyridine (5.00 mL) was added EDCl (2.20 g, 11.5 mmol, 3.00 eq). The mixture was stirred at 25° C. for 2 hrs. The reaction mixture was diluted with H₂O (30.0 mL) and extracted with DCM (30.0 mL*3). The combined organic layers were washed with sat.aq NaHCO₃ (30.0 mL*3), dried over Na₂SO₄, filtered and concentrated under reduced pressure to give a residue. The crude product was triturated with MTBE (50.0 mL) at 25° C. for 2 hrs. Compound 15 (2.00 g, 3.16 mmol, 82.3% yield) was obtained as a white solid. LC-MS: rt=0.96 min; m/z=634.4 for [M+H]⁺.

Step 8: To a solution of compound 15 (2.00 g, 3.16 mmol, 1.00 eq) in MeOH (10.0 mL) and THF (10.0 mL) was added a solution of NaOH (378 mg, 9.47 mmol, 3.00 eq) in H₂O (2.00 mL) at 0° C. The mixture was stirred at 30° C. for 2 hrs. The reaction mixture was diluted with H₂O (30.0 mL) and added 1 M HCl to adjusted pH to 2. The mixture was extracted with DCM (50.0 mL*3). The combined organic layers were dried over Na₂SO₄, filtered and concentrated under reduced pressure to give a residue. Compound 16 (1.90 g, 3.07 mmol, 97.1% yield) was obtained as a white solid. LC-MS: rt=0.96 min; m/z=620.3 for [M+H]⁺.

Step 9: To a solution of compound 16 (80.0 mg, 129 umol, 1.00 eq) and (2S,6R)-1,2,6-trimethylpiperazine (“H—B”) (24.8 mg, 193 umol, 1.50 eq), DIEA (83.4 mg, 645.4 umol, 112.3 μL, 5.00 eq) in DCM (5.00 mL) was added T3P (246 mg, 387 umol, 230 μL, 50% purity, 3.00 eq) at 0° C. The mixture was stirred at 25° C. for 2 hrs. The reaction mixture was diluted with H₂O (30.0 mL) and extracted with DCM (30.0 mL*3). The combined organic layers were washed with sat.aq NaHCO₃ (30.0 mL*2), dried over Na₂SO₄, filtered and concentrated under reduced pressure to give a residue. The residue was purified by SFC (EW27685-21-P1A_c10) (column: DAICEL CHIRALPAK AD (250 mm*30 mm, 10 um); mobile phase: [0.1% NH₃H₂O IPA]; B %: 35%-35%, 3; 40 min, Rt=1.929). Compound 17 (50.0 mg, 68.5 umol, 53.0% yield) was obtained as a white solid. LC-MS: rt=1.06 min; m/z=730.6 for [M+H]⁺.

Step 10: To a solution of compound 17 (50.0 mg, 68.5 umol, 1.00 eq) in DCM (5.00 mL) was added Pd/C (5.00 mg, 10% purity) under N₂ atmosphere. The suspension was degassed and purged with H₂ for 3 times. The mixture was stirred under H₂ at 25° C. for 2 hrs. The reaction mixture was filtered and the cake was washed with 100 mL MeOH, then concentrated under reduced pressure to give a residue. Int. C (40.0 mg, 67.1 umol, 98.0% yield) was obtained as a white solid. LC-MS: rt=0.91 min; m/z=596.5 for [M+H]⁺.

As described in Example 2,

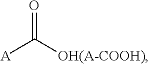

is represented by:

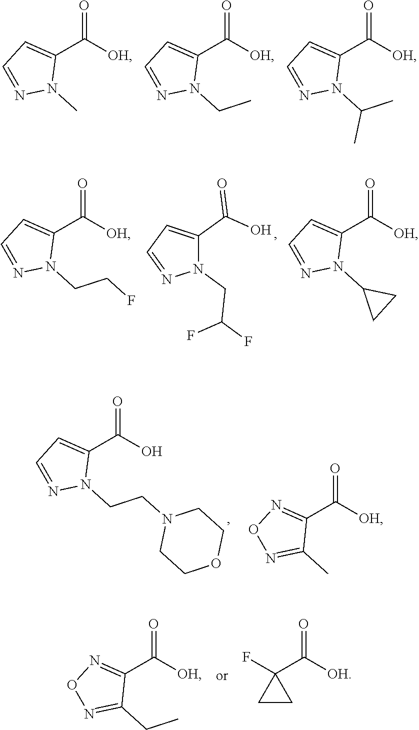

In some embodiments, A of

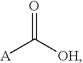

is represented by A of Formula (I) or (I-A), and is an optionally substituted C₃₋₆ carbocycle or an optionally substituted 5- to 6-membered heterocycle as described herein. In some embodiments, A of A-COOH is an optionally substituted C₃₋₆ carbocycle or an optionally substituted 5- to 6-membered heterocycle as described in Formula (I) or (I-A).

As described in Example 2, H—B is represented by:

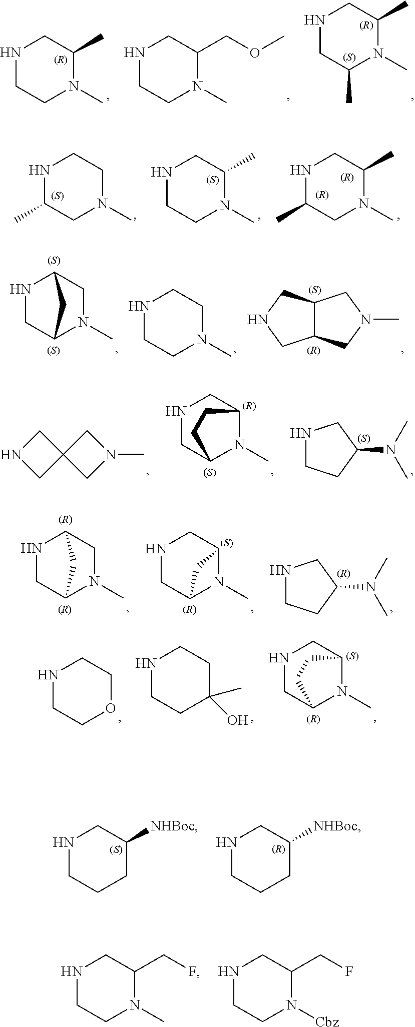

or a stereoisomer thereof. In some embodiments, as described in Example 2, H—B is represented by:

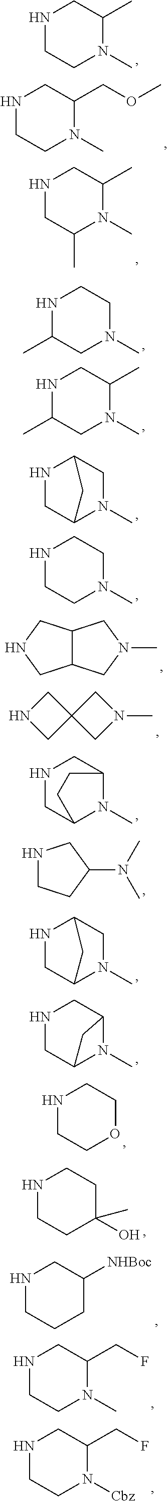

or a stereoisomer thereof. In some embodiments, B of H—B is represented by

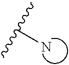 [SMILES: *N]

of Formula (I) or (I-A), and is an optionally substituted 4- to 12-membered heterocycle as described herein.

### Example 3. Exemplary Scheme—Synthesis of Selected Compounds

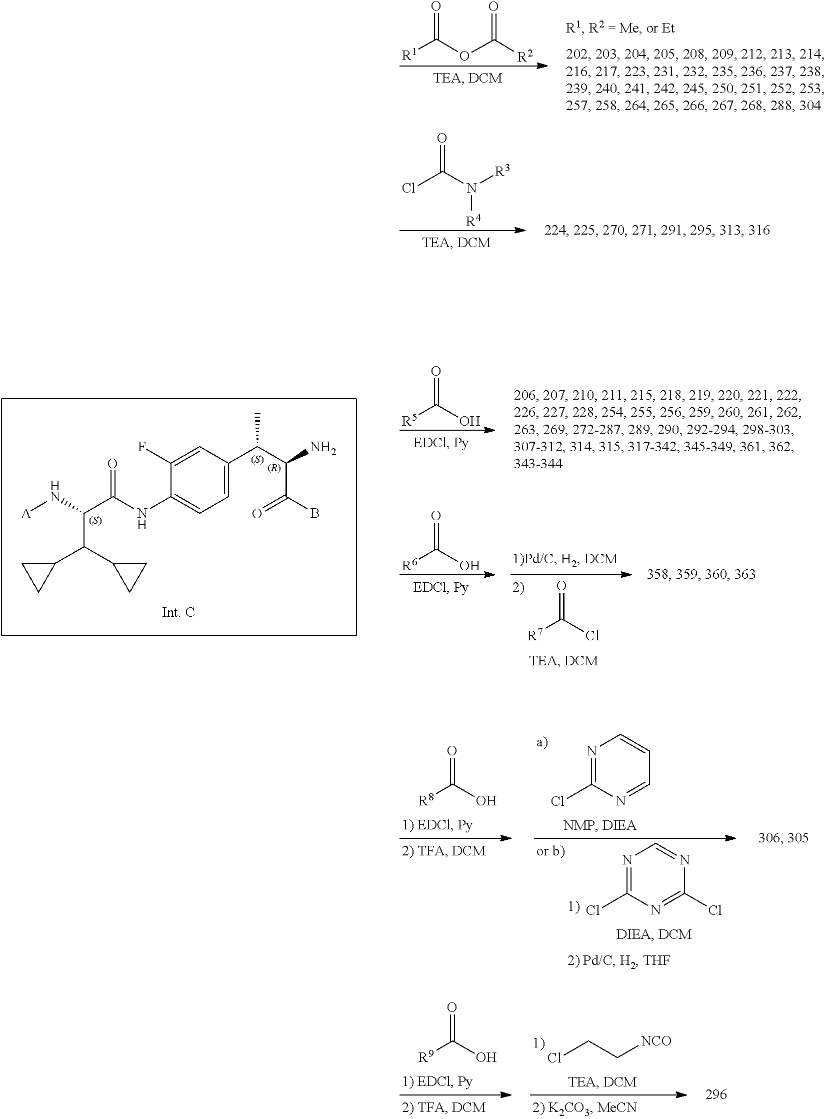

### Example 3.1.1—Synthesis of Compound 223

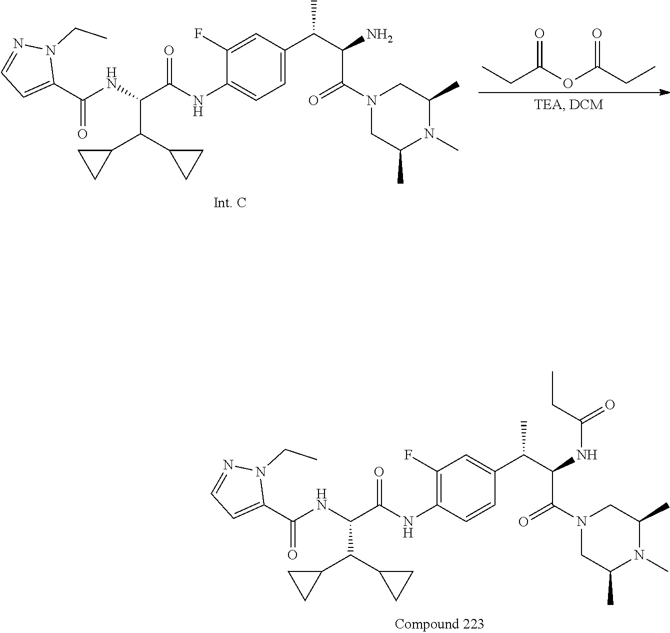

To a solution of Int. C (40.0 mg, 67.0 umol, 1.00 eq) and TEA (20.3 mg, 201 umol, 28.0 uL, 3.00 eq) in DCM (5.00 mL) was added propionic anhydride (17.4 mg, 134 umol, 17.3 uL, 2.00 eq) at 0° C. The mixture was stirred at 25° C. for 2 hrs. The reaction mixture was diluted with H₂O (30.0 mL) and extracted with DCM (30.0 mL*3). The combined organic layers were dried over Na₂SO₄, filtered and concentrated under reduced pressure to give a residue. The residue was purified by Prep-TLC (SiO₂, DCM:MeOH=10:1). Compound 223 (35.2 mg, 51.4 umol, 76.6% yield, 95.3% purity) was obtained as a white solid. LC-MS: rt=0.95 min; m/z=652.6 for [M+H]⁺.

Compounds 202, 203, 204, 205, 208, 209, 212, 213, 214, 216, 217, 231, 232, 235, 236, 237, 238, 239, 240, 241, 242, 245, 250, 251, 252, 253, 257, 258, 264, 265, 266, 267, 268, 288, and 304 were similarly synthesized using similar reaction conditions with the appropriate anhydride reagent.

### Example 3.1.2—Synthesis of Compound 204

To a solution of appropriately substituted Int. C (50.0 mg, 87.7 umol, 1.00 eq) and TEA (26.6 mg, 263 umol, 36.6 uL, 3.00 eq) in DCM (3.00 mL) was added propionic anhydride (13.7 mg, 105 umol, 13.5 uL, 1.20 eq) at 0° C. The mixture was stirred at 25° C. for 1 hr. The reaction mixture was concentrated under reduced pressure to give a residue. The residue was purified by Prep-TLC (SiO₂, DCM:MeOH=10:1). Compound 204 (34.6 mg, 53.5 umol, 60.9% yield, 96.7% purity) was obtained as a white solid. LC-MS: rt=0.84 min; m/z=626.2 for [M+H]⁺.

### Example 3.1.3—Synthesis of Compound 205

To a solution of appropriately substituted Int. C (40.0 mg, 66.9 umol, 1.00 eq) and TEA (20.3 mg, 200 umol, 27.9 uL, 3.00 eq) in DCM (5.00 mL) was added propionic anhydride (13.0 mg, 100 umol, 12.9 uL, 1.50 eq) at 25° C. The mixture was stirred at 25° C. for 2 hr. The reaction mixture was concentrated under reduced pressure to give a residue. The residue was purified by Prep-TLC (SiO₂, DCM:MeOH=10:1). Compound 205 (41.8 mg, 63.3 umol, 94.6% yield, 99.1% purity) was obtained as a white solid. LC-MS: rt=0.84 min; m/z=654.3 for [M+H]⁺.

### Example 3.1.4—Synthesis of Compound 208

To a solution of appropriately substituted Int. C (30.0 mg, 51.5 umol, 1.00 eq) and TEA (26.0 mg, 257 umol, 35.8 uL, 5.00 eq) in DCM (2.0 mL) was added propionic anhydride (10.0 mg, 77.3 umol, 9.97 uL, 1.50 eq) at 0° C. The mixture was stirred at 25° C. for 2 hr. The reaction was diluted with H₂O (20.0 mL), and extracted with DCM (20.0 mL*3), dried with anhydrous Na₂SO₄, filtrated and concentrated under reduced pressure to give a residue. The residue was purified by Prep-TLC (SiO₂, DCM:MeOH=10:1). Compound 208 (34.0 mg, 50.0 umol, 93.8% yield, 99.6% purity) was obtained as a white solid. LC-MS: rt=0.73 min; m/z=638.4 for [M+H]⁺.

### Example 3.1.5—Synthesis of Compound 209

To a solution of appropriately substituted Int. C (50.0 mg, 85.9 umol, 1.00 eq) and TEA (26 mg, 257 umol, 35.8 uL, 3.00 eq) in DCM (5.00 mL) was added propionic anhydride (22.3 mg, 171 umol, 22.1 uL, 2.00 eq) at 0° C. The mixture was stirred at 25° C. for 2 hrs. The reaction mixture was diluted with H₂O (30.0 mL) and extracted with DCM (30.0 mL*3). The combined organic layers were dried over Na₂SO₄, filtered and concentrated under reduced pressure to give a residue. The residue was purified by Prep-TLC (SiO₂, DCM:MeOH=10:1). The residue was purified by Prep-HPLC (column: Phenomenex Gemini-NX C18 75*30 mm*3 um; mobile phase: [water (0.05% ammonia hydroxide v/v)-ACN]; B %: 23%-53%, 7 min). Compound 209 (20.6 mg, 31.6 umol, 36.8% yield, 97.9% purity) was obtained as a white solid. LC-MS: rt=0.95 min; m/z=638.6 for [M+H]⁺.

### Example 3.1.16—Synthesis of Compound 212

To a solution of appropriately substituted Int. C (40.0 mg, 67.0 umol, 1.00 eq) and TEA (20.3 mg, 201 umol, 28.0 uL, 3.00 eq) in DCM (5.00 mL) was added propionic anhydride (11.3 mg, 87.2 umol, 11.2 uL, 1.30 eq). The mixture was stirred at 25° C. for hr. The reaction mixture was diluted with H₂O (30.0 mL) and extracted with DCM (30.0 mL*3). The combined organic layers were dried over Na₂SO₄, filtered and concentrated under reduced pressure to give a residue. The residue was purified by Prep-TLC (SiO₂, DCM:MeOH=10:1). Compound 212 (21.8 mg, 32.8 umol, 48.9% yield, 98.1% purity) was obtained as a white solid. LC-MS: rt=0.81 min; m/z=652.2 for [M+H]⁺.

### Example 3.1.17—Synthesis of Compound 213

To a solution of appropriately substituted Int. C (35.0 mg, 60.2 umol, 1.00 eq) and TEA (18.3 mg, 181 umol, 25.1 uL, 3.00 eq) in DCM (2.00 mL) was added propionic anhydride (8.61 mg, 66.2 umol, 8.52 uL, 1.10 eq) at 0° C. The mixture was stirred at 15° C. for 1 hr. The reaction mixture was diluted with H₂O (10.0 mL) and extracted with DCM (10.0 mL*3). The combined organic layers were dried over Na₂SO₄, filtered and concentrated under reduced pressure to give a residue. The residue was purified by Prep-TLC (SiO₂, DCM:MeOH=10:1). Compound 213 (28.0 mg, 41.8 umol, 69.4% yield, 95.1% purity) was obtained as a white solid. LC-MS: rt=0.84 min; m/z=638.4 for [M+H]⁺.

### Example 3.1.8—Synthesis of Compound 214

To a solution of appropriately substituted Int. C (120 mg, 205 umol, 1.00 eq) and TEA (62.4 mg, 616 umol, 85.8 uL, 3.00 eq) in DCM (5.00 mL) was added acetic anhydride (41.9 mg, 411 umol, 38.5 uL, 2.00 eq) at 0° C. The mixture was stirred at 25° C. for 2 hrs. The reaction mixture was diluted with H₂O (30.0 mL) and extracted with DCM (30.0 mL*3). The combined organic layers were dried over Na₂SO₄, filtered and concentrated under reduced pressure to give a residue. The residue was purified by Prep-TLC (SiO₂, DCM:MeOH=10:1). Compound 214 (37.1 mg, 57.7 umol, 28.1% yield, 97.4% purity) was obtained as a white solid. LC-MS: rt=0.88 min; m/z=626.1 for [M+H]⁺.

### Example 3.1.9—Synthesis of Compound 216

To a solution of appropriately substituted Int. C (50.0 mg, 85.3 umol, 1.00 eq) and TEA (25.9 mg, 256 umol, 35.6 uL, 3.00 eq) in DCM (5.0 mL) was added propionic anhydride (14.4 mg, 111 umol, 14.3 uL, 1.30 eq) at 25° C. The mixture was stirred at 25° C. for 1 hr. The reaction mixture was concentrated under reduced pressure to give a residue. The residue was purified by Prep-TLC (SiO₂, DCM:MeOH=10:1). Compound 216 (54.0 mg, 80.6 umol, 94.5% yield, 95.8% purity) was obtained as a white solid. LC-MS: rt=0.73 min; m/z=642.5 for [M+H]⁺.

### Example 3.1.10—Synthesis of Compound 217

To a solution of appropriately substituted Int. C (65.0 mg, 108 umol, 1.00 eq) and TEA (32.9 mg, 325 umol, 45.2 uL, 3.00 eq) in DCM (5.0 mL) was added propionic anhydride (21.1 mg, 162 umol, 20.9 uL, 1.50 eq) at 25° C. The mixture was stirred at 25° C. for 1 hr. The reaction mixture was diluted with H₂O (40.0 mL) and extracted with DCM (40.0 mL*3). The combined organic layers were washed with brine (40.0 mL*3), dried over Na₂SO₄, filtered and concentrated under reduced pressure to give a residue. The residue was purified by Prep-TLC (SiO₂, DCM:MeOH=10:1). Compound 217 (69.6 mg, 103 umol, 95.4% yield, 97.5% purity) was obtained as a white solid. LC-MS: rt=0.73 min; m/z=656.5 for [M+H]⁺.

### Example 3.1.11—Synthesis of Compound 232

To a solution of appropriately substituted Int. C (60.0 mg, 99.3 umol, 1.00 eq) and TEA (30.1 mg, 298 umol, 41.5 uL, 3.00 eq) in DCM (5.0 mL) was added propionic anhydride (15.5 mg, 119 umol, 15.3 uL, 1.20 eq). The mixture was stirred at 25° C. for 1 hr. The reaction mixture was diluted with H₂O (40.0 mL) and extracted with DCM (40.0 mL*3). The combined organic layers were washed with brine (40.0 mL*3), dried over Na₂SO₄, filtered and concentrated under reduced pressure to give a residue. The residue was purified by Prep-TLC (SiO₂, DCM:MeOH=10:1). Compound 232 (49.4 mg, 72.5 umol, 73.0% yield, 96.8% purity) was obtained as a white solid. LC-MS: rt=0.81 min; m/z=674.2 for [M+H]⁺.

### Example 3.1.12—Synthesis of Compound 239

To a solution of appropriately substituted Int. C (89.0 mg, 149 umol, 1.00 eq) and TEA (45.5 mg, 449 umol, 62.5 uL, 3.00 eq) in DCM (5.0 mL) was added propionic anhydride (39.0 mg, 299 umol, 38.6 uL, 2.00 eq) at 0° C. The mixture was stirred at 25° C. for 2 hr. The reaction mixture was diluted with H₂O (30.0 mL) and extracted with DCM (30.0 mL*3). The combined organic layers were washed with sat.aq. NaHCO₃ (30.0 mL*2), dried over Na₂SO₄, filtered and concentrated under reduced pressure to give a residue. The residue was purified by Prep-TLC (SiO₂, DCM:MeOH=10:1). Compound 239 (43.5 mg, 66.4 umol, 44.2% yield, 99.0% purity) was obtained as a white solid. LC-MS: rt=0.84 min; m/z=650.3 for [M+H]⁺.

### Example 3.1.13—Synthesis of Compound 245

To a solution of appropriately substituted Int. C (80.0 mg, 146 umol, 1.00 eq) and TEA (14.8 mg, 146 umol, 20.4 uL, 1.00 eq) in DCM (5.00 mL) was added propionic anhydride (28.6 mg, 219 umol, 28.3 uL, 1.50 eq) at 15° C. The mixture was stirred at 15° C. for 12 hrs. The reaction mixture was diluted with H₂O (10.0 mL) and extracted with DCM (20.0 mL*2). The combined organic layers were dried over Na₂SO₄, filtered and concentrated under reduced pressure to give a residue. The residue was purified by Prep-TLC (SiO₂, DCM:MeOH=10:1). Compound 245 (49.3 mg, 76.6 umol, 52.2% yield, 93.5% purity) was obtained as a white solid. LC-MS: rt=0.74 min; m/z=602.4 for [M+H]⁺.

### Example 3.1.14—Synthesis of Compound 250

To a solution of appropriately substituted Int. C (50.0 mg, 87.7 umol, 1.00 eq) and TEA (26.6 mg, 263 umol, 36.6 uL, 3.00 eq) in DCM (3.00 mL) was added propionic anhydride (13.7 mg, 105 umol, 13.5 uL, 1.20 eq) at 0° C. The mixture was stirred at 25° C. for 1 hr. The reaction mixture was concentrated under reduced pressure to give a residue. The residue was purified by Prep-TLC (SiO₂, DCM:MeOH=10:1). Compound 250 (34.6 mg, 53.5 umol, 60.9% yield, 96.7% purity) was obtained as a white solid. LC-MS: rt=0.84 min; m/z=626.2 for [M+H]⁺.

### Example 3.1.15—Synthesis of Compound 257

To a solution of appropriately substituted Int. C (100.0 mg, 172 umol, 1.00 eq) and TEA (34.7 mg, 343 umol, 47.8 uL, 2.00 eq) in DCM (1.0 mL) was added propionic anhydride (33.5 mg, 257 umol, 33.2 uL, 1.50 eq). The mixture was stirred at 20° C. for 2 hr. The reaction mixture was diluted with sat.aq. NaHCO₃ (3.0 mL) and water (3.0 mL), extracted with DCM (3.0 mL*3), the combined organic layer was washed with brine (5.0 mL), dried over Na₂SO₄, filtered and concentrated under reduced pressure to give a residue. The residue was purified by Prep-TLC (SiO₂, EtOAc:MeOH=10:1). Compound 257 (38.4 mg, 58.7 umol, 34.1% yield, 97.6% purity) was obtained as a white solid. LC-MS: rt=0.74 min; m/z=638.4 for [M+H]⁺.

### Example 3.1.16—Synthesis of Compound 258

To a solution of appropriately substituted Int. C (100.0 mg, 168 umol, 1.00 eq) and TEA (33.9 mg, 335 umol, 46.7 uL, 2.00 eq) in DCM (1.0 mL) was added propionic anhydride (32.7 mg, 252 umol, 32.4 uL, 1.50 eq) at 20° C. The mixture was stirred at 20° C. for 2 hr. The reaction mixture was diluted with sat.aq. NaHCO₃ (3.0 mL) and water (3.0 mL), extracted with DCM (3.0 mL*3), the combined organic layer was washed with brine (5.0 mL), dried over Na₂SO₄, filtered and concentrated under reduced pressure to give a residue. The residue was purified by Prep-HPLC (column: Phenomenex Synergi C18 150*25 mm*10 um; mobile phase: [water (0.225% FA)-ACN]; B %: 13%-43%, 10 min) to get the desired product. Compound 258 (60.2 mg, 91.5 umol, 54.5% yield, 99.1% purity) was obtained as a white solid. LC-MS: rt=0.74 min; m/z=652.5 for [M+H]⁺.

### Example 3.1.17—Synthesis of Compound 264

To a solution of appropriately substituted Int. C (100.0 mg, 164 umol, 1.00 eq) and TEA (33.3 mg, 329 umol, 45.8 uL, 2.00 eq) in DCM (1.0 mL) was added propionic anhydride (32.1 mg, 247 umol, 31.8 uL, 1.50 eq) at 20° C. The mixture was stirred at 20° C. for 2 hr. The reaction mixture was diluted with sat.aq. NaHCO₃ (3.0 mL) and water (3.0 mL), extracted with DCM (3.0 mL*3), the combined organic layer was washed with brine (5.0 mL), dried over Na₂SO₄, filtered and concentrated under reduced pressure to give a residue. The residue was purified by Prep-HPLC (column: Phenomenex Synergi C18 150*25 mm*10 um; mobile phase: [water (0.225% FA)-ACN]; B %: 14%-44%, 10 min) to get the desired product. Compound 264 (75.1 mg, 111 umol, 67.7% yield, 98.5% purity) was obtained as a white solid. LC-MS: rt=0.75 min; m/z=664.4 for [M+H]⁺.

### Example 3.1.18—Synthesis of Compound 265

To a solution of appropriately substituted Int. C (100.0 mg, 168 umol, 1.00 eq) and TEA (33.9 mg, 336 umol, 46.7 uL, 2.00 eq) in DCM (1.0 mL) was added propionic anhydride (32.7 mg, 252 umol, 32.4 uL, 1.50 eq). The mixture was stirred at 20° C. for 2 hr. The reaction mixture was diluted with sat.aq. NaHCO₃ (3.0 mL) and water (3.0 mL), extracted with DCM (3.0 mL*3), the combined organic layer was washed with brine (5.0 mL), dried over Na₂SO₄, filtered and concentrated under reduced pressure to give a residue. The residue was purified by Prep-TLC (SiO₂, EtOAc:MeOH=10:1). Compound 265 (32.0 mg, 47.6 umol, 28.3% yield, 97.0% purity) was obtained as a white solid. LC-MS: rt=0.74 min; m/z=652.5 for [M+H]⁺.

### Example 3.1.19—Synthesis of Compound 288

To a solution of appropriately substituted Int. C (130.0 mg, 232 umol, 1.00 eq) and TEA (47.0 mg, 464 umol, 64.6 uL, 2.00 eq) in DCM (2.0 mL) was added propionic anhydride (45.3 mg, 348 umol, 44.8 uL, 1.50 eq) at 20° C. The mixture was stirred at 20° C. for 2 hr. The reaction mixture was diluted with sat.aq. NaHCO₃ (3.0 mL) and water (3.0 mL), extracted with DCM (3.0 mL*3), the combined organic layer was washed with brine (5.0 mL), dried over Na₂SO₄, filtered and concentrated under reduced pressure to give a residue. The residue was purified by Prep-TLC (SiO₂, Plate 1, EtOAc:MeOH=5:1, R_{f}=0.5). The residue was then purified by Prep-HPLC (column: Phenomenex Synergi C18 150*25 mm*10 um; mobile phase: [water (0.225% FA)-ACN]; B %: 13%-43%, 10 min) to get the desired product. Compound 288 (57.4 mg, 92.6 umol, 39.8% yield, 99.3% purity) was obtained as a white solid. LC-MS: rt=0.73 min; m/z=616.4 for [M+H]⁺.

### Example 3.2.1—Synthesis of Compound 224

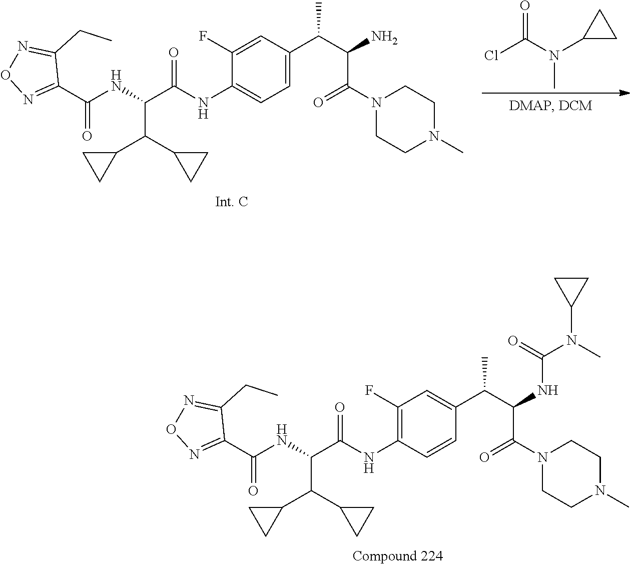

To a solution of Int. C (80.0 mg, 140 umol, 1.00 eq) and N-cyclopropyl-N-methylcarbamoyl chloride (37.5 mg, 280 umol, 2.00 eq) in DCM (3.00 mL) was added DMAP (85.7 mg, 702 umol, 5.00 eq). The mixture was stirred at 25° C. for 8 hrs. The reaction mixture was diluted with H₂O (40.0 mL) and extracted with DCM (40.0 mL*3). The combined organic layers were washed with brine (40.0 mL*3), dried over Na₂SO₄, filtered and concentrated under reduced pressure to give a residue. The residue was purified by Prep-TLC (SiO₂, Ethyl acetate:Methanol=5:1). Compound 224 (37.0 mg, 54.1 umol, 38.5% yield, 97.5% purity) was obtained as a white solid. LC-MS: rt=0.79 min; m/z=667.4 for [M+H]⁺.

Compounds 225, 270, 271, 291, 295, 313, and 316 were similarly synthesized using similar reaction conditions with the appropriate carbamoyl chloride reagent.

### Example 3.2.2—Synthesis of Compound 225

To a solution of appropriately substituted Int. C (200 mg, 352 umol, 1.00 eq) and N-cyclopropyl-N-methylcarbamoyl chloride (470 mg, 3,520 umol, 10.00 eq) in DCM (5.00 mL) was added DMAP (150 mg, 1,230 umol, 3.50 eq). The mixture was stirred at 20° C. for 52 hrs. The reaction mixture was diluted with H₂O (20 mL) and extracted with DCM (20 mL*3). The combined organic layers were washed with brine (40.0 mL), dried over Na₂SO₄, filtered and concentrated under reduced pressure to give a residue. The residue was purified by Prep-TLC (SiO₂, DCM:Methanol=10:1). Compound 225 (120 mg, 177 umol, 50.4% yield, 98.3% purity) was obtained as a white solid. LC-MS: rt=0.90 min; m/z=665.1 for [M+H]⁺.

### Example 3.3.1—Synthesis of Compound 206

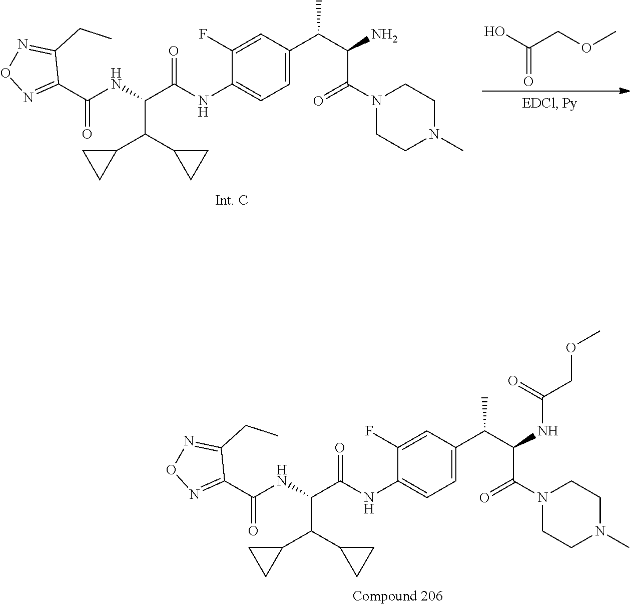

To a solution of Int. C (50.0 mg, 87.7 umol, 1.00 eq) and methoxyacetic acid (10.2 mg, 114 umol, 8.71 uL, 1.30 eq) in pyridine (3.00 mL) was added EDCl (50.4 mg, 263 umol, 3.00 eq). The mixture was stirred at 25° C. for 3 hrs. The reaction mixture was concentrated under reduced pressure to give a residue. The residue was purified by Prep-HPLC (column: Waters Xbridge 150*25 mm*5 um; mobile phase: [water (0.05% ammonia hydroxide v/v)-ACN]; B %: 35%-62%, 9 min). Compound 206 (30.8 mg, 46.9 umol, 53.5% yield, 97.6% purity) was obtained as a white solid. LC-MS: rt=0.83 min; m/z=642.2 for [M+H]⁺.

Compounds 207, 210, 211, 215, 218, 219, 220, 221, 222, 226, 227, 228, 254, 255, 256, 259, 260, 261, 262, 263, 269, 272-287, 289, 290, 292-294, 298-303, 307-312, 314, 315, 317-342, 345-349, 361, 362, 343, and 344 were similarly synthesized using similar reaction conditions with the appropriate carboxylic acid reagent.

### Example 3.3.2—Synthesis of Compound 207

To a solution of appropriately substituted Int. C (50.0 mg, 85.9 umol, 1.00 eq) and methoxyacetic acid (11.6 mg, 128 umol, 9.84 uL, 1.50 eq) in pyridine (5.00 mL) was added EDCl (49.4 mg, 257 umol, 3.00 eq). The mixture was stirred at 25° C. for 2 hrs. The reaction mixture was diluted with H₂O (30.0 mL) and extracted with ethyl acetate (30.0 mL*3). The combined organic layers were dried over Na₂SO₄, filtered and concentrated under reduced pressure to give a residue. The residue was purified by Prep-TLC (SiO₂, DCM:MeOH=10:1). Compound 207 (49.3 mg, 71.2 umol, 82.9% yield, 94.4% purity) was obtained as a white solid. LC-MS: rt=0.94 min; m/z=654.6 for [M+H]⁺.

### Example 3.3.3—Synthesis of Compound 210

To a solution of appropriately substituted Int. C (50.0 mg, 85.9 umol, 1.00 eq) and N-(ethoxycarbonyl)-N-methyl-L-alanine (18.0 mg, 103 umol, 1.20 eq) in pyridine (5.0 mL) was added EDCl (49.4 mg, 257 umol, 3.00 eq). The mixture was stirred at 25° C. for 2 hrs. The reaction mixture was diluted with H₂O (30.0 mL) and extracted with ethyl acetate (30.0 mL*3). The combined organic layers were dried over Na₂SO₄, filtered and concentrated under reduced pressure to give a residue. The residue was purified by Prep-TLC (SiO₂, DCM:MeOH=10:1). Then the residue was purified by Prep-HPLC (basic condition, column: Phenomenex Gemini-NX C18 75*30 mm*3 um; mobile phase: [water (0.225% FA)-ACN]; B %: 20%-50%, 7 min). Compound 210 (13.2 mg, 17.0 umol, 19.8% yield, 94.8% purity) was obtained as a white solid. LC-MS: rt=0.99 min; m/z=739.6 for [M+H]⁺.

### Example 3.3.4—Synthesis of Compound 211

To a solution of appropriately substituted Int. C (60.0 mg, 105 umol, 1.00 eq) and N-(ethoxycarbonyl)-N-methyl-L-alanine (24.1 mg, 137 umol, 1.30 eq) in pyridine (2.0 mL) was added EDCl (40.5 mg, 211 umol, 2.00 eq). The mixture was stirred at 20° C. for 1 hr. The reaction mixture was diluted with DCM (30.0 mL) and washed with water (20.0 mL) and brine (20.0 mL), dried over Na₂SO₄ and concentrated under reduced pressure to give a residue. The residue was purified by Prep-TLC (SiO₂, DCM:MeOH=10:1). Compound 211 (42.0 mg, 57.9 umol, 54.8% yield, 91.7% purity) was obtained as a white solid. LC-MS: rt=0.97 min; m/z=725.6 for [M+H]⁺.

### Example 3.3.5—Synthesis of Compound 215

To a solution of appropriately substituted Int. C (60.0 mg, 106 umol, 1.00 eq) and methoxyacetic acid (14.3 mg, 159 umol, 12.1 uL, 1.50 eq) in pyridine (1.00 mL) was added EDCl (60.8 mg, 318 umol, 3.00 eq). The mixture was stirred at 15° C. for 3 hrs. The reaction mixture was diluted with H₂O (15 mL) and extracted with ethyl acetate (20 mL*3). The combined organic layers were dried over Na₂SO₄, filtered and concentrated under reduced pressure to give a residue. The residue was purified by Prep-TLC (SiO₂, DCM:MeOH=10:1). Compound 215 (43.7 mg, 67.9 umol, 64.2% yield, 99.4% purity) was obtained as a white solid. LC-MS: rt=0.74 min; m/z=640.4 for [M+H]⁺.

### Example 3.3.6—Synthesis of Compound 219

To a solution of appropriately substituted Int. C (45.0 mg, 77.3 umol, 1.00 eq) and the sodium salt of 2-fluoropropanoic acid (10.6 mg, 92.8 umol, 1.20 eq) in DMF (5.0 mL) was added HOBT (20.9 mg, 154 umol, 2.00 eq) and DIEA (20.0 mg, 154 umol, 26.9 uL, 2.00 eq) and EDCl (29.6 mg, 154 umol, 2.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq NaHCO₃ (30.0 mL*2), dried over Na₂SO₄, filtered and concentrated under reduced pressure to give a residue. The residue was purified by Prep-TLC (SiO₂, DCM:MeOH=10:1). Compound 219 (39.0 mg, 57.2 umol, 74.0% yield, 96.1% purity) was obtained as a white solid. LC-MS: rt=0.96 min; m/z=656.5 for [M+H]⁺.

### Example 3.3.7—Synthesis of Compound 220

To a solution of appropriately substituted Int. C (50.0 mg, 87.7 umol, 1.00 eq) and (S)-2-methoxypropanoic acid (11.8 mg, 114 umol, 1.30 eq) in pyridine (4.0 mL) was added EDCl (50.4 mg, 263 umol, 3.00 eq). The mixture was stirred at 25° C. for 3 hrs. The reaction mixture was concentrated under reduced pressure to give a residue. The residue was purified by Prep-HPLC (column: Waters Xbridge 150*25 mm*5 um; mobile phase: [water (0.05% ammonia hydroxide v/v)-ACN]; B %: 37%-64%, 9 min). Compound 220 (33.6 mg, 51.1 umol, 58.3% yield, 99.8% purity) was obtained as a white solid. LC-MS: rt=0.85 min; m/z=656.3 for [M+H]⁺.

### Example 3.3.8—Synthesis of Compound 221

To a solution of appropriately substituted Int. C (50.0 mg, 85.9 umol, 1.00 eq) and (S)-2-methoxypropanoic acid (13.4 mg, 128 umol, 1.50 eq) in pyridine (5.00 mL) was added EDCl (49.4 mg, 257 umol, 3.00 eq). The mixture was stirred at 25° C. for 2 hrs. The reaction mixture was diluted with H₂O (30.0 mL) and extracted with ethyl acetate (30.0 mL*3). The combined organic layers were dried over Na₂SO₄, filtered and concentrated under reduced pressure to give a residue. The residue was purified by Prep-TLC (SiO₂, DCM:MeOH=10:1). Compound 221 (37.7 mg, 54.1 umol, 62.9% yield, 95.7% purity) was obtained as a white solid. LC-MS: rt=0.94 min; m/z=668.5 for [M+H]⁺.

### Example 3.3.9—Synthesis of Compound 222

To a solution of appropriately substituted Int. C (60.0 mg, 106 umol, 1.00 eq) and (S)-2-methoxypropanoic acid (22.0 mg, 211 umol, 2.00 eq) in pyridine (2.00 mL) was added EDCl (60.8 mg, 317 umol, 3.00 eq). The mixture was stirred at 15° C. for 3 hrs. The reaction mixture was diluted with H₂O (15 mL) and extracted with ethyl acetate (20 mL*3). The combined organic layers were dried over Na₂SO₄, filtered and concentrated under reduced pressure to give a residue. The residue was purified by Prep-TLC (SiO₂, DCM:MeOH=10:1). Compound 222 (40.4 mg, 60.8 umol, 57.5% yield, 98.4% purity) was obtained as a white solid. LC-MS: rt=0.75 min; m/z=654.4 for [M+H]⁺.

### Example 3.3.10—Synthesis of Compound 226

To a solution of appropriately substituted Int. C (50.0 mg, 85.9 umol, 1.00 eq) and 2-cyclopropylacetic acid (12.9 mg, 128 umol, 1.50 eq) in pyridine (5.0 mL) was added EDCl (49.4 mg, 257 umol, 3.00 eq). The mixture was stirred at 25° C. for 2 hrs. The reaction mixture was diluted with H₂O (30.0 mL) and extracted with ethyl acetate (30.0 mL*3). The combined organic layers were dried over Na₂SO₄, filtered and concentrated under reduced pressure to give a residue. The residue was purified by Prep-TLC (SiO₂, DCM:MeOH=10:1). Compound 226 (45.4 mg, 66.8 umol, 77.7% yield, 97.7% purity) was obtained as a white solid. LC-MS: rt=0.97 min; m/z=664.6 for [M+H]⁺.

### Example 3.3.11—Synthesis of Compound 227

To a solution of appropriately substituted Int. C (60.0 mg, 85.9 umol, 1.00 eq) and 2-cyclopropylacetic acid (13.7 mg, 137 umol, 1.30 eq) in pyridine (2.0 mL) was added EDCl (40.5 mg, 211 umol, 2.00 eq). The mixture was stirred at 20° C. for 1 hr. The reaction mixture was diluted with DCM (30.0 mL) and washed with water (20.0 mL) and brine (20.0 mL), dried over Na₂SO₄ and concentrated under reduced pressure to give a residue. The residue was purified by Prep-TLC (SiO₂, DCM:MeOH=10:1). Compound 227 (60.0 mg, 92.3 umol, 87.3% yield, 96.6% purity) was obtained as a white solid. LC-MS: rt=0.95 min; m/z=650.6 for [M+H]⁺.

### Example 3.3.12—Synthesis of Compound 228

To a solution of appropriately substituted Int. C (70.0 mg, 120 umol, 1.00 eq) and the sodium salt of 2-fluoroacetic acid (19.1 mg, 180 umol, 1.50 eq) in DMF (5.0 mL) was added HOBT (32.5 mg, 240 umol, 2.00 eq) and DIEA (31.1 mg, 240 umol, 41.9 uL, 2.00 eq) and EDCl (46.1 mg, 240 umol, 2.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq NaHCO₃ (30.0 mL*2), dried over Na₂SO₄, filtered and concentrated under reduced pressure to give a residue. The residue was purified by Prep-HPLC (FA condition, column: Phenomenex Gemini-NX C18 75*30 mm*3 um; mobile phase: [water (0.225% FA)-ACN]; B %: 15%-45%, 7 min). Compound 228 (26.7 mg, 39.3 umol, 32.7% yield, 94.4% purity) was obtained as a white solid. LC-MS: rt=0.94 min; m/z=642.5 for [M+H]⁺.

### Example 3.3.13—Synthesis of Compound 260

To a solution of appropriately substituted Int. C (100.0 mg, 168 umol, 1.00 eq) and (S)-2-methoxypropanoic acid (20.9 mg, 201 umol, 1.20 eq) in pyridine (1.0 mL) was added EDCl (64.3 mg, 335 umol, 2.00 eq). The mixture was stirred at 20° C. for 2 hrs. The reaction mixture was diluted with sat.aq. NaHCO₃ (3.0 mL) and H₂O (3.0 mL) and extracted with DCM (3.0 mL*3). The combined organic layers were dried over Na₂SO₄, filtered and concentrated under reduced pressure to give a residue. The residue was purified by Prep-HPLC (column: Phenomenex Synergi C18 150*25 mm*10 um; mobile phase: [water (0.225% FA)-ACN]; B %: 14%-44%, 10 min) to get the desired product. Compound 260 (61.8 mg, 88.9 umol, 52.9% yield, 98.2% purity) was obtained as a white solid. LC-MS: rt=0.74 min; m/z=682.6 for [M+H]⁺.

### Example 3.3.14—Synthesis of Compound 261

To a solution of appropriately substituted Int. C (100.0 mg, 164 umol, 1.00 eq) and (S)-2-methoxypropanoic acid (20.5 mg, 197 umol, 25.4 uL, 1.20 eq) in pyridine (1.0 mL) was added EDCl (63.0 mg, 329 umol, 2.00 eq). The mixture was stirred at 20° C. for 2 hrs. The reaction mixture was diluted with sat.aq. NaHCO₃ (3.0 mL) and water (3.0 mL), extracted with DCM (3.0 mL*3), the combined organic layer was washed with brine (5.0 mL), dried over Na₂SO₄, filtered and concentrated under reduced pressure to give a residue. The residue was purified by Prep-HPLC (column: Phenomenex Synergi C18 150*25 mm*10 um; mobile phase: [water (0.225% FA)-ACN]; B %: 14%-44%, 10 min) to get the desired product. Compound 261 (72.9 mg, 104 umol, 63.5% yield, 99.4% purity) was obtained as a white solid. LC-MS: rt=0.75 min; m/z=694.4 for [M+H]⁺.

### Example 3.3.15—Synthesis of Compound 269

To a solution of appropriately substituted Int. C (70.0 mg, 131 umol, 1.00 eq) and (ethoxycarbonyl)-L-proline (42.4 mg, 263 umol, 2.00 eq) in pyridine (5.0 mL) was added EDCl (75.7 mg, 395 umol, 3.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq NaHCO₃ (30 mL*2), dried over Na₂SO₄, filtered and concentrated under reduced pressure to give a residue. The residue was purified by Prep-TLC (SiO₂, DCM:MeOH=10:1). Compound 269 (34.5 mg, 50.8 umol, 38.6% yield, 99.2% purity) was obtained as a white solid. LC-MS: rt=0.83 min; m/z=675.5 for [M+H]⁺.

### Example 3.3.16—Synthesis of Compound 272

To a solution of appropriately substituted Int. C (50.0 mg, 94.0 umol, 1.00 eq) and (S)-2-methoxypropanoic acid (14.6 mg, 141 umol, 1.50 eq) in pyridine (5.0 mL) was added EDCl (54.0 mg, 282 umol, 3.00 eq). The mixture was stirred at 25° C. for 2 hrs. The reaction mixture was diluted with H₂O (30.0 mL) and extracted with DCM (30.0 mL*3). The combined organic layers were washed with sat.aq. NaHCO₃ (30.0 mL*2), dried over Na₂SO₄, filtered and concentrated under reduced pressure to give a residue. The residue was purified by Prep-TLC (SiO₂, DCM:MeOH=10:1). Compound 272 (29.5 mg, 45.5 umol, 48.4% yield, 95.2% purity) was obtained as a white solid. LC-MS: rt=0.82 min; m/z=618.2 for [M+H]⁺.

### Example 3.3.17—Synthesis of Compound 273

To a solution of appropriately substituted Int. C (50.0 mg, 91.6 umol, 1.00 eq) and (S)-2-methoxypropanoic acid (14.3 mg, 137 umol, 1.50 eq) in DCM (5.00 mL) was added EDCl (52.7 mg, 274 umol, 3.00 eq). The mixture was stirred at 25° C. for 2 hrs. The reaction mixture was diluted with H₂O (30.0 mL) and extracted with ethyl acetate (30.0 mL*3). The combined organic layers were dried over Na₂SO₄, filtered and concentrated under reduced pressure to give a residue. The residue was purified by Prep-TLC (SiO₂, DCM:MeOH=10:1). Compound 273 (32.5 mg, 48.8 umol, 53.3% yield, 94.7% purity) was obtained as a white solid. LC-MS: rt=0.83 min; m/z=632.2 for [M+H]⁺.

### Example 3.3.18—Synthesis of Compound 274

To a solution of appropriately substituted Int. C (60.0 mg, 112 umol, 1.00 eq) and (S)-1-(ethoxycarbonyl)azetidine-2-carboxylic acid (29.3 mg, 169 umol, 1.50 eq) in pyridine (5.0 mL) was added EDCl (64.9 mg, 338 umol, 3.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq. NaHCO₃ (30.0 mL*2), dried over Na₂SO₄, filtered and concentrated under reduced pressure to give a residue. The residue was purified by Prep-TLC (SiO₂, DCM:MeOH=10:1). Compound 274 (32.8 mg, 46.7 umol, 41.4% yield, 97.9% purity) was obtained as a white solid. LC-MS: rt=0.97 min; m/z=687.5 for [M+H]⁺.

### Example 3.3.19—Synthesis of Compound 275

To a solution of appropriately substituted Int. C (190.0 mg, 348 umol, 1.00 eq) and (S)-1-(ethoxycarbonyl)azetidine-2-carboxylic acid (90.4 mg, 522 mmol, 1.50 eq) in pyridine (5.0 mL) was added EDCl (200 mg, 1.04 mmol, 3.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq NaHCO₃ (30.0 mL*2), dried over Na₂SO₄, filtered and concentrated under reduced pressure to give a residue. The residue was purified by Prep-TLC (SiO₂, DCM:MeOH=10:1). Compound 275 (85.9 mg, 118 umol, 34.1% yield, 97.0% purity) was obtained as a white solid. LC-MS: rt=0.82 min; m/z=701.3 for [M+H]⁺.

### Example 3.3.20—Synthesis of Compound 276

To a solution of appropriately substituted Int. C (70.0 mg, 131 umol, 1.00 eq) and (ethoxycarbonyl)-L-proline (49.2 mg, 263 umol, 2.00 eq) in pyridine (5.0 mL) was added EDCl (75.7 mg, 395 umol, 3.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq. NaHCO₃ (30.0 mL*2), dried over Na₂SO₄, filtered and concentrated under reduced pressure to give a residue. The residue was purified by Prep-TLC (SiO₂, DCM:MeOH=10:1). Compound 276 (52.2 mg, 72.2 umol, 54.8% yield, 96.9% purity) was obtained as a white solid. LC-MS: rt=0.98 min; m/z=701.5 for [M+H]⁺.

### Example 3.3.21—Synthesis of Compound 278

To a solution of appropriately substituted Int. C (70.0 mg, 128 umol, 1.00 eq) and (S)-1-(methoxycarbonyl)azetidine-2-carboxylic acid (30.6 mg, 192 umol, 1.50 eq) in pyridine (2.0 mL) was added EDCl (49.2 mg, 257 umol, 2.00 eq). The mixture was stirred at 20° C. for 2 hrs. The reaction mixture was diluted with DCM (20.0 mL), and extracted with sat.aq. NaHCO₃ (10.0 mL) and DCM (10.0 mL*2), the organic phase was washed with water (20.0 mL) and brine (20.0 mL), then dried over Na₂SO₄ and concentrated under reduced pressure to give a residue. The residue was purified by Prep-TLC (SiO₂, DCM:MeOH=10:1). Compound 278 (55.0 mg, 78.9 umol, 61.5% yield, 98.5% purity) was obtained as a white solid. LC-MS: rt=0.73 min; m/z=687.4 for [M+H]⁺.

### Example 3.3.22—Synthesis of Compound 279

To a solution of appropriately substituted Int. C (70.0 mg, 128 umol, 1.00 eq) and (ethoxycarbonyl)-L-proline (48.0 mg, 256 umol, 2.00 eq) in pyridine (5.0 mL) was added EDCl (73.7 mg, 384 umol, 3.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq NaHCO₃ (30 mL*2), dried over Na₂SO₄, filtered and concentrated under reduced pressure to give a residue. The residue was purified by Prep-TLC (SiO₂, DCM:MeOH=10:1). Compound 279 (54.1 mg, 72.6 umol, 56.6% yield, 96.0% purity) was obtained as a white solid. LC-MS: rt=0.99 min; m/z=715.5 for [M+H]⁺.

### Example 3.3.23—Synthesis of Compound 280

To a solution of appropriately substituted Int. C (70.0 mg, 132 umol, 1.00 eq) and (methoxycarbonyl)-L-proline (34.2 mg, 198 umol, 1.50 eq) in pyridine (2.0 mL) was added EDCl (50.5 mg, 263 umol, 2.00 eq). The mixture was stirred at 20° C. for 12 hrs. The reaction mixture was diluted with sat.aq. NaHCO₃ (10.0 mL) and extracted with DCM (10.0 mL*5), the combined organic phase was washed with water (10.0 mL) and brine (10.0 mL), then dried over Na₂SO₄ and concentrated under reduced pressure to give a residue. The residue was purified by Prep-TLC (SiO₂, DCM:MeOH=10:1). Compound 280 (60.0 mg, 87.4 umol, 66.4% yield, 98.3% purity) was obtained as a white solid. LC-MS: rt=0.74 min; m/z=687.4 for [M+H].

### Example 3.3.24—Synthesis of Compound 281

To a solution of appropriately substituted Int. C (70.0 mg, 128 umol, 1.00 eq) and (methoxycarbonyl)-L-proline (33.3 mg, 192 umol, 1.50 eq) in pyridine (2.0 mL) was added EDCl (49.2 mg, 257 umol, 2.00 eq). The mixture was stirred at 20° C. for 12 hrs. The reaction mixture was diluted with sat.aq. NaHCO₃ (10.0 mL) and extracted with DCM (10.0 mL*5), the combined organic phase was washed with water (10.0 mL) and brine (10.0 mL), then dried over Na₂SO₄ and concentrated under reduced pressure to give a residue. The residue was purified by Prep-TLC (SiO₂, DCM:MeOH=10:1). Compound 281 (55.0 mg, 77.9 umol, 60.7% yield, 99.3% purity) was obtained as a white solid. LC-MS: rt=0.81 min; m/z=701.2 for [M+H]⁺.

### Example 3.3.25—Synthesis of Compound 282

To a solution of appropriately substituted Int. C (70.0 mg, 128 umol, 1.00 eq) and (ethoxycarbonyl)-L-alanine (41.3 mg, 256 umol, 2.00 eq) in pyridine (5.0 mL) was added EDCl (73.7 mg, 384 umol, 3.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq NaHCO₃ (30.0 mL*2), dried over Na₂SO₄, filtered and concentrated under reduced pressure to give a residue. The residue was purified by Prep-TLC (SiO₂, DCM:MeOH=10:1). Compound 282 (39.1 mg, 55.6 umol, 43.3% yield, 98.1% purity) was obtained as a white solid. LC-MS: rt=0.83 min; m/z=689.5 for [M+H]⁺.

### Example 3.3.26—Synthesis of Compound 286

To a solution of appropriately substituted Int. C (90.0 mg, 151 umol, 1.00 eq) and 2-methoxyacetic acid (20.4 mg, 226 umol, 1.50 eq) in pyridine (5.0 mL) was added EDCl (86.8 mg, 453 umol, 3.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq. NaHCO₃ (30.0 mL*2), dried over Na₂SO₄, filtered and concentrated under reduced pressure to give a residue. The residue was purified by Prep-TLC (SiO₂, DCM:MeOH=10:1). Compound 286 (100 mg, 148 umol, 98.0% yield, 97.9% purity) was obtained as a white solid. LC-MS: rt=0.82 min; m/z=668.3 for [M+H]⁺.

### Example 3.3.27—Synthesis of Compound 287

To a solution of appropriately substituted Int. C (90.0 mg, 151 umol, 1.00 eq) and (S)-2-methoxypropanoic acid (23.5 mg, 226 umol, 1.50 eq) in pyridine (5.0 mL) was added EDCl (86.8 mg, 453 umol, 3.00 eq). The mixture was stirred at 25° C. for 2 hrs. The reaction mixture was diluted with H₂O (30.0 mL) and extracted with DCM (30.0 mL*3). The combined organic layers were washed with sat.aq. NaHCO₃ (30.0 mL*2), dried over Na₂SO₄, filtered and concentrated under reduced pressure to give a residue. The residue was purified by Prep-TLC (SiO₂, DCM:MeOH=10:1). Compound 287 (39.3 mg, 57.3 umol, 37.9% yield, 99.4% purity) was obtained as a white solid. LC-MS: rt=0.82 min; m/z=682.3 for [M+H]⁺.

### Example 3.3.28—Synthesis of Compound 289

To a solution of appropriately substituted Int. C (130.0 mg, 232 umol, 1.00 eq) and methoxyacetic acid (25.1 mg, 278 umol, 21.2 uL, 1.20 eq) in pyridine (5.0 mL) was added EDCl (89.0 mg, 464 umol, 2.00 eq). The mixture was stirred at 20° C. for 2 hrs. The reaction mixture was diluted with sat.aq. NaHCO₃ (3.0 mL) and H₂O (3.0 mL) and extracted with DCM (3.0 mL*3). The combined organic layers were dried over Na₂SO₄, filtered and concentrated under reduced pressure to give a residue. The residue was purified by Prep-TLC (SiO₂, Plate 1, EtOAc:MeOH=5:1, R_{f}=0.5). Then the residue was purified by Prep-HPLC (column: Phenomenex Synergi C18 150*25 mm*10 um; mobile phase: [water (0.225% FA)-ACN]; B %: 14%-44%, 10 min) to get the desired product. Compound 289 (27.7 mg, 43.1 umol, 18.6% yield, 98.4% purity) was obtained as a white solid. LC-MS: rt=0.73 min; m/z=632.4 for [M+H]⁺.

### Example 3.3.29—Synthesis of Compound 290

To a solution of appropriately substituted Int. C (130.0 mg, 232 umol, 1.00 eq) and (S)-2-methoxypropanoic acid (29.0 mg, 279 umol, 1.20 eq) in pyridine (2.0 mL) was added EDCl (89.0 mg, 464 umol, 2.00 eq). The mixture was stirred at 20° C. for 2 hrs. The reaction mixture was diluted with sat.aq. NaHCO₃ (3.0 mL) and H₂O (3.0 mL) and extracted with DCM (3.0 mL*3). The combined organic layers were dried over Na₂SO₄, filtered and concentrated under reduced pressure to give a residue. The residue was purified by Prep-TLC (SiO₂, Plate 1, EtOAc:MeOH=5:1, R_{f}=0.5). Then the residue was purified by Prep-HPLC (column: Phenomenex Synergi C18 150*25 mm*10 um; mobile phase: [water (0.225% FA)-ACN]; B %: 14%-44%, 10 min) to get the desired product. Compound 290 (52.9 mg, 81.1 umol, 34.9% yield, 99.0% purity) was obtained as a white solid. LC-MS: rt=0.74 min; m/z=646.4 for [M+H]⁺.

### Example 3.3.30—Synthesis of Compound 308

To a solution of appropriately substituted Int. C (70.0 mg, 122 umol, 1.00 eq) and (S)-2-methoxypropanoic acid (25.5 mg, 245 umol, 2.00 eq) in pyridine (5.0 mL) was added EDCl (70.6 mg, 368 umol, 3.00 eq). The mixture was stirred at 25° C. for 1 hr. The reaction mixture was diluted with H₂O (40.0 mL) and extracted with DCM (40.0 mL*3). The combined organic layers were washed with brine (40.0 mL*3), dried over Na₂SO₄, filtered and concentrated under reduced pressure to give a residue. The residue was purified by Prep-TLC (SiO₂, DCM:MeOH=10:1). Compound 308 (63.5 mg, 93.5 umol, 76.0% yield, 96.5% purity) was obtained as a white solid. LC-MS: rt=0.76 min; m/z=656.3 for [M+H]⁺.

### Example 3.3.31—Synthesis of Compound 319

To a solution of appropriately substituted Int. C (70.0 mg, 120 umol, 1.00 eq) and 2,2-dicyclopropylacetic acid (20.2 mg, 144 umol, 1.20 eq) in pyridine (1.0 mL) was added EDCl (46.1 mg, 240 umol, 2.00 eq). The mixture was stirred at 20° C. for 2 hrs. The reaction mixture was diluted with sat.aq. NaHCO₃ (3.0 mL) and water (3.0 mL), extracted with DCM (3.0 mL*3), the combined organic layer was washed with brine (5 mL), dried over Na₂SO₄, filtered and concentrated under reduced pressure to give a residue. The residue was purified by Prep-TLC (SiO₂, EtOAc:MeOH=10:1). Compound 319 (50.6 mg, 69.4 umol, 57.6% yield, 96.5% purity) was obtained as a white solid. LC-MS: rt=0.78 min; m/z=704.4 for [M+H]⁺.

### Example 3.3.32—Synthesis of Compound 342

To a solution of appropriately substituted Int. C (120.0 mg, 202 umol, 1.00 eq) and (S)-2-methoxypropanoic acid (31.5 mg, 303 umol, 1.50 eq) in pyridine (5.0 mL) was added EDCl (116 mg, 606 umol, 3.00 eq). The mixture was stirred at 25° C. for 2 hrs. The reaction mixture was diluted with H₂O (30.0 mL) and extracted with ethyl acetate (30.0 mL*3). The combined organic layers were washed with sat.aq. NaHCO₃ (30.0 mL*2), dried over Na₂SO₄, filtered and concentrated under reduced pressure to give a residue. The residue was purified by Prep-TLC (SiO₂, DCM:MeOH=10:1). Compound 342 (84.7 mg, 119 umol, 59.0% yield, 95.7% purity) was obtained as a white solid. LC-MS: rt=0.82 min; m/z=680.5 for [M+H]⁺.

### Example 3.3.33—Synthesis of Compound 347

To a solution of appropriately substituted Int. C (100.0 mg, 179 umol, 1.00 eq) and (S)-2-methoxypropanoic acid (28.1 mg, 269 umol, 1.50 eq) in pyridine (5.0 mL) was added EDCl (103 mg, 539 umol, 3.00 eq). The mixture was stirred at 20° C. for 3 hrs. The reaction mixture was concentrated under reduced pressure to give a residue and diluted with DCM (30.0 mL), and the combined organic phase was washed with water (10.0 mL) and brine (10.0 mL), then dried over Na₂SO₄ and concentrated under reduced pressure to give a residue. The residue was purified by Prep-TLC (SiO₂, DCM:MeOH=10:1). Compound 347 (34.4 mg, 52.9 umol, 29.4% yield, 98.6% purity) was obtained as a white solid. LC-MS: rt=0.98 min; m/z=642.5 for [M+H]⁺.

### Example 3.3.34—Synthesis of Compound 348

To a solution of appropriately substituted Int. C (80.0 mg, 140 umol, 1.00 eq) and 2-methoxyacetic acid (18.9 mg, 210 umol, 16.0 uL, 1.50 eq) in pyridine (5.0 mL) was added EDCl (80.7 mg, 421 umol, 3.00 eq). The mixture was stirred at 20° C. for 2 hrs. The reaction mixture was concentrated under reduced pressure to give a residue, and the residue was diluted with DCM (30.0 mL) and the combined organic phase was washed with water (10.0 mL) and brine (10.0 mL), then dried over Na₂SO₄ and concentrated under reduced pressure to give a residue. The residue was purified by Prep-TLC (SiO₂, DCM:MeOH=10:1). Compound 348 (45.0 mg, 68.2 umol, 48.5% yield, 97.3% purity) was obtained as a white solid. LC-MS: rt=0.97 min; m/z=642.5 for [M+H]⁺.

### Example 3.3.35—Synthesis of Compound 361

To a solution of appropriately substituted Int. C (75.0 mg, 134 umol, 1.00 eq) and (ethoxycarbonyl)-L-alanine (43.5 mg, 269 umol, 2.00 eq) in pyridine (3.0 mL) was added EDCl (51.7 mg, 269 umol, 2.00 eq). The mixture was stirred at 25° C. for 2 hrs. The reaction mixture was diluted with H₂O (30.0 mL) and extracted with ethyl acetate (30.0 mL*3). The combined organic layers were dried over Na₂SO₄, filtered and concentrated under reduced pressure to give a residue. The residue was purified by Prep-HPLC (neutral condition; column: Waters Xbridge 150*25 mm*5 um; mobile phase: [water (10 mM NH₄HCO₃)-ACN]; B %: 40%-70%, 10 min). Compound 361 (17.6 mg, 23.1 umol, 17.1% yield, 92.1% purity) was obtained as a white solid. LC-MS: rt=0.75 min; m/z=699.4 for [M+H]⁺.

### Example 3.3.36—Synthesis of Compound 362

To a solution of appropriately substituted Int. C (75.0 mg, 131 umol, 1.00 eq) and (ethoxycarbonyl)-L-alanine (42.4 mg, 263 umol, 2.00 eq) in pyridine (2.0 mL) was added EDCl (75.7 mg, 394 umol, 3.00 eq). The mixture was stirred at 25° C. for 2 hrs. The reaction mixture was diluted with H₂O (30.0 mL) and extracted with ethyl acetate (30.0 mL*3). The combined organic layers were dried over Na₂SO₄, filtered and concentrated under reduced pressure to give a residue. The residue was purified by Prep-HPLC (neutral condition; column: Waters Xbridge 150*25 mm*5 um; mobile phase: [water (10 mM NH₄HCO₃)-ACN]; B %: 42%-72%, 10 min). Compound 362 (22.8 mg, 30.8 umol, 23.4% yield, 96.10% purity) was obtained as a white solid. LC-MS: rt=0.85 min; m/z=713.3 for [M+H]⁺.

### Example 3.4—Synthesis of Compound 360

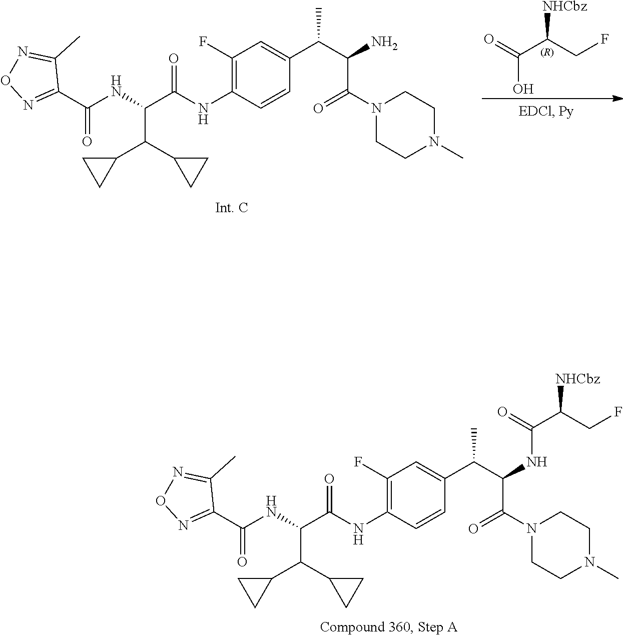

Step A: To a solution of Int. C (70.0 mg, 122 umol, 1.00 eq) and (2R)-2-{[(benzyloxy)carbonyl]amino}-3-fluoropropanoic acid (59.2 mg, 245 umol, 2.00 eq) in pyridine (2.00 mL) was added EDCl (70.6 mg, 368 umol, 3.00 eq). The mixture was stirred at 25° C. for 2 hrs. The reaction was dilute with H₂O (20.0 mL), filtered and concentrated in vacuum. Compound 360, Step A 90.0 g, 113 umol, 92.3% yield) was obtained as yellow solid. LC-MS: rt=0.87 min; m/z=779.2 for [M+H]⁺.

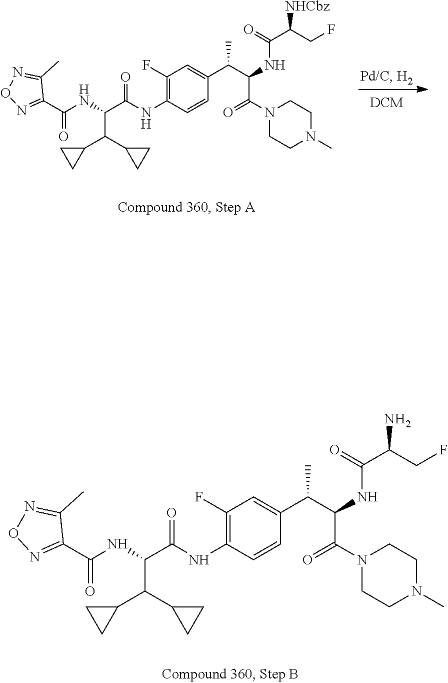 [SMILES: Cc1nonc1C(=O)N[C@H](C(=O)Nc1ccc([C@H](C)[C@@H](NC(=O)[C@H](CF)NC(=O)OCc2ccccc2)C(=O)N2CCN(C)CC2)cc1F)C(C1CC1)C1CC1 ; Cc1nonc1C(=O)N[C@H](C(=O)Nc1ccc([C@H](C)[C@@H](NC(=O)[C@@H](N)CF)C(=O)N2CCN(C)CC2)cc1F)C(C1CC1)C1CC1]

Step B: To a solution of compound 360, Step A (90.0 mg, 113 umol, 1.00 eq) in DCM (2.00 mL) was added Pd/C (10.0 mg, 10% purity) under N₂. The suspension was degassed under vacuum and purged with H₂ several times. The mixture was stirred under H₂ (15 psi) at 25° C. for 2 hrs. The reaction was filtrated and concentrated in vacuum. The residue was purified by Prep-HPLC (neutral condition; column: Waters Xbridge 150*25 mm*5 um; mobile phase: [water (NH₄HCO₃)-ACN]; B %: 36%-66%, min). Compound 360, Step B (70.0 mg, 106 umol, 93.6% yield) was obtained as white solid. LC-MS: rt=0.68 min; m/z=645.4 for [M+H]⁺.

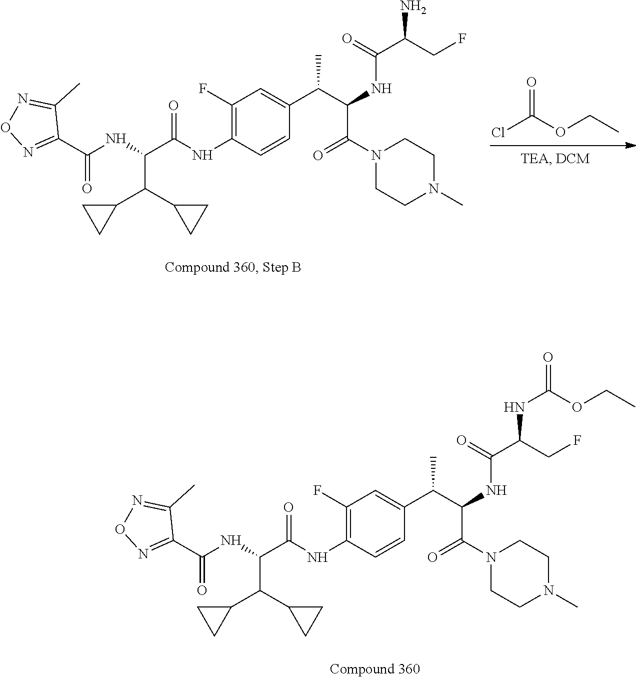

Step C: To a solution of compound 360, Step B (70.0 mg, 108 umol, 1.00 eq) in DCM (2.00 mL) was added TEA (32.9 mg, 325 umol, 45.3 uL, 3.00 eq) and ethyl chloroformate (17.6 mg, 162 umol, 15.5 uL, 1.50 eq) at 0° C. The mixture was stirred at 0° C. for 0.5 hr. LC-MS (EW24650-429-P1A1) showed desired mass was detected. The reaction was diluted with H₂O (30.0 mL), and extracted with EtOAc (30.0 mL*3), dried with anhydrous Na₂SO₄, filtrated and concentrated in vacuum. The residue was purified by Prep-TLC (SiO₂, DCM:MeOH=10:1). 360 (30.6 mg, 41.9 umol, 38.6% yield, 98.0% purity) was obtained as white solid. LC-MS: rt=0.83 min; m/z=717.4 for [M+H]⁺.

Compounds 358, 359, and 363 were similarly synthesized using similar reaction conditions with the appropriate carboxylic acid and haloformate reagents.

### Example 3.5—Synthesis of Compound 306

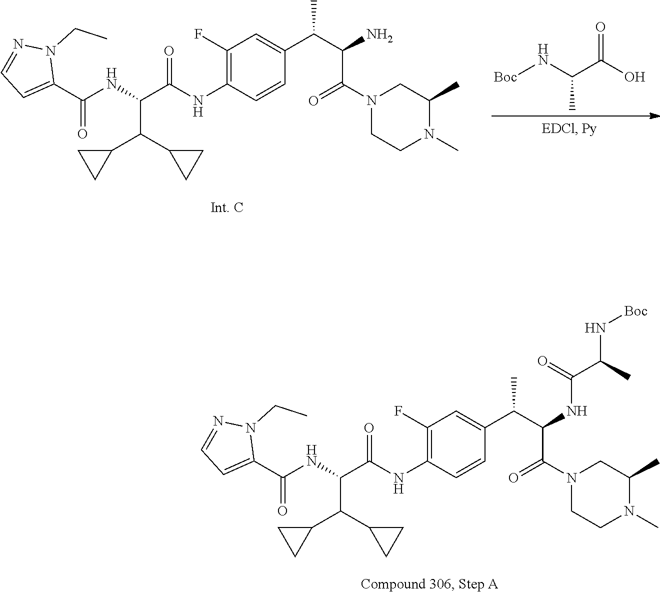 [SMILES: CCn1nccc1C(=O)N[C@H](C(=O)Nc1ccc([C@H](C)[C@@H](N)C(=O)N2CCN(C)[C@H](C)C2)cc1F)C(C1CC1)C1CC1 ; C[C@H](NC(=O)OC(C)(C)C)C(=O)O ; CCn1nccc1C(=O)N[C@H](C(=O)Nc1ccc([C@H](C)[C@@H](NC(=O)[C@H](C)NC(=O)OC(C)(C)C)C(=O)N2CCN(C)[C@H](C)C2)cc1F)C(C1CC1)C1CC1]

Step A: To a mixture of compound Int. C (500 mg, 860 umol, 1.00 eq) and (2S)-2-{[(tert-butoxy)carbonyl]amino}propanoic acid (244 mg, 1.29 mmol, 1.50 eq) in pyridine (4.00 mL) was added EDCl (330 mg, 1.72 mmol, 2.00 eq) and stirred at 20° C. for 3 hrs. The reaction mixture was concentrated under reduced pressure to give a residue, then diluted with DCM (20.0 mL), and the combined organic phase was washed with water (10.0 mL) and brine (10.0 mL), then dried over Na₂SO₄, filtered, and concentrated under reduced pressure to give a residue. The residue was purified by prep-TLC (plate 1, DCM:MeOH=10:1, R_{f}=0.48). To get the desired product compound 306, Step A (458 mg, 608 umol, 70.8% yield, 100% purity) as white solid. LC-MS: rt=0.95 min; m/z=753.3 for [M+H]⁺.

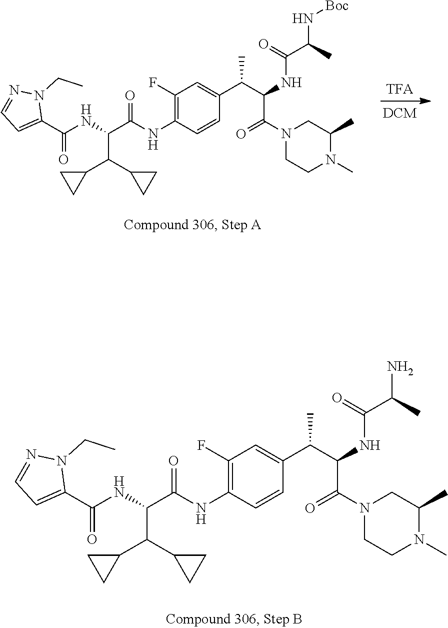 [SMILES: CCn1nccc1C(=O)N[C@H](C(=O)Nc1ccc([C@H](C)[C@@H](NC(=O)[C@H](C)NC(=O)OC(C)(C)C)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)[C@H](C)N)C(=O)N2CCN(C)[C@H](C)C2)cc1F)C(C1CC1)C1CC1]

Step B: To a solution of compound 306, Step A (450 mg, 598 umol, 1.00 eq) in DCM (4.00 mL) was added TFA (2.04 g, 17.9 mmol, 1.33 mL, 30.0 eq) at 0° C., then warmed to 20° C. and stirred for 1 hr. The reaction mixture was concentrated under reduced pressure to give a residue, and the residue was diluted with sat.aq. NaHCO₃ (20.0 mL), then extracted with DCM (10.0 mL*5), and the combined organic phase was washed with water (10.0 mL) and brine (10.0 mL), then dried over Na₂SO₄, filtered, and concentrated under reduced pressure to give a residue of desired product compound 306, Step B (340 mg, 521 umol, 87.1% yield) as a white solid. LC-MS: rt=0.85 min; m/z=653.3 for [M+H]⁺.

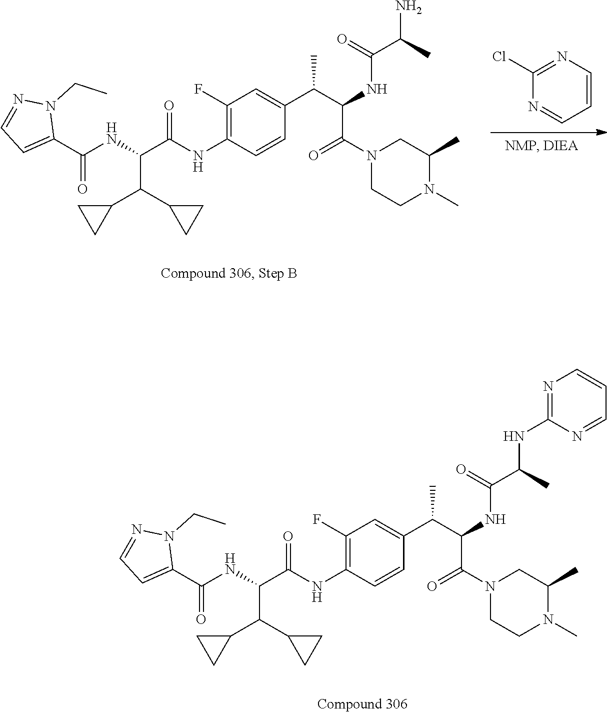 [SMILES: CCn1nccc1C(=O)N[C@H](C(=O)Nc1ccc([C@H](C)[C@@H](NC(=O)[C@H](C)N)C(=O)N2CCN(C)[C@H](C)C2)cc1F)C(C1CC1)C1CC1 ; Clc1ncccn1 ; CCn1nccc1C(=O)N[C@H](C(=O)Nc1ccc([C@H](C)[C@@H](NC(=O)[C@H](C)Nc2ncccn2)C(=O)N2CCN(C)[C@H](C)C2)cc1F)C(C1CC1)C1CC1]

Step C: To a mixture of compound 306, Step B (150 mg, 230 umol, 1.00 eq) and 2-chloropyrimidine (52.6 mg, 460 umol, 2.00 eq) in NMP (2.00 mL) was added DIEA (149 mg, 1.15 mmol, 200. uL, 5.00 eq) and heated to 170° C. with microwave for 20 hrs. The reaction mixture was diluted with water (20.0 mL) and extracted with DCM (10.0 mL*4), the combined organic layer was washed with water (20.0 mL) and brine (20.0 mL), dried over Na₂SO₄, filtered, and concentrated under reduced pressure to give a residue. The residue was purified by prep-HPLC (column: Waters Xbridge 150*25 mm*5 um; mobile phase: [water (10 mM NH₄HCO₃)-ACN]; B %: 32%-62%, min) to get the desired product compound 306 (80.0 mg, 109 umol, 47.6% yield) as a white solid. LC-MS: rt=0.95 min; m/z=731.5 for [M+H]⁺.

Compound 305 was synthesized using similar reaction conditions with the appropriate reagent.

### Example 3.6—Synthesis of Compound 296

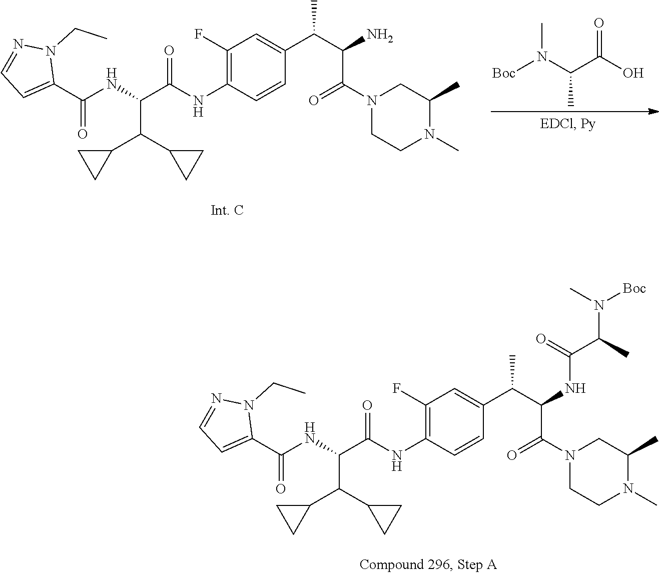 [SMILES: CCn1nccc1C(=O)N[C@H](C(=O)Nc1ccc([C@H](C)[C@@H](N)C(=O)N2CCN(C)[C@H](C)C2)cc1F)C(C1CC1)C1CC1 ; C[C@@H](C(=O)O)N(C)C(=O)OC(C)(C)C ; CCn1nccc1C(=O)N[C@H](C(=O)Nc1ccc([C@H](C)[C@@H](NC(=O)[C@H](C)N(C)C(=O)OC(C)(C)C)C(=O)N2CCN(C)[C@H](C)C2)cc1F)C(C1CC1)C1CC1]

Step A: To a solution of Int. C (150 mg, 257 umol, 1.00 eq) and (2S)-2-{[(tert-butoxy)carbonyl](methyl)amino}propanoic acid (78.6 mg, 386 umol, 1.50 eq) in pyridine (5.00 mL) was added EDCl (148 mg, 773 umol, 3.00 eq). The mixture was stirred at 15° C. for 12 hrs. The reaction mixture was added H₂O (10.0 mL) and extracted with DCM (20.0 mL*2). The combined organic layers were washed with sat.aq NaHCO₃ (40 mL), dried over Na₂SO₄, filtered, and concentrated under reduced pressure to give a residue. Compound 296, Step A (200 mg, crude) was obtained as a white solid. LC-MS: rt=0.78 min; m/z=767.5 for [M+H]⁺.

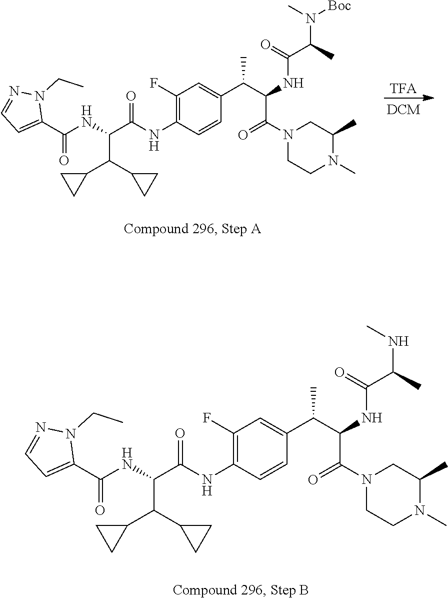 [SMILES: CCn1nccc1C(=O)N[C@H](C(=O)Nc1ccc([C@H](C)[C@@H](NC(=O)[C@H](C)N(C)C(=O)OC(C)(C)C)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)[C@H](C)NC)C(=O)N2CCN(C)[C@H](C)C2)cc1F)C(C1CC1)C1CC1]

Step B: To a solution of compound 296, Step A (200 mg, 260 umol, 1.00 eq) in DCM (5.00 mL) was added TFA (594 mg, 5.22 mmol, 386 μL, 20.0 eq) at 0° C. The mixture was stirred at 15° C. for 2 hrs. The reaction mixture was diluted with H₂O (10.0 mL) and added sat.aq NaHCO₃ to adjusted pH to 9. The mixture was extracted with DCM (50.0 m*2). The combined organic layers were washed with sat.aq NaHCO₃ (100 mL*4), dried over Na₂SO₄, filtered, and concentrated under reduced pressure to give a residue. Compound 296, Step B (150 mg, 224 umol, 86.2% yield) was obtained as a yellow solid. LC-MS: rt=0.92 min; m/z=667.5 for [M+H]+

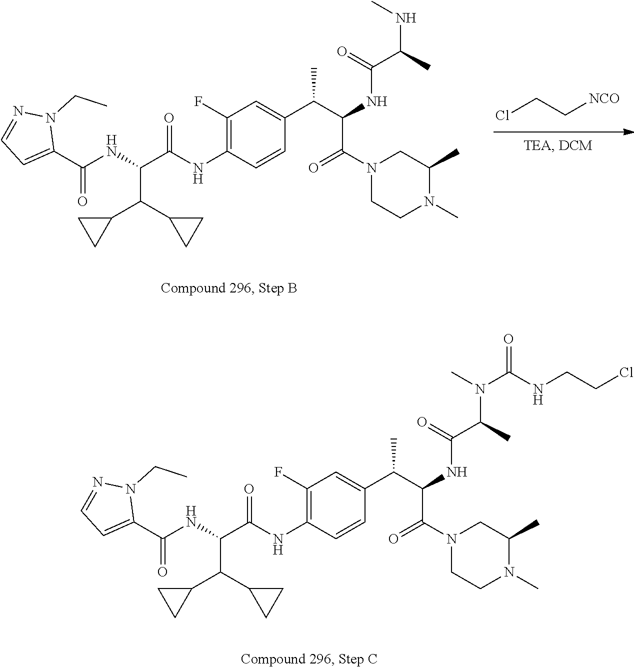

Step C: To a solution compound 296, Step B (130 mg, 194 umol, 1.00 eq) and TEA (59.1 mg, 584 umol, 81.4 uL, 3.00 eq) in DCM (2.00 mL) was added 2-chloroethyl isocyanate (30.8 mg, 292 umol, 1.50 eq). The mixture was stirred at 25° C. for 12 hrs. The reaction mixture was added H₂O (10.0 mL) and extracted with DCM (20.0 mL*2). The combined organic layers were washed with sat.aq NaHCO₃ (40.0 mL), dried over Na₂SO₄, filtered, and concentrated under reduced pressure to give a residue. Compound 296, Step C (120 mg, 155 umol, 79.7% yield) was obtained as yellow oil. LC-MS: rt=0.95 min; m/z=772.5 for [M+H]r.

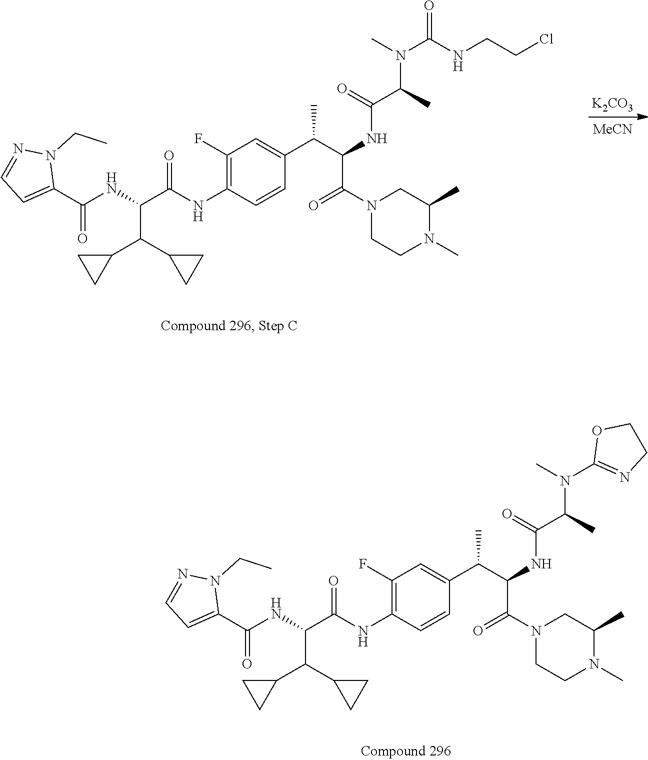 [SMILES: "MeCN" ; CCn1nccc1C(=O)N[C@H](C(=O)Nc1ccc([C@H](C)[C@@H](NC(=O)[C@H](C)N(C)C(=O)NCCCl)C(=O)N2CCN(C)[C@H](C)C2)cc1F)C(C1CC1)C1CC1 ; "K2CO3" ; CCn1nccc1C(=O)N[C@H](C(=O)Nc1ccc([C@H](C)[C@@H](NC(=O)[C@H](C)N(C)C2=NCCO2)C(=O)N2CCN(C)[C@H](C)C2)cc1F)C(C1CC1)C1CC1]

Step D: To a solution of compound 296, Step C (120 mg, 155 umol, 1.00 eq) in MeCN (10.0 mL) was added K₂CO₃ (64.4 mg, 466 umol, 3.00 eq). The mixture was stirred at 70° C. for 12 hrs. The reaction mixture was filtered and the filtrate was concentrated to give a crude product. The residue was purified by prep-HPLC (neutral condition; column: Waters Xbridge 150*25 mm*5 um; mobile phase: [water (10 mM NH₄HCO₃)-ACN]; B %: 26%-56%, 2 min). The residue was separated by SFC (condition: column: DAICEL CHIRALCEL OD-H (250 mm*30 mm, 5 um); mobile phase: [0.1% NH₃H₂O ETOH]; B %: 25%-25%, 5.0 min; 40 min min). Compound 296 (24.38 mg, 31.84 umol, 20.49% yield, 96.1% purity) was obtained as a white solid. LC-MS: rt=0.95 min; m/z=736.6 for [M+H]⁺.

### Example 4. Exemplary Scheme—Synthesis of Compounds 200, 229-230, 233-234, 243-244, 246-249, and 297

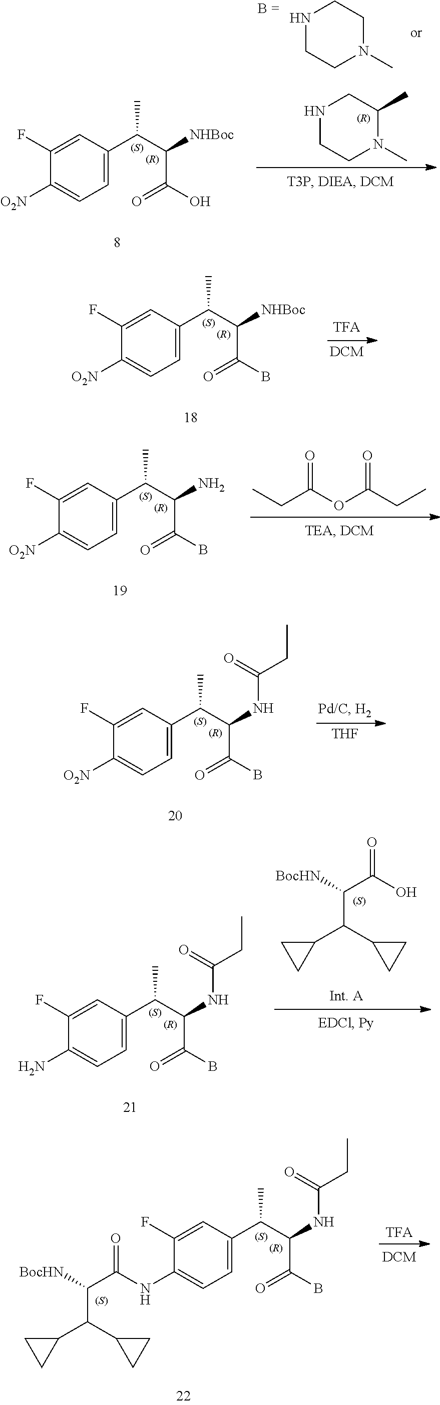

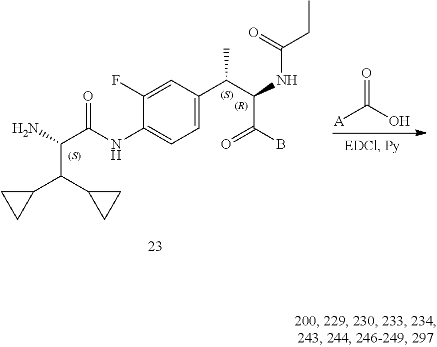

Step 1: To a solution of compound 8 (5.00 g, 14.6 mmol, 1.00 eq), (2R)-1,2-dimethylpiperazine (1.83 g, 16.0 mmol, 1.10 eq) and DIEA (9.65 g, 74.6 mmol, 13.0 mL, 5.11 eq) in DCM (50.0 mL) was added T3P (11.7 g, 18.5 mmol, 11.0 mL, 50% purity, 1.27 eq) at −20° C., then the mixture was stirred for 12 hrs. The reaction mixture was diluted by DCM (200 mL) and washed by saturated aq. NaHCO₃ (100 mL), H₂O (100 mL) and brine (50.0 mL), the organic layer was dried over anhydrous Na₂SO₄, filtered, and concentrated under reduced pressure to give a residue, to get the desired product compound 18, which was used without further purification or analysis.

Step 2: To a solution of compound 18 (6.31 g, 14.3 mmol, 1.00 eq) in DCM (70.0 mL) was added TFA (16.4 g, 143 mmol, 10.6 mL, 10.0 eq) at 0° C., then the mixture was warmed to 20° C. and stirred for 12 hrs. The reaction mixture was concentrated under reduced pressure to give a residue. To get the crude product compound 19 (6.51 g, crude, TFA) as yellow oil, which was used in the following step.

Step 3: To a solution of compound 19 (6.51 g, 14.3 mmol, 1.00 eq, TFA) in DCM (100 mL) was added TEA (14.5 g, 143 mmol, 20.0 mL, 10.0 eq) and propionic anhydride (3.75 g, 28.7 mmol, 3.71 mL, 2.00 eq) at 0° C., then the mixture was stirred for 1 hr at 20° C. The reaction mixture was washed by saturated aq. NaHCO₃ (50.0 ml), H₂O (50.0 mL) and brine, the organic layer was dried over anhydrous Na₂SO₄, filtered, and concentrated under reduced pressure to give a residue. The residue was purified by Prep-HPLC (column: Welch Ultimate XB—CN 250*70*10 um; mobile phase: [Heptane-EtOH (0.1% NH₃H₂O)]; B %: 5%-45%, 12 min) to get the desired product compound 20 (4.00 g, 10.1 mmol, 70.4% yield) as a yellow solid. LC-MS: rt=0.56 min; m/z=395.1 for [M+H]⁺.

Step 4: To a solution of compound 20 (4.00 g, 10.1 mmol, 1.00 eq) in THF (40.0 mL) was added Pd/C (400 mg, 10% purity) under N₂. The suspension was degassed under vacuum and purged with H₂ several times. The mixture was stirred under H₂ (15.0 psi) at 15° C. for 12 hrs. The reaction mixture was filtered, and the cake was washed with MeOH (100 mL), and then concentrated under reduced pressure to give a residue. Compound 21 (3.20 g, 8.78 mmol, 86.5% yield) was obtained as a white solid, which was used without further characterization.

Step 5: To a solution of compound 21 (310 mg, 1.15 mmol, 1.20 eq) and Int. A (350 mg, 960 umol, 1.00 eq) in pyridine (5.00 mL) was added EDCl (368 mg, 1.92 mmol, 2.00 eq). The mixture was stirred at 15° C. for 10 hrs. The reaction mixture was diluted with H₂O (50.0 mL) and extracted with EtOAc (50.0 mL*2). The combined organic layers were washed with sat.aq NaHCO₃ (70.0 mL*2), dried over Na₂SO₄, filtered, and concentrated under reduced pressure to give a residue. Compound 22 (400 mg, 649.59 umol, 67.64% yield, N/A purity) was obtained as a white solid. LC-MS: rt=1.00 min; m/z=616.6 for [M+H]⁺.

Step 6: To a solution of compound 22 (400 mg, 649. umol, 1.00 eq) in DCM (5.00 mL) was added TFA (1.48 g, 12.9 mmol, 961 μL, 20.0 eq) at 0° C. The mixture was stirred at 15° C. for 2 hrs. The reaction mixture was diluted with H₂O (10.0 mL) and added sat.aq NaHCO₃ to adjusted pH to 9. The mixture was extracted with DCM (20 mL*2). The combined organic layers were dried over Na₂SO₄, filtered, and concentrated under reduced pressure to give a residue. Compound 23 (288 mg, 558.51 umol, 85.98% yield) was obtained as a yellow solid. LC-MS: rt=0.90 min; m/z=516.5 for [M+H]⁺.

### Example 4.1.1—Synthesis of Compound 229

Step 7: To a solution of compound 23 (50.0 mg, 96.9 umol, 1.00 eq) and 1-cyclopropyl-1H-pyrazole-5-carboxylic acid (22.1 mg, 145 umol, 1.50 eq) in pyridine (3.00 mL) was added EDCl (37.1 mg, 193 umol, 2.00 eq). The mixture was stirred at 15° C. for 10 hrs. The reaction mixture was diluted with H₂O (10.0 mL) and extracted with EtOAc (20.0 mL*2). The combined organic layers were washed with sat.aq NaHCO₃ (20 mL*3), dried over Na₂SO₄, filtered, and concentrated under reduced pressure to give a residue. The residue was purified by prep-TLC (SiO₂, DCM:MeOH=10:1). Compound 229 (37.6 mg, 55.9 umol, 57.6% yield, 96.4% purity) was obtained as a white solid. LC-MS: rt=0.74 min; m/z=650.4 for [M+H]⁺.

Compounds 200, 230, 233, 234, 243, 244, 246-249, and 297, were synthesized using similar reaction conditions with the appropriate carboxylic acid reagent.

### Example 4.3.2—Synthesis of Compound 200

To a solution of appropriately substituted compound 23 (50.0 mg, 96.9 umol, 1.00 eq) and 1-ethyl-1H-pyrazole-5-carboxylic acid (16.7 mg, 119 umol, 1.20 eq) in pyridine (5.00 mL) was added EDCl (37.1 mg, 193 umol, 2.00 eq). The mixture was stirred at 25° C. for 12 hrs. The reaction mixture was diluted with sat.aq NaHCO₃ (30.0 mL) and extracted with EtOAc (30.0 mL*3). The combined organic layers were washed with brine (30 mL*3), dried over Na₂SO₄, filtered, and concentrated under reduced pressure to give a residue. The residue was purified by prep-HPLC (column: Phenomenex Gemini-NX C18 75*30 mm*3 um; mobile phase: [water (0.05% ammonia hydroxide v/v)-ACN]; B %: 20%-50%, 7 min). Compound 200 (31.0 mg, 49.7 umol, 49.8% yield, 100% purity) was obtained as a white solid. LC-MS: rt=0.93 min; m/z=624.4 for [M+H]⁺.

### Example 4.2.3—Synthesis of Compound 246

To a solution of appropriately substituted compound 23 (50.0 mg, 96.9 umol, 1.00 eq) and 4-methyl-1,2,5-oxadiazole-3-carboxylic acid (18.6 mg, 145 umol, 1.50 eq) in pyridine (3.00 mL) was added EDCl (37.1 mg, 193 umol, 2.00 eq). The mixture was stirred at 15° C. for 10 hrs. The reaction mixture was diluted with H₂O (10.0 mL) and extracted with EtOAc (20.0 mL*2). The combined organic layers were washed with sat.aq NaHCO₃ (20 mL*3), dried over Na₂SO₄, filtered, and concentrated under reduced pressure to give a residue. The residue was purified by prep-TLC (SiO₂, DCM:MeOH=10:1). Compound 246 (27.3 mg, 41.6 umol, 42.9% yield, 95.4% purity) was obtained as a white solid. LC-MS: rt=0.76 min; m/z=626.4 for [M+H]⁺.

As described in Example 4,

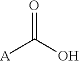

is represented by:

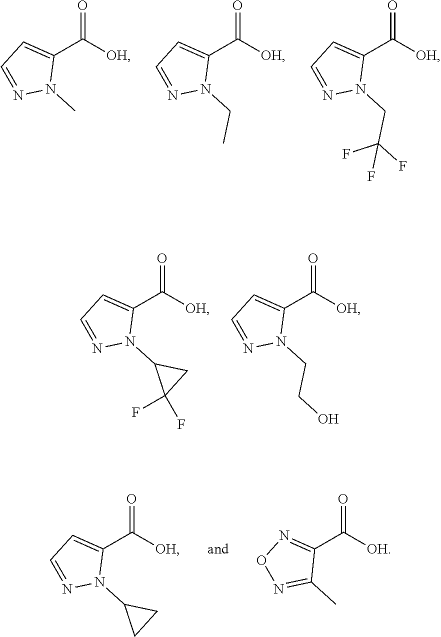

In some embodiments, A of

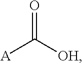

is represented by A of Formula (I) or (I-A), and is an optionally substituted C₃₋₆ carbocycle or an optionally substituted 5- to 6-membered heterocycle as described herein.

In some embodiments, B of Example 4 is represented by

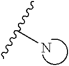 [SMILES: *N]

of Formula (I) or (I-A), and is an optionally substituted 4- to 12-membered heterocycle as described herein.

### Example 5—Synthesis of Compound 201

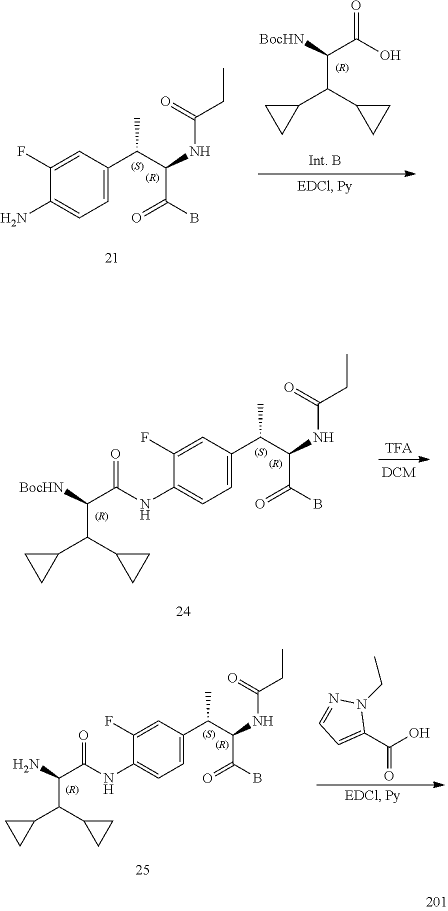

Step 1: To a solution of Intermediate B (140 mg, 519 umol, 1.01 eq) and compound 21 (with B=1-methylpiperazine) (180 mg, 513 umol, 1.00 eq) in pyridine (5.00 mL) was added EDCl (200 mg, 1.04 mmol, 2.03 eq) at 25° C., then the mixture was stirred at 25° C. for 12 hrs. The mixture was combined with a similar reaction run on a 30.0 mg scale. The residue was diluted with H₂O (40.0 mL) and extracted with ethyl acetate (20.0 mL*3). The combined organic layers were washed with brine (20.0 mL*3), dried over Na₂SO₄, filtered, and concentrated under reduced pressure to give a residue. The residue was purified by Prep-HPLC (column: Phenomenex Gemini-NX C18 75*30 mm*3 um; mobile phase: [water (0.05% ammonia hydroxide v/v)-ACN]; B %: 25%-55%, 7 min). Compound 24 (0.150 g, 249 umol, 40.0% yield) was obtained as white solid. LC-MS: rt=0.76 min; m/z=602.4 for [M+H]⁺.

Step 2: To a solution of compound 24 (120 mg, 199 umol, 1.00 eq) in DCM (3.00 mL) was added dropwise TFA (454 mg, 3.99 mmol, 295 μL, 20.0 eq) at 0° C., then the mixture was stirred at 25° C. for 2 hrs. The mixture was combined with a similar reaction run on a 30.0 mg scale. The reaction mixture was concentrated under reduced pressure to remove DCM. The residue was diluted with sat.NaHCO₃ (20.0 mL) and extracted with ethyl acetate (20.0 mL*3). The combined organic layers were washed with brine (20.0 mL*3), dried over Na₂SO₄, filtered, and concentrated under reduced pressure to give a residue. Compound 25 (110 mg) was obtained as white solid. LC-MS: rt=0.91 min; m/z=502.5 for [M+H]⁺.

Step 3: To a solution of compound 25 (80.0 mg, 159 umol, 1.00 eq) and 1-ethyl-1H-pyrazole-5-carboxylic acid (27.0 mg, 192 umol, 1.21 eq) in pyridine (5.00 mL) was added EDCl (61.1 mg, 318 umol, 2.00 eq) at 25° C., then the mixture was stirred at 25° C. for 6 hrs. The residue was diluted with sat.aq.NaHCO₃ (30.0 mL) and extracted with ethyl acetate (30.0 mL*3). The combined organic layers were washed with brine (30.0 mL*3), dried over Na₂SO₄, filtered, and concentrated under reduced pressure to give a residue. The residue was purified by Prep-TLC (SiO₂, DCM:MeOH=10:1). The residue was purified by Prep-HPLC (column: Phenomenex Gemini-NX C18 75*30 mm*3 um; mobile phase: [water (0.05% ammonia hydroxide v/v)-ACN]; B %: 18%-48%, 7 min). Compound 201 (35.0 mg, 56.1 umol, 35.1% yield, 100% purity) was obtained as white solid. LC-MS: rt=0.93 min; m/z=624.5 for [M+H]⁺.

### Example 6—Synthesis of Intermediate D

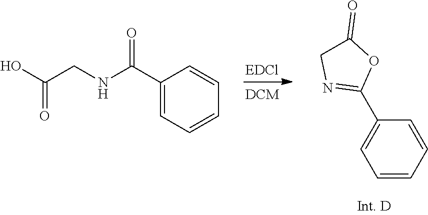 [SMILES: O=C(O)CNC(=O)c1ccccc1 ; O=C1CN=C(c2ccccc2)O1]

To a solution of (benzoylamino)acetic acid (52 g, 290.22 mmol, 1 eq) in dichloromethane (400 mL) was added EDCl (61.20 g, 319.25 mmol, 1.1 eq). The mixture was stirred at 25° C. for 2 hrs. Water (200 mL) was added to the reaction mixture and the water phase was extracted with dichloromethane (50 mL*2). The combined organic phase was washed with brine (100 mL), dried over Na₂SO₄, filtered, and concentrated in vacuum to get the product. The product was slurried with a mixture of Dichloromethane and Petroleum ether (V:V=1:4, 500 mL) at 20° C. for 30 min. The filter cake was obtained as product and the filtrate was purified by column chromatography (SiO₂, Petroleum ether/Ethyl acetate=50/1 to 5/1). Int. D (42 g, 258.01 mmol, 88.90% yield, 99% purity) was obtained as a yellow solid. LC-MS: rt=0.93 min; m/z=624.5 for [M+H]⁺. ¹H NMR: (400 MHz, CDCl₃) δ 8.01-7.99 (m, 2H), 7.59-7.52 (m, 1H), 7.51-7.48 (m, 2H), 4.43 (s, 2H).

### Example 7—Synthesis of Intermediate E

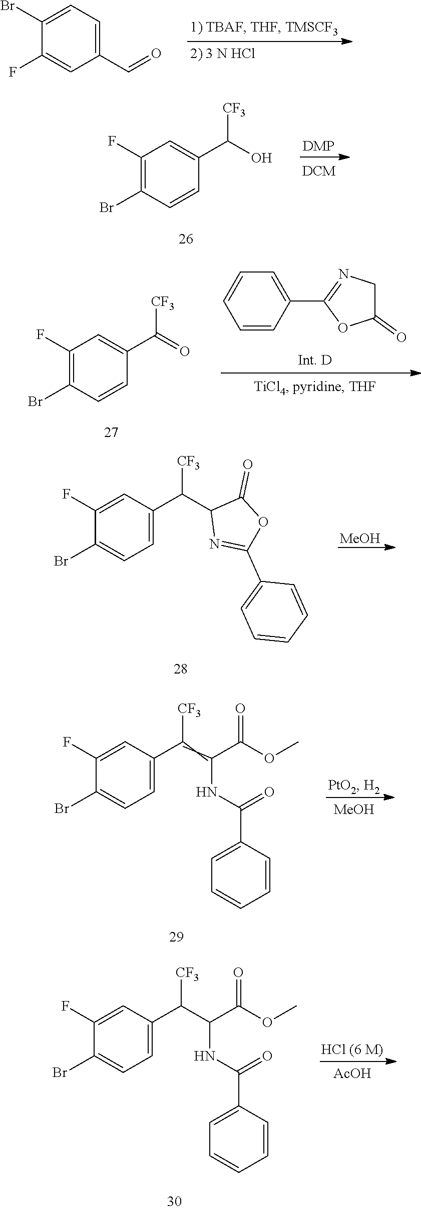

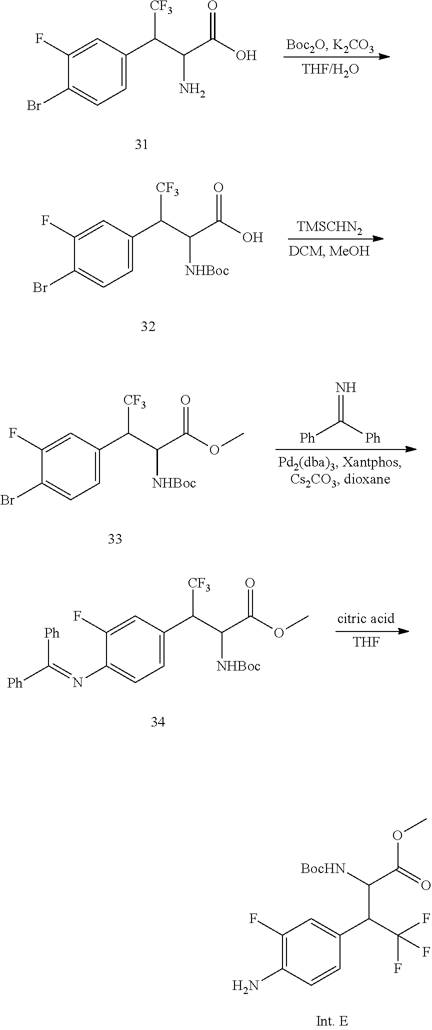

Step 1: To a solution of 4-Bromo-3-fluorobenzaldehyde (115 g, 566.48 mmol, 1 eq) in THF (1000 mL) was added TMSCF₃ (96.66 g, 679.78 mmol, 1.2 eq) and then TBAF (1 M, 11.33 mL, 0.02 eq) was added at 0° C. The mixture was stirred at 20° C. for 1 hr. HCl (3 M, 283.24 mL, 1.5 eq) was added at 10° C. and then the mixture was stirred at 20° C. for 12 hrs. MTBE (200 mL) was added to the mixture and the aqueous phase was extracted with MTBE (100 mL). The organic phase was washed with brine (100 mL), dried over Na₂SO₄, filtered, and then concentrated in vacuum to get the crude product. The residue was purified by column chromatography (SiO₂, Petroleum ether/Ethyl acetate=100/1 to 50/1). Compound 26 (150 g, 533.47 mmol, 94.17% yield, 97.1% purity) was obtained as a colorless oil. LC-MS: rt=0.98 min; m/z=271.0 for [M−H]—. ¹H NMR: (400 MHz, CDCl₃) δ: 7.62-7.58 (m, 1H), 7.32-7.29 (m, 1H), 7.16 (d, J=8.4 Hz, 1H), 5.04-5.01 (m, 1H), 2.78-2.77 (d, J=3.6 Hz, 1H).

Step 2: To a solution of compound 26 (75 g, 274.70 mmol, I eq) in dichloromethane (700 mL) was added DMP (139.82 g, 329.64 mmol, 102.06 mL, 1.2 eq) at 0° C. The mixture was stirred at 20° C. for 4 hrs. H₂O (500 mL) was added to the reaction mixture and then filtered. The filtrate was washed successively with Sat. NaHCO₃ (200 mL), Sat. Na₂SO₃ (100 mL*2), brine (100 mL). The organic phase was dried over Na₂SO₄, filtered, and concentrated in vacuum to get the crude product. The crude product 27 was used for next step without purification.

Step 3: To a solution of TiCl₄ (60.89 g, 321.03 mmol, 1.5 eq) in THF (600 mL) was added a solution of compound 27 (58 g, 214.02 mmol, 1 eq) in THE (100 mL) at −10° C. A solution Int. D (41.39 g, 256.82 mmol, 1.2 eq) in THF (100 mL) was added to the mixture at −10° C. The mixture was stirred at −10˜−0° C. for 30 min. Pyridine (33.86 g, 428.03 mmol, 34.55 mL, 2 eq) was added at 0° C. and then the mixture was stirred at 20° C. for 12 hrs. H₂O (200 mL) was added to the mixture and the aqueous phase was extracted with ethyl acetate (100 mL*2). The combined organic layers were washed with brine (200 mL), dried over Na₂SO₄, filtered, and concentrated in vacuum to get the crude product. The residue was purified by column chromatography (SiO₂, Petroleum ether/Ethyl acetate=10/1 to 20/1). Compound 28 (54 g, 130.39 mmol, 60.92% yield, 100% purity) was obtained as a yellow solid. LC-MS: rt=1.09 min; m/z=414.1 for [M+H]⁺. ¹H NMR: (400 MHz, CDCl₃) δ 8.21-8.19 (m, 2H), 7.70-7.66 (m, 2H), 7.59-7.57 (m, 2H), 7.17-7.15 (m, 1H), 7.07-7.05 (m, 1H).

Step 4: A solution of compound 28 (40 g, 96.58 mmol, 1 eq) in MeOH (100 mL) was stirred at 50° C. for 4 hrs. The mixture was concentrated in vacuum to get the product. The residue was purified by column chromatography (SiO₂, Petroleum ether/Ethyl acetate=100/1 to 5/1). Compound 29 (40 g, 88.75 mmol, 91.89% yield, 99% purity) was obtained as a white solid. ¹H NMR: (400 MHz, CDCl₃) δ 8.46 (s, 1H), 7.85-7.83 (m, 2H), 7.64-7.52 (m, 4H), 7.12-7.10 (d, J=8.8 Hz, 1H), 7.01-6.81 (d, J=8.0 Hz, 1H), 3.60 (s, 3H).

Step 5: To a solution of compound 29 (10 g, 22.41 mmol, 1 eq) in MeOH (100 mL) was added PtO₂ (1.00 g, 4.40 mmol, 1.96e−1 eq) under N₂. The suspension was degassed under vacuum and purged with H₂ several times. The mixture was stirred under H₂ (50 psi) at 20° C. for 16 hours. The mixture was filtered and the filtrate was concentrated in vacuum to get the crude product. The crude product was used for next step without purification. Compound 30 (10 g, crude) was obtained as a white solid. LC-MS: rt=1.10 min; m/z=452.0 for [M−H].

Step 6: A solution of compound 30 (10 g, 22.01 mmol, 1 eq) in HCl (6 M, 98.67 mL, 26.89 eq) and AcOH (51.80 g, 862.60 mmol, 49.33 mL, 39.18 eq) was stirred at 120° C. for 12 hrs. The reaction mixture was concentrated in vacuum to get the crude product. The crude product was used for next step without purification. Compound 31 (8 g, crude, HCl) was obtained as a yellow oil. LC-MS: rt=0.70 min; m/z=330.1 for [M+H]⁺.

Step 7: To a solution of compound 31 (8 g, 21.83 mmol, 1 eq, HCl) in THF (90 mL) was added a solution of K₂CO₃ (12.07 g, 87.30 mmol, 4 eq) in H₂O (20 mL). Boc₂O (7.15 g, 32.74 mmol, 7.52 mL, 1.5 eq) was added to the mixture. The mixture was stirred at 20° C. for 12 hrs. The mixture was adjusted pH to 4 with HCl (1 M) and then extracted with Ethyl acetate (50 mL*2). The combined organic layers were washed with brine (50 mL), dried over Na₂SO₄, filtered, and concentrated in vacuum to get the crude product. The crude product was used for next step without purification. Compound 32 (9 g, crude) was obtained as a yellow oil.

Step 8: To a solution of compound 32 (9 g, 20.92 mmol, 1 eq) in dichloromethane (150 mL) and MeOH (15 mL) was added drop wise TMSCHN₂ (2 M, 11.51 mL, 1.1 eq). The mixture was stirred at 20° C. for 2 hrs. The mixture was concentrated in vacuum to get the crude product. The residue was purified by column chromatography (SiO₂, Petroleum ether/Ethyl acetate=100/1 to 5/1). Compound 33 (9 g, 19.33 mmol, 92.39% yield, 95.4% purity) was obtained as a light-yellow oil.

Step 9: A mixture of compound 33 (12.5 g, 28.14 mmol, 1 eq), diphenylmethanimine (7.65 g, 42.21 mmol, 7.08 mL, 1.5 eq) and Cs₂CO₃ (18.34 g, 56.28 mmol, 2 eq) in dioxane (200 mL) was added Pd₂(dba)₃ (1.29 g, 1.41 mmol, 0.05 eq) and Xantphos (1.63 g, 2.81 mmol, 0.1 eq). The mixture was stirred at 100° C. for 14 hrs. The mixture was filtered and the filtrate was concentrated in vacuum to get the crude product. The crude product was used for next step without purification. Compound 34 (20 g, crude) was obtained as a black-brown oil. LC-MS: rt=1.12 min; m/z=545.3 for [M+H]⁺.

Step 10: To a solution of compound 34 (20 g, 36.73 mmol, 1 eq) in THF (150 mL) was added citric acid (10 M, 140.00 mL, 38.12 eq). The mixture was stirred at 20° C. for 16 hrs. The mixture was extracted with Ethyl acetate (150 mL*2) and the organic layers were washed successively with Sat. NaHCO₃ (150 mL), brine (100 mL), dried over Na₂SO₄, filtered, and concentrated in vacuum to get the crude product. The residue was purified by column chromatography (SiO₂, Petroleum ether/Ethyl acetate=100/1 to 3/1). Int. E (5 g, 12.90 mmol, 35.11% yield, 98.1% purity) was obtained as yellow oil. LC-MS: rt=0.92 min; m/z=281.1 for [M+H-boc]^{+ 1}H NMR: (400 MHz, CDCl₃) δ 7.02-6.98 (m, 1H), 5.95-6.88 (m, 1H), 6.77-6.75 (m, 1H), 5.18-5.04 (m, 1H), 4.87-4.81 (m, 11H), 3.86-3.79 (m, 1H), 3.71-3.68 (m, 3H), 1.43 (s, 9H).

### Example 8. Exemplary Scheme—Synthesis of Compounds 350-357

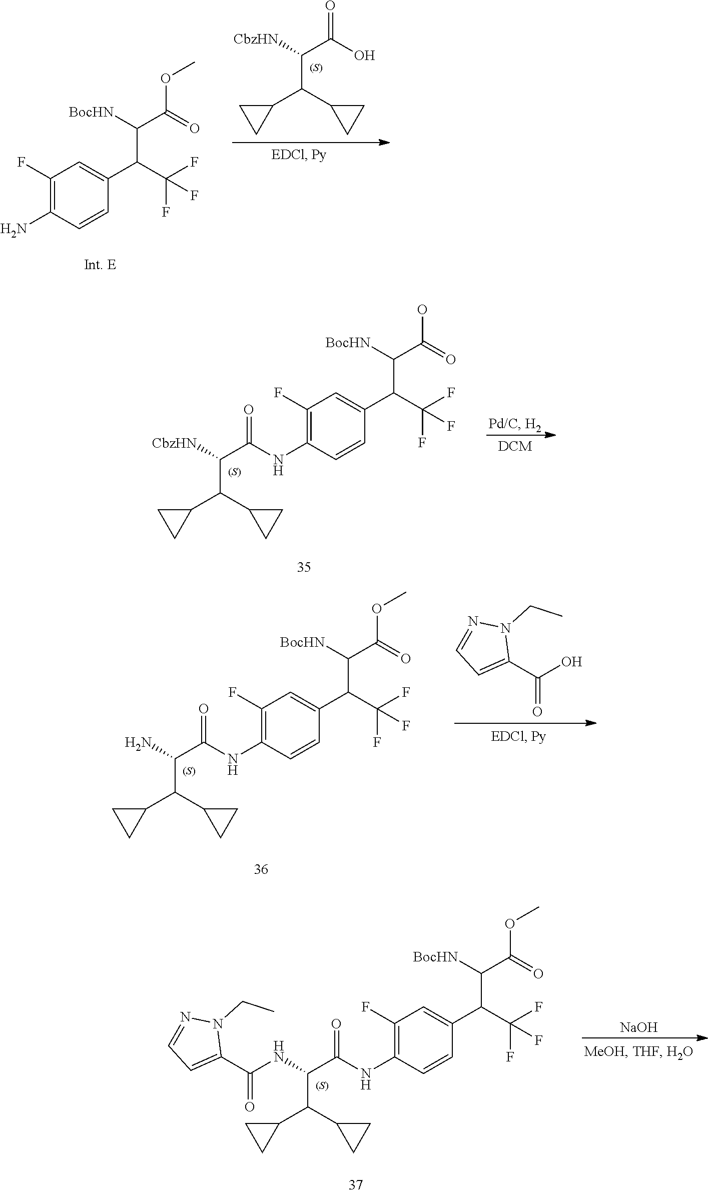

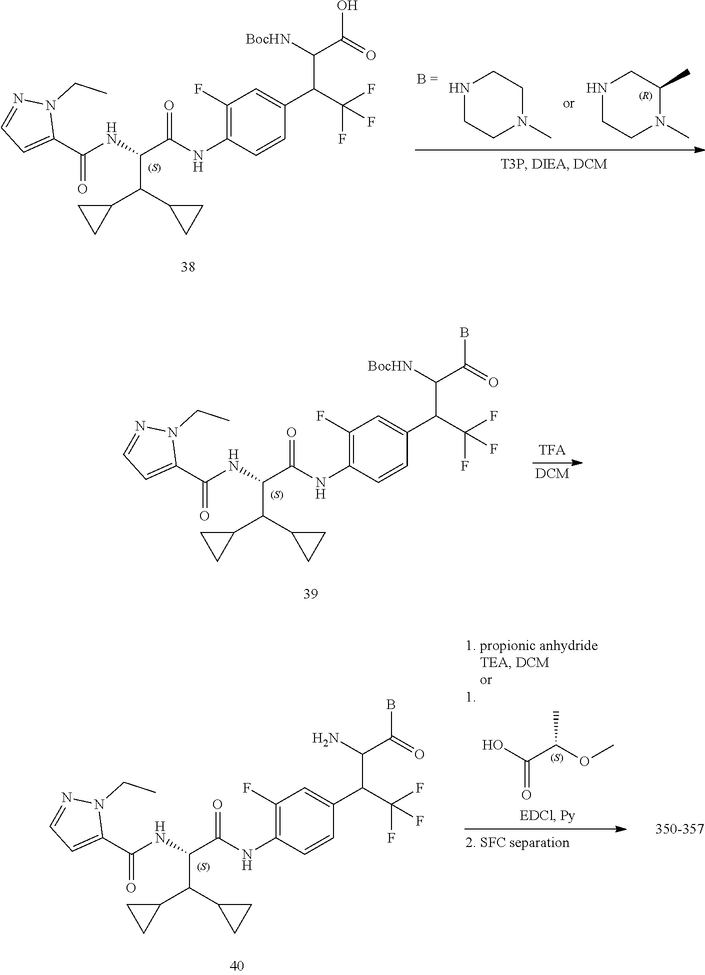

Step 1: To a solution of Intermediate E (500 mg, 1.31 mmol, 1.00 eq) and Cbz-amino acid (518 mg, 1.71 mmol, 1.30 eq) in pyridine (10.0 mL) was added EDCl (756 mg, 3.94 mmol, 3.00 eq) at 25° C. for 2 hrs. The reaction mixture was added 40.0 mL H₂O and extracted with EtOAc 100 mL (50.0 mL*2). The combined organic layers were washed with sat.aq NaHCO₃ 100 mL*2, dried over Na₂SO₄, filtered and concentrated under reduced pressure to give a residue. The residue was purified by Prep-HPLC (basic condition, column: Welch Ultimate XB—CN 250*50*10 um; mobile phase: [Hexane-EtOH (0.1% NH₃120)]; B %: 1%-40%, 15 min). Compound 35 (400 mg, 600 umol, 45.7% yield) was obtained as a white solid. LC-MS: rt=1.08 min; m/z=566.3 for [M+H-Boc]⁺.

Step 2: To a solution of compound 35 (400 mg, 600 umol, 1.00 eq) in DCM (5.00 mL) was added Pd/C (40.0 mg, 10.0% purity) under N₂ atmosphere. The suspension was degassed and purged with H₂ for 3 times. The mixture was stirred under H₂ (15 Psi) at 25° C. for 2 hrs. The reaction mixture was filtered, and the cake was washed with 100 mL MeOH, concentrated under reduced pressure to give a residue. Compound 36 (319 mg, 600 umol, 99.8% yield) was obtained as a white solid. LC-MS: rt=0.84 min; m/z=554.1 for [M+H]⁺.

Step 3: To a solution of compound 36 (319 mg, 600 umol, 1.00 eq) and 1-ethyl-1H-pyrazole-5-carboxylic acid (126 mg, 900 umol, 1.50 eq) in pyridine (5.00 mL) was added EDCl (345 mg, 1.80 mmol, 3.00 eq). The mixture was stirred at 25° C. for 2 hrs. The reaction mixture was diluted with H₂O (30.0 mL) and extracted with EtOAc (30.0 mL*3). The combined organic layers were washed with sat.aq NaHCO₃ (30.0 mL*2), dried over Na₂SO₄, filtered and concentrated under reduced pressure to give a residue. Compound 37 (340 mg, 520 umol, 86.6% yield) was obtained as a white solid. LC-MS: rt=0.99 min; m/z=654.5 for [M+H]⁺.

Step 4: To a solution of compound 37 (340 mg, 520 umol, 1.00 eq) in MeOH (2.00 mL) and THF (2.00 mL) was added NaOH (83.2 mg, 2.08 mmol, 4.00 eq) in H₂O (1.00 mL) at 0° C. The mixture was stirred at 25° C. for 2 hrs. The reaction mixture was diluted with H₂O (10.0 mL) and added 1 M HCl to adjusted pH to 2. The mixture was extracted with DCM (20.0 mL*3). The combined organic layers were dried over Na₂SO₄, filtered and concentrated under reduced pressure to give a residue. Compound 38 (320 mg, 500 umol, 96.1% yield) was obtained as a yellow solid. LC-MS: rt=0.59 min; m/z=640.4 for [M+H]⁺.

Step 5: To a solution of compound 38 (160 mg, 250 umol, 1.00 eq) and 1-methylpiperazine (37.5 mg, 375 umol, 41.6 uL, 1.50 eq) in DCM (5.00 mL) was added DIEA (161 mg, 1.25 mmol, 217 μL, 5.00 eq) and T3P (477 mg, 750 umol, 446 μL, 50.0% purity, 3.00 eq) at 0° C. The mixture was stirred at 25° C. for 2 hrs. The reaction mixture was diluted with H₂O (30.0 mL) and extracted with DCM (30.0 mL*3). The combined organic layers were washed with sat.aq NH₄Cl (30.0 mL*2), dried over Na₂SO₄, filtered and concentrated under reduced pressure to give a residue. Compound 39 (with B=1-methylpiperazine) (180 mg, 249 umol, 99.7% yield) was obtained as a yellow solid. LC-MS: rt=0.84 min; m/z=722.5 for [M+H]⁺.

Step 6: To a solution of compound 39 (with B=1-methylpiperazine) (180 mg, 249 umol, 1.00 eq) in DCM (5.00 mL) was added TFA (568 mg, 4.99 mmol, 369 μL, 20.0 eq) at 0° C. The mixture was stirred at 25° C. for 2 hrs. The reaction mixture was diluted with H₂O (10.0 mL) and added sat.aq NaHCO₃ to adjusted pH to 9. The mixture was extracted with DCM (20.0 mL*2). The combined organic layers were dried over Na₂SO₄, filtered and concentrated under reduced pressure to give a residue. Compound 40 (with B=1-methylpiperazine) (150 mg, 241 umol, 96.7% yield) was obtained as a white solid. LC-MS: rt=0.77 min; m/z=622.3 for [M+H]⁺.

Step 7: To a solution of compound 40 (with B=1-methylpiperazine) (140 mg, 225 umol, 1.00 eq) in DCM (5.00 mL) was added TEA (45.5 mg, 450 mol, 62.6 uL, 2.00 eq) and propanoyl propanoate (37.6 mg, 289 umol, 37.3 uL, 1.20 eq) at 0° C. The mixture was stirred at 25° C. for 2 hrs. The reaction mixture was diluted with H₂O (30.0 mL) and extracted with EtOAc (30.0 mL*3). The combined organic layers were washed with sat.aq NaHCO₃ (30.0 mL*2), dried over Na₂SO₄, filtered and concentrated under reduced pressure to give a residue.

The residue was purified by SFC (column: DAICEL CHIRALPAK IE (250 mm*30 mm, 10 um); mobile phase: [Hexane-IPA]; B %: 25%-25%, 15 min) to obtain Compound 350. The residue was further purified by SFC (column: DAICEL CHIRALCEL OD-H (250 mm*30 mm, 5 um); mobile phase: [0.1% NH₃H₂O ETOH]; B %: 15%-15%, 2.2; 90 min, Rt=1.143, 1.191 min) to obtain Compound 352. The residue was further purified by SFC (column: DAICEL CHIRALPAK AD-H (250 mm*30 mm, 5 um); mobile phase: [0.1% NH₃H₂O IPA]; B %: 20%-20%, 5.2 min; 70 min, Rt=1.216, 1.348 min) to obtain Compound 354. The residue was further purified by SFC (column: DAICEL CHIRALPAK AD-H (250 mm*30 mm, 5 um); mobile phase: [0.1% NH₃H₂O IPA]; B %: 20%-20%, 5.2 min; 40 min, Rt=1.324 min) to obtain Compound 356.

Compound 350 (9.10 mg, 12.4 umol, 5.53% yield, 92.7% purity) was obtained as a white solid. LC-MS: rt=0.97 min; m/z=678.5 for [M+H]⁺. ¹H NMR: (400 MHz, CDCl₃) δ 8.48-8.44 (t, J=8.8 Hz, 1H), 8.35-8.31 (m, 1H), 7.63 (d, J=2.0 Hz, 1H), 7.40 (s, 1H), 7.25-7.19 (m, 2H), 6.73 (d, J=2.0 Hz, 1H), 6.34 (d, J=9.6 Hz, 1H), 5.80-5.75 (t, J=10 Hz, 1H), 4.99-4.96 (m, 1H), 4.76-4.70 (m, 2H), 4.09-3.98 (m, 1H), 3.70-3.65 (m, 1H), 3.57-3.53 (m, 1H), 3.47-3.42 (m, 1H), 2.40-2.35 (m, 3H), 2.27 (s, 3H), 2.13-2.00 (m, 1H), 1.59-1.55 (t, J=7.2 Hz, 3H), 1.39 (s, 3H), 1.31-1.27 (t, J=8.0 Hz, 3H), 1.07-0.91 (m, 3H), 0.62-0.66 (m, 4H), 0.54-0.36 (m, 4H).

Compound 352 (22.67 mg, 31.2 umol, 13.8% yield, 93.4% purity) was obtained as a white solid. LC-MS: rt=0.98 min; m/z=678.4 for [M+H]⁺. ¹H NMR: (400 MHz, CDCl₃) δ 8.29-8.25 (m, 2H), 7.48 (d, J=2.0 Hz, 1H), 7.17-7.08 (m, 3H), 6.59 (d, J=2.0 Hz, 1H), 6.17-6.15 (m, 1H), 5.72-5.67 (t, J=9.6 Hz, 1H), 4.90-4.87 (m, 1H), 4.60-4.54 (m, 2H), 4.07-3.99 (m, 1H), 3.78-3.63 (m, 3H), 2.48-2.39 (m, 3H), 2.31 (s, 3H), 2.02-1.83 (m, 4H), 1.43-1.39 (t, J=6.8 Hz, 3H), 1.28-1.24 (m, 1H), 0.91-0.80 (m, 5H), 0.67-0.52 (m, 4H), 0.42-0.23 (m, 4H).

Compound 354 (16.3 mg, 23.6 umol, 10.5% yield, 98.4% purity) was obtained as a white solid. LC-MS: rt=0.98 min; m/z=678.4 for [M+H]⁺. ¹H NMR: (400 MHz, CDCl₃) δ 8.39 (s, 1H), 8.28-8.24 (t, J=8.4 Hz, 1H), 7.49 (d, J=2.0 Hz, 1H), 7.30-7.27 (m, 1H), 7.09-7.06 (m, 2H), 6.65 (d, J=2.0 Hz, 11H), 6.25 (d, J=9.2 Hz, 11H), 5.75-5.70 (t, J=9.6 Hz, 11H), 4.94-4.91 (m, 11H), 4.65-4.52 (m, 2H), 4.06-3.97 (m, 1H), 3.81-3.71 (m, 2H), 3.64-3.58 (m, 1H), 2.47-2.39 (m, 3H), 2.32 (s, 3H), 1.96-1.82 (m, 3H), 1.44-1.40 (t, J=7.2 Hz, 3H), 1.31-1.25 (m, 2H), 0.88-0.76 (m, 5H), 0.67-0.49 (m, 4H), 0.43-0.17 (m, 4H).

Compound 356 (11.94 mg, 17.4 umol, 7.76% yield, 99.2% purity) was obtained as a white solid. LC-MS: rt=0.82 min; m/z=678.6 for [M+H]⁺. ¹H NMR: (400 MHz, CD₃CN) S 8.76 (s, 1H), 7.99-7.94 (t, J=8.4 Hz, 1H), 7.48 (d, J=1.6 Hz, 1H), 7.39-7.31 (m, 1H), 7.26-7.18 (m, 2H), 7.06 (d, J=9.6 Hz, 11H), 6.85 (d, J=2.0 Hz, 11H), 5.64-5.59 (t, J=10.8 Hz, 11H), 4.93-4.89 (m, 1H), 4.57-4.51 (m, 2H), 4.13-4.03 (m, 1H), 3.82 (s, 2H), 3.56 (s, 1H), 2.60-2.55 (m, 2H), 2.52 (s, 3H), 2.20-2.15 (m, 2H), 1.45-1.37 (m, 3H), 1.27-1.24 (m, 2H), 1.00-1.11 (m, 3H), 0.93-0.82 (m, 4H), 0.51-0.36 (m, 4H), 0.29-0.22 (m, 4H).

Compounds 351, 353, 355, and 357 were similarly synthesized and purified using (R)-1,2-dimethylpiperazine as B from Example 8.

In some embodiments, B of Example 8 is represented by

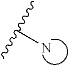 [SMILES: *N]

of Formula (I) or (I-A), and is an optionally substituted 4- to 12-membered heterocycle as described herein.

### Example 9: IL-17A/A HEK-Blue Cell Assay

The HEK-Blue IL-17 reporter cell line (Fisher #NC1408637) was used for cell-based IL-17A/A inhibition assays. Cells were grown and prepared for assays according to the manufacturer's instructions. This cell line consists of HEK 293 cells that were designed to expressed IL-17R^{A}, IL-17R^{C}, and the ActI adapter molecule, the combination of which, when stimulated by IL-17A/A activates a NFκB promoter and drives expression of a recombinant Secreted Alkaline Phosphatase (SEAP) protein. Media from the cells is then added to a development reagent (Quanti-Blue Substrate, Fisher #NC9711613), and read at A₆₃₀.

Compounds were titrated in DMSO, with a top final compound concentration of 10 uM and added to the cells immediately before adding IL-17A/A (Genscript #Z03228). The cells, compound, and IL-17A/A were then incubated for 20 hours before media was removed for SEAP analysis. The resulting inhibition curve was then analyzed using a Dotmatics screening protocol, and IC₅₀ values were determined using a 4-parameter nonlinear fit. DMSO was added to a universal final concentration of 0.1% to optimize background. IL-17A/A inhibition data is provided in Table 1 for selected compounds.

### Example 10: IL-17A/F HEK-Blue Cell Assay

The HEK-Blue IL-17 reporter cell line (Fisher #NC1408637) was used for cell-based IL-17A/F inhibition assays. Cells were grown and prepared for assays according to the manufacturer's instructions. This cell line consists of HEK 293 cells that were designed to expressed IL-17RA, IL-17R^{C}, and the ActI adapter molecule, the combination of which, when stimulated by IL-17A/A activates a NFκB promoter and drives expression of a recombinant Secreted Alkaline Phosphatase (SEAP) geneprotein. Media from the cells is then added to a development reagent (Quanti-Blue Substrate, Fisher #NC9711613), and read at A₆₃₀.

Compounds were titrated in DMSO and added to the cells immediately before adding IL-17A/A (Custom from R&D systems, untagged IL-17A/F+BSA as a carrier protein). The cells, compound, and IL-17A/A were then incubated for 20 hours before media was removed for SEAP analysis. The resulting inhibition curve was then analyzed using a Dotmatics screening protocol, and IC₅₀ values were determined using a 4-parameter nonlinear fit. DMSO was added to a universal final concentration of 0.1% to optimize background. IL-17A/F inhibition data is provided in Table 1 for selected compounds. Table 1 includes pIC₅₀ values for IL-17A/A and IL-17A/F inhibition of selected compounds; with compounds having a pIC₅₀ of greater than or equal 8 as A; 8>B≥7 as B; and 7>C≥5. Table 1 also includes synthesis and spectroscopic data for the claimed IL-17 modulators.

TABLE 1
Bioassay Inhibition data and Spectrospcopic data of Compounds 200-363

| | | IL17 AA | IL17AF | |
|---|---|---|---|---|
| Compound | | HEK-5 | HEK | LCMS |
| No. | Structure | pIC₅₀ | Blue pIC₅₀ | [M + H] |
| 200 | 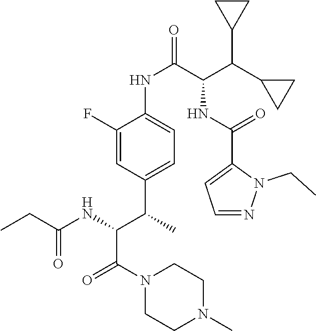 [SMILES: CCC(=O)N[C@@H](C(=O)N1CCN(C)CC1)[C@@H](C)c1ccc(NC(=O)[C@@H](NC(=O)c2ccnn2CC)C(C2CC2)C2CC2)c(F)c1] | B | B | 624.4 |
| 201 | 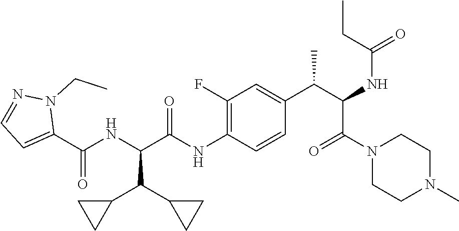 [SMILES: CCC(=O)N[C@@H](C(=O)N1CCN(C)CC1)[C@@H](C)c1ccc(NC(=O)[C@H](NC(=O)c2ccnn2CC)C(C2CC2)C2CC2)c(F)c1] | B | | 624.6 |
| 202 | 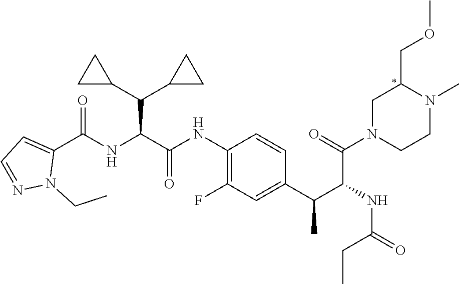 [SMILES: CCC(=O)N[C@@H](C(=O)N1CCN(C)C(COC)C1)[C@@H](C)c1ccc(NC(=O)[C@@H](NC(=O)c2ccnn2CC)C(C2CC2)C2CC2)c(F)c1] | B | | 668.6 |
| | Single diastereomer with undetermined | | | |
| | absolute stereochemistry at * | | | |
| 203 | 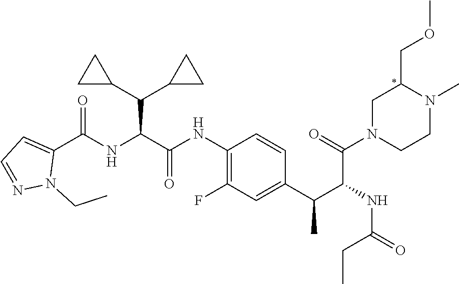 [SMILES: CCC(=O)N[C@@H](C(=O)N1CCN(C)C(COC)C1)[C@@H](C)c1ccc(NC(=O)[C@@H](NC(=O)c2ccnn2CC)C(C2CC2)C2CC2)c(F)c1] | B | | 668.6 |
| | Single diastereomer with undetermined | | | |
| | absolute stereochemistry at * | | | |
| 204 | 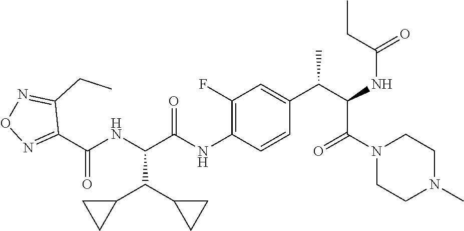 [SMILES: CCC(=O)N[C@@H](C(=O)N1CCN(C)CC1)[C@@H](C)c1ccc(NC(=O)[C@@H](NC(=O)c2nonc2CC)C(C2CC2)C2CC2)c(F)c1] | A | | 626.2 |
| 205 | 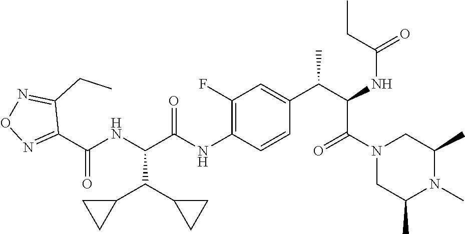 [SMILES: CCC(=O)N[C@@H](C(=O)N1C[C@@H](C)N(C)[C@@H](C)C1)[C@@H](C)c1ccc(NC(=O)[C@@H](NC(=O)c2nonc2CC)C(C2CC2)C2CC2)c(F)c1] | A | | 654.3 |
| 206 | 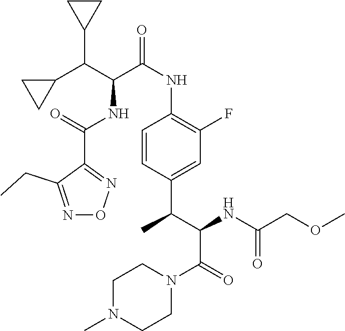 [SMILES: CCc1nonc1C(=O)N[C@H](C(=O)Nc1ccc([C@H](C)[C@@H](NC(=O)COC)C(=O)N2CCN(C)CC2)cc1F)C(C1CC1)C1CC1] | A | | 642.5 |
| 207 | 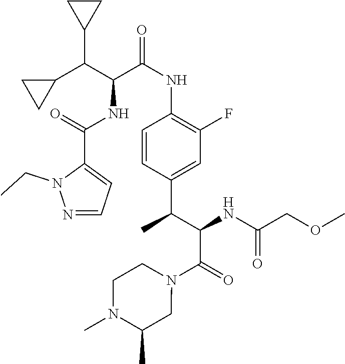 [SMILES: CCn1nccc1C(=O)N[C@H](C(=O)Nc1ccc([C@H](C)[C@@H](NC(=O)COC)C(=O)N2CCN(C)[C@H](C)C2)cc1F)C(C1CC1)C1CC1] | A | B | 654.6 |
| 208 | 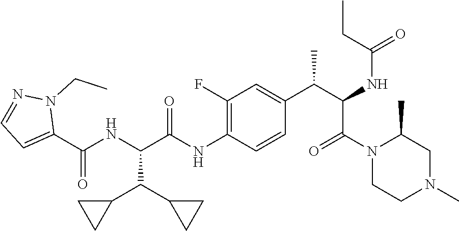 [SMILES: CCC(=O)N[C@@H](C(=O)N1CCN(C)C[C@@H]1C)[C@@H](C)c1ccc(NC(=O)[C@@H](NC(=O)c2ccnn2CC)C(C2CC2)C2CC2)c(F)c1] | A | | 638.4 |
| 209 | 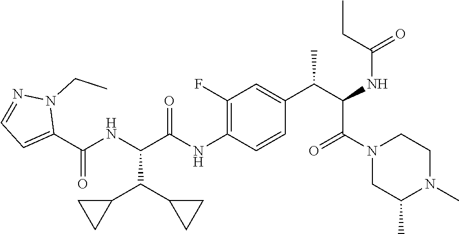 [SMILES: CCC(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1] | A | B | 638.6 |
| 210 | 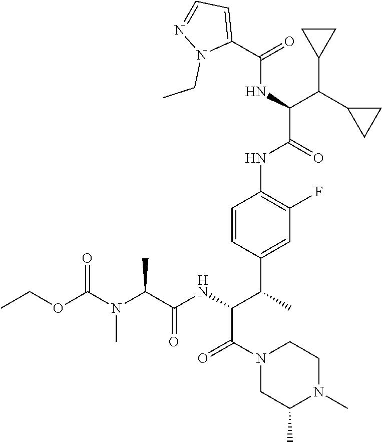 [SMILES: CCOC(=O)N(C)[C@@H](C)C(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1] | A | | 739.6 |
| 211 | 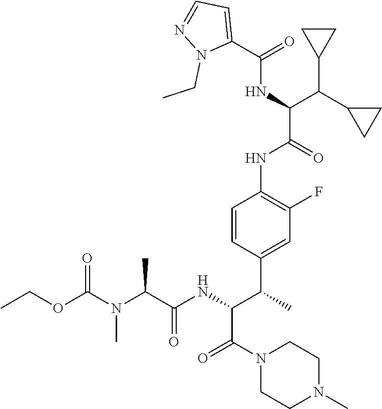 [SMILES: CCOC(=O)N(C)[C@@H](C)C(=O)N[C@@H](C(=O)N1CCN(C)CC1)[C@@H](C)c1ccc(NC(=O)[C@@H](NC(=O)c2ccnn2CC)C(C2CC2)C2CC2)c(F)c1] | A | | 725.6 |
| 212 | 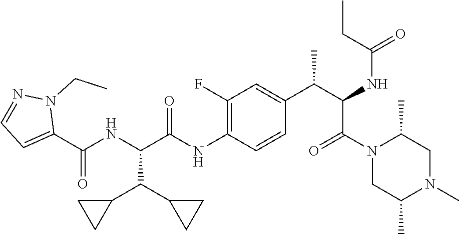 [SMILES: CCC(=O)N[C@@H](C(=O)N1C[C@@H](C)N(C)C[C@H]1C)[C@@H](C)c1ccc(NC(=O)[C@@H](NC(=O)c2ccnn2CC)C(C2CC2)C2CC2)c(F)c1] | A | B | 652.2 |
| 213 | 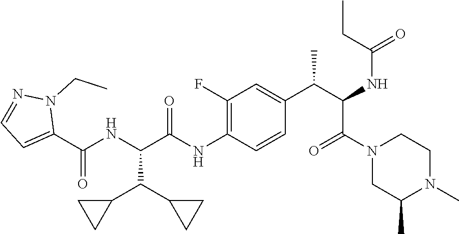 [SMILES: CCC(=O)N[C@@H](C(=O)N1CCN(C)[C@@H](C)C1)[C@@H](C)c1ccc(NC(=O)[C@@H](NC(=O)c2ccnn2CC)C(C2CC2)C2CC2)c(F)c1] | A | B | 638.4 |
| 214 | 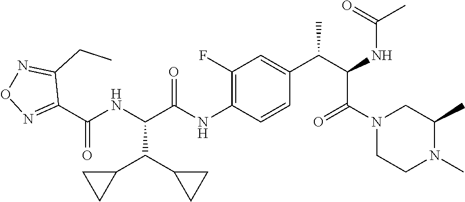 [SMILES: CCc1nonc1C(=O)N[C@H](C(=O)Nc1ccc([C@H](C)[C@@H](NC(C)=O)C(=O)N2CCN(C)[C@H](C)C2)cc1F)C(C1CC1)C1CC1] | A | | 626.1 |
| 215 | 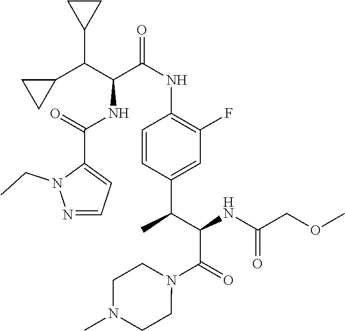 [SMILES: CCn1nccc1C(=O)N[C@H](C(=O)Nc1ccc([C@H](C)[C@@H](NC(=O)COC)C(=O)N2CCN(C)CC2)cc1F)C(C1CC1)C1CC1] | B | B | 640.4 |
| 216 | 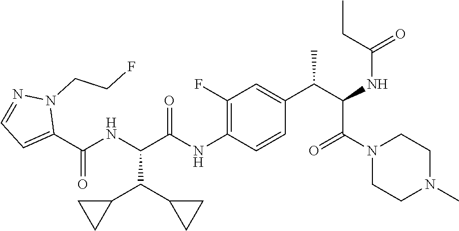 [SMILES: CCC(=O)N[C@@H](C(=O)N1CCN(C)CC1)[C@@H](C)c1ccc(NC(=O)[C@@H](NC(=O)c2ccnn2CCF)C(C2CC2)C2CC2)c(F)c1] | B | B | 642.5 |
| 217 | 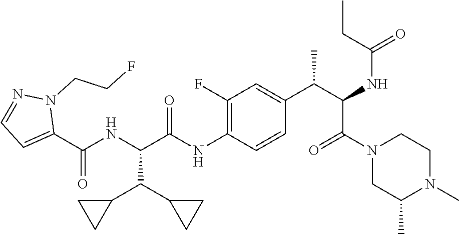 [SMILES: CCC(=O)N[C@@H](C(=O)N1CCN(C)[C@H](C)C1)[C@@H](C)c1ccc(NC(=O)[C@@H](NC(=O)c2ccnn2CCF)C(C2CC2)C2CC2)c(F)c1] | A | | 656.5 |
| 218 | 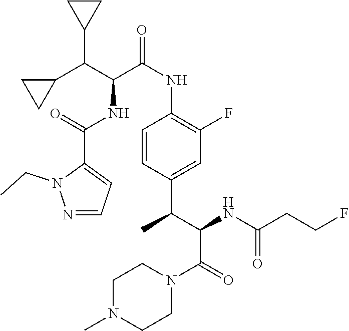 [SMILES: CCn1nccc1C(=O)N[C@H](C(=O)Nc1ccc([C@H](C)[C@@H](NC(=O)CCF)C(=O)N2CCN(C)CC2)cc1F)C(C1CC1)C1CC1] | B | | 642.4 |
| 219 | 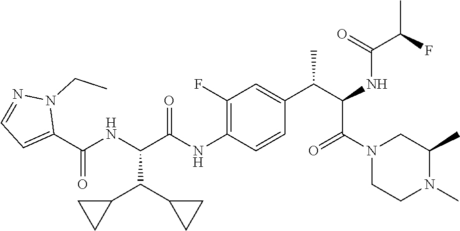 [SMILES: CCn1nccc1C(=O)N[C@H](C(=O)Nc1ccc([C@H](C)[C@@H](NC(=O)[C@@H](C)F)C(=O)N2CCN(C)[C@H](C)C2)cc1F)C(C1CC1)C1CC1] | A | | 656.5 |
| 220 | 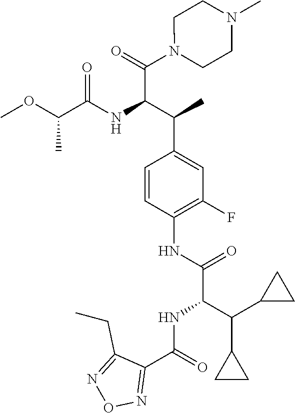 [SMILES: CCc1nonc1C(=O)N[C@H](C(=O)Nc1ccc([C@H](C)[C@@H](NC(=O)[C@H](C)OC)C(=O)N2CCN(C)CC2)cc1F)C(C1CC1)C1CC1] | A | | 656.6 |
| 221 | 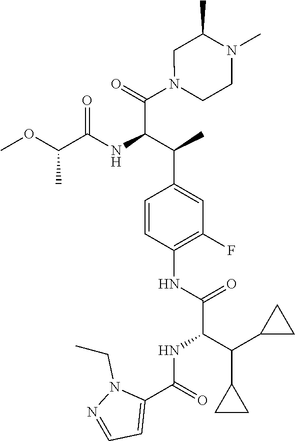 [SMILES: CCn1nccc1C(=O)N[C@H](C(=O)Nc1ccc([C@H](C)[C@@H](NC(=O)[C@H](C)OC)C(=O)N2CCN(C)[C@H](C)C2)cc1F)C(C1CC1)C1CC1] | A | B | 668.5 |
| 222 | 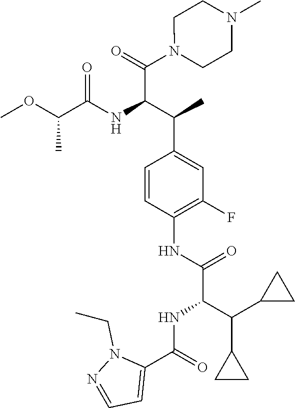 [SMILES: CCn1nccc1C(=O)N[C@H](C(=O)Nc1ccc([C@H](C)[C@@H](NC(=O)[C@H](C)OC)C(=O)N2CCN(C)CC2)cc1F)C(C1CC1)C1CC1] | A | B | 654.4 |
| 223 | 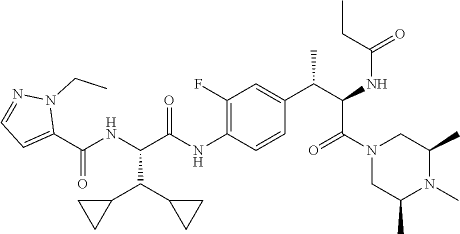 [SMILES: CCC(=O)N[C@@H](C(=O)N1C[C@@H](C)N(C)[C@@H](C)C1)[C@@H](C)c1ccc(NC(=O)[C@@H](NC(=O)c2ccnn2CC)C(C2CC2)C2CC2)c(F)c1] | A | | 652.6 |
| 224 | 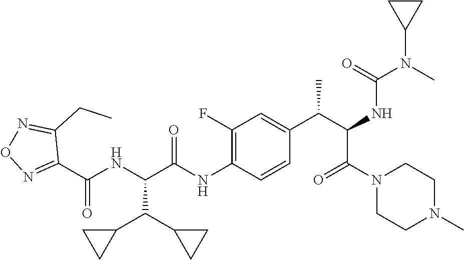 [SMILES: CCc1nonc1C(=O)N[C@H](C(=O)Nc1ccc([C@H](C)[C@@H](NC(=O)N(C)C2CC2)C(=O)N2CCN(C)CC2)cc1F)C(C1CC1)C1CC1] | A | | 667.4 |
| 225 | 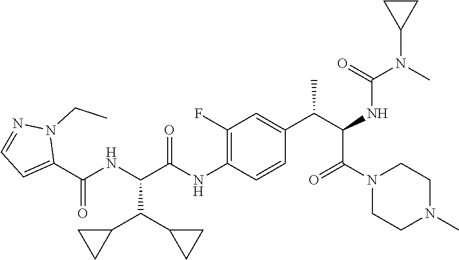 [SMILES: CCn1nccc1C(=O)N[C@H](C(=O)Nc1ccc([C@H](C)[C@@H](NC(=O)N(C)C2CC2)C(=O)N2CCN(C)CC2)cc1F)C(C1CC1)C1CC1] | A | | 665.1 |
| 226 | 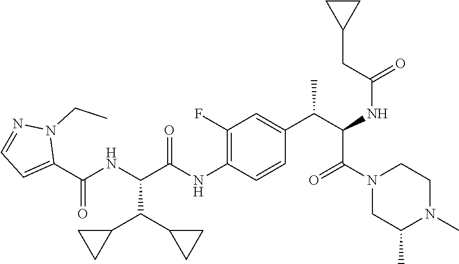 [SMILES: CCn1nccc1C(=O)N[C@H](C(=O)Nc1ccc([C@H](C)[C@@H](NC(=O)CC2CC2)C(=O)N2CCN(C)[C@H](C)C2)cc1F)C(C1CC1)C1CC1] | A | | 664.6 |
| 227 | 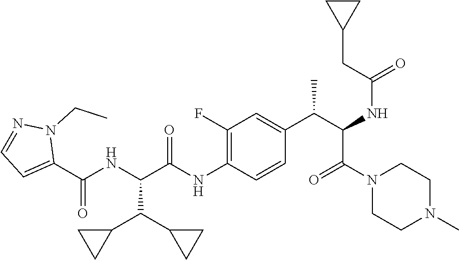 [SMILES: CCn1nccc1C(=O)N[C@H](C(=O)Nc1ccc([C@H](C)[C@@H](NC(=O)CC2CC2)C(=O)N2CCN(C)CC2)cc1F)C(C1CC1)C1CC1] | A | | 650.6 |
| 228 | 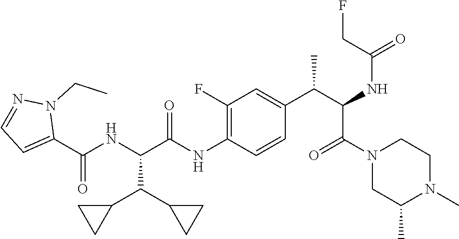 [SMILES: CCn1nccc1C(=O)N[C@H](C(=O)Nc1ccc([C@H](C)[C@@H](NC(=O)CF)C(=O)N2CCN(C)[C@H](C)C2)cc1F)C(C1CC1)C1CC1] | B | | 642.5 |
| 229 | 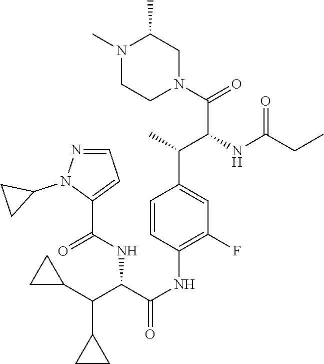 [SMILES: CCC(=O)N[C@@H](C(=O)N1CCN(C)[C@H](C)C1)[C@@H](C)c1ccc(NC(=O)[C@@H](NC(=O)c2ccnn2C2CC2)C(C2CC2)C2CC2)c(F)c1] | A | | 650.4 |
| 230 | 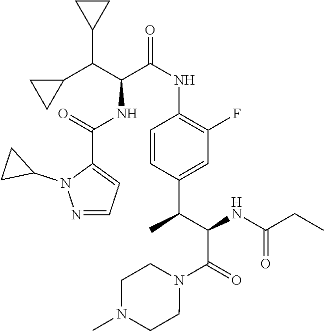 [SMILES: CCC(=O)N[C@@H](C(=O)N1CCN(C)CC1)[C@@H](C)c1ccc(NC(=O)[C@@H](NC(=O)c2ccnn2C2CC2)C(C2CC2)C2CC2)c(F)c1] | B | | 636.4 |
| 231 | 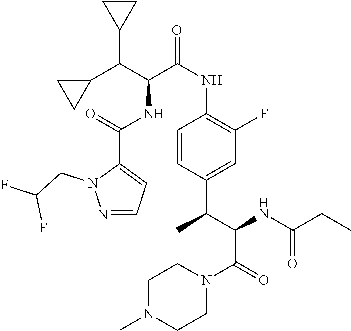 [SMILES: CCC(=O)N[C@@H](C(=O)N1CCN(C)CC1)[C@@H](C)c1ccc(NC(=O)[C@@H](NC(=O)c2ccnn2CC(F)F)C(C2CC2)C2CC2)c(F)c1] | B | | 660.3 |
| 232 | 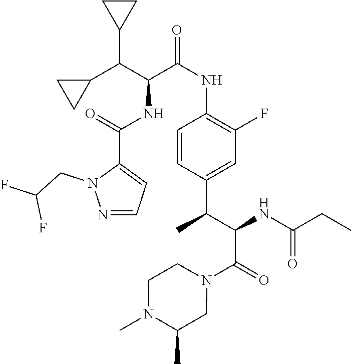 [SMILES: CCC(=O)N[C@@H](C(=O)N1CCN(C)[C@H](C)C1)[C@@H](C)c1ccc(NC(=O)[C@@H](NC(=O)c2ccnn2CC(F)F)C(C2CC2)C2CC2)c(F)c1] | A | | 674.2 |
| 233 | 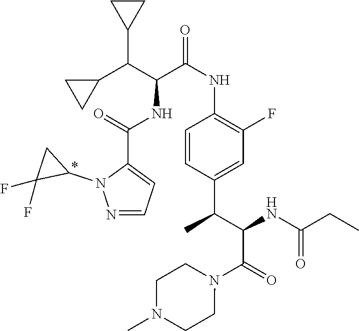 [SMILES: CCC(=O)N[C@@H](C(=O)N1CCN(C)CC1)[C@@H](C)c1ccc(NC(=O)[C@@H](NC(=O)c2ccnn2C2CC2(F)F)C(C2CC2)C2CC2)c(F)c1] | B | | 672.4 |
| | Single diastereomer with undetermined | | | |
| | absolute stereochemistry at * | | | |
| 234 | 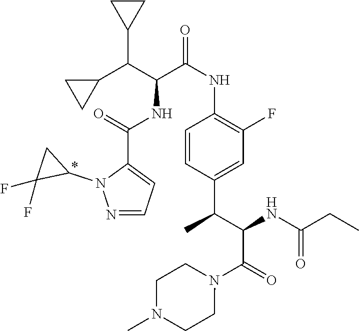 [SMILES: CCC(=O)N[C@@H](C(=O)N1CCN(C)CC1)[C@@H](C)c1ccc(NC(=O)[C@@H](NC(=O)c2ccnn2C2CC2(F)F)C(C2CC2)C2CC2)c(F)c1] | A | | 672.4 |
| | Single diastereomer with undetermined | | | |
| | absolute stereochemistry at * | | | |
| 235 | 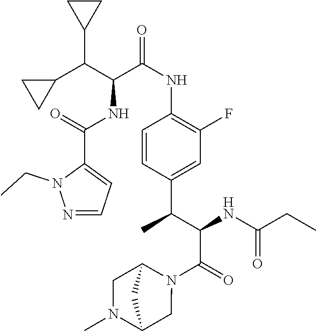 [SMILES: CCC(=O)N[C@@H](C(=O)N1C[C@@H]2C[C@H]1CN2C)[C@@H](C)c1ccc(NC(=O)[C@@H](NC(=O)c2ccnn2CC)C(C2CC2)C2CC2)c(F)c1] | B | | 636.1 |
| 236 | 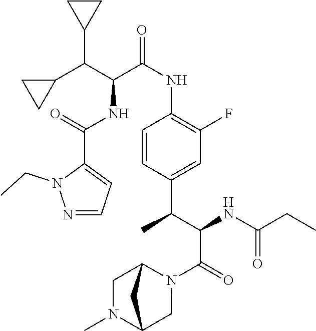 [SMILES: CCC(=O)N[C@@H](C(=O)N1C[C@H]2C[C@@H]1CN2C)[C@@H](C)c1ccc(NC(=O)[C@@H](NC(=O)c2ccnn2CC)C(C2CC2)C2CC2)c(F)c1] | B | | 636.4 |
| 237 | 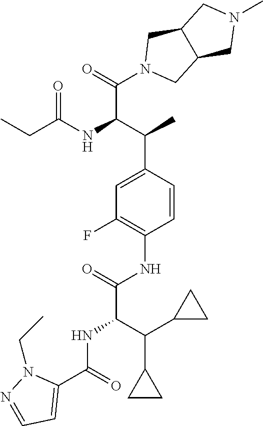 [SMILES: CCC(=O)N[C@@H](C(=O)N1C[C@H]2CN(C)C[C@H]2C1)[C@@H](C)c1ccc(NC(=O)[C@@H](NC(=O)c2ccnn2CC)C(C2CC2)C2CC2)c(F)c1] | C | | 650.5 |
| 238 | 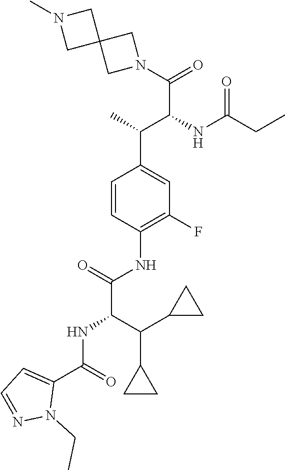 [SMILES: CCC(=O)N[C@@H](C(=O)N1CC2(CN(C)C2)C1)[C@@H](C)c1ccc(NC(=O)[C@@H](NC(=O)c2ccnn2CC)C(C2CC2)C2CC2)c(F)c1] | C | | 636.2 |
| 239 | 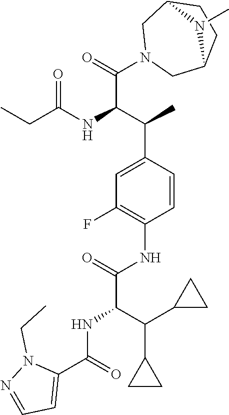 [SMILES: CCC(=O)N[C@@H](C(=O)N1C[C@H]2CC[C@@H](C1)N2C)[C@@H](C)c1ccc(NC(=O)[C@@H](NC(=O)c2ccnn2CC)C(C2CC2)C2CC2)c(F)c1] | A | | 650.3 |
| 240 | 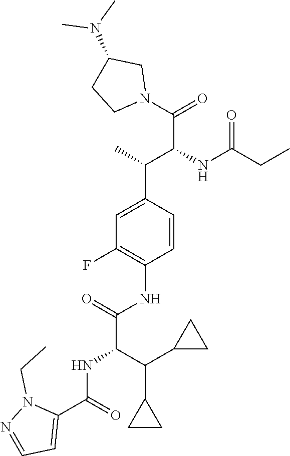 [SMILES: CCC(=O)N[C@@H](C(=O)N1CC[C@H](N(C)C)C1)[C@@H](C)c1ccc(NC(=O)[C@@H](NC(=O)c2ccnn2CC)C(C2CC2)C2CC2)c(F)c1] | B | | 638.4 |
| 241 | 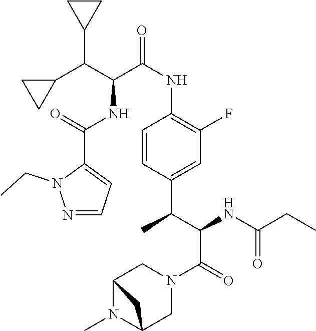 [SMILES: CCC(=O)N[C@@H](C(=O)N1C[C@H]2C[C@@H](C1)N2C)[C@@H](C)c1ccc(NC(=O)[C@@H](NC(=O)c2ccnn2CC)C(C2CC2)C2CC2)c(F)c1] | B | | 636.4 |
| 242 | 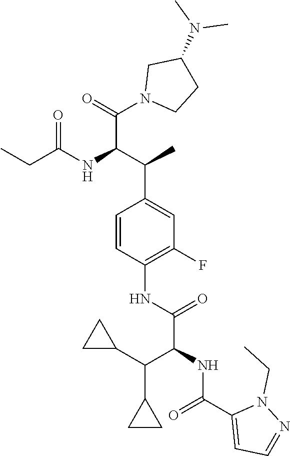 [SMILES: CCC(=O)N[C@@H](C(=O)N1CC[C@@H](N(C)C)C1)[C@@H](C)c1ccc(NC(=O)[C@@H](NC(=O)c2ccnn2CC)C(C2CC2)C2CC2)c(F)c1] | B | | 638.2 |
| 243 | 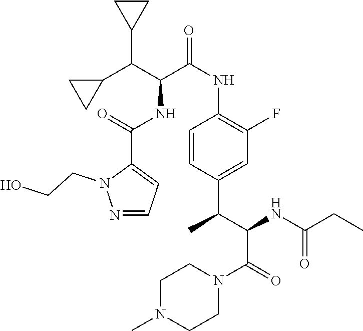 [SMILES: CCC(=O)N[C@@H](C(=O)N1CCN(C)CC1)[C@@H](C)c1ccc(NC(=O)[C@@H](NC(=O)c2ccnn2CCO)C(C2CC2)C2CC2)c(F)c1] | C | | 640.4 |
| 244 | 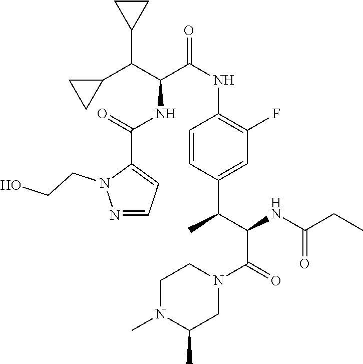 [SMILES: CCC(=O)N[C@@H](C(=O)N1CCN(C)[C@H](C)C1)[C@@H](C)c1ccc(NC(=O)[C@@H](NC(=O)c2ccnn2CCO)C(C2CC2)C2CC2)c(F)c1] | C | | 654.4 |
| 245 | 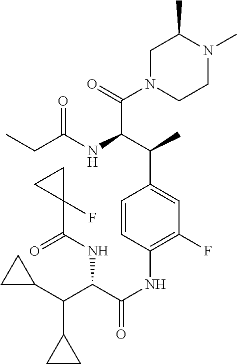 [SMILES: CCC(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | B | C | 602.4 |
| 246 | 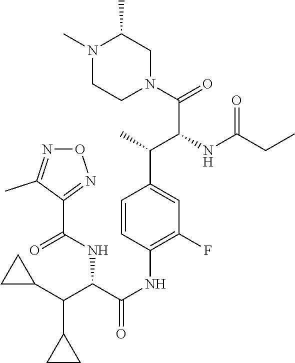 [SMILES: CCC(=O)N[C@@H](C(=O)N1CCN(C)[C@H](C)C1)[C@@H](C)c1ccc(NC(=O)[C@@H](NC(=O)c2nonc2C)C(C2CC2)C2CC2)c(F)c1] | A | C | 626.4 |
| 247 | 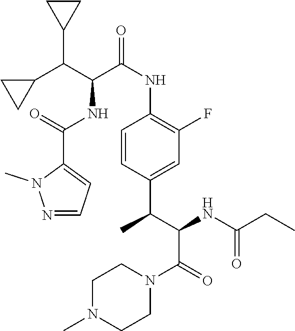 [SMILES: CCC(=O)N[C@@H](C(=O)N1CCN(C)CC1)[C@@H](C)c1ccc(NC(=O)[C@@H](NC(=O)c2ccnn2C)C(C2CC2)C2CC2)c(F)c1] | B | | 610.4 |
| 248 | 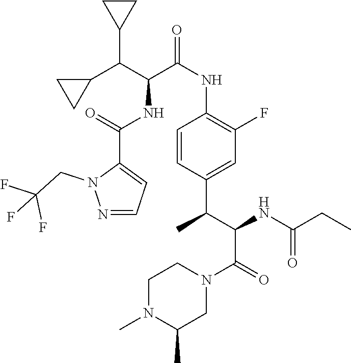 [SMILES: CCC(=O)N[C@@H](C(=O)N1CCN(C)[C@H](C)C1)[C@@H](C)c1ccc(NC(=O)[C@@H](NC(=O)c2ccnn2CC(F)(F)F)C(C2CC2)C2CC2)c(F)c1] | B | | 692.4 |
| 249 | 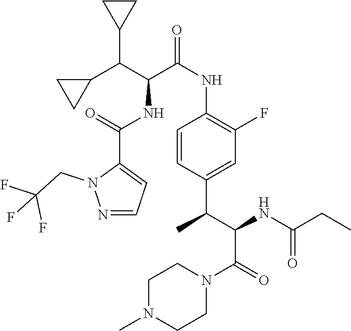 [SMILES: CCC(=O)N[C@@H](C(=O)N1CCN(C)CC1)[C@@H](C)c1ccc(NC(=O)[C@@H](NC(=O)c2ccnn2CC(F)(F)F)C(C2CC2)C2CC2)c(F)c1] | B | | 678.4 |
| 250 | 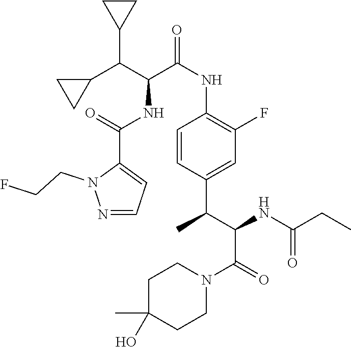 [SMILES: CCC(=O)N[C@@H](C(=O)N1CCC(C)(O)CC1)[C@@H](C)c1ccc(NC(=O)[C@@H](NC(=O)c2ccnn2CCF)C(C2CC2)C2CC2)c(F)c1] | A | | 657.3 |
| 251 | 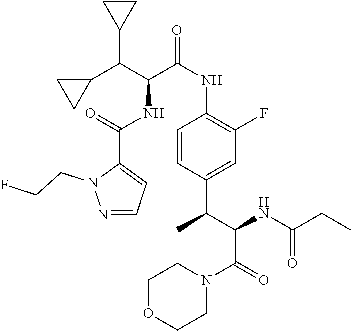 [SMILES: CCC(=O)N[C@@H](C(=O)N1CCOCC1)[C@@H](C)c1ccc(NC(=O)[C@@H](NC(=O)c2ccnn2CCF)C(C2CC2)C2CC2)c(F)c1] | B | | 629.2 |
| 252 | 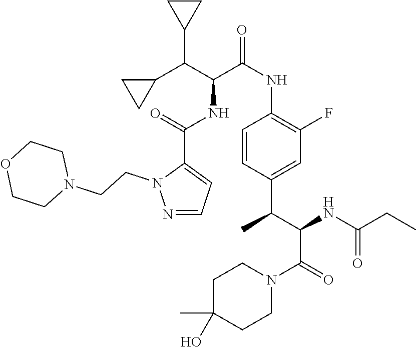 [SMILES: CCC(=O)N[C@@H](C(=O)N1CCC(C)(O)CC1)[C@@H](C)c1ccc(NC(=O)[C@@H](NC(=O)c2ccnn2CCN2CCOCC2)C(C2CC2)C2CC2)c(F)c1] | B | | 724.5 |
| 253 | 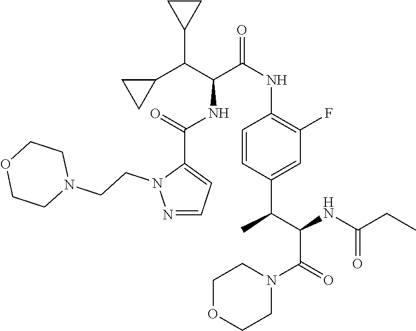 [SMILES: CCC(=O)N[C@@H](C(=O)N1CCOCC1)[C@@H](C)c1ccc(NC(=O)[C@@H](NC(=O)c2ccnn2CCN2CCOCC2)C(C2CC2)C2CC2)c(F)c1] | C | | 696.4 |
| 254 | 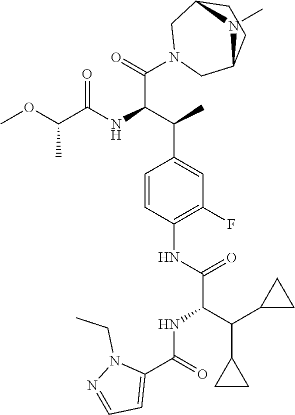 [SMILES: CCn1nccc1C(=O)N[C@H](C(=O)Nc1ccc([C@H](C)[C@@H](NC(=O)[C@H](C)OC)C(=O)N2C[C@H]3CC[C@@H](C2)N3C)cc1F)C(C1CC1)C1CC1] | A | | 680.3 |
| 255 | 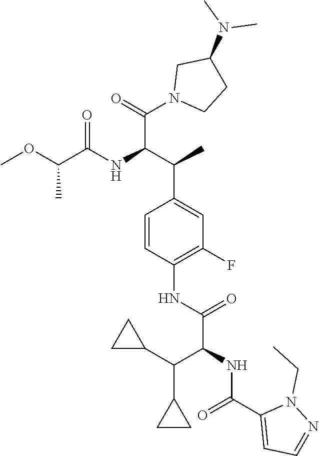 [SMILES: CCn1nccc1C(=O)N[C@H](C(=O)Nc1ccc([C@H](C)[C@@H](NC(=O)[C@H](C)OC)C(=O)N2CC[C@H](N(C)C)C2)cc1F)C(C1CC1)C1CC1] | A | | 668.2 |
| 256 | 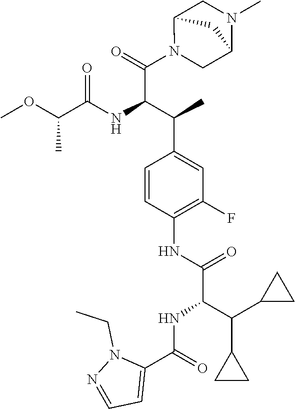 [SMILES: CCn1nccc1C(=O)N[C@H](C(=O)Nc1ccc([C@H](C)[C@@H](NC(=O)[C@H](C)OC)C(=O)N2C[C@H]3C[C@@H]2CN3C)cc1F)C(C1CC1)C1CC1] | B | | 666.5 |
| 257 | 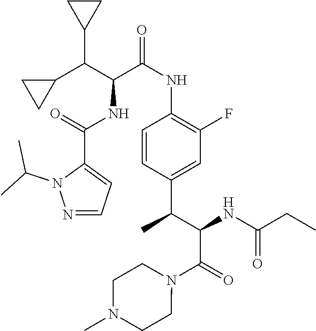 [SMILES: CCC(=O)N[C@@H](C(=O)N1CCN(C)CC1)[C@@H](C)c1ccc(NC(=O)[C@@H](NC(=O)c2ccnn2C(C)C)C(C2CC2)C2CC2)c(F)c1] | A | | 638.4 |
| 258 | 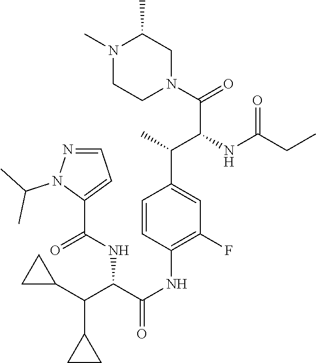 [SMILES: CCC(=O)N[C@@H](C(=O)N1CCN(C)[C@H](C)C1)[C@@H](C)c1ccc(NC(=O)[C@@H](NC(=O)c2ccnn2C(C)C)C(C2CC2)C2CC2)c(F)c1] | A | | 652.5 |
| 259 | 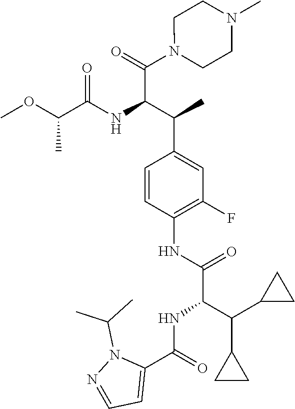 [SMILES: CO[C@@H](C)C(=O)N[C@@H](C(=O)N1CCN(C)CC1)[C@@H](C)c1ccc(NC(=O)[C@@H](NC(=O)c2ccnn2C(C)C)C(C2CC2)C2CC2)c(F)c1] | A | | 668.4 |
| 260 | 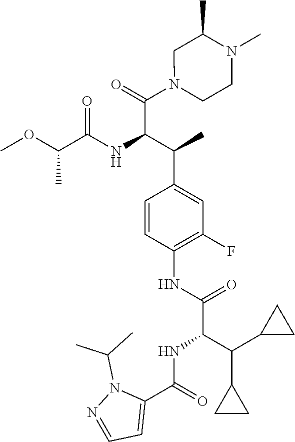 [SMILES: CO[C@@H](C)C(=O)N[C@@H](C(=O)N1CCN(C)[C@H](C)C1)[C@@H](C)c1ccc(NC(=O)[C@@H](NC(=O)c2ccnn2C(C)C)C(C2CC2)C2CC2)c(F)c1] | A | | 682.5 |
| 261 | 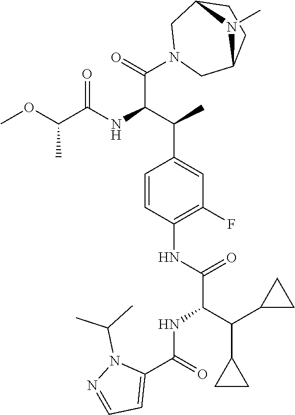 [SMILES: CO[C@@H](C)C(=O)N[C@@H](C(=O)N1C[C@H]2CC[C@@H](C1)N2C)[C@@H](C)c1ccc(NC(=O)[C@@H](NC(=O)c2ccnn2C(C)C)C(C2CC2)C2CC2)c(F)c1] | A | | 694.4 |
| 262 | 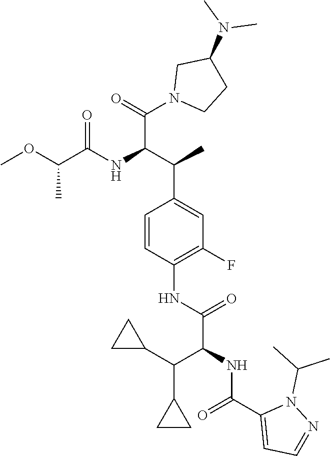 [SMILES: CO[C@@H](C)C(=O)N[C@@H](C(=O)N1CC[C@H](N(C)C)C1)[C@@H](C)c1ccc(NC(=O)[C@@H](NC(=O)c2ccnn2C(C)C)C(C2CC2)C2CC2)c(F)c1] | A | | 682.5 |
| 263 | 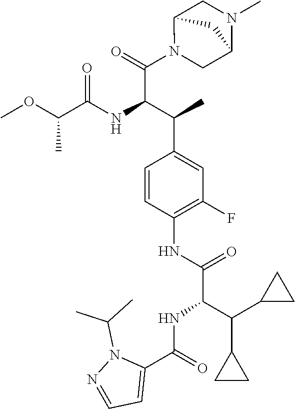 [SMILES: CO[C@@H](C)C(=O)N[C@@H](C(=O)N1C[C@H]2C[C@@H]1CN2C)[C@@H](C)c1ccc(NC(=O)[C@@H](NC(=O)c2ccnn2C(C)C)C(C2CC2)C2CC2)c(F)c1] | B | | 680.4 |
| 264 | 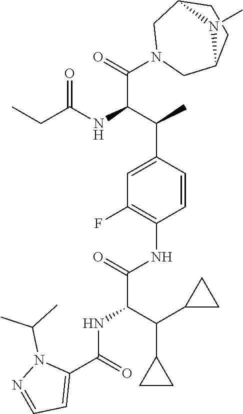 [SMILES: CCC(=O)N[C@@H](C(=O)N1C[C@H]2CC[C@@H](C1)N2C)[C@@H](C)c1ccc(NC(=O)[C@@H](NC(=O)c2ccnn2C(C)C)C(C2CC2)C2CC2)c(F)c1] | A | | 664.4 |
| 265 | 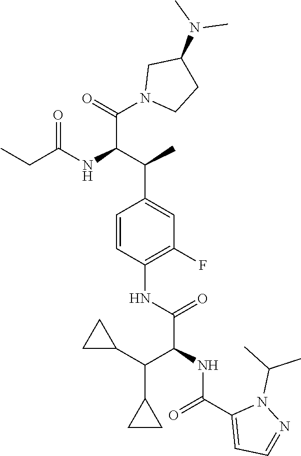 [SMILES: CCC(=O)N[C@@H](C(=O)N1CC[C@H](N(C)C)C1)[C@@H](C)c1ccc(NC(=O)[C@@H](NC(=O)c2ccnn2C(C)C)C(C2CC2)C2CC2)c(F)c1] | A | | 652.5 |
| 266 | 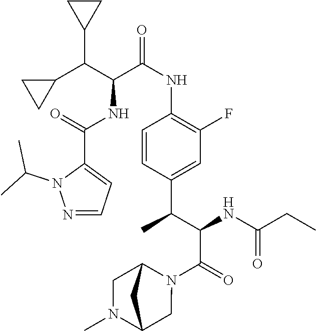 [SMILES: CCC(=O)N[C@@H](C(=O)N1C[C@H]2C[C@@H]1CN2C)[C@@H](C)c1ccc(NC(=O)[C@@H](NC(=O)c2ccnn2C(C)C)C(C2CC2)C2CC2)c(F)c1] | B | | 650.4 |
| 267 | 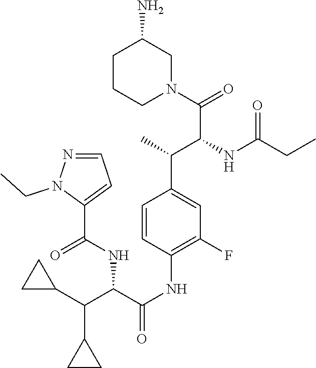 [SMILES: CCC(=O)N[C@@H](C(=O)N1CCC[C@H](N)C1)[C@@H](C)c1ccc(NC(=O)[C@@H](NC(=O)c2ccnn2CC)C(C2CC2)C2CC2)c(F)c1] | B | C | 624.3 |
| 268 | 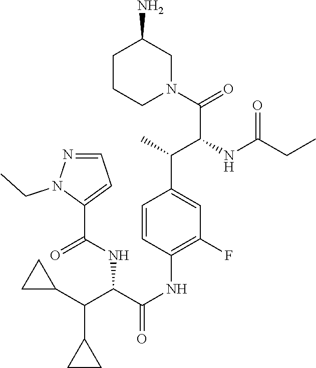 [SMILES: CCC(=O)N[C@@H](C(=O)N1CCC[C@@H](N)C1)[C@@H](C)c1ccc(NC(=O)[C@@H](NC(=O)c2ccnn2CC)C(C2CC2)C2CC2)c(F)c1] | B | | 624.1 |
| 269 | 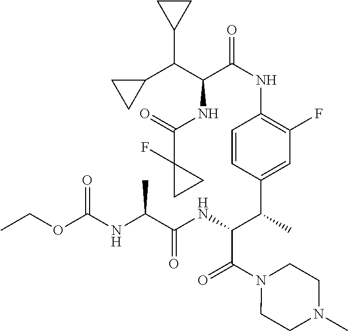 [SMILES: CCOC(=O)N[C@@H](C)C(=O)N[C@@H](C(=O)N1CCN(C)CC1)[C@@H](C)c1ccc(NC(=O)[C@@H](NC(=O)C2(F)CC2)C(C2CC2)C2CC2)c(F)c1] | A | C | 675.5 |
| 270 | 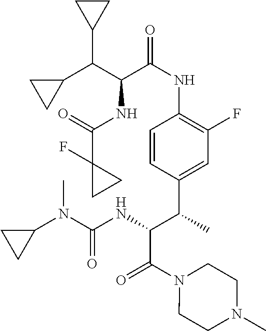 [SMILES: C[C@@H](c1ccc(NC(=O)[C@@H](NC(=O)C2(F)CC2)C(C2CC2)C2CC2)c(F)c1)[C@@H](NC(=O)N(C)C1CC1)C(=O)N1CCN(C)CC1] | A | C | 629.4 |
| 271 | 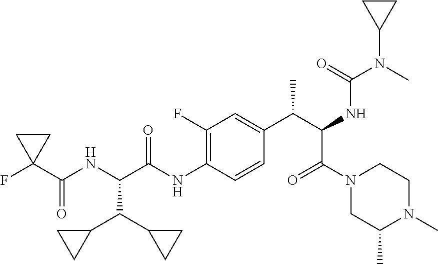 [SMILES: C[C@@H]1CN(C(=O)[C@H](NC(=O)N(C)C2CC2)[C@@H](C)c2ccc(NC(=O)[C@@H](NC(=O)C3(F)CC3)C(C3CC3)C3CC3)c(F)c2)CCN1C] | A | C | 643.4 |
| 272 | 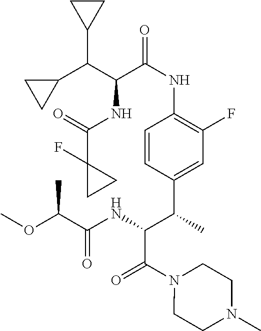 | B | | 618.2 |
| 273 | 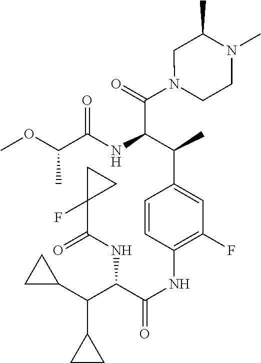 | A | C | 632.2 |
| 274 | 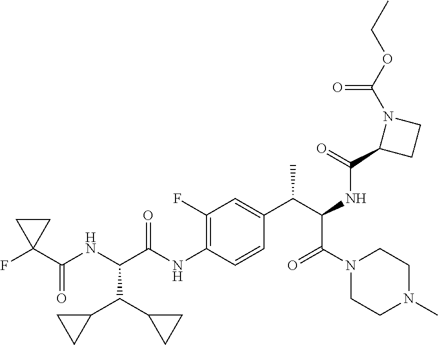 [SMILES: CCOC(=O)N1CC[C@H]1C(=O)N[C@@H](C(=O)N1CCN(C)CC1)[C@@H](C)c1ccc(NC(=O)[C@@H](NC(=O)C2(F)CC2)C(C2CC2)C2CC2)c(F)c1] | B | | 687.5 |
| 275 | 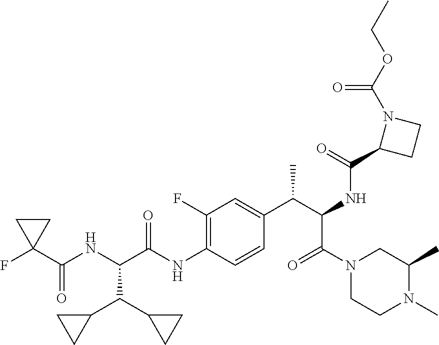 [SMILES: CCOC(=O)N1CC[C@H]1C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | A | C | 701.3 |
| 276 | 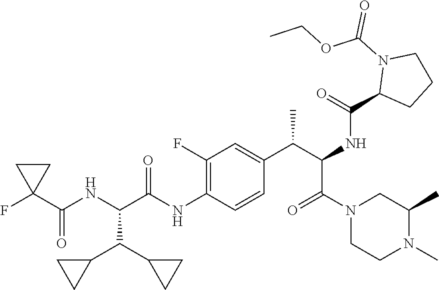 [SMILES: CCOC(=O)N1CCC[C@H]1C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | A | C | 701.5 |
| 277 | 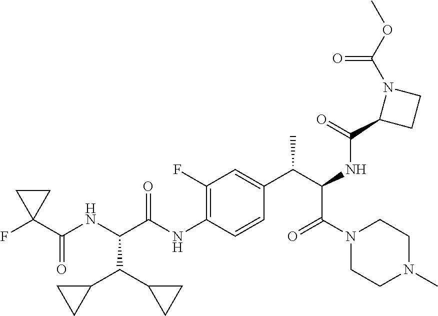 [SMILES: COC(=O)N1CC[C@H]1C(=O)N[C@@H](C(=O)N1CCN(C)CC1)[C@@H](C)c1ccc(NC(=O)[C@@H](NC(=O)C2(F)CC2)C(C2CC2)C2CC2)c(F)c1] | B | C | 673.4 |
| 278 | 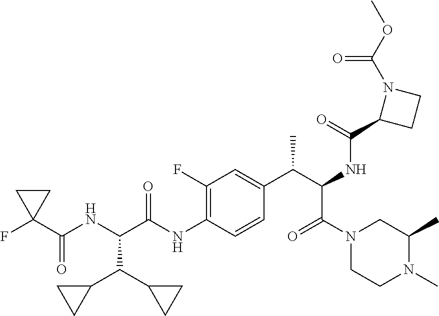 [SMILES: COC(=O)N1CC[C@H]1C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | A | C | 687.4 |
| 279 | 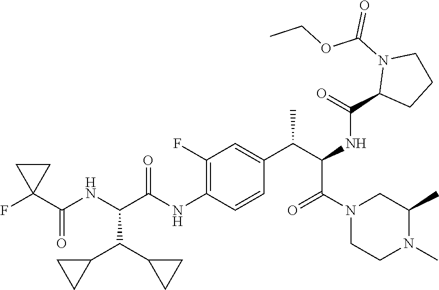 [SMILES: CCOC(=O)N1CCC[C@H]1C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | A | C | 715.5 |
| 280 | 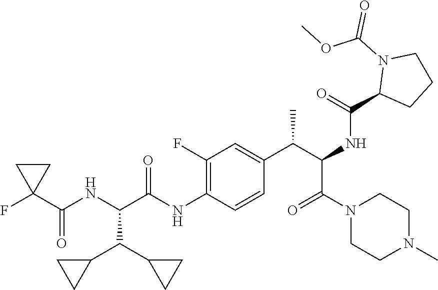 [SMILES: COC(=O)N1CCC[C@H]1C(=O)N[C@@H](C(=O)N1CCN(C)CC1)[C@@H](C)c1ccc(NC(=O)[C@@H](NC(=O)C2(F)CC2)C(C2CC2)C2CC2)c(F)c1] | B | C | 687.4 |
| 281 | 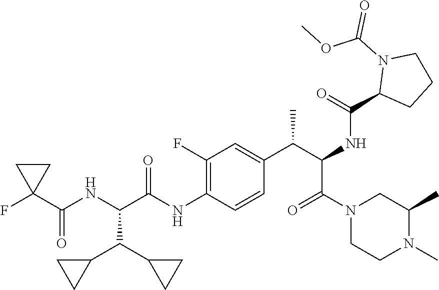 [SMILES: COC(=O)N1CCC[C@H]1C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | A | C | 701.2 |
| 282 | 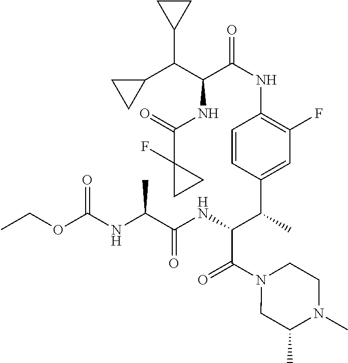 [SMILES: CCOC(=O)N[C@@H](C)C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | A | B | 689.5 |
| 283 | 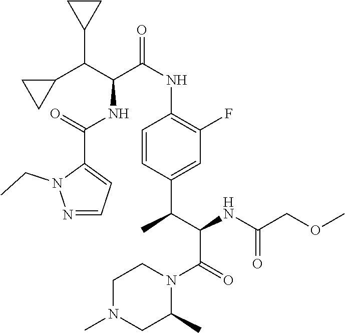 [SMILES: CCn1nccc1C(=O)N[C@H](C(=O)Nc1ccc([C@H](C)[C@@H](NC(=O)COC)C(=O)N2CCN(C)C[C@@H]2C)cc1F)C(C1CC1)C1CC1] | A | B | 654.4 |
| 284 | 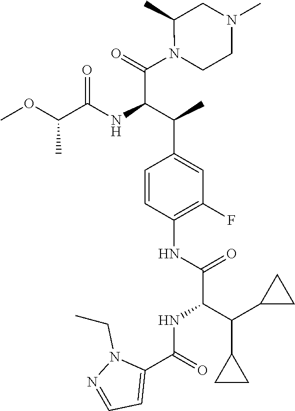 [SMILES: CCn1nccc1C(=O)N[C@H](C(=O)Nc1ccc([C@H](C)[C@@H](NC(=O)[C@H](C)OC)C(=O)N2CCN(C)C[C@@H]2C)cc1F)C(C1CC1)C1CC1] | A | B | 668.5 |
| 285 | 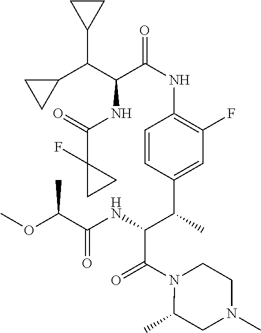 | B | C | 632.4 |
| 286 | 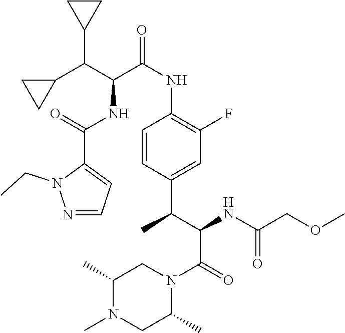 [SMILES: CCn1nccc1C(=O)N[C@H](C(=O)Nc1ccc([C@H](C)[C@@H](NC(=O)COC)C(=O)N2C[C@@H](C)N(C)C[C@H]2C)cc1F)C(C1CC1)C1CC1] | A | B | 668.3 |
| 287 | 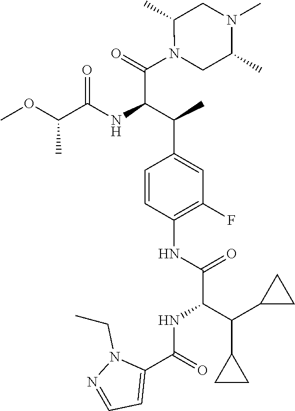 [SMILES: CCn1nccc1C(=O)N[C@H](C(=O)Nc1ccc([C@H](C)[C@@H](NC(=O)[C@H](C)OC)C(=O)N2C[C@@H](C)N(C)C[C@H]2C)cc1F)C(C1CC1)C1CC1] | A | A | 682.3 |
| 288 | 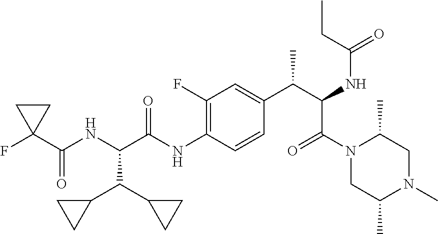 [SMILES: CCC(=O)N[C@@H](C(=O)N1C[C@@H](C)N(C)C[C@H]1C)[C@@H](C)c1ccc(NC(=O)[C@@H](NC(=O)C2(F)CC2)C(C2CC2)C2CC2)c(F)c1] | A | C | 616.4 |
| 289 | 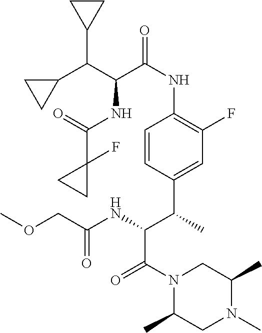 | A | C | 632.4 |
| 290 | 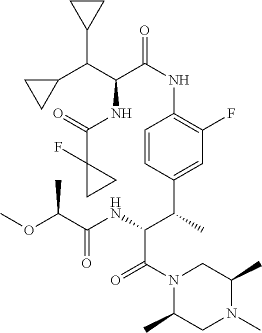 | A | C | 646.4 |
| 291 | 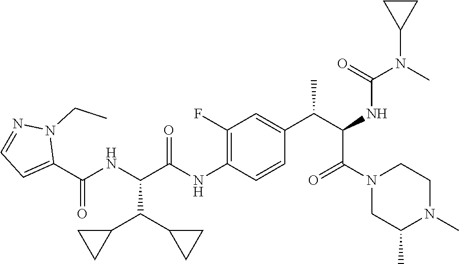 [SMILES: CCn1nccc1C(=O)N[C@H](C(=O)Nc1ccc([C@H](C)[C@@H](NC(=O)N(C)C2CC2)C(=O)N2CCN(C)[C@H](C)C2)cc1F)C(C1CC1)C1CC1] | A | A | 679.5 |
| 292 | 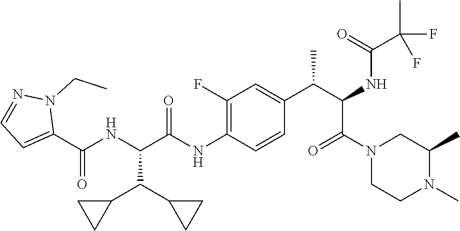 [SMILES: CCn1nccc1C(=O)N[C@H](C(=O)Nc1ccc([C@H](C)[C@@H](NC(=O)C(C)(F)F)C(=O)N2CCN(C)[C@H](C)C2)cc1F)C(C1CC1)C1CC1] | A | B | 674.2 |
| 293 | 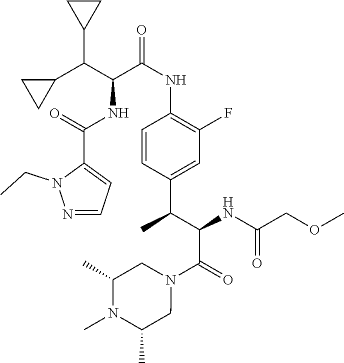 [SMILES: CCn1nccc1C(=O)N[C@H](C(=O)Nc1ccc([C@H](C)[C@@H](NC(=O)COC)C(=O)N2C[C@@H](C)N(C)[C@@H](C)C2)cc1F)C(C1CC1)C1CC1] | A | B | 668.5 |
| 294 | 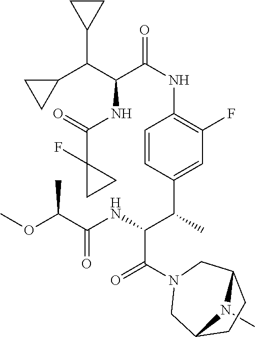 | B | C | 644.3 |
| 295 | 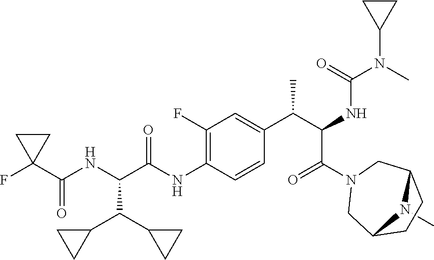 [SMILES: C[C@@H](c1ccc(NC(=O)[C@@H](NC(=O)C2(F)CC2)C(C2CC2)C2CC2)c(F)c1)[C@@H](NC(=O)N(C)C1CC1)C(=O)N1C[C@H]2CC[C@@H](C1)N2C] | A | C | 655.4 |
| 296 | 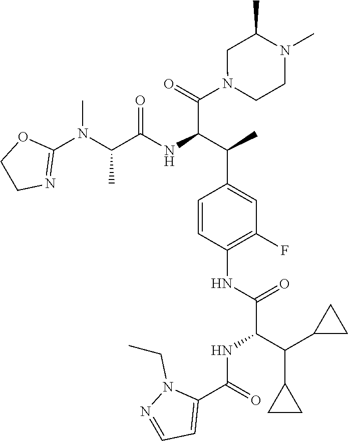 [SMILES: CCn1nccc1C(=O)N[C@H](C(=O)Nc1ccc([C@H](C)[C@@H](NC(=O)[C@H](C)N(C)C2=NCCO2)C(=O)N2CCN(C)[C@H](C)C2)cc1F)C(C1CC1)C1CC1] | A | B | 736.6 |
| 297 | 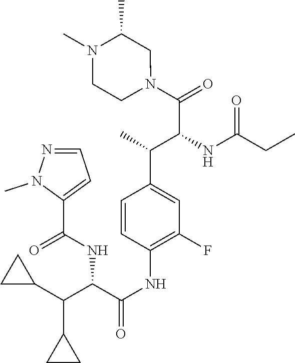 [SMILES: CCC(=O)N[C@@H](C(=O)N1CCN(C)[C@H](C)C1)[C@@H](C)c1ccc(NC(=O)[C@@H](NC(=O)c2ccnn2C)C(C2CC2)C2CC2)c(F)c1] | A | B | 624.5 |
| 298 | 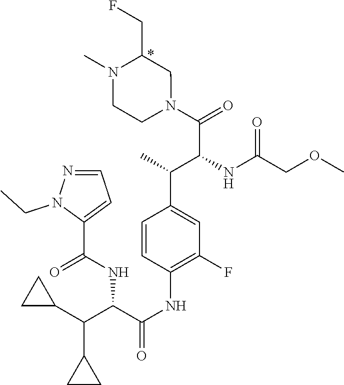 [SMILES: CCn1nccc1C(=O)N[C@H](C(=O)Nc1ccc([C@H](C)[C@@H](NC(=O)COC)C(=O)N2CCN(C)C(CF)C2)cc1F)C(C1CC1)C1CC1] | A | B | 672.3 |
| | Single diastereomer with undetermined | | | |
| | absolute stereochemistry at * | | | |
| 299 | 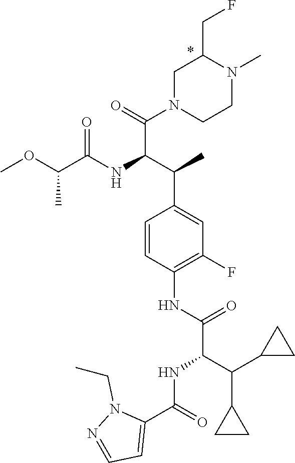 [SMILES: CCn1nccc1C(=O)N[C@H](C(=O)Nc1ccc([C@H](C)[C@@H](NC(=O)[C@H](C)OC)C(=O)N2CCN(C)C(CF)C2)cc1F)C(C1CC1)C1CC1] | A | B | 686.3 |
| | Single diastereomer with undetermined | | | |
| | absolute stereochemistry at * | | | |
| 300 | 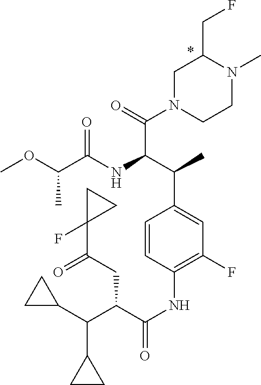 | A | C | 650.4 |
| | Single diastereomer with undetermined | | | |
| | absolute stereochemistry at * | | | |
| 301 | 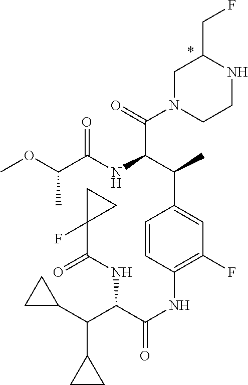 [SMILES: CO[C@@H](C)C(=O)N[C@@H](C(=O)N1CCNC(CF)C1)[C@@H](C)c1ccc(NC(=O)[C@@H](NC(=O)C2(F)CC2)C(C2CC2)C2CC2)c(F)c1] | B | C | 636.5 |
| | Single diastereomer with undetermined | | | |
| | absolute stereochemistry at * | | | |
| 302 | 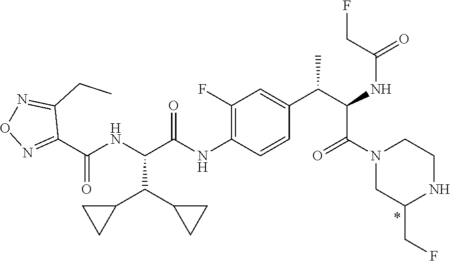 [SMILES: CCc1nonc1C(=O)N[C@H](C(=O)Nc1ccc([C@H](C)[C@@H](NC(=O)CF)C(=O)N2CCNC(CF)C2)cc1F)C(C1CC1)C1CC1] | B | C | 648.4 |
| | Single diastereomer with undetermined | | | |
| | absolute stereochemistry at * | | | |
| 303 | 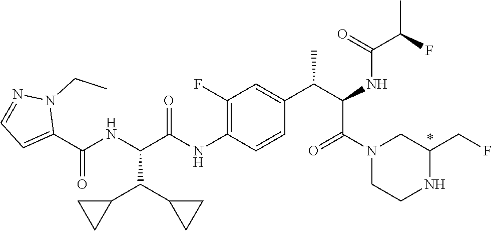 [SMILES: CCn1nccc1C(=O)N[C@H](C(=O)Nc1ccc([C@H](C)[C@@H](NC(=O)[C@@H](C)F)C(=O)N2CCNC(CF)C2)cc1F)C(C1CC1)C1CC1] | B | C | 660.5 |
| | Single diastereomer with undetermined | | | |
| | absolute stereochemistry at * | | | |
| 304 | 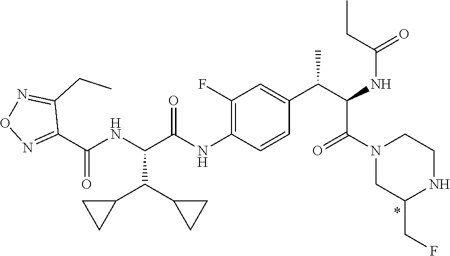 [SMILES: CCC(=O)N[C@@H](C(=O)N1CCNC(CF)C1)[C@@H](C)c1ccc(NC(=O)[C@@H](NC(=O)c2nonc2CC)C(C2CC2)C2CC2)c(F)c1] | A | C | 644.5 |
| | Single diastereomer with undetermined | | | |
| | absolute stereochemistry at * | | | |
| 305 | 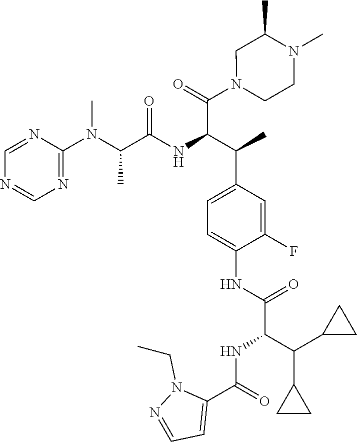 [SMILES: CCn1nccc1C(=O)N[C@H](C(=O)Nc1ccc([C@H](C)[C@@H](NC(=O)[C@H](C)N(C)c2ncncn2)C(=O)N2CCN(C)[C@H](C)C2)cc1F)C(C1CC1)C1CC1] | A | A | 746.4 |
| 306 | 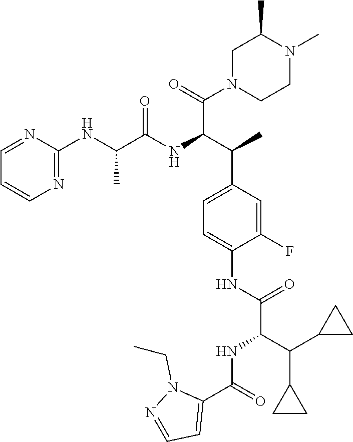 [SMILES: CCn1nccc1C(=O)N[C@H](C(=O)Nc1ccc([C@H](C)[C@@H](NC(=O)[C@H](C)Nc2ncccn2)C(=O)N2CCN(C)[C@H](C)C2)cc1F)C(C1CC1)C1CC1] | A | A | 731.5 |
| 307 | 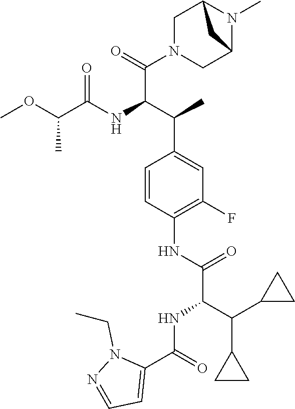 [SMILES: CCn1nccc1C(=O)N[C@H](C(=O)Nc1ccc([C@H](C)[C@@H](NC(=O)[C@H](C)OC)C(=O)N2C[C@H]3C[C@@H](C2)N3C)cc1F)C(C1CC1)C1CC1] | B | C | 666.1 |
| 308 | 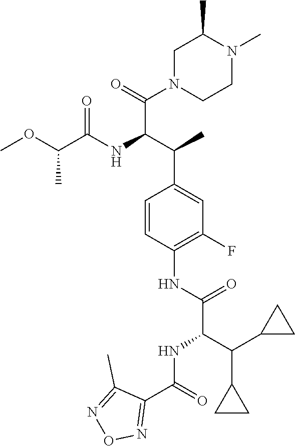 [SMILES: CO[C@@H](C)C(=O)N[C@@H](C(=O)N1CCN(C)[C@H](C)C1)[C@@H](C)c1ccc(NC(=O)[C@@H](NC(=O)c2nonc2C)C(C2CC2)C2CC2)c(F)c1] | A | C | 656.2 |
| 309 | 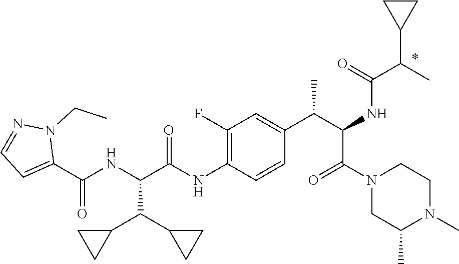 [SMILES: CCn1nccc1C(=O)N[C@H](C(=O)Nc1ccc([C@H](C)[C@@H](NC(=O)C(C)C2CC2)C(=O)N2CCN(C)[C@H](C)C2)cc1F)C(C1CC1)C1CC1] | A | A | 678.1 |
| | Single diastereomer with undetermined | | | |
| | absolute stereochemistry at * | | | |
| 310 | 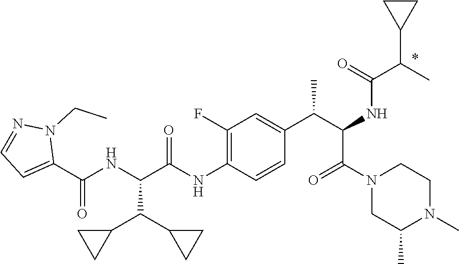 [SMILES: CCn1nccc1C(=O)N[C@H](C(=O)Nc1ccc([C@H](C)[C@@H](NC(=O)C(C)C2CC2)C(=O)N2CCN(C)[C@H](C)C2)cc1F)C(C1CC1)C1CC1] | A | B | 678.2 |
| | Single diastereomer with undetermined | | | |
| | absolute stereochemistry at * | | | |
| 311 | 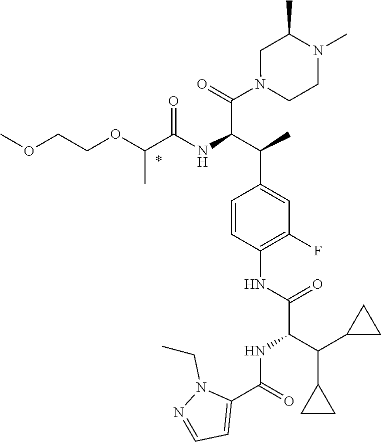 [SMILES: CCn1nccc1C(=O)N[C@H](C(=O)Nc1ccc([C@H](C)[C@@H](NC(=O)C(C)OCCOC)C(=O)N2CCN(C)[C@H](C)C2)cc1F)C(C1CC1)C1CC1] | B | C | 712.4 |
| | Single diastereomer with undetermined | | | |
| | absolute stereochemistry at * | | | |
| 312 | 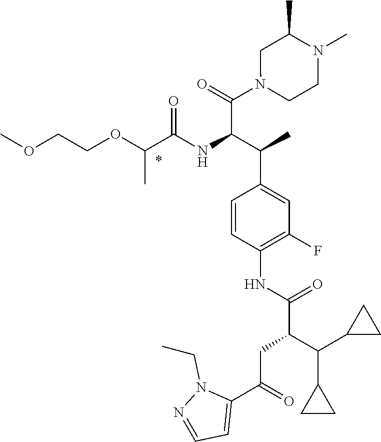 [SMILES: CCn1nccc1C(=O)C[C@H](C(=O)Nc1ccc([C@H](C)[C@@H](NC(=O)C(C)OCCOC)C(=O)N2CCN(C)[C@H](C)C2)cc1F)C(C1CC1)C1CC1] | A | C | 712.3 |
| | Single diastereomer with undetermined | | | |
| | absolute stereochemistry at * | | | |
| 313 | 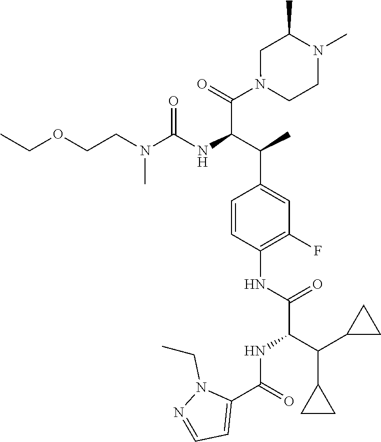 [SMILES: CCOCCN(C)C(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1] | B | C | 711.4 |
| 314 | 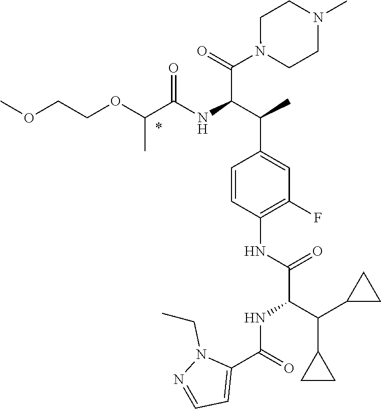 [SMILES: CCn1nccc1C(=O)N[C@H](C(=O)Nc1ccc([C@H](C)[C@@H](NC(=O)C(C)OCCOC)C(=O)N2CCN(C)CC2)cc1F)C(C1CC1)C1CC1] | C | C | 698.3 |
| | Single diastereomer with undetermined | | | |
| | absolute stereochemistry at * | | | |
| 315 | 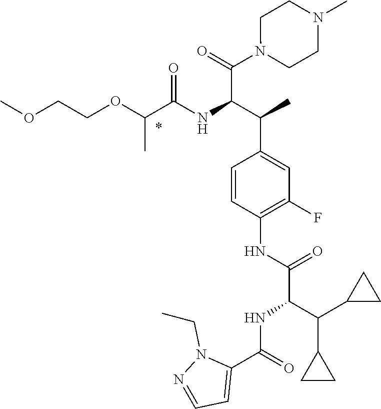 [SMILES: CCn1nccc1C(=O)N[C@H](C(=O)Nc1ccc([C@H](C)[C@@H](NC(=O)C(C)OCCOC)C(=O)N2CCN(C)CC2)cc1F)C(C1CC1)C1CC1] | B | C | 698.3 |
| | Single diastereomer with undetermined | | | |
| | absolute stereochemistry at * | | | |
| 316 | 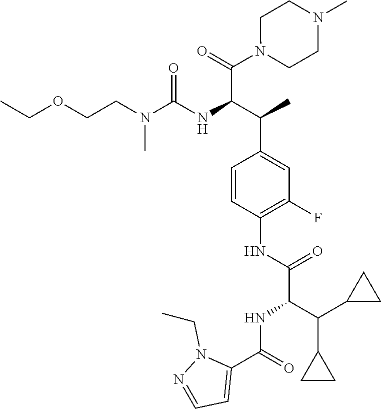 [SMILES: CCOCCN(C)C(=O)N[C@@H](C(=O)N1CCN(C)CC1)[C@@H](C)c1ccc(NC(=O)[C@@H](NC(=O)c2ccnn2CC)C(C2CC2)C2CC2)c(F)c1] | B | C | 697.5 |
| 317 | 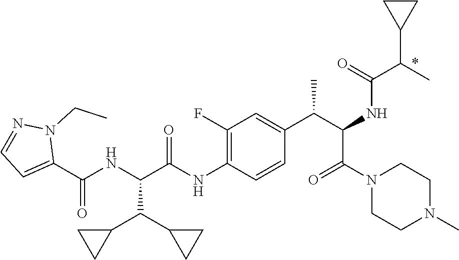 [SMILES: CCn1nccc1C(=O)N[C@H](C(=O)Nc1ccc([C@H](C)[C@@H](NC(=O)C(C)C2CC2)C(=O)N2CCN(C)CC2)cc1F)C(C1CC1)C1CC1] | A | B | 664.5 |
| | Single diastereomer with undetermined | | | |
| | absolute stereochemistry at * | | | |
| 318 | 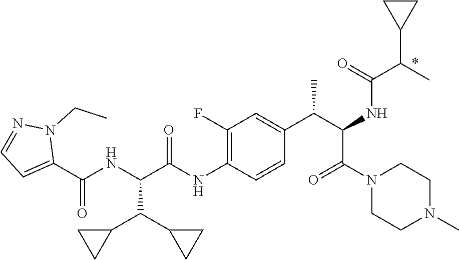 [SMILES: CCn1nccc1C(=O)N[C@H](C(=O)Nc1ccc([C@H](C)[C@@H](NC(=O)C(C)C2CC2)C(=O)N2CCN(C)CC2)cc1F)C(C1CC1)C1CC1] | A | B | 664.5 |
| | Single diastereomer with undetermined | | | |
| | absolute stereochemistry at * | | | |
| 319 | 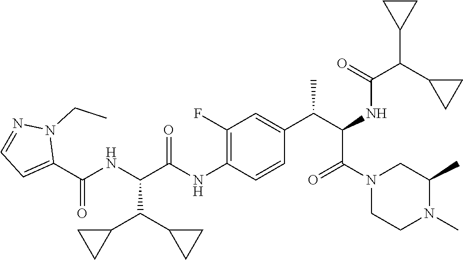 [SMILES: CCn1nccc1C(=O)N[C@H](C(=O)Nc1ccc([C@H](C)[C@@H](NC(=O)C(C2CC2)C2CC2)C(=O)N2CCN(C)[C@H](C)C2)cc1F)C(C1CC1)C1CC1] | A | B | 704.4 |
| 320 | 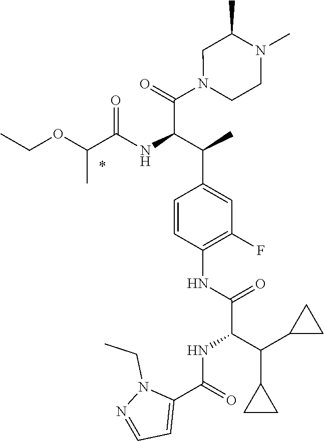 [SMILES: CCOC(C)C(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1] | B | C | 682.5 |
| | Single diastereomer with undetermined | | | |
| | absolute stereochemistry at * | | | |
| 321 | 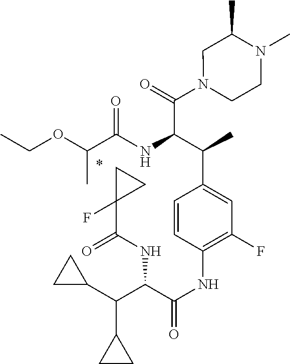 [SMILES: CCOC(C)C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | B | C | 646.2 |
| | Single diastereomer with undetermined | | | |
| | absolute stereochemistry at * | | | |
| 322 | 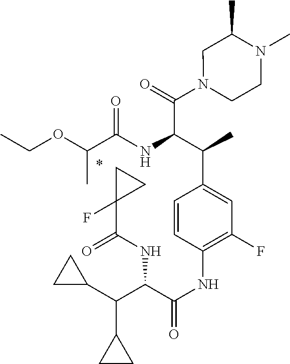 [SMILES: CCOC(C)C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | A | C | 646.5 |
| | Single diastereomer with undetermined | | | |
| | absolute stereochemistry at * | | | |
| 323 | 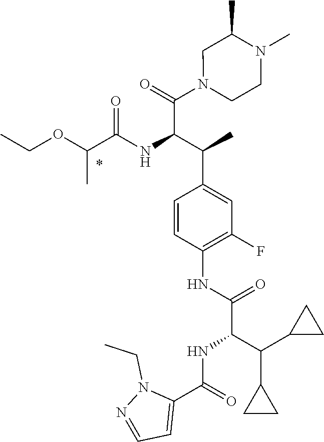 [SMILES: CCOC(C)C(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1] | A | B | 682.3 |
| | Single diastereomer with undetermined | | | |
| | absolute stereochemistry at * | | | |
| 324 | 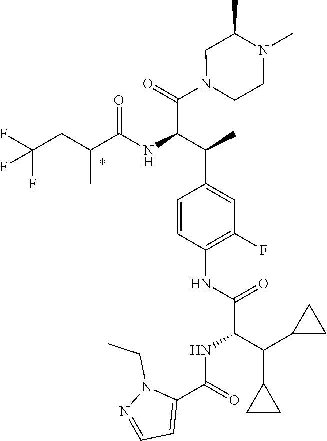 [SMILES: CCn1nccc1C(=O)N[C@H](C(=O)Nc1ccc([C@H](C)[C@@H](NC(=O)C(C)CC(F)(F)F)C(=O)N2CCN(C)[C@H](C)C2)cc1F)C(C1CC1)C1CC1] | A | B | 720.3 |
| | Single diastereomer with undetermined | | | |
| | absolute stereochemistry at * | | | |
| 325 | 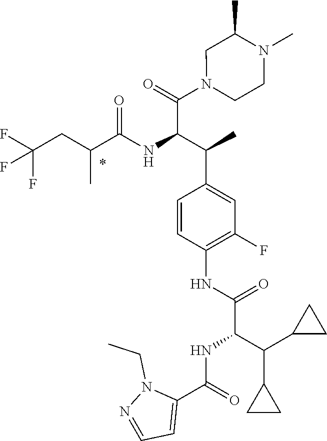 [SMILES: CCn1nccc1C(=O)N[C@H](C(=O)Nc1ccc([C@H](C)[C@@H](NC(=O)C(C)CC(F)(F)F)C(=O)N2CCN(C)[C@H](C)C2)cc1F)C(C1CC1)C1CC1] | A | B | 720.3 |
| | Single diastereomer with undetermined | | | |
| | absolute stereochemistry at * | | | |
| 326 | 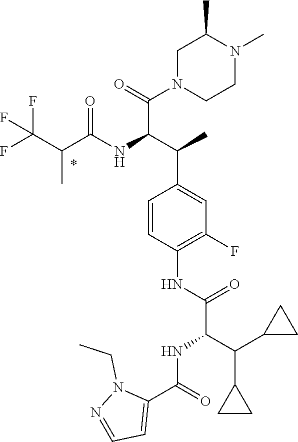 [SMILES: CCn1nccc1C(=O)N[C@H](C(=O)Nc1ccc([C@H](C)[C@@H](NC(=O)C(C)C(F)(F)F)C(=O)N2CCN(C)[C@H](C)C2)cc1F)C(C1CC1)C1CC1] | B | C | 706.3 |
| | Single diastereomer with undetermined | | | |
| | absolute stereochemistry at * | | | |
| 327 | 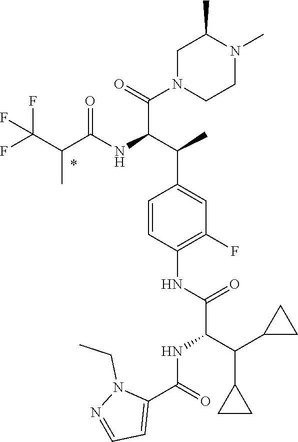 [SMILES: CCn1nccc1C(=O)N[C@H](C(=O)Nc1ccc([C@H](C)[C@@H](NC(=O)C(C)C(F)(F)F)C(=O)N2CCN(C)[C@H](C)C2)cc1F)C(C1CC1)C1CC1] | B | C | 706.3 |
| | Single diastereomer with undetermined | | | |
| | absolute stereochemistry at * | | | |
| 328 | 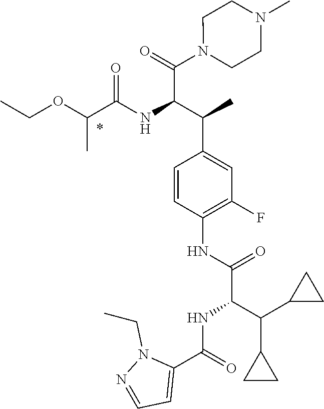 [SMILES: CCOC(C)C(=O)N[C@@H](C(=O)N1CCN(C)CC1)[C@@H](C)c1ccc(NC(=O)[C@@H](NC(=O)c2ccnn2CC)C(C2CC2)C2CC2)c(F)c1] | A | B | 668.2 |
| | Single diastereomer with undetermined | | | |
| | absolute stereochemistry at * | | | |
| 329 | 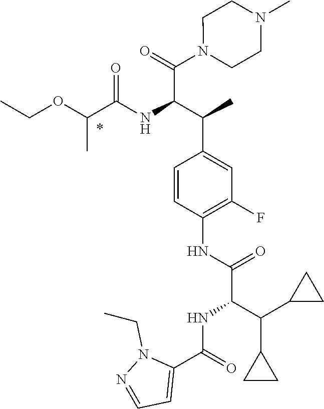 [SMILES: CCOC(C)C(=O)N[C@@H](C(=O)N1CCN(C)CC1)[C@@H](C)c1ccc(NC(=O)[C@@H](NC(=O)c2ccnn2CC)C(C2CC2)C2CC2)c(F)c1] | B | C | 668.2 |
| | Single diastereomer with undetermined | | | |
| | absolute stereochemistry at * | | | |
| 330 | 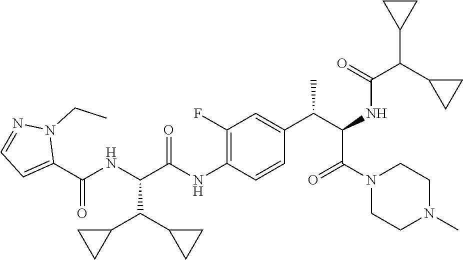 [SMILES: CCn1nccc1C(=O)N[C@H](C(=O)Nc1ccc([C@H](C)[C@@H](NC(=O)C(C2CC2)C2CC2)C(=O)N2CCN(C)CC2)cc1F)C(C1CC1)C1CC1] | A | B | 690.5 |
| 331 | 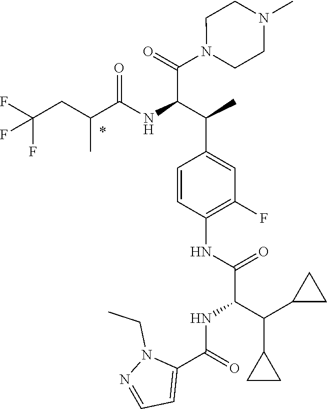 [SMILES: CCn1nccc1C(=O)N[C@H](C(=O)Nc1ccc([C@H](C)[C@@H](NC(=O)C(C)CC(F)(F)F)C(=O)N2CCN(C)CC2)cc1F)C(C1CC1)C1CC1] | B | C | 706.3 |
| | Single diastereomer with undetermined | | | |
| | absolute stereochemistry at * | | | |
| 332 | 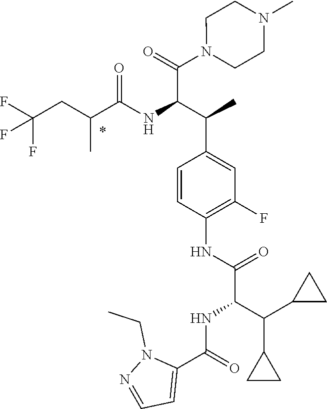 [SMILES: CCn1nccc1C(=O)N[C@H](C(=O)Nc1ccc([C@H](C)[C@@H](NC(=O)C(C)CC(F)(F)F)C(=O)N2CCN(C)CC2)cc1F)C(C1CC1)C1CC1] | B | B | 706.3 |
| | Single diastereomer with undetermined | | | |
| | absolute stereochemistry at * | | | |
| 333 | 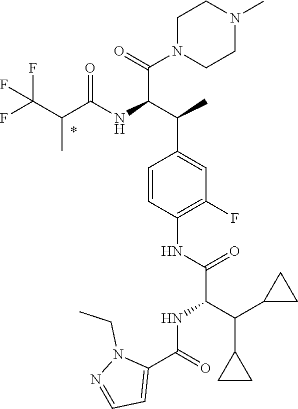 [SMILES: CCn1nccc1C(=O)N[C@H](C(=O)Nc1ccc([C@H](C)[C@@H](NC(=O)C(C)C(F)(F)F)C(=O)N2CCN(C)CC2)cc1F)C(C1CC1)C1CC1] | B | C | 692.1 |
| | Single diastereomer with undetermined | | | |
| | absolute stereochemistry at * | | | |
| 334 | 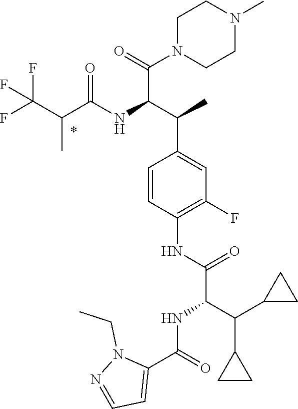 [SMILES: CCn1nccc1C(=O)N[C@H](C(=O)Nc1ccc([C@H](C)[C@@H](NC(=O)C(C)C(F)(F)F)C(=O)N2CCN(C)CC2)cc1F)C(C1CC1)C1CC1] | B | C | 692.1 |
| | Single diastereomer with undetermined | | | |
| | absolute stereochemistry at * | | | |
| 335 | 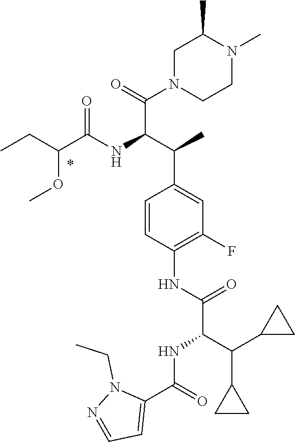 [SMILES: CCC(OC)C(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1] | B | C | 682.4 |
| | Single diastereomer with undetermined | | | |
| | absolute stereochemistry at * | | | |
| 336 | 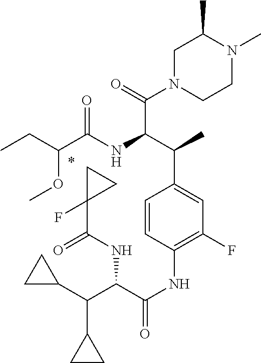 [SMILES: CCC(OC)C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | B | C | 646.2 |
| | Single diastereomer with undetermined | | | |
| | absolute stereochemistry at * | | | |
| 337 | 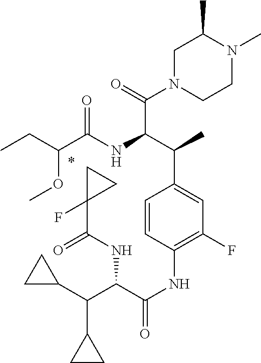 [SMILES: CCC(OC)C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | A | C | 646.4 |
| | Single diastereomer with undetermined | | | |
| | absolute stereochemistry at * | | | |
| 338 | 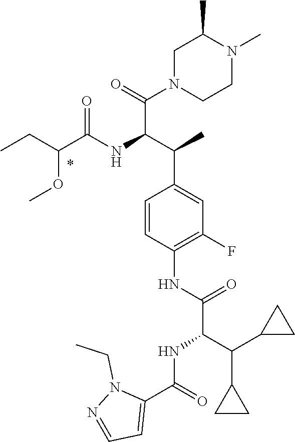 [SMILES: CCC(OC)C(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1] | A | B | 682.4 |
| | Single diastereomer with undetermined | | | |
| | absolute stereochemistry at * | | | |
| 339 | 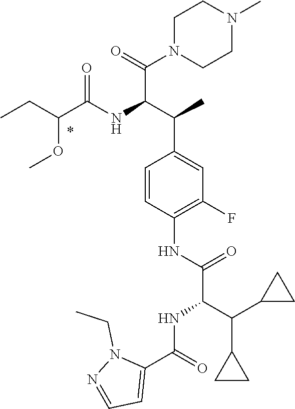 [SMILES: CCC(OC)C(=O)N[C@@H](C(=O)N1CCN(C)CC1)[C@@H](C)c1ccc(NC(=O)[C@@H](NC(=O)c2ccnn2CC)C(C2CC2)C2CC2)c(F)c1] | B | C | 668.4 |
| | Single diastereomer with undetermined | | | |
| | absolute stereochemistry at * | | | |
| 340 | 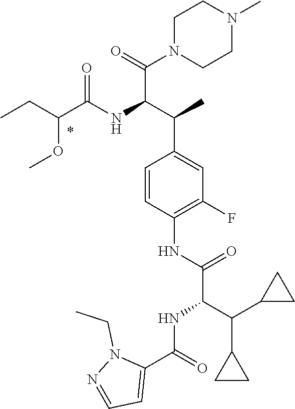 [SMILES: CCC(OC)C(=O)N[C@@H](C(=O)N1CCN(C)CC1)[C@@H](C)c1ccc(NC(=O)[C@@H](NC(=O)c2ccnn2CC)C(C2CC2)C2CC2)c(F)c1] | A | B | 668.5 |
| | Single diastereomer with undetermined | | | |
| | absolute stereochemistry at * | | | |
| 341 | 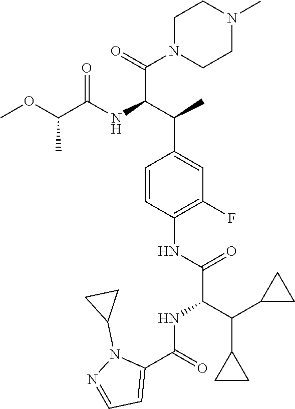 [SMILES: CO[C@@H](C)C(=O)N[C@@H](C(=O)N1CCN(C)CC1)[C@@H](C)c1ccc(NC(=O)[C@@H](NC(=O)c2ccnn2C2CC2)C(C2CC2)C2CC2)c(F)c1] | A | C | 666.4 |
| 342 | 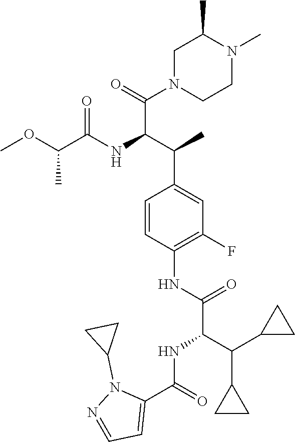 [SMILES: CO[C@@H](C)C(=O)N[C@@H](C(=O)N1CCN(C)[C@H](C)C1)[C@@H](C)c1ccc(NC(=O)[C@@H](NC(=O)c2ccnn2C2CC2)C(C2CC2)C2CC2)c(F)c1] | A | B | 680.5 |
| 343 | 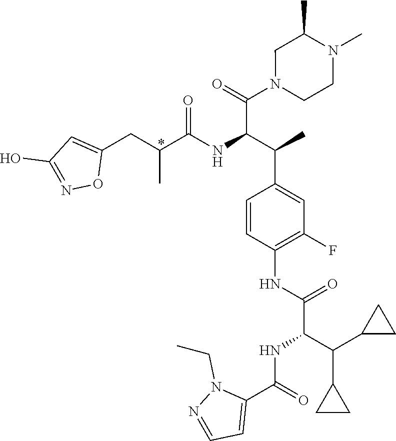 [SMILES: CCn1nccc1C(=O)N[C@H](C(=O)Nc1ccc([C@H](C)[C@@H](NC(=O)C(C)Cc2cc(O)no2)C(=O)N2CCN(C)[C@H](C)C2)cc1F)C(C1CC1)C1CC1] | B | C | 735.4 |
| | Single diastereomer with undetermined | | | |
| | absolute stereochemistry at * | | | |
| 344 | 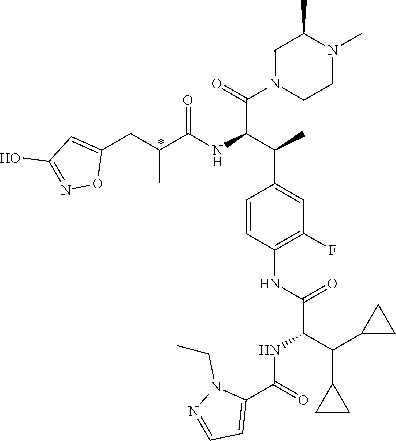 [SMILES: CCn1nccc1C(=O)N[C@H](C(=O)Nc1ccc([C@H](C)[C@@H](NC(=O)C(C)Cc2cc(O)no2)C(=O)N2CCN(C)[C@H](C)C2)cc1F)C(C1CC1)C1CC1] | A | B | 735.4 |
| | Single diastereomer with undetermined | | | |
| | absolute stereochemistry at * | | | |
| 345 | 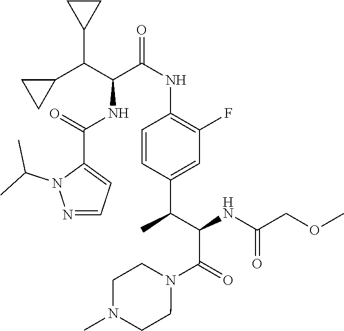 [SMILES: COCC(=O)N[C@@H](C(=O)N1CCN(C)CC1)[C@@H](C)c1ccc(NC(=O)[C@@H](NC(=O)c2ccnn2C(C)C)C(C2CC2)C2CC2)c(F)c1] | A | B | 654.5 |
| 346 | 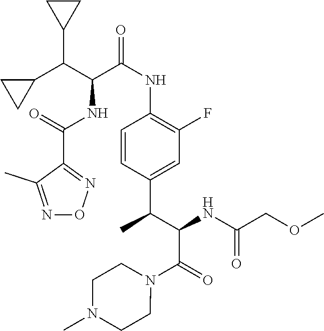 [SMILES: COCC(=O)N[C@@H](C(=O)N1CCN(C)CC1)[C@@H](C)c1ccc(NC(=O)[C@@H](NC(=O)c2nonc2C)C(C2CC2)C2CC2)c(F)c1] | B | C | 628.5 |
| 347 | 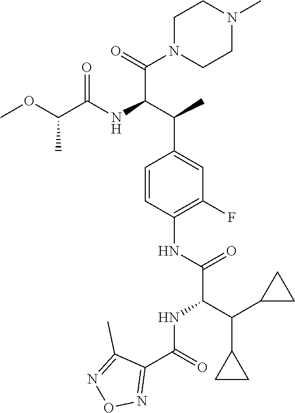 [SMILES: CO[C@@H](C)C(=O)N[C@@H](C(=O)N1CCN(C)CC1)[C@@H](C)c1ccc(NC(=O)[C@@H](NC(=O)c2nonc2C)C(C2CC2)C2CC2)c(F)c1] | A | C | 642.5 |
| 348 | 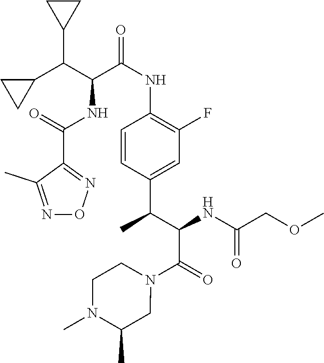 [SMILES: COCC(=O)N[C@@H](C(=O)N1CCN(C)[C@H](C)C1)[C@@H](C)c1ccc(NC(=O)[C@@H](NC(=O)c2nonc2C)C(C2CC2)C2CC2)c(F)c1] | A | C | 642.5 |
| 349 | 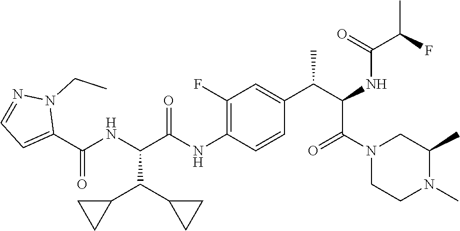 [SMILES: CCn1nccc1C(=O)N[C@H](C(=O)Nc1ccc([C@H](C)[C@@H](NC(=O)[C@@H](C)F)C(=O)N2CCN(C)[C@H](C)C2)cc1F)C(C1CC1)C1CC1] | A | C | 656.5 |
| 350 | 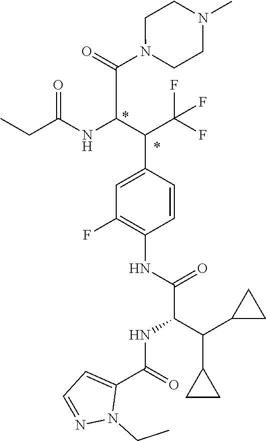 [SMILES: CCC(=O)NC(C(=O)N1CCN(C)CC1)C(c1ccc(NC(=O)[C@@H](NC(=O)c2ccnn2CC)C(C2CC2)C2CC2)c(F)c1)C(F)(F)F] | C | C | 678.5 |
| | Single diastereomer with undetermined | | | |
| | absolute stereochemistry at * | | | |
| 351 | 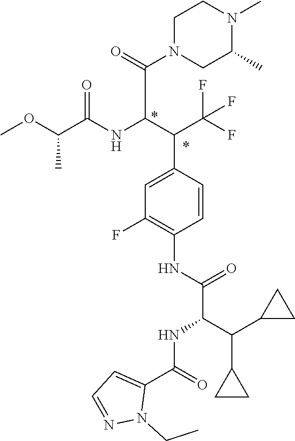 [SMILES: CCn1nccc1C(=O)N[C@H](C(=O)Nc1ccc(C(C(NC(=O)[C@H](C)OC)C(=O)N2CCN(C)[C@H](C)C2)C(F)(F)F)cc1F)C(C1CC1)C1CC1] | C | C | 722.5 |
| | Single diastereomer with undetermined | | | |
| | absolute stereochemistry at * | | | |
| 352 | 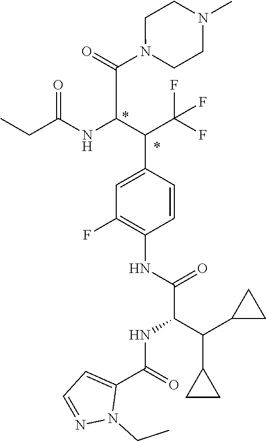 [SMILES: CCC(=O)NC(C(=O)N1CCN(C)CC1)C(c1ccc(NC(=O)[C@@H](NC(=O)c2ccnn2CC)C(C2CC2)C2CC2)c(F)c1)C(F)(F)F] | C | C | 678.4 |
| | Single diastereomer with undetermined | | | |
| | absolute stereochemistry at * | | | |
| 353 | 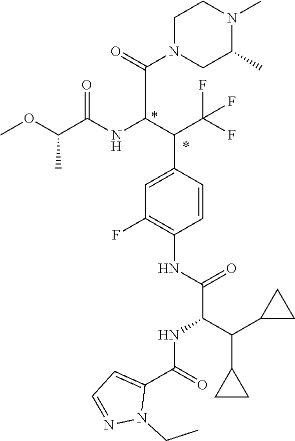 [SMILES: CCn1nccc1C(=O)N[C@H](C(=O)Nc1ccc(C(C(NC(=O)[C@H](C)OC)C(=O)N2CCN(C)[C@H](C)C2)C(F)(F)F)cc1F)C(C1CC1)C1CC1] | A | B | 722.5 |
| | Single diastereomer with undetermined | | | |
| | absolute stereochemistry at * | | | |
| 354 | 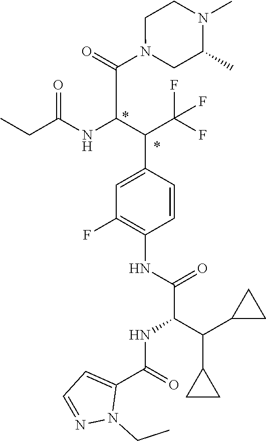 [SMILES: CCC(=O)NC(C(=O)N1CCN(C)[C@H](C)C1)C(c1ccc(NC(=O)[C@@H](NC(=O)c2ccnn2CC)C(C2CC2)C2CC2)c(F)c1)C(F)(F)F] | C | C | 678.4 |
| | Single diastereomer with undetermined | | | |
| | absolute stereochemistry at * | | | |
| 355 | 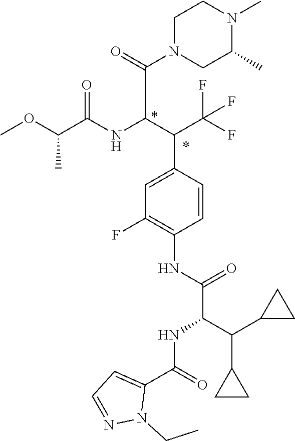 [SMILES: CCn1nccc1C(=O)N[C@H](C(=O)Nc1ccc(C(C(NC(=O)[C@H](C)OC)C(=O)N2CCN(C)[C@H](C)C2)C(F)(F)F)cc1F)C(C1CC1)C1CC1] | C | C | 722.4 |
| | Single diastereomer with undetermined | | | |
| | absolute stereochemistry at * | | | |
| 356 | 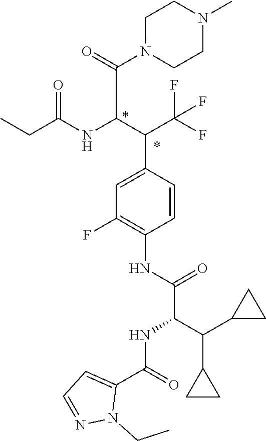 [SMILES: CCC(=O)NC(C(=O)N1CCN(C)CC1)C(c1ccc(NC(=O)[C@@H](NC(=O)c2ccnn2CC)C(C2CC2)C2CC2)c(F)c1)C(F)(F)F] | B | C | 678.6 |
| | Single diastereomer with undetermined | | | |
| | absolute stereochemistry at * | | | |
| 357 | 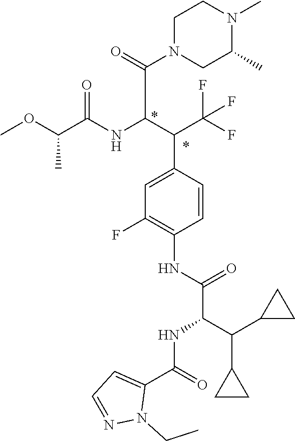 [SMILES: CCn1nccc1C(=O)N[C@H](C(=O)Nc1ccc(C(C(NC(=O)[C@H](C)OC)C(=O)N2CCN(C)[C@H](C)C2)C(F)(F)F)cc1F)C(C1CC1)C1CC1] | C | C | 722.3 |
| | Single diastereomer with undetermined | | | |
| | absolute stereochemistry at * | | | |
| 358 | 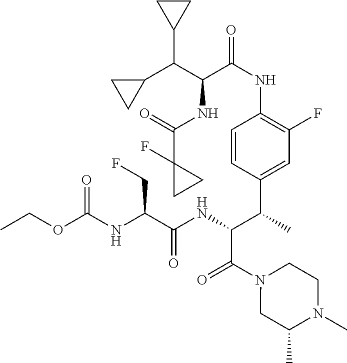 [SMILES: CCOC(=O)N[C@@H](CF)C(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | A | C | 707.5 |
| 359 | 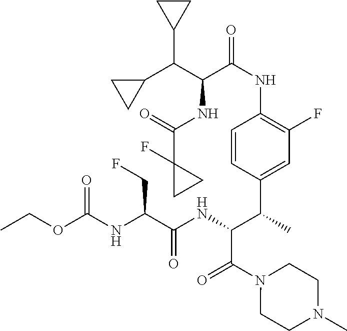 [SMILES: CCOC(=O)N[C@@H](CF)C(=O)N[C@@H](C(=O)N1CCN(C)CC1)[C@@H](C)c1ccc(NC(=O)[C@@H](NC(=O)C2(F)CC2)C(C2CC2)C2CC2)c(F)c1] | A | C | 693.6 |
| 360 | 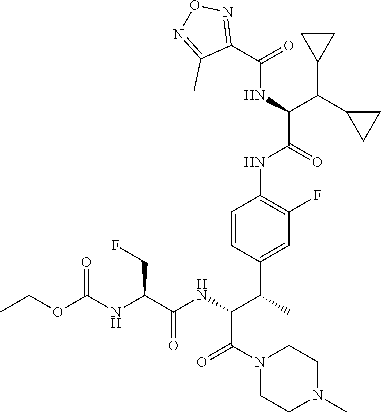 [SMILES: CCOC(=O)N[C@@H](CF)C(=O)N[C@@H](C(=O)N1CCN(C)CC1)[C@@H](C)c1ccc(NC(=O)[C@@H](NC(=O)c2nonc2C)C(C2CC2)C2CC2)c(F)c1] | A | B | 717.4 |
| 361 | 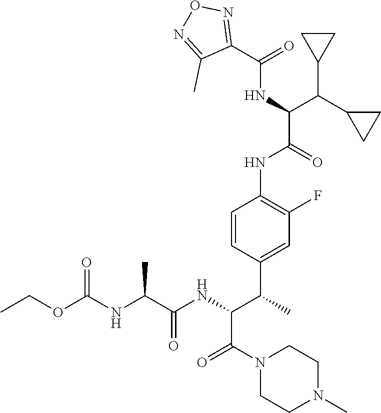 [SMILES: CCOC(=O)N[C@@H](C)C(=O)N[C@@H](C(=O)N1CCN(C)CC1)[C@@H](C)c1ccc(NC(=O)[C@@H](NC(=O)c2nonc2C)C(C2CC2)C2CC2)c(F)c1] | A | B | 699.4 |
| 362 | 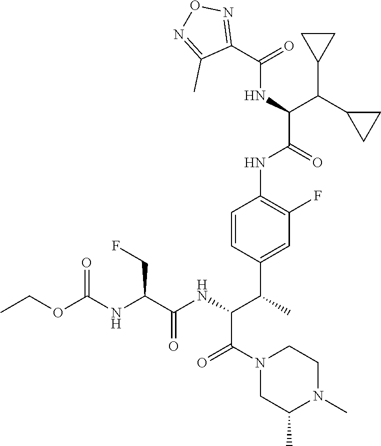 [SMILES: CCOC(=O)N[C@@H](CF)C(=O)N[C@@H](C(=O)N1CCN(C)[C@H](C)C1)[C@@H](C)c1ccc(NC(=O)[C@@H](NC(=O)c2nonc2C)C(C2CC2)C2CC2)c(F)c1] | A | B | 713.3 |
| 363 | 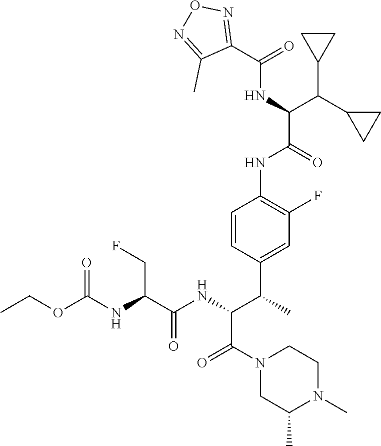 [SMILES: CCOC(=O)N[C@@H](CF)C(=O)N[C@@H](C(=O)N1CCN(C)[C@H](C)C1)[C@@H](C)c1ccc(NC(=O)[C@@H](NC(=O)c2nonc2C)C(C2CC2)C2CC2)c(F)c1] | A | B | 731.5 |

### Example 11: Comparison of Selected IL-17 Modulators Against Reference Compounds A-M

### Microsomal Stability Studies

Test compound and control working solution preparation. 5 μL of a compound and control stock solution (10 mM in dimethyl sulfoxide (DMSO)) were diluted with 495 μL of acetonitrile (ACN).

NADPH cofactor preparation. The appropriate amount of NADPH powder was weighed and diluted into a 10 mM MgCl₂ solution (working solution concentration: 10 mM; final concentration in reaction system: 1 mM). Materials: NADPH powder: β-Nicotinamide adenine dinucleotide phosphate reduced form, tetrasodium salt; NADPHA4Na (Vendor: Chem-Impex International, Cat. No. 00616).

Liver Microsome Preparation. The appropriate concentrations of microsome working solutions were prepared in 100 mM potassium phosphate buffer. Materials: Human Liver Microsomes (HLM) Cat No. 452117 and Lot No. 38295 (Corning); SD Rat Liver Microsomes (RLM) Cat No. R1000 and Lot No. 1910100 (Xenotech).

Stop solution preparation. Cold (4° C.) acetonitrile (ACN) containing 200 ng/mL tolbutamide and 200 ng/mL labetalol as internal standards (IS) were used as the stop solution.

Assay Procedure. Empty incubation plates (T60 and NCF60) were warmed for 10 mins prior to use. Liver microsomes were diluted to 0.56 mg/mL in 100 mM phosphate buffer. 445 uL microsome working solutions (0.56 mg/mL) were transferred into pre-warmed ‘Incubation’ plates T60 and NCF60, Then ‘Incubation’ plates T60 and NCF60 were pre-incubated for 10 min at 37° C. with constant shaking. 54 μL liver microsomes were transferred to the blank plate, then 6 μL NAPDH cofactor were added to the blank plate, followed by the addition 180 μL quenching solution to the blank plate. 5 μL compound working solution (100 μM) were added into ‘incubation’ plates (T60 and NCF60) containing microsomes and mixed thoroughly 3 times. To the NCF60 plate 50 μL of buffer were added and mixed 3 times thoroughly. Start timing; plate was incubated at 37° C. for 60 min while shaking. In ‘Quenching’ plate TO, were added 180 μL quenching solution and 6 μL NAPDH cofactor. Plate is chilled to prevent evaporation. The T60 plate, was mixed 3 times thoroughly, and immediately 54 μL of the mixture was removed for the 0-min time point to ‘Quenching’ plate. Then 44 μL NAPDH cofactor was added to the incubation plate (T60). Start timing; plate was incubated at 37° C. for 60 min while shaking. Final concentrations of each component in the incubation media: Microsome (0.5 mg protein/mL); Test compound (1 μM); control compound (1 M); ACN (0.99%); and DMSO (0.01%). At 5, 15, 30, 45, and 60 min, 180 μL quenching solution was added to ‘Quenching’ plates, mixed once, and serially transferred 60 μL of sample from T60 plate per time point to ‘Quenching’ plates. For the NCF60 plate, mix once and 60 μL of sample was transferred from the NCF60 incubation to ‘Quenching’ plate containing quenching solution at the 60-min time point (Start time 1:00:00 and End Time 0:00:00). Table 2.1 provides reaction plate incubation times.

TABLE 2.1
Reaction Plates Incubation

| Time Point | Start Time | End Time |
|---|---|---|
| Blank | 1:00:00 | 0:00:00 |
| T60 | 1:00:00 | 0:00:00 |
| T45 | 0:45:00 | 0:00:00 |
| T30 | 0:30:00 | 0:00:00 |
| T15 | 0:15:00 | 0:00:00 |
| T5 | 0:05:00 | 0:00:00 |

| | |
|---|---|
| T0 | mix 3 times and remove out |
| | to ′Quenching′ plate |

All sampling plates were shaken for 10 min, then centrifuged at 4000 rpm for 20 minutes at 4° C. 80 μL of supernatant were transferred into 240 μL HPLC water, and mixed by plate shaker for 10 min. Each bioanalysis plate was sealed and shaken for 10 minutes prior to LC-MS/MS analysis. First order kinetics were used to calculate half-life and CL_{int(mic)} (μL/min/mg) as reported in Table 2.6.

### Rat Pharmacokinetics

Formulations for PO. An appropriate amount of the test article is weighed and mixed with the appropriate volume of vehicle to get a clear solution or a uniform suspension, vortexing or sonication in water bath may used. Animals are dosed within four hours after the formulation is prepared.

Formulation samples are removed from each of the formulation solutions or suspensions, transferred into 1.5 mL of polypropylene microcentrifuge tubes and run dose validated by LC/UV or LC-MS/MS.

Dose administration. For PO dosing, the dose formulation is administered via oral gavage.

Blood Collection. Each blood collection (about 0.2 mL per time point) is performed from carotid artery cannula (CAC) of each animal into a pre-chilled commercial EDTA-K2 tubes as anti-coagulant, then placed on wet ice until centrifugation. Blood samples are processed for plasma by centrifugation at approximately 4° C., 3,200 g for 10 min. Plasma is collected respectively and a certain amount of plasma from rat is transferred into pre-labeled 96 well plate or polypropylene tubes, quick frozen over dry ice and kept at −60° C. or lower until LC-MS/MS analysis. After terminal collection, all plasmas are stored at approximately −80° C. freezer until final analysis.

Plasma concentration versus time data are plotted in graph and analyzed by non-compartmental approaches using the Phoenix WinNonlin 6.3 software program. PO AUC calculation method used is Linear/log trapezoidal and values from the method are reported in Table 2.6. Trapezoids: Before TMax (Linear); After Tmax (Log). Partial Area Interpolations: Before TMax (Linear); After Tmax (Log).

Table 2.2 provides the study design for determining rat pharmacokinetics. Table 2.3 provides the dosing schedule for the PK analysis. Table 2.4 provides the sample collection schedule.

TABLE 2.2
Rat Pharmacokinetics Study Design

| Group | Phase | No. of | | Dose | Dose | Conc | Dose | Is | | |
|---|---|---|---|---|---|---|---|---|---|---|
| No. | No. | animals | Sex | Route | (mg/kg) | (mg/mL | Volume | Cassette | Is Diff | Vehicle |
| 01 | NA | 3 | Male | IV | 1 | 1 | 1 | False | False | 25 mM citrate buffer |
| | | | | bolus | | | | | | pH 5: 10% |
| | | | | | | | | | | cyclodextrin in PBS |
| 02 | NA | 3 | Male | PO | 5 | 1 | 5 | False | False | 25 mM citrate buffer |
| | | | | | | | | | | pH 5: 10% |
| | | | | | | | | | | cyclodextrin in PBS |

Is cassette: True means cassette compounds dosing for this group. False means single compound dosing. Is Diff: True means cassette compounds dosing and dose level are different for each compound. False means dose level are same for each compound or single compound dosing.

TABLE 2.3
Rat Pharmacokinetics Dose Schedule

| Group No. | Day | Time(h) |
|---|---|---|
| 01 | 1 | 0 |
| 02 | 1 | 0 |

TABLE 2.4
Rat Pharmacokinetics Sample Collection Schedule

| Group | Phase | Dosage (mg/kg) | A1 | A2 | A3 | A4 | A5 | A6 | Day | Time Point (hr) |
|---|---|---|---|---|---|---|---|---|---|---|
| 01 | NA | 1 | R01 | R02 | R03 | | | | 1 | 0.083 |
| | | | | | | | | | | 0.25 |
| | | | | | | | | | | 0.5 |
| | | | | | | | | | | 1 |
| | | | | | | | | | | 2 |
| | | | | | | | | | | 4 |
| | | | | | | | | | | 8 |
| | | | | | | | | | | 24 |
| 02 | NA | 5 | R04 | R05 | R06 | | | | 1 | 0.25 |
| | | | | | | | | | | 0.5 |
| | | | | | | | | | | 1 |
| | | | | | | | | | | 2 |
| | | | | | | | | | | 4 |
| | | | | | | | | | | 8 |
| | | | | | | | | | | 24 |

Table 2.5 provides a listing of selected Th-17 Modulators as disclosed herein used for comparative studies against reference compounds A to M.

Table 2.6 provides microsomal stability data and rat pharmacokinetic data for the selected Th-17 Modulators against reference compounds A to M. The data demonstrates enhanced microsomal stability and rat pharmacokinetic of the selected IL-17 modulators against reference compounds A to M.

TABLE 2.5
Listing Of Comparative Compounds

| No. | Compound | No. | Compound |
|---|---|---|---|
| 212 | 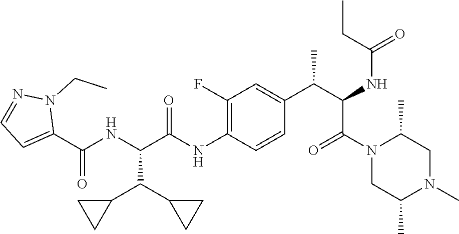 [SMILES: CCC(=O)N[C@@H](C(=O)N1C[C@@H](C)N(C)C[C@H]1C)[C@@H](C)c1ccc(NC(=O)[C@@H](NC(=O)c2ccnn2CC)C(C2CC2)C2CC2)c(F)c1] | A | 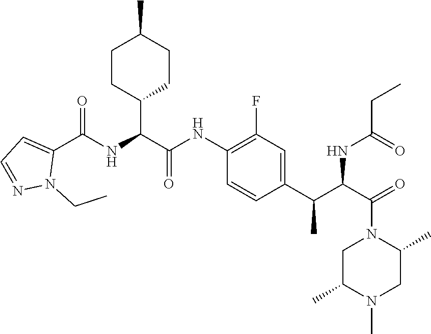 [SMILES: CCC(=O)N[C@@H](C(=O)N1C[C@@H](C)N(C)C[C@H]1C)[C@@H](C)c1ccc(NC(=O)[C@@H](NC(=O)c2ccnn2CC)[C@H]2CC[C@H](C)CC2)c(F)c1] |
| 298 | 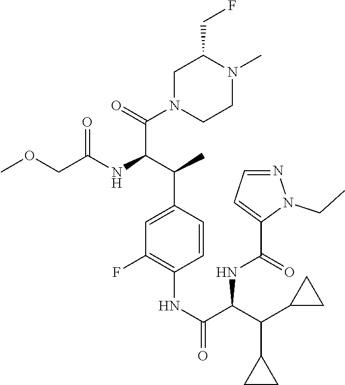 [SMILES: CCn1nccc1C(=O)N[C@H](C(=O)Nc1ccc([C@H](C)[C@@H](NC(=O)COC)C(=O)N2CCN(C)[C@@H](CF)C2)cc1F)C(C1CC1)C1CC1] | B | 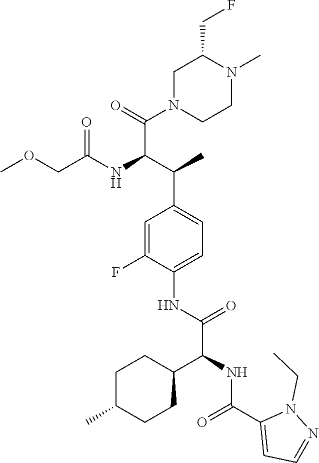 [SMILES: CCn1nccc1C(=O)N[C@H](C(=O)Nc1ccc([C@H](C)[C@@H](NC(=O)COC)C(=O)N2CCN(C)[C@@H](CF)C2)cc1F)[C@H]1CC[C@H](C)CC1] |
| 214 | 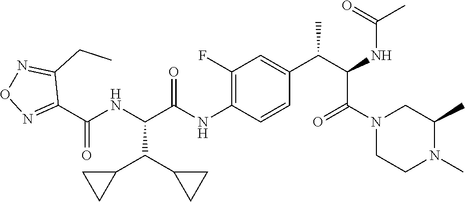 [SMILES: CCc1nonc1C(=O)N[C@H](C(=O)Nc1ccc([C@H](C)[C@@H](NC(C)=O)C(=O)N2CCN(C)[C@H](C)C2)cc1F)C(C1CC1)C1CC1] | C | 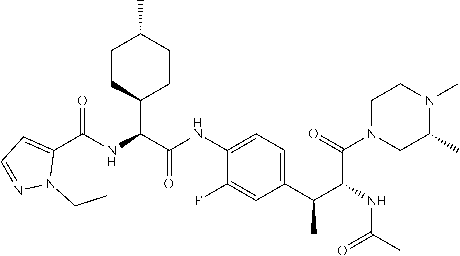 [SMILES: CCn1nccc1C(=O)N[C@H](C(=O)Nc1ccc([C@H](C)[C@@H](NC(C)=O)C(=O)N2CCN(C)[C@H](C)C2)cc1F)[C@H]1CC[C@H](C)CC1] |
| 207 | 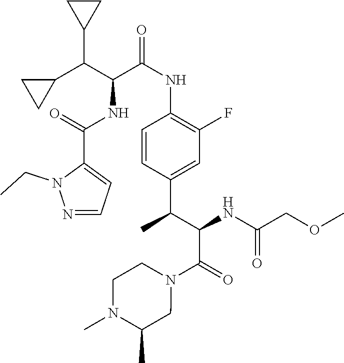 [SMILES: CCn1nccc1C(=O)N[C@H](C(=O)Nc1ccc([C@H](C)[C@@H](NC(=O)COC)C(=O)N2CCN(C)[C@H](C)C2)cc1F)C(C1CC1)C1CC1] | D | 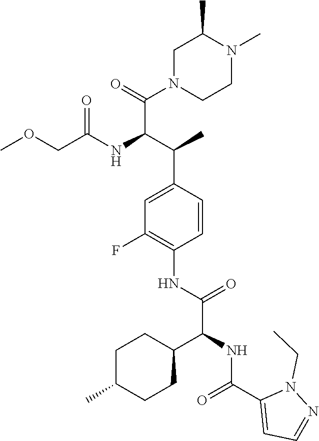 [SMILES: CCn1nccc1C(=O)N[C@H](C(=O)Nc1ccc([C@H](C)[C@@H](NC(=O)COC)C(=O)N2CCN(C)[C@H](C)C2)cc1F)[C@H]1CC[C@H](C)CC1] |
| 232 | 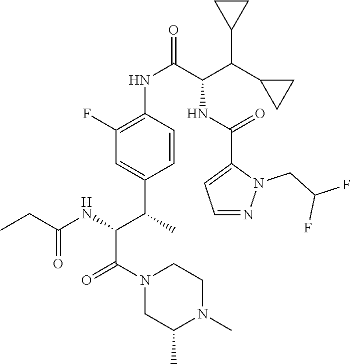 [SMILES: CCC(=O)N[C@@H](C(=O)N1CCN(C)[C@H](C)C1)[C@@H](C)c1ccc(NC(=O)[C@@H](NC(=O)c2ccnn2CC(F)F)C(C2CC2)C2CC2)c(F)c1] | E | 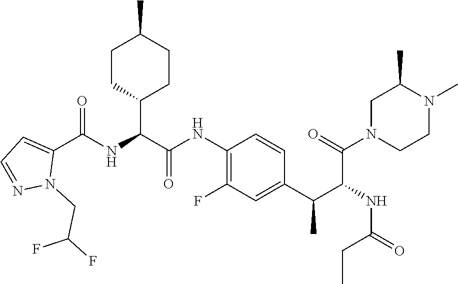 [SMILES: CCC(=O)N[C@@H](C(=O)N1CCN(C)[C@H](C)C1)[C@@H](C)c1ccc(NC(=O)[C@@H](NC(=O)c2ccnn2CC(F)F)[C@H]2CC[C@H](C)CC2)c(F)c1] |
| 217 | 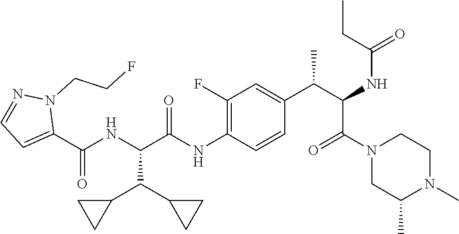 [SMILES: CCC(=O)N[C@@H](C(=O)N1CCN(C)[C@H](C)C1)[C@@H](C)c1ccc(NC(=O)[C@@H](NC(=O)c2ccnn2CCF)C(C2CC2)C2CC2)c(F)c1] | F | 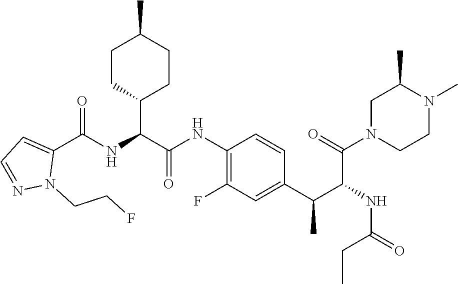 [SMILES: CCC(=O)N[C@@H](C(=O)N1CCN(C)[C@H](C)C1)[C@@H](C)c1ccc(NC(=O)[C@@H](NC(=O)c2ccnn2CCF)[C@H]2CC[C@H](C)CC2)c(F)c1] |
| 209 | 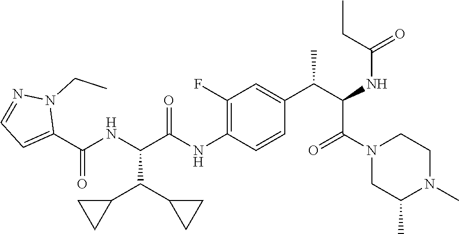 [SMILES: CCC(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1] | G | 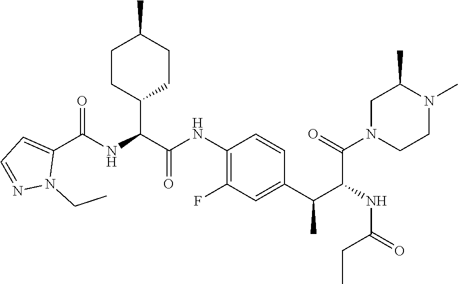 [SMILES: CCC(=O)N[C@@H](C(=O)N1CCN(C)[C@H](C)C1)[C@@H](C)c1ccc(NC(=O)[C@@H](NC(=O)c2ccnn2CC)[C@H]2CC[C@H](C)CC2)c(F)c1] |
| 245 | 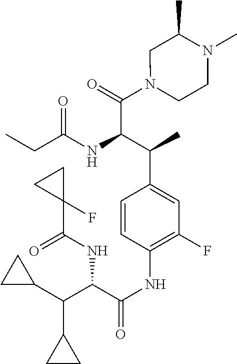 [SMILES: CCC(=O)N[C@@H](C(=O)N1CCN(C)[C@H](C)C1)[C@@H](C)c1ccc(NC(=O)[C@@H](NC(=O)C2(F)CC2)C(C2CC2)C2CC2)c(F)c1] | H | 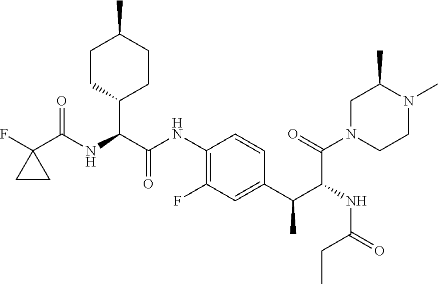 [SMILES: CCC(=O)N[C@@H](C(=O)N1CCN(C)[C@H](C)C1)[C@@H](C)c1ccc(NC(=O)[C@@H](NC(=O)C2(F)CC2)[C@H]2CC[C@H](C)CC2)c(F)c1] |
| 213 | 344 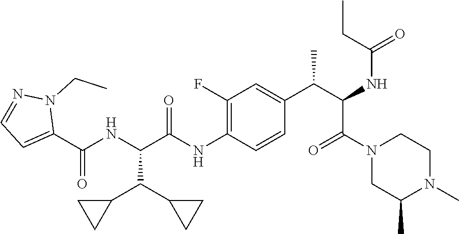 [SMILES: CCC(=O)N[C@@H](C(=O)N1CCN(C)[C@@H](C)C1)[C@@H](C)c1ccc(NC(=O)[C@@H](NC(=O)c2ccnn2CC)C(C2CC2)C2CC2)c(F)c1] | I | 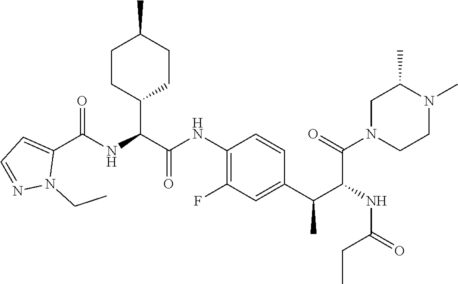 [SMILES: CCC(=O)N[C@@H](C(=O)N1CCN(C)[C@@H](C)C1)[C@@H](C)c1ccc(NC(=O)[C@@H](NC(=O)c2ccnn2CC)[C@H]2CC[C@H](C)CC2)c(F)c1] |
| 225 | 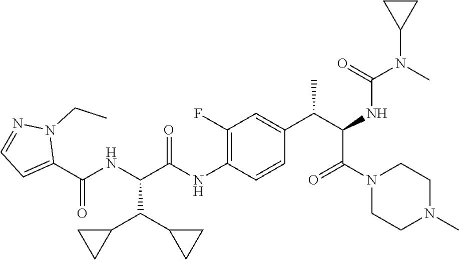 [SMILES: CCn1nccc1C(=O)N[C@H](C(=O)Nc1ccc([C@H](C)[C@@H](NC(=O)N(C)C2CC2)C(=O)N2CCN(C)CC2)cc1F)C(C1CC1)C1CC1] | J | 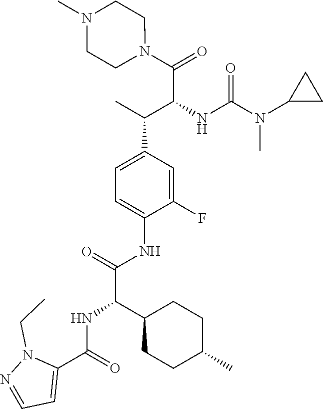 [SMILES: CCn1nccc1C(=O)N[C@H](C(=O)Nc1ccc([C@H](C)[C@@H](NC(=O)N(C)C2CC2)C(=O)N2CCN(C)CC2)cc1F)[C@H]1CC[C@H](C)CC1] |
| 215 | 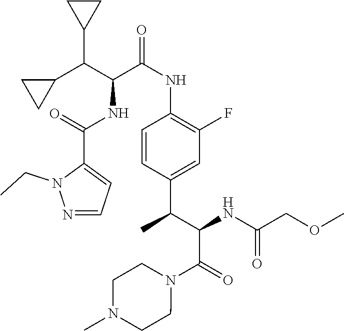 [SMILES: CCn1nccc1C(=O)N[C@H](C(=O)Nc1ccc([C@H](C)[C@@H](NC(=O)COC)C(=O)N2CCN(C)CC2)cc1F)C(C1CC1)C1CC1] | K | 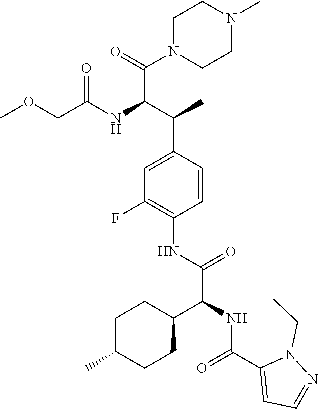 [SMILES: CCn1nccc1C(=O)N[C@H](C(=O)Nc1ccc([C@H](C)[C@@H](NC(=O)COC)C(=O)N2CCN(C)CC2)cc1F)[C@H]1CC[C@H](C)CC1] |
| 257 | 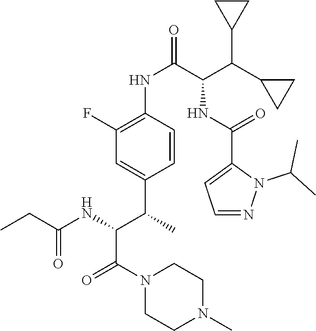 [SMILES: CCC(=O)N[C@@H](C(=O)N1CCN(C)CC1)[C@@H](C)c1ccc(NC(=O)[C@@H](NC(=O)c2ccnn2C(C)C)C(C2CC2)C2CC2)c(F)c1] | L | 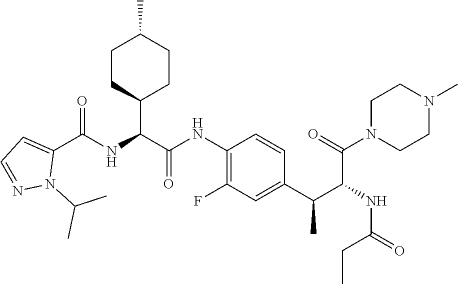 [SMILES: CCC(=O)N[C@@H](C(=O)N1CCN(C)CC1)[C@@H](C)c1ccc(NC(=O)[C@@H](NC(=O)c2ccnn2C(C)C)[C@H]2CC[C@H](C)CC2)c(F)c1] |
| 223 | 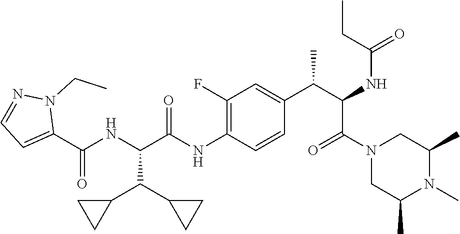 [SMILES: CCC(=O)N[C@@H](C(=O)N1C[C@@H](C)N(C)[C@@H](C)C1)[C@@H](C)c1ccc(NC(=O)[C@@H](NC(=O)c2ccnn2CC)C(C2CC2)C2CC2)c(F)c1] | | |

TABLE 2.6
Comparative Compounds-Microsomal Stability Data and Rat Pharmocokinetics

| | HLM Clint | RLM Clint | Rat PO AUC0-t | | HLM Clint | RLM Clint | Rat PO AUC0-t |
|---|---|---|---|---|---|---|---|
| No. | (uL/min/mg protein)* | (uL/min/mg protein)** | (ng*hr/mL)*** | No. | (uL/min/mg protein)* | (uL/min/mg protein)** | (ng*hr/mL)*** |
| 212 | A | B | B | A | B | C | B |
| 298 | A | A | A | B | B | B | C |
| 214 | B | B | C | C | C | C | C |
| 207 | B | A | A | D | B | B | B |
| 232 | A | A | C | E | C | B | B |
| 217 | A | A | C | F | B | B | B |
| 209 | B | A | B | G | C | C | B |
| 245 | A | A | A | H | B | B | C |
| 213 | B | A | B | I | C | B | C |
| 225 | B | C | C | J | C | C | C |
| 215 | B | A | A | K | C | A | B |
| 257 | B | A | B | L | C | B | B |
| 223 | B | B | C | M | C | B | C |

*A is <50; B is 50-150; and C is >150.
**A is <9.6; B is 9.6-20; and C is >20
***A is >1,000; B is 500-1,000; and C is <500

We claim:

1. A compound represented by the structure of the Formula: 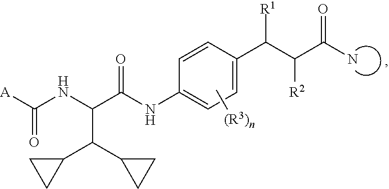
or a pharmaceutically acceptable salt thereof, wherein:
A is selected from a C₃₋₆ carbocycle and 5- to 6-membered heterocycle, any one of which is optionally substituted with one or more substituents independently selected from (a), (b), and (c):
(a) halogen, —OR¹¹, —N(R¹¹)₂, —C(O)R¹¹, —C(O)OR¹¹, —NO₂, and —CN;
(b) C₁₋₆ alkyl optionally substituted with one or more substituents independently selected from: halogen, —OR¹¹, —N(R¹¹)₂, —C(O)R¹¹, —C(O)OR¹¹, —NO₂, —CN, C₃₋₆ carbocycle and 3- to 6-membered heterocycle, wherein each C₃₋₆ carbocycle and 3- to 6-membered heterocycle is optionally substituted with one or more substituents independently selected from halogen, —OR¹¹, —N(R¹¹)₂, —C(O)R¹¹, —C(O)OR¹¹, —NO₂, and —CN; and
(c) C₃₋₆ carbocycle optionally substituted with one or more substituents independently selected from halogen, C₁₋₄ alkyl, C₁₋₄ haloalkyl, —OR¹¹, —N(R¹¹)₂, —C(O)R¹¹, —C(O)OR¹¹, —NO₂, and —CN;
R¹ is selected from hydrogen, halogen, —OR¹², —N(R¹²)₂, —C(O)R¹², —C(O)OR¹², —NO₂, —CN, and C₁₋₆ alkyl optionally substituted with one or more substituents independently selected from halogen, —OR¹², —N(R¹²)₂, —C(O)R¹², —C(O)OR¹², —NO₂, and —CN;
R² is selected from —N(R^{A})C(O)(R^{B}) and —N(R^{C})C(O)N(R^{D})(R^{E}); 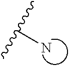 [SMILES: *N]
is selected from a 4- to 12-membered heterocycle optionally substituted with one or more substituents independently selected from:
halogen, —OR¹³, —N(R¹³)₂, —C(O)R¹³, —C(O)OR¹³, —NO₂, —CN, and C₁₋₆ alkyl optionally substituted with one or more substituents independently selected from halogen, —OR¹³, —N(R¹³)₂, —C(O)R¹³, —C(O)OR¹³, —NO₂, and —CN;
R³ is selected from halogen, —OR¹⁴, —N(R¹⁴)₂, —C(O)R¹⁴, —C(O)OR¹⁴, —NO₂, —CN, C₁₋₆ alkyl and C₃₋₆ carbocycle, wherein each C₁₋₆ alkyl and C₃₋₆ carbocycle of which is optionally substituted with one or more substituents independently selected from halogen, —OR¹⁴, —N(R¹⁴)₂, —C(O)R¹⁴, —C(O)OR¹⁴, —NO₂, and —CN;
R^{A} is selected from hydrogen and C₁₋₆ alkyl optionally substituted with one or more substituents independently selected from halogen, —OR¹, —N(R¹¹)₂, —C(O)R, —C(O)OR¹¹, —NO₂, and —CN;
R^{B} is selected from:
C₁₋₆ alkyl optionally substituted with one or more substituents independently selected from:
halogen, —OR¹⁶, —N(R¹⁶)₂, —C(O)R¹⁶, —C(O)OR¹⁶, —N(R¹⁶)C(O)OR¹⁶, —NO₂, —CN;
C₃₋₆ carbocycle and 5- to 6-membered heterocycle, any of which is optionally substituted with one or more substituents independently selected from halogen, —OR¹⁶, —N(R¹⁶)₂, —C(O)R¹⁶, —C(O)OR¹⁶, —NO₂, and —CN; and
C₃₋₆ carbocycle or 4- to 6-membered heterocycle, each of which is optionally substituted with one or more substituents independently selected from:
halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN;
R^{C} is selected from hydrogen and C₁₋₆ alkyl optionally substituted with one or more substituents independently selected from: halogen, —OR¹⁷, —N(R¹⁷)₂, —C(O)R¹⁷, —C(O)OR¹⁷, —NO₂, and —CN;
R^{D} is selected from C₁₋₆ alkyl and C₃₋₆ carbocycle, any of which is optionally substituted with one or more substituents independently selected from: halogen, —OR¹⁸, —N(R¹⁸)₂, —C(O)R¹⁸, —C(O)OR¹⁸, —NO₂, and —CN;
R^{E} is selected from hydrogen, C₁₋₆ alkyl and C₃₋₆carbocycle, any of which is optionally substituted with one or more substituents independently selected from:
halogen, —OR¹⁹, —N(R¹⁹)₂, —C(O)R¹⁹, —C(O)OR¹⁹, —NO₂, and —CN;
R¹¹, R¹², R¹³, R¹⁴, R¹⁵, R¹⁶, R¹⁷, R¹⁸, and R¹⁹ are each independently selected at each occurrence from:
hydrogen;
C₁₋₆ alkyl optionally substituted with one more substituents independently selected from halogen, —O—C₁₋₆ alkyl, —O—C₁₋₆ haloalkyl, —NH₂, —NO₂, ═O, —CN, C₃₋₁₀ carbocycle and 3- to 10-membered heterocycle, wherein each C₃₋₁₀ carbocycle and 3- to 10-membered heterocycle are optionally substituted with one or more substituents independently selected from: halogen, —OH, —O—C₁₋₆ alkyl, —O—C₁₋₆ haloalkyl, —NH₂, —NO₂, ═O, and —CN; and
C₃₋₁₀ carbocycle and 3- to 10-membered heterocycle optionally substituted with one or more substituents independently selected from: halogen, —OH, —O—C₁₋₆ alkyl, —O—C₁₋₆ haloalkyl, —NH₂, —NO₂, ═O, and —CN; and
n is selected from 0, 1, 2, 3, and 4.

2. The compound or salt of claim 1, wherein n is 1.

3. The compound or salt of claim 2, wherein each R³ is independently selected from fluorine, chlorine, —OR¹⁴, —N(R¹⁴)₂, —CN, C₁₋₃ alkyl and C₁₋₃ haloalkyl.

4. The compound or salt of claim 3, wherein each R³ is selected from fluorine.

5. The compound or salt of claim represented by the Formula:

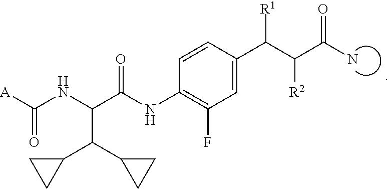

6. The compound or salt of claim 5, wherein R¹ is selected from selected from hydrogen, halogen, —OR¹², —N(R¹²)₂, —CN, C₁₋₃ alkyl and C₁₋₃ haloalkyl.

7. The compound or salt of claim 6, wherein R¹ is selected from methyl, ethyl, propyl, and trifluoromethyl.

8. The compound or salt of claim 7, wherein R¹ is selected from selected from methyl.

9. The compound or salt of claim 7, wherein R¹ is selected from selected from trifluoromethyl.

10. The compound or salt of claim 6, wherein A is selected from a saturated C₃₋₆ carbocycle and a 5-membered heteroaryl, any of which is optionally substituted with one or more substituents independently selected from: from (a), (b), and (c):
(a) halogen, —OR¹¹, —N(R¹¹)₂, —C(O)R¹¹, —C(O)OR¹¹, —NO₂, and —CN;
(b) C₁₋₃ alkyl optionally substituted with one or more substituents independently selected from halogen, —OR¹¹, —N(R¹¹)₂, —C(O)R¹¹, —C(O)OR¹¹, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹, —N(R¹¹)₂, —C(O)R¹, —C(O)OR¹¹, —NO₂, and —CN; and
(c) C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

11. The compound or salt of claim 10, wherein A is selected saturated C₃₋₆ carbocycle, any one of which is optionally substituted with one or more substituents independently selected from halogen, —OR¹¹, —N(R¹¹)₂, —CN, C₁₋₃ alkyl, and C₁₋₃ haloalkyl.

12. The compound or salt of claim 11, wherein A is cyclopropyl optionally substituted with halogen, C₁₋₃ alkyl, and C₁₋₃ haloalkyl.

13. The compound or salt of claim 12, wherein A is

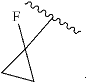 [SMILES: *C1(F)CC1]

14. The compound or salt of claim 10, wherein A is a 5-membered heteroaryl optionally substituted with one or more substituents independently selected from:
C₁₋₆ alkyl optionally substituted with one or more substituents independently selected from: halogen, —OR¹¹, —N(R¹¹)₂, —C(O)R¹¹, —C(O)OR¹¹, —NO₂, —CN; and 5- to 6-membered heterocycle each of which is optionally substituted with one or more substituents selected from: halogen, —OR¹¹, —N(R¹¹)₂, —C(O)R¹¹, —C(O)OR¹¹, —NO₂, and —CN; and
C₃₋₆ carbocycle each of which is optionally substituted with one or more substituents selected from: halogen, —OR¹¹, —N(R¹¹)₂, —C(O)R¹¹, —C(O)OR¹¹, —NO₂, and —CN.

15. The compound or salt of claim 14, wherein A is selected from pyrazole, imidazole, oxazole, isoxazole, oxadiazole, triazole, tetrazole, any one of which is optionally substituted with one or more substituents independently selected from:
C₁₋₃ alkyl optionally substituted with one or more substituents independently selected from: halogen, —OR¹¹, and 5- to 6-membered saturated heterocycle each of which is optionally substituted with one or more substituents selected from: halogen, —OR¹¹, —N(R¹¹)₂, —C(O)R¹¹, —C(O)OR¹¹, —NO₂, and —CN; and
saturated C₃₋₆ carbocycle optionally substituted with one or more substituents selected from: halogen, —OR¹¹, —N(R¹¹)₂, —C(O)R¹¹, —C(O)OR¹¹, —NO₂, and —CN.

16. The compound or salt of claim 15, wherein A is selected from pyrazole and oxadiazole, each of which is optionally substituted with one or more substituents independently selected from C₁₋₃ alkyl, C₁₋₃ haloalkyl, C₁₋₃ alkyl-OR¹¹, and C₁₋₃ alkyl substituted with 5- to 6-membered saturated heterocycle.

17. The compound or salt of claim 16, wherein A is selected from pyrazole and oxadiazole, each of which is optionally substituted with methyl, ethyl, propyl, and isopropyl.

18. The compound or salt of claim 17, wherein A is selected from:

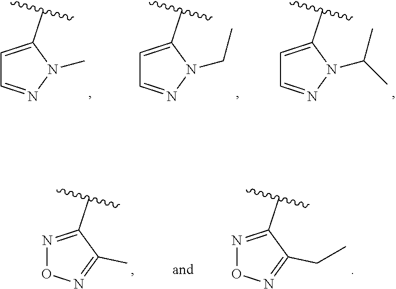

19. The compound or salt of claim 16, wherein A is selected from pyrazole and oxadiazole, each of which is optionally substituted with C₁₋₃ haloalkyl, and C₁₋₃ alkyl-OR¹¹.

20. The compound or salt of claim 19, wherein A is selected from:

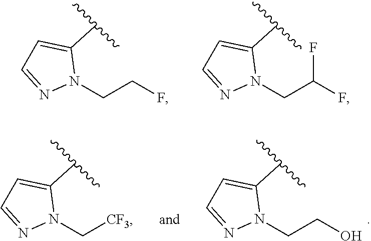 [SMILES: *c1ccnn1CCF ; *c1ccnn1CC(F)F ; *c1ccnn1CC(F)(F)F ; "and" ; *c1ccnn1CCO ; "."]

21. The compound or salt of claim 16, wherein A is from pyrazole and oxadiazole, each of which is optionally substituted with C₁₋₃ alkyl substituted with 5- to 6-membered saturated heterocycle.

22. The compound or salt of claim 21, wherein A is

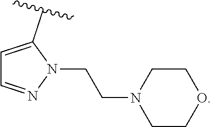 [SMILES: *c1ccnn1CCN1CCOCC1]

23. The compound or salt of claim 15, wherein A is selected from pyrazole and oxadiazole, each of which is optionally substituted with one or more substituents independently selected from saturated C₃₋₆ carbocycle optionally substituted with one or more substituents selected from: halogen, —OR¹¹, —N(R¹¹)₂, —C(O)R¹¹, —C(O)OR¹¹, —NO₂, and —CN.

24. The compound or salt of claim 23, wherein A is selected from pyrazole and oxadiazole, each of which is optionally substituted by cyclopropyl, wherein the cyclopropyl is optionally substituted with fluorine or chlorine.

25. The compound or salt of claim 24, wherein A is selected from:

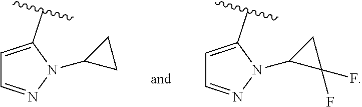 [SMILES: *c1ccnn1C1CC1 ; "and" ; *c1ccnn1C1CC1(F)F]

26. The compound or salt of claim 5, wherein R² is selected from —N(R^{C})C(O)N(R^{D})(R^{E}), wherein:
R^{C} is selected from hydrogen;
R^{D} is selected from C₁₋₆ alkyl and saturated C₃₋₆ carbocycle, any of which is optionally substituted with one or more substituents independently selected from: halogen, —OR¹⁸, —N(R¹⁸)₂, —C(O)R¹⁸, —C(O)OR¹⁸, —NO₂, and —CN; and
R^{E} is selected from C₁₋₆ alkyl and saturated C₃₋₆carbocycle, any of which is optionally substituted with one or more substituents independently selected from: halogen, —OR¹⁹, —N(R¹⁹)₂, —C(O)R¹⁹, —C(O)OR¹⁹, —NO₂, and —CN.

27. The compound or salt of claim 26, wherein
R^{D} is selected from C₁₋₃ alkyl and cyclopropyl, any of which is optionally substituted with one or more substituents independently selected from: halogen, —OR¹⁸, —N(R¹⁸)₂, —NO₂, and —CN; and
R^{E} is selected from C₁₋₃ alkyl.

28. The compound or salt of claim 27, wherein R² is selected from:

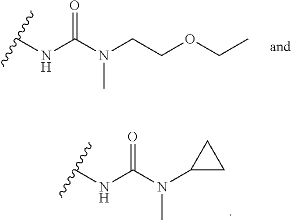

29. The compound or salt of claim 5, wherein R² is selected from —N(R^{A})C(O)(R^{B}), wherein:
R^{A} is selected from hydrogen, C₁₋₆ alkyl, and C₁₋₆ haloalkyl;
R^{B} is selected from:
C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, —CN;
C₃₋₆ carbocycle and 5- to 6-membered heterocycle, any of which is optionally substituted with one or more substituents independently selected from: halogen, —OR¹⁶, —N(R¹⁶)₂, —C(O)R¹⁶, —C(O)OR¹⁶, —NO₂, and —CN; and
4- to 6-membered heterocycle optionally substituted with one or more substituents independently selected from: halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from: halogen, —OR¹⁶, —N(R¹⁶)₂, —C(O)R¹⁶, —C(O)OR¹⁶, —NO₂, and —CN.

30. The compound or salt of claim 29, wherein R^{A} is hydrogen; and R^{B} is selected from: C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, —CN, C₃₋₆ carbocycle and 5- to 6-membered heterocycle, wherein each C₃₋₆ carbocycle and 5- to 6-membered heterocycle is optionally substituted with one or more substituents independently selected from: halogen, —OR¹⁶, —N(R¹⁶)₂, —C(O)R¹⁶, —C(O)OR¹⁶, —NO₂, and —CN.

31. The compound or salt of claim 30, wherein R^{A} is hydrogen; and R^{B} is selected from: C₁₋₆ alkyl optionally substituted with one or more substituents independently selected from halogen, —OR¹⁶, —N(R¹⁶)₂, —C(O)R¹⁶, —C(O)OR¹⁶, —N(R¹⁶)C(O)OR¹⁶, —NO₂, and —CN.

32. The compound or salt of claim 31, wherein R^{A} is hydrogen; and R^{B} is selected from C₁₋₆ alkyl and C₁₋₆ haloalkyl.

33. The compound or salt of claim 32, wherein R² is selected from:

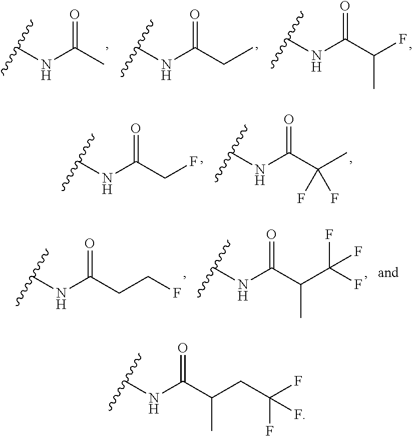

34. The compound or salt of claim 31, wherein R^{A} is hydrogen; and R^{B} is selected from: C₁₋₆ alkyl optionally substituted with one or more substituents independently selected from halogen, —OR¹⁶ and N(R¹⁶)C(O)OR¹⁶.

35. The compound or salt of claim 34, wherein R² is selected from:

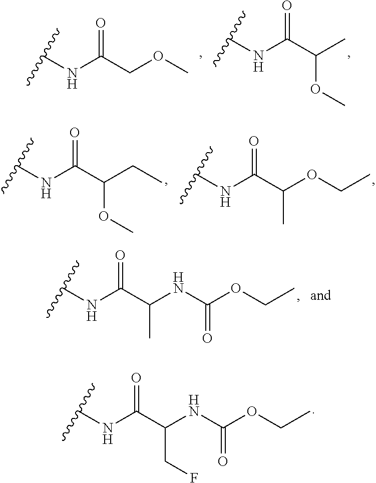

36. The compound or salt of claim 31, wherein R^{A} is hydrogen; and R^{B} is C₁₋₆ alkyl optionally substituted with —OR¹⁶, wherein R¹⁶ is C₁₋₆ alkyl substituted with —O—C₁₋₆ alkyl.

37. The compound or salt of claim 36, wherein R² is

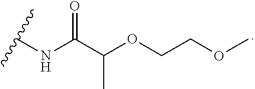 [SMILES: *NC(=O)C(C)OCCOC]

38. The compound or salt of claim 30, wherein R^{A} is hydrogen; and R^{B} is selected from: C₁₋₆ alkyl optionally substituted with one or more substituents independently selected from C₃₋₆ carbocycle and 5- to 6-membered heterocycle, any one of which is optionally substituted with one or more substituents independently selected from halogen, —OR¹⁶, —N(R¹⁶)₂, —NO₂, and —CN.

39. The compound or salt of claim 38, wherein R^{A} is hydrogen; and R^{B} is selected from: C₁₋₆ alkyl optionally substituted with one or more substituents independently selected from optionally substituted C₃₋₆ carbocycle.

40. The compound or salt of claim 39, wherein R² is selected from:

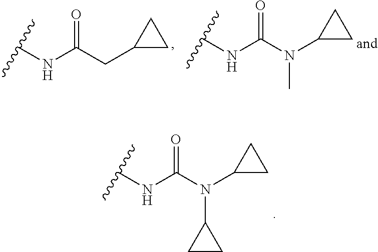

41. The compound or salt of claim 38, wherein R^{A} is hydrogen; and R^{B} is selected from: C₁₋₆ alkyl optionally substituted with one or more substituents independently selected from 5-membered heterocycle optionally substituted with one or more substituents independently selected from halogen, —OR¹⁶, —N(R¹⁶)₂, —NO₂, and —CN.

42. The compound or salt of claim 41, wherein R² is

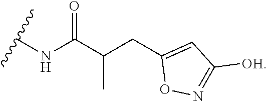 [SMILES: *NC(=O)C(C)Cc1cc(O)no1]

43. The compound or salt of claim 29, wherein R^{A} is hydrogen; and R^{B} is selected from: C₁₋₆ alkyl optionally substituted with —N(R¹⁶)₂; wherein R¹⁶ is selected at each occurrence from hydrogen, C₁₋₃ alkyl, and 3- to 6-membered heterocycle, where each C₁₋₃ alkyl and 3- to 6-membered heterocycle is optionally substituted.

44. The compound or salt of claim 43, wherein R² is selected from:

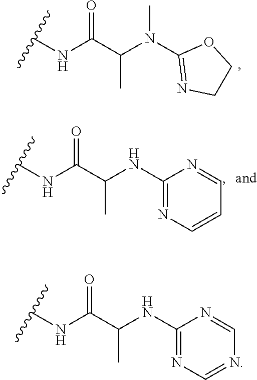 [SMILES: *NC(=O)C(C)N(C)C1=NCCO1 ; *NC(=O)C(C)Nc1ncccn1 ; *NC(=O)C(C)Nc1ncncn1]

45. The compound or salt of claim 29, wherein R^{A} is hydrogen; and R^{B} is selected from a 4- to 6-membered heterocycle optionally substituted with one or more substituents independently selected from halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN.

46. The compound or salt of claim 45, wherein R^{A} is hydrogen; and R^{B} is selected from a 4- to 6-membered saturated heterocycle, any one of which is optionally substituted with one or more substituents independently selected from halogen, —OR¹³, —N(R¹⁶)₂, —C(O)R¹⁶, —C(O)OR¹⁶, —NO₂, —CN, and C₁₋₆ alkyl optionally substituted with one or more substituents independently selected from halogen, —OR¹⁶, —N(R¹⁶)₂, —C(O)R¹⁶, —C(O)OR¹⁶, —NO₂, and —CN.

47. The compound or salt of claim 46, wherein R^{A} is hydrogen; and R^{B} is selected from azetidine and pyrrolidine, each of which is optionally substituted with halogen, —OR¹, —C(O)R¹⁶, —C(O)OR¹⁶.

48. The compound or salt of claim 47, wherein R² is selected from:

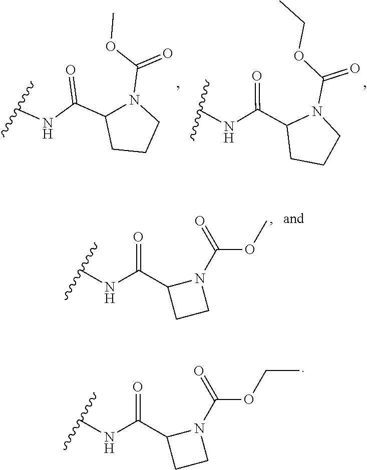

49. The compound or salt of claim 5, wherein 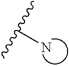 [SMILES: *N]
is selected from a 4- to 6-membered monocyclic heterocycle and 5- to 10-membered bicyclic fused heterocycle each of which is optionally substituted with one or more substituents independently selected from: halogen, —OR¹³, —N(R¹³)₂, —NO₂, —CN, and C₁₋₆ alkyl optionally substituted with one or more substituents independently selected from halogen, —OR¹³, —N(R¹³)₂, —NO₂, and —CN.

50. The compound or salt of claim 49, wherein 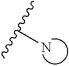 [SMILES: *N]
is selected from 4- to 6-membered monocyclic heterocycle, any of which is optionally substituted with one or more substituents independently selected from halogen, —OR¹³, —N(R¹³)₂, and C₁₋₆ alkyl optionally substituted with one or more substituents independently selected from: halogen, and —OR¹³.

51. The compound or salt of claim 50, wherein 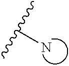 [SMILES: *N]
is selected from azetidine, pyrrolidine, pyrazolidine, imidazolidine, piperidine, morpholine, and piperazine, each of which is optionally substituted with one or more substituents independently selected from halogen, —OR¹³, —N(R¹³)₂, and C₁₋₆ alkyl optionally substituted with one or more substituents independently selected from: halogen, and —OR¹³.

52. The compound or salt of claim 51, wherein 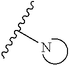 [SMILES: *N]
is selected from pyrrolidine, piperidine, morpholine, and piperazine, each of which is optionally substituted with one or more substituents selected from —OR¹³, —N(R¹³)₂, and C₁₋₃ alkyl.

53. The compound or salt of claim 52, wherein 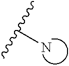 [SMILES: *N]
is selected from:

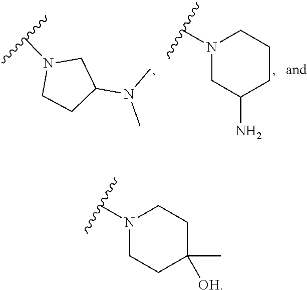

54. The compound or salt of claim 51, wherein 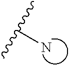 [SMILES: *N]
is selected from pyrrolidine, piperidine, morpholine, and piperazine, each of which is optionally substituted with one or more substituents selected from C₁₋₃ alkyl optionally substituted with one or more substituents independently selected from: halogen and —OR¹³.

55. The compound or salt of claim 54, wherein 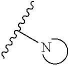 [SMILES: *N]
is selected from:

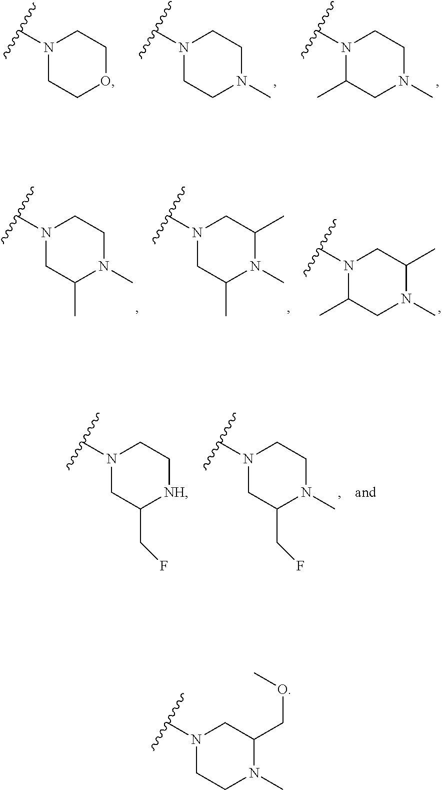

56. The compound or salt of claim 49, wherein 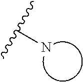 [SMILES: *N]
is selected from 7- to 10-membered bicyclic heterocycle, any one of which is optionally substituted with one or more substituents independently selected from C₁₋₆ alkyl optionally substituted with halogen, —OR¹³, —N(R¹³)₂, —NO₂, and —CN.

57. The compound or salt of claim 56, wherein 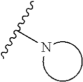 [SMILES: *N]
is selected from 7- to 10-membered bicyclic heterocycle, any one of which is optionally substituted with C₁₋₃ alkyl.

58. The compound or salt of claim 57, wherein 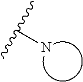 [SMILES: *N]
is selected from:

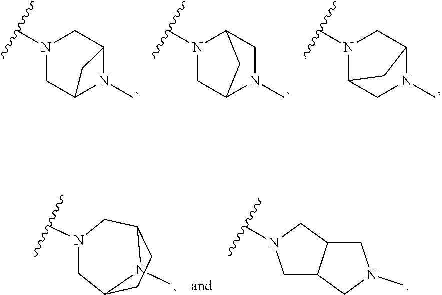

59. The compound or salt of claim 49, wherein 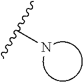 [SMILES: *N]
is selected from 5- to 8-membered spirocyclic heterocycle, any one of which is optionally substituted with one or more substituents independently selected from C₁₋₆ alkyl optionally substituted with halogen, —OR¹³, —N(R¹³)₂, —NO₂, and —CN.

60. The compound or salt of claim 59, wherein

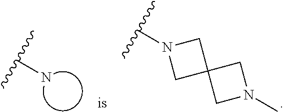

61. The compound or salt of claim 1 of the Formula selected from: 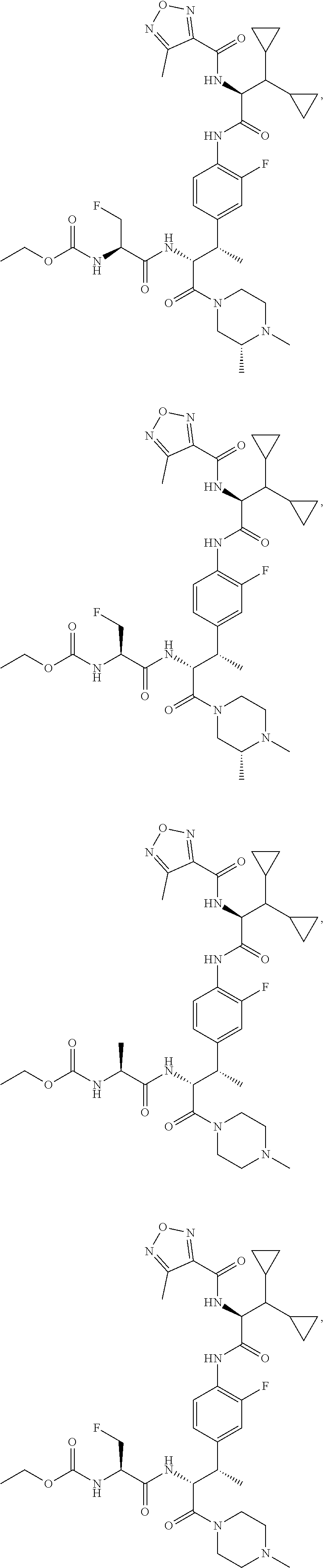 [SMILES: CCOC(=O)N[C@@H](CF)C(=O)N[C@@H](C(=O)N1CCN(C)[C@H](C)C1)[C@@H](C)c1ccc(NC(=O)[C@@H](NC(=O)c2nonc2C)C(C2CC2)C2CC2)c(F)c1 ; CCOC(=O)N[C@@H](CF)C(=O)N[C@@H](C(=O)N1CCN(C)[C@H](C)C1)[C@@H](C)c1ccc(NC(=O)[C@@H](NC(=O)c2nonc2C)C(C2CC2)C2CC2)c(F)c1 ; CCOC(=O)N[C@@H](C)C(=O)N[C@@H](C(=O)N1CCN(C)CC1)[C@@H](C)c1ccc(NC(=O)[C@@H](NC(=O)c2nonc2C)C(C2CC2)C2CC2)c(F)c1 ; CCOC(=O)N[C@@H](CF)C(=O)N[C@@H](C(=O)N1CCN(C)CC1)[C@@H](C)c1ccc(NC(=O)[C@@H](NC(=O)c2nonc2C)C(C2CC2)C2CC2)c(F)c1] 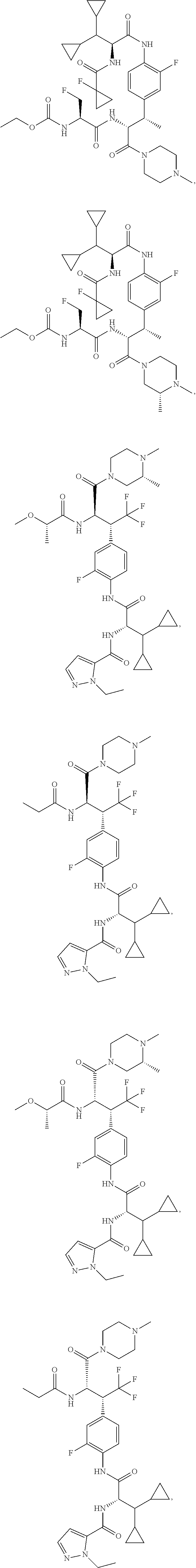 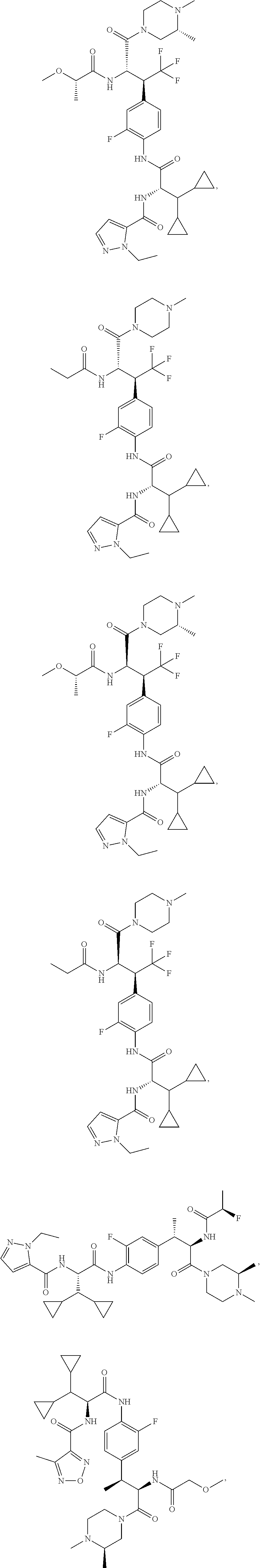 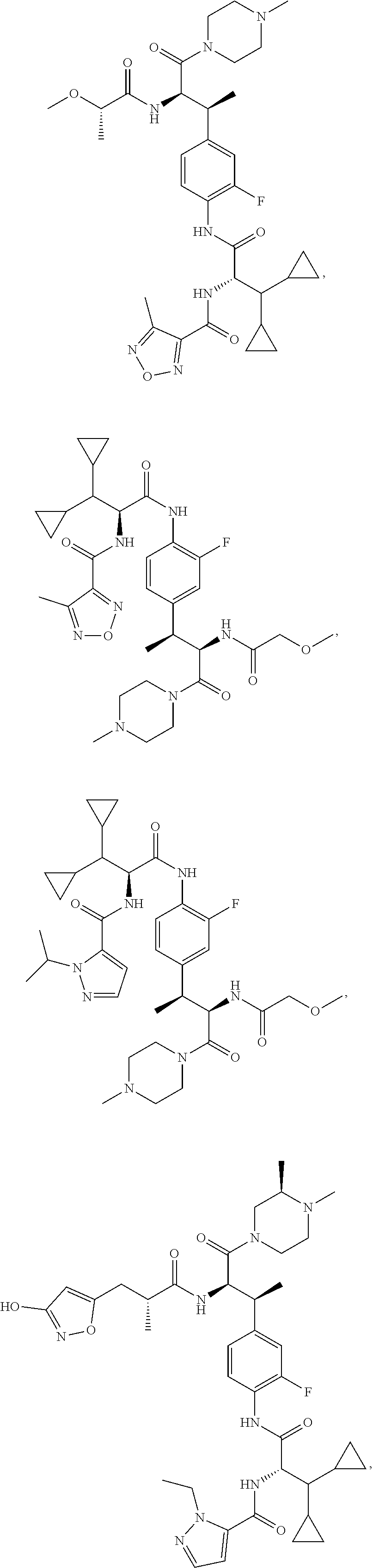 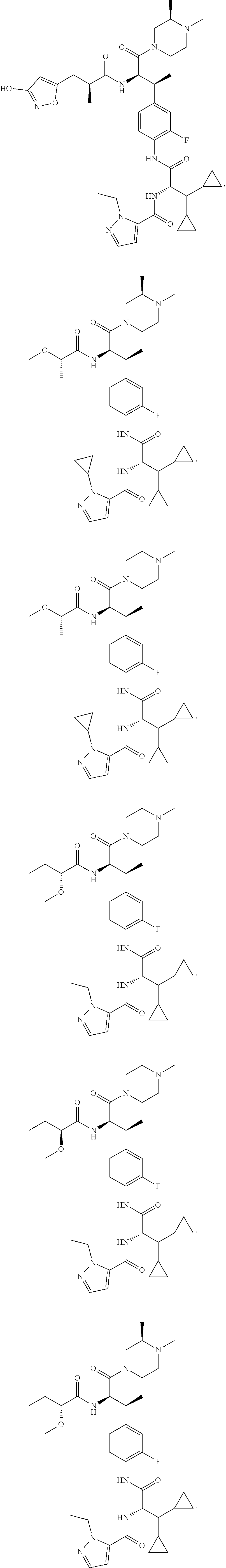 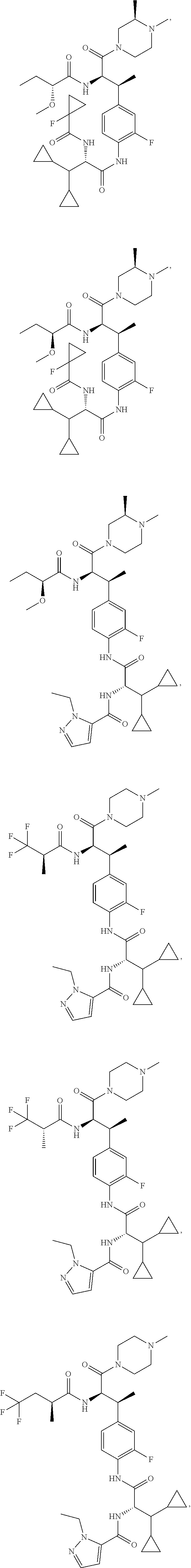 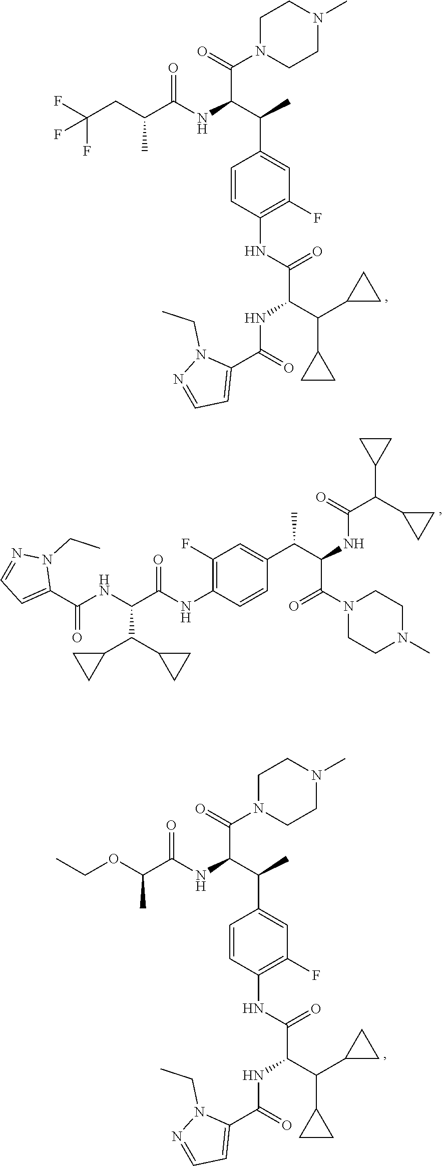 [SMILES: CCn1nccc1C(=O)N[C@H](C(=O)Nc1ccc([C@H](C)[C@@H](NC(=O)[C@H](C)CC(F)(F)F)C(=O)N2CCN(C)CC2)cc1F)C(C1CC1)C1CC1 ; CCn1nccc1C(=O)N[C@H](C(=O)Nc1ccc([C@H](C)[C@@H](NC(=O)C(C2CC2)C2CC2)C(=O)N2CCN(C)CC2)cc1F)C(C1CC1)C1CC1 ; CCO[C@H](C)C(=O)N[C@@H](C(=O)N1CCN(C)CC1)[C@@H](C)c1ccc(NC(=O)[C@@H](NC(=O)c2ccnn2CC)C(C2CC2)C2CC2)c(F)c1] 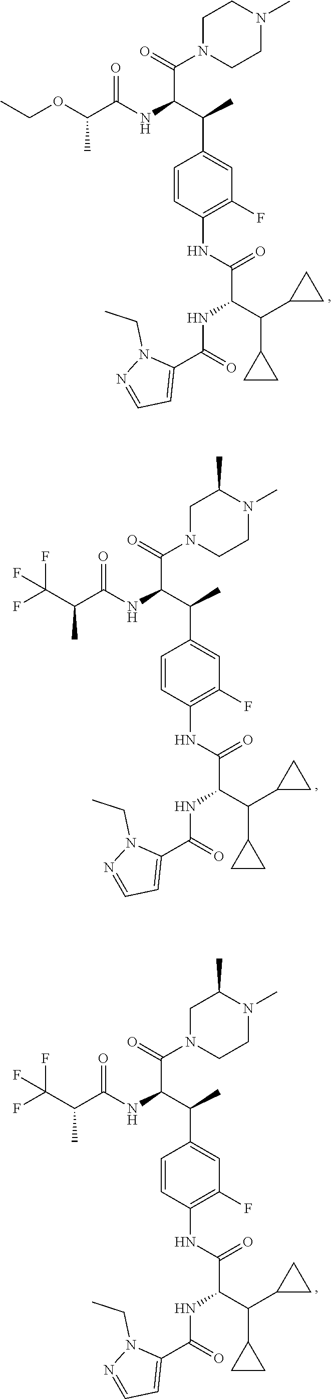 [SMILES: CCO[C@@H](C)C(=O)N[C@@H](C(=O)N1CCN(C)CC1)[C@@H](C)c1ccc(NC(=O)[C@@H](NC(=O)c2ccnn2CC)C(C2CC2)C2CC2)c(F)c1 ; CCn1nccc1C(=O)N[C@H](C(=O)Nc1ccc([C@H](C)[C@@H](NC(=O)[C@@H](C)C(F)(F)F)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)[C@H](C)C(F)(F)F)C(=O)N2CCN(C)[C@H](C)C2)cc1F)C(C1CC1)C1CC1] 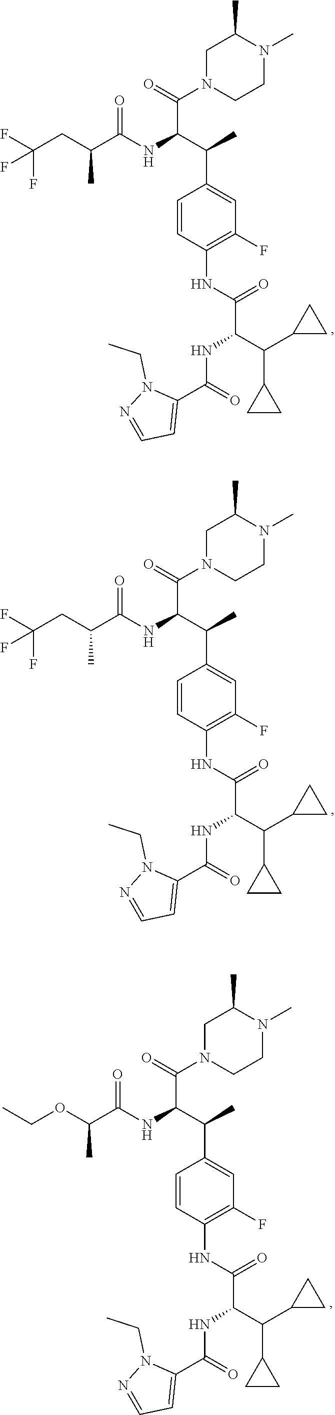 [SMILES: CCn1nccc1C(=O)N[C@H](C(=O)Nc1ccc([C@H](C)[C@@H](NC(=O)[C@@H](C)CC(F)(F)F)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)[C@H](C)CC(F)(F)F)C(=O)N2CCN(C)[C@H](C)C2)cc1F)C(C1CC1)C1CC1 ; CCO[C@H](C)C(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1] 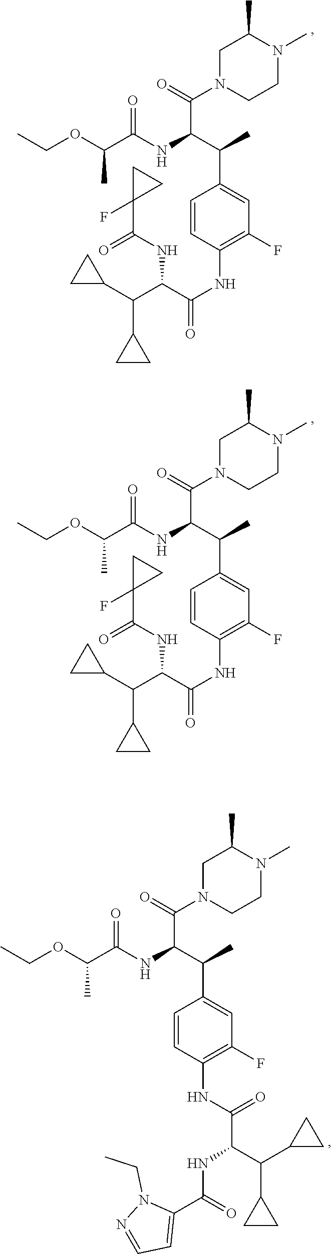 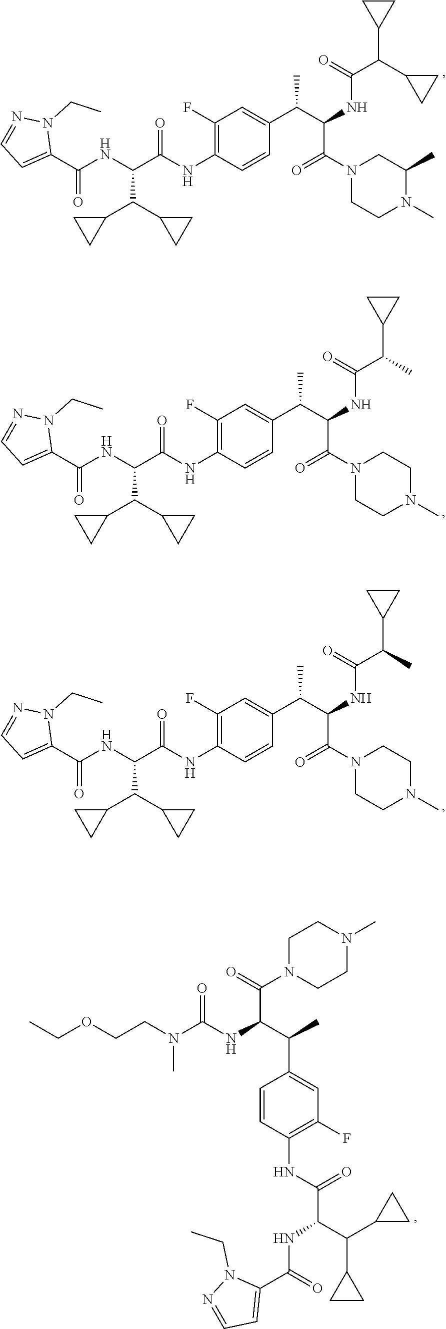 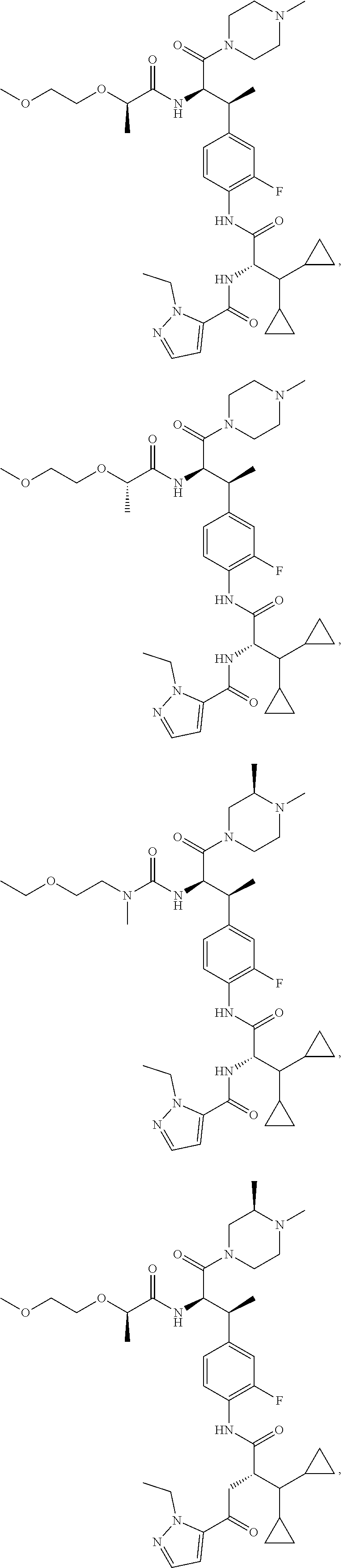 [SMILES: CCn1nccc1C(=O)N[C@H](C(=O)Nc1ccc([C@H](C)[C@@H](NC(=O)[C@@H](C)OCCOC)C(=O)N2CCN(C)CC2)cc1F)C(C1CC1)C1CC1 ; CCn1nccc1C(=O)N[C@H](C(=O)Nc1ccc([C@H](C)[C@@H](NC(=O)[C@H](C)OCCOC)C(=O)N2CCN(C)CC2)cc1F)C(C1CC1)C1CC1 ; CCOCCN(C)C(=O)N[C@@H](C(=O)N1CCN(C)[C@H](C)C1)[C@@H](C)c1ccc(NC(=O)[C@@H](NC(=O)c2ccnn2CC)C(C2CC2)C2CC2)c(F)c1 ; CCn1nccc1C(=O)C[C@H](C(=O)Nc1ccc([C@H](C)[C@@H](NC(=O)[C@@H](C)OCCOC)C(=O)N2CCN(C)[C@H](C)C2)cc1F)C(C1CC1)C1CC1] 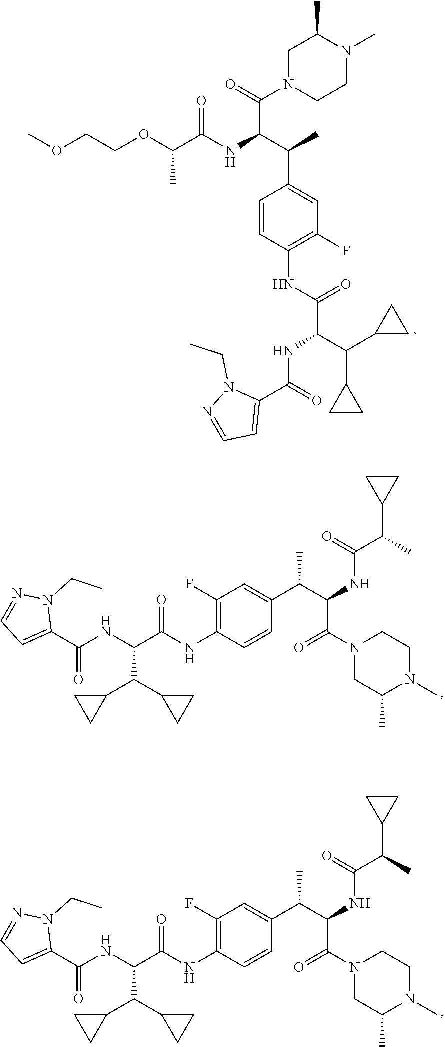 [SMILES: CCn1nccc1C(=O)N[C@H](C(=O)Nc1ccc([C@H](C)[C@@H](NC(=O)[C@H](C)OCCOC)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)[C@@H](C)C2CC2)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)[C@H](C)C2CC2)C(=O)N2CCN(C)[C@H](C)C2)cc1F)C(C1CC1)C1CC1] 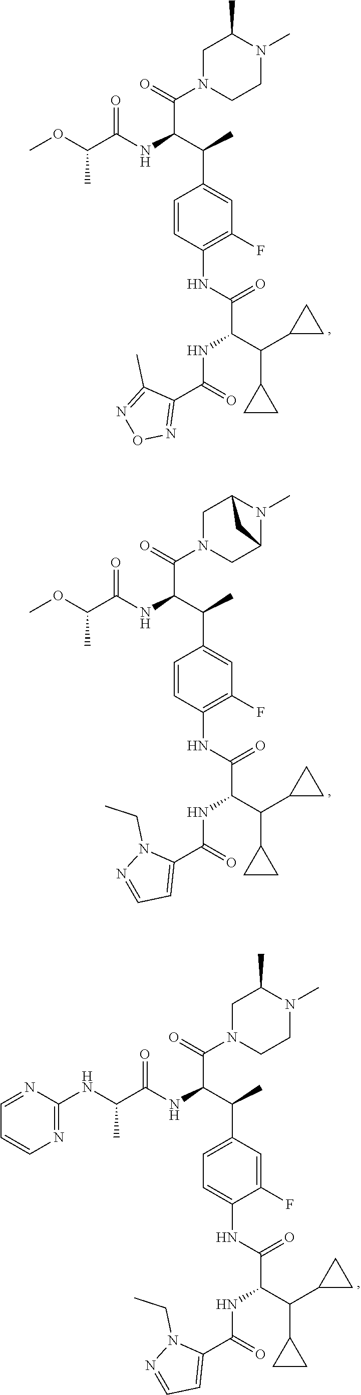 [SMILES: CO[C@@H](C)C(=O)N[C@@H](C(=O)N1CCN(C)[C@H](C)C1)[C@@H](C)c1ccc(NC(=O)[C@@H](NC(=O)c2nonc2C)C(C2CC2)C2CC2)c(F)c1 ; CCn1nccc1C(=O)N[C@H](C(=O)Nc1ccc([C@H](C)[C@@H](NC(=O)[C@H](C)OC)C(=O)N2C[C@H]3C[C@@H](C2)N3C)cc1F)C(C1CC1)C1CC1 ; CCn1nccc1C(=O)N[C@H](C(=O)Nc1ccc([C@H](C)[C@@H](NC(=O)[C@H](C)Nc2ncccn2)C(=O)N2CCN(C)[C@H](C)C2)cc1F)C(C1CC1)C1CC1] 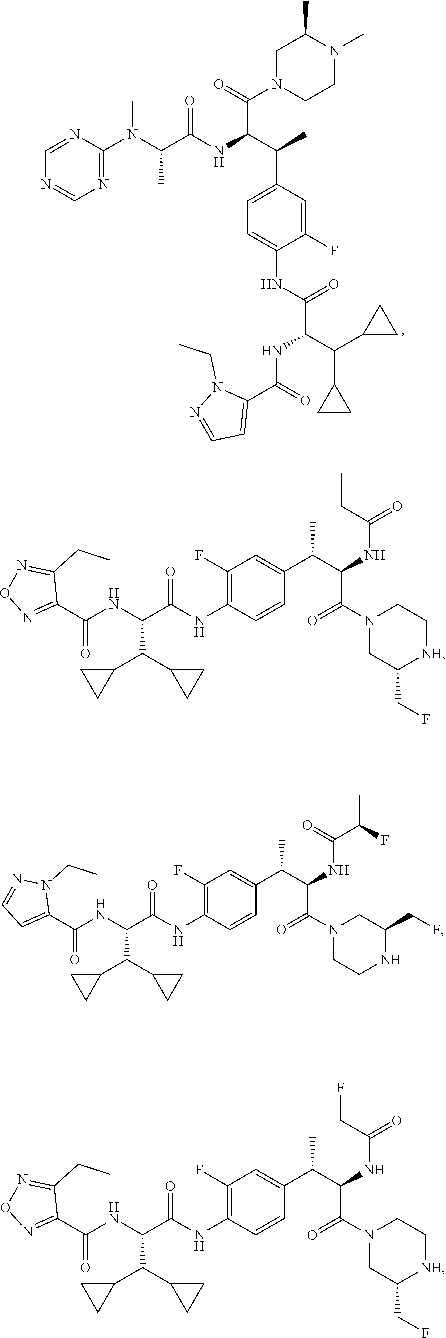 [SMILES: CCn1nccc1C(=O)N[C@H](C(=O)Nc1ccc([C@H](C)[C@@H](NC(=O)[C@H](C)N(C)c2ncncn2)C(=O)N2CCN(C)[C@H](C)C2)cc1F)C(C1CC1)C1CC1 ; CCC(=O)N[C@@H](C(=O)N1CCN[C@H](CF)C1)[C@@H](C)c1ccc(NC(=O)[C@@H](NC(=O)c2nonc2CC)C(C2CC2)C2CC2)c(F)c1 ; CCn1nccc1C(=O)N[C@H](C(=O)Nc1ccc([C@H](C)[C@@H](NC(=O)[C@@H](C)F)C(=O)N2CCN[C@H](CF)C2)cc1F)C(C1CC1)C1CC1 ; CCc1nonc1C(=O)N[C@H](C(=O)Nc1ccc([C@H](C)[C@@H](NC(=O)CF)C(=O)N2CCN[C@H](CF)C2)cc1F)C(C1CC1)C1CC1] 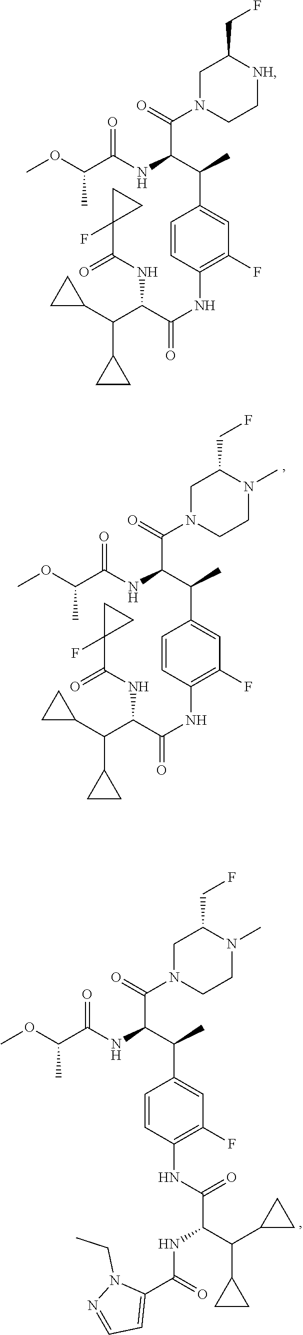 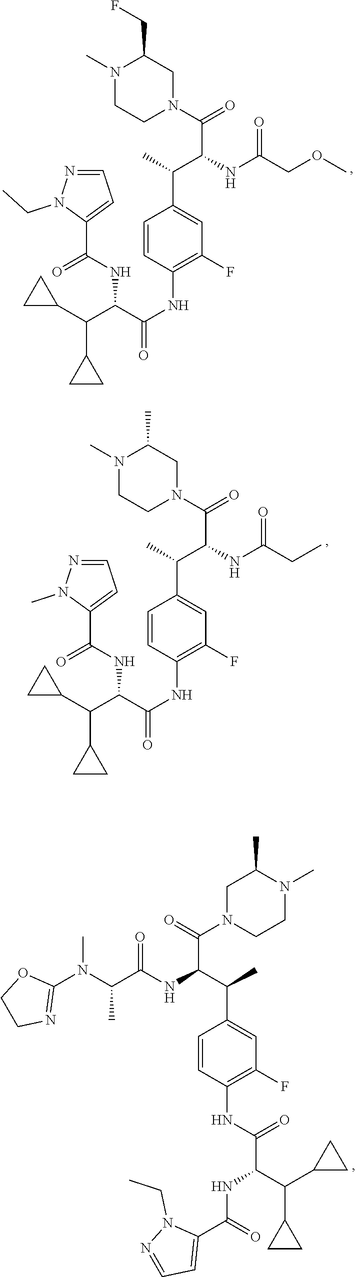 [SMILES: CCn1nccc1C(=O)N[C@H](C(=O)Nc1ccc([C@H](C)[C@@H](NC(=O)COC)C(=O)N2CCN(C)[C@@H](CF)C2)cc1F)C(C1CC1)C1CC1 ; CCC(=O)N[C@@H](C(=O)N1CCN(C)[C@H](C)C1)[C@@H](C)c1ccc(NC(=O)[C@@H](NC(=O)c2ccnn2C)C(C2CC2)C2CC2)c(F)c1 ; CCn1nccc1C(=O)N[C@H](C(=O)Nc1ccc([C@H](C)[C@@H](NC(=O)[C@H](C)N(C)C2=NCCO2)C(=O)N2CCN(C)[C@H](C)C2)cc1F)C(C1CC1)C1CC1] 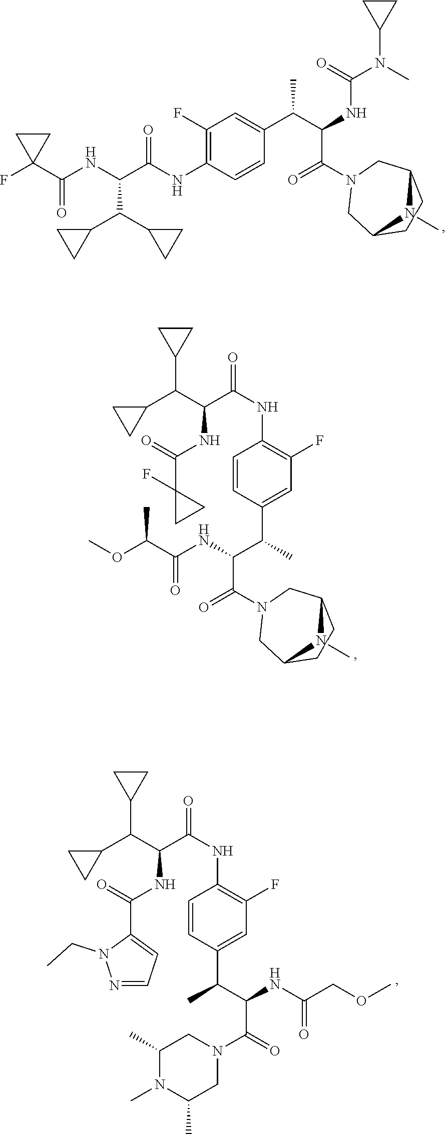 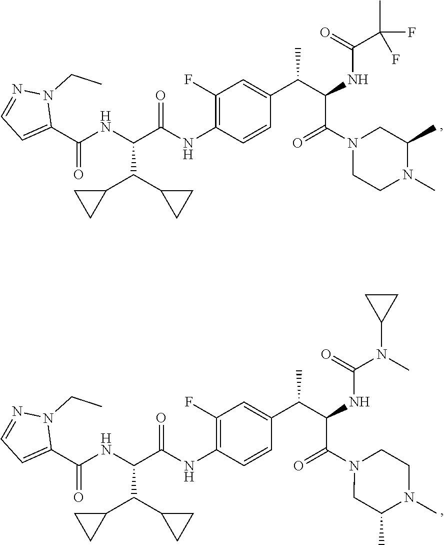 [SMILES: CCn1nccc1C(=O)N[C@H](C(=O)Nc1ccc([C@H](C)[C@@H](NC(=O)C(C)(F)F)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)N(C)C2CC2)C(=O)N2CCN(C)[C@H](C)C2)cc1F)C(C1CC1)C1CC1] 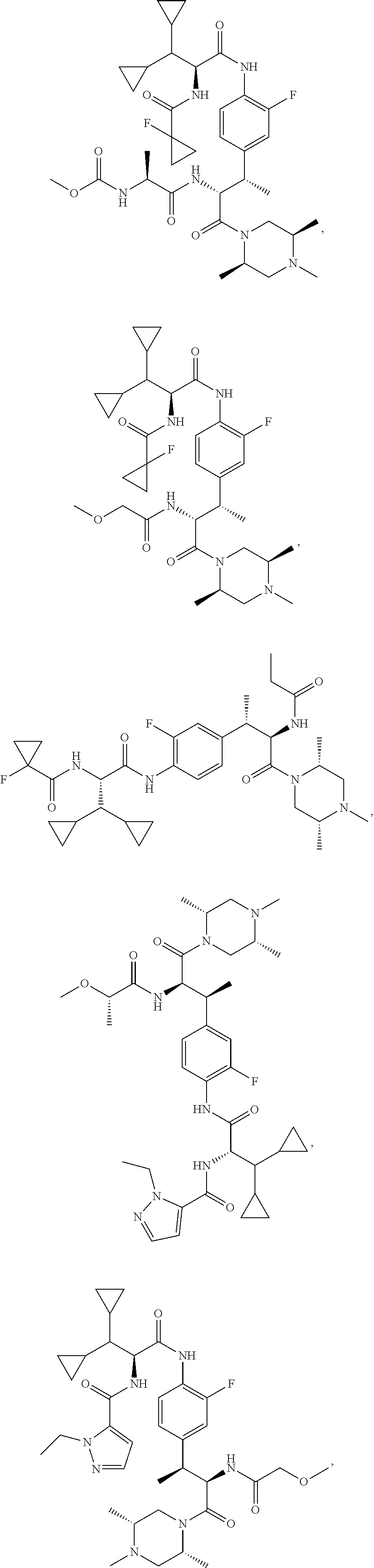 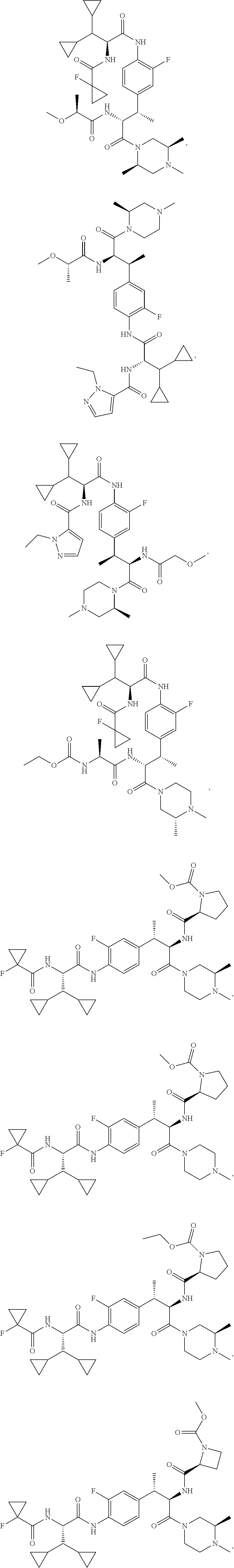 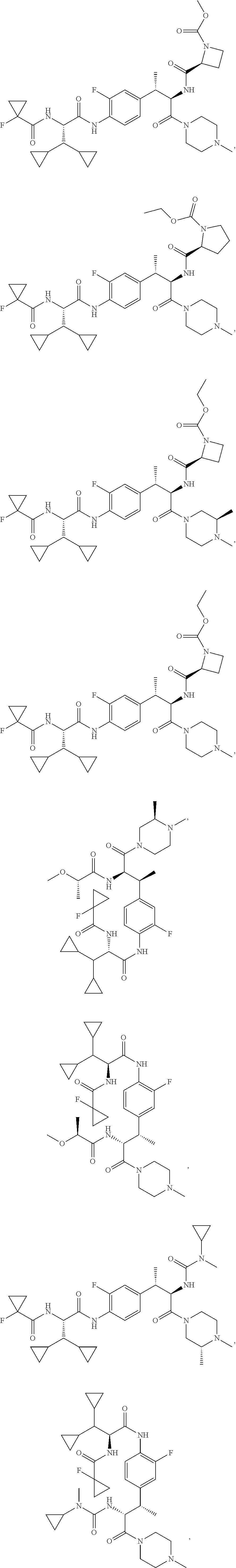 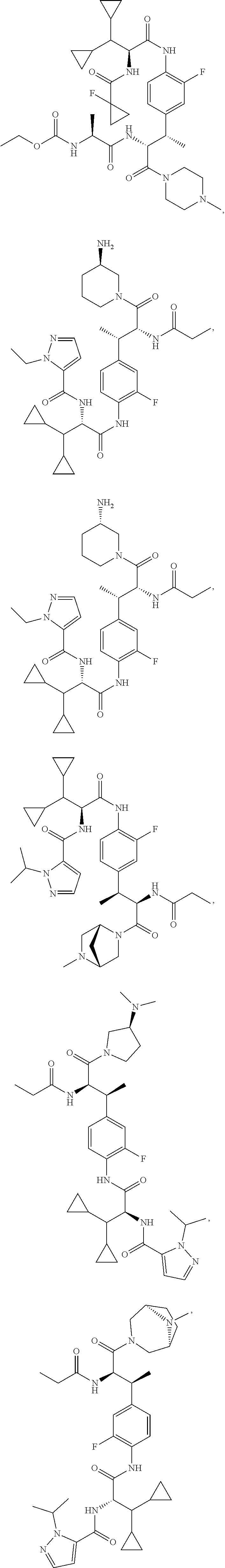 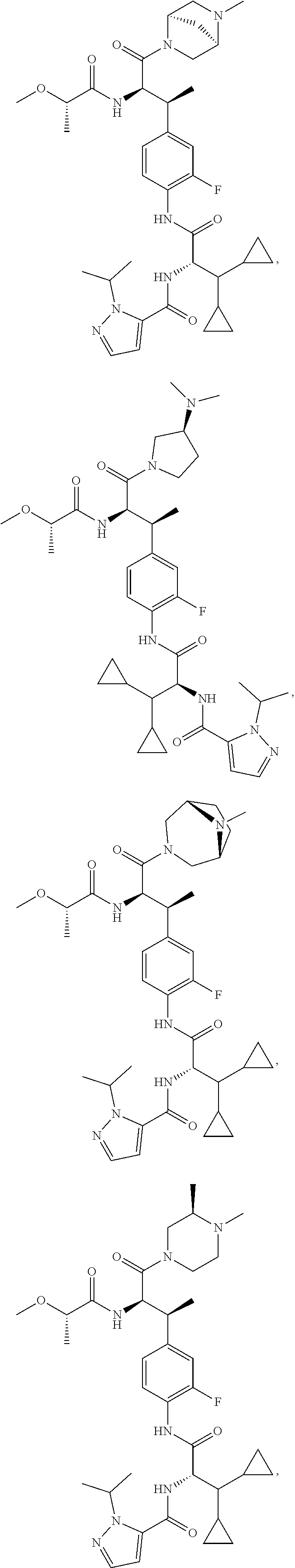 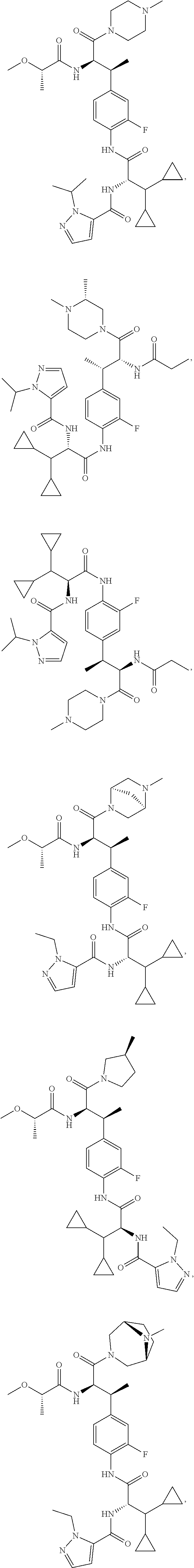 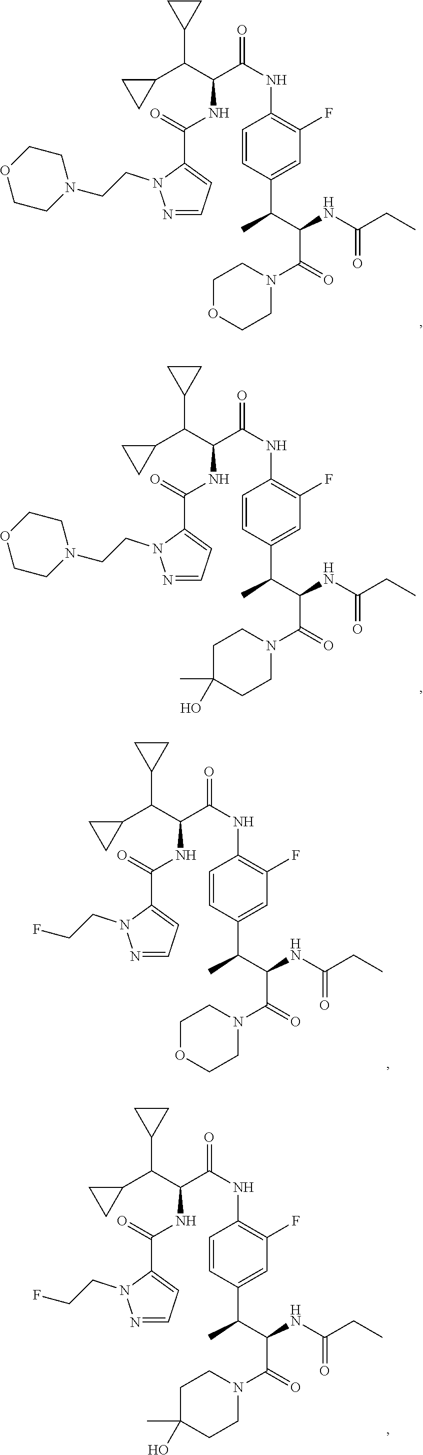 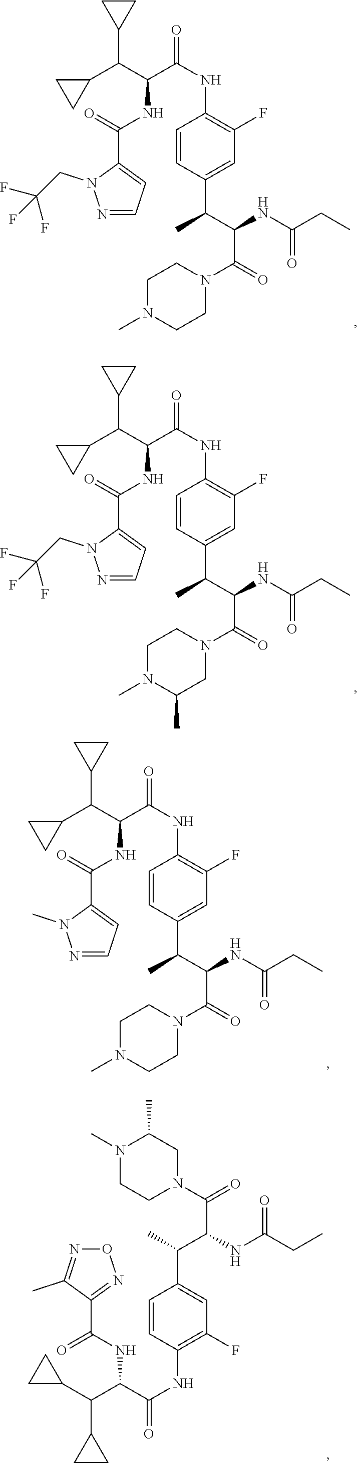 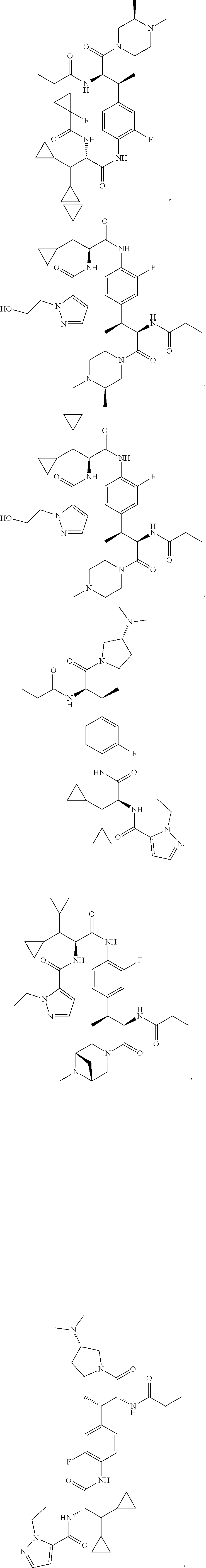 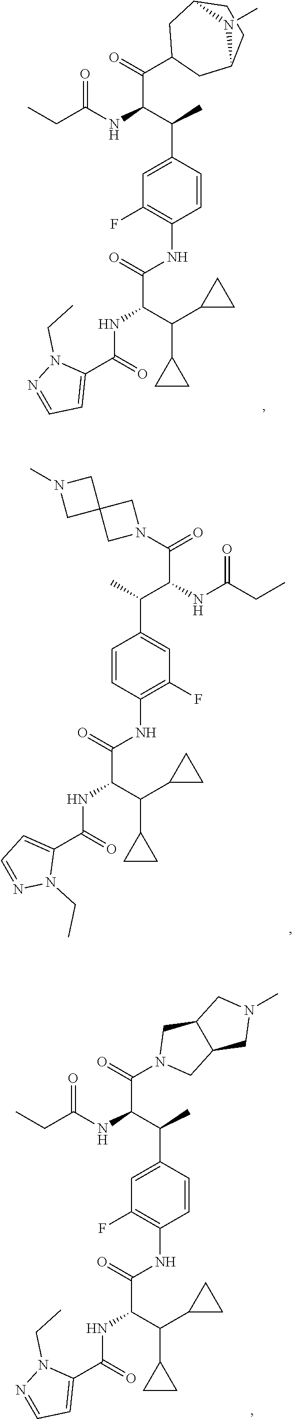 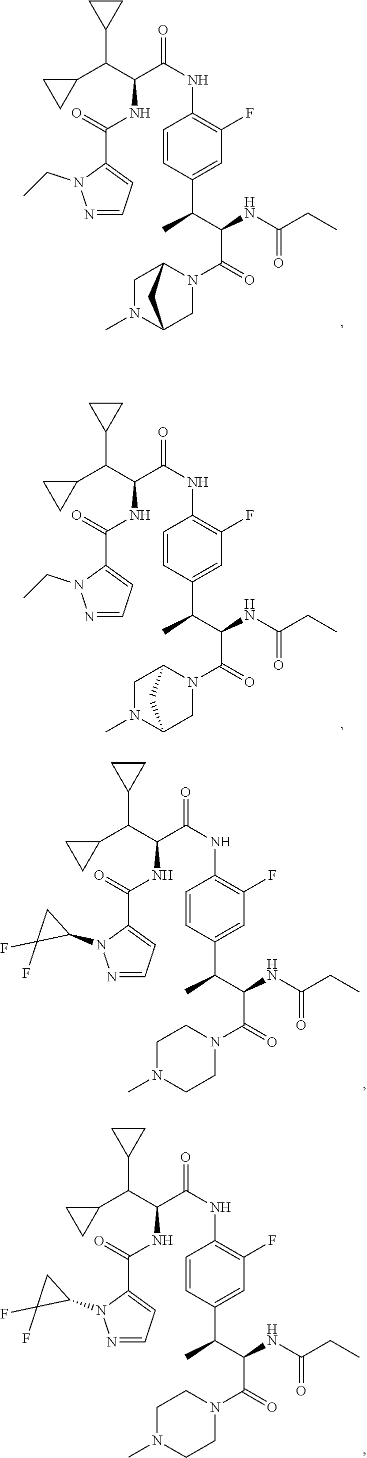 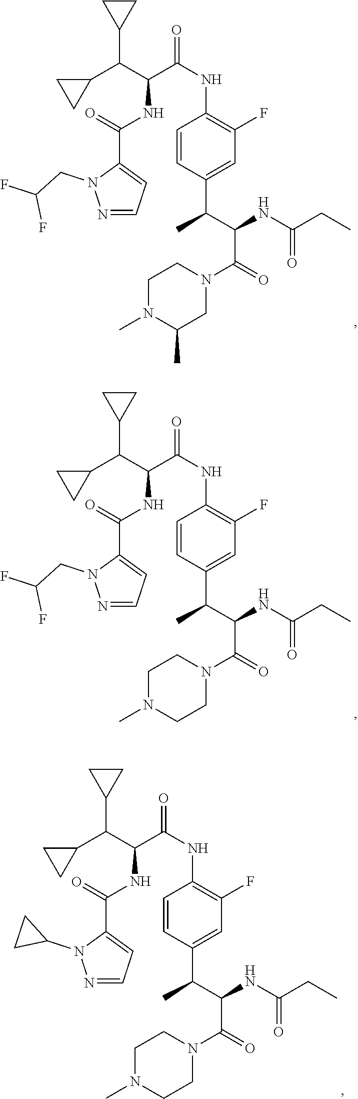 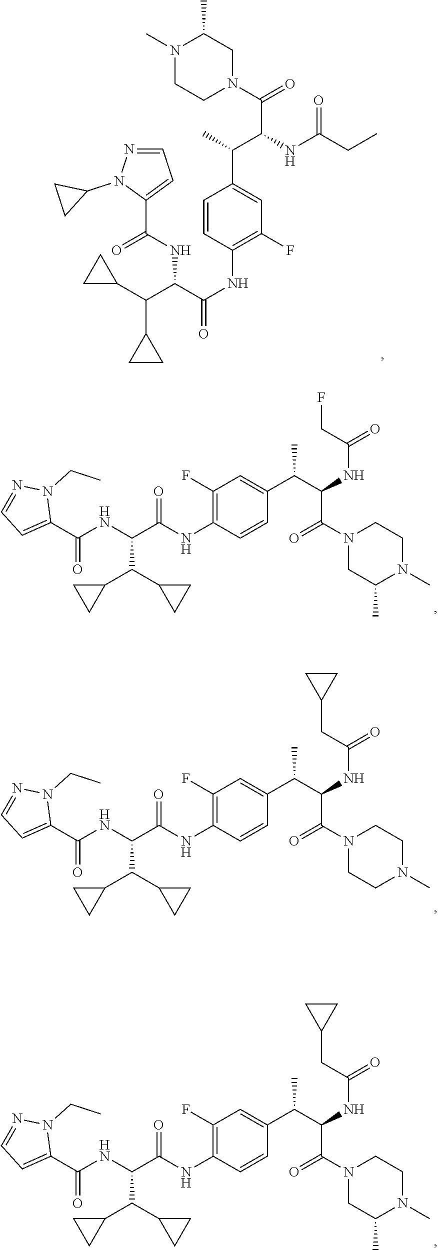 [SMILES: CCC(=O)N[C@@H](C(=O)N1CCN(C)[C@H](C)C1)[C@@H](C)c1ccc(NC(=O)[C@@H](NC(=O)c2ccnn2C2CC2)C(C2CC2)C2CC2)c(F)c1 ; CCn1nccc1C(=O)N[C@H](C(=O)Nc1ccc([C@H](C)[C@@H](NC(=O)CF)C(=O)N2CCN(C)[C@H](C)C2)cc1F)C(C1CC1)C1CC1 ; CCn1nccc1C(=O)N[C@H](C(=O)Nc1ccc([C@H](C)[C@@H](NC(=O)CC2CC2)C(=O)N2CCN(C)CC2)cc1F)C(C1CC1)C1CC1 ; CCn1nccc1C(=O)N[C@H](C(=O)Nc1ccc([C@H](C)[C@@H](NC(=O)CC2CC2)C(=O)N2CCN(C)[C@H](C)C2)cc1F)C(C1CC1)C1CC1] 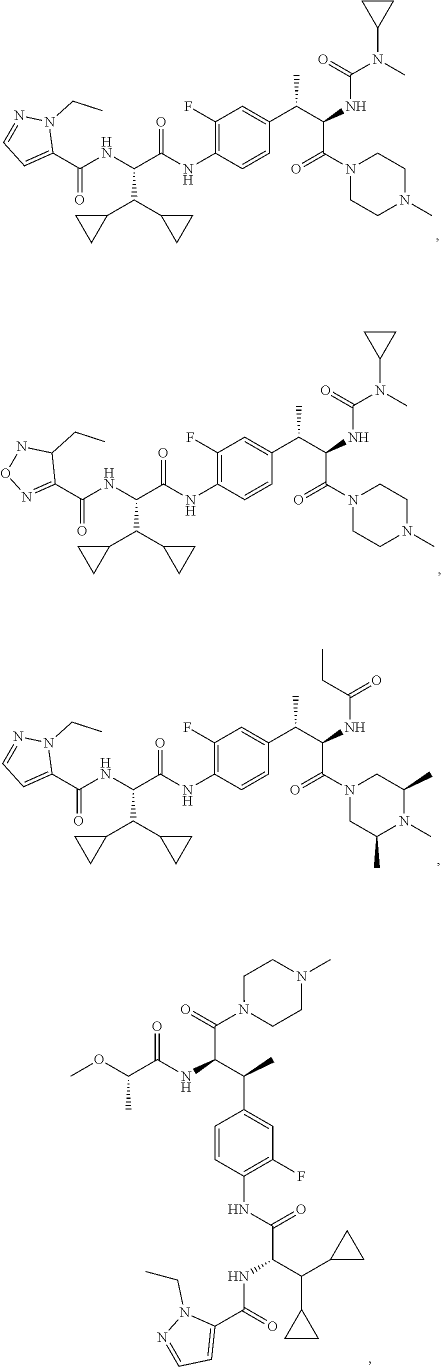 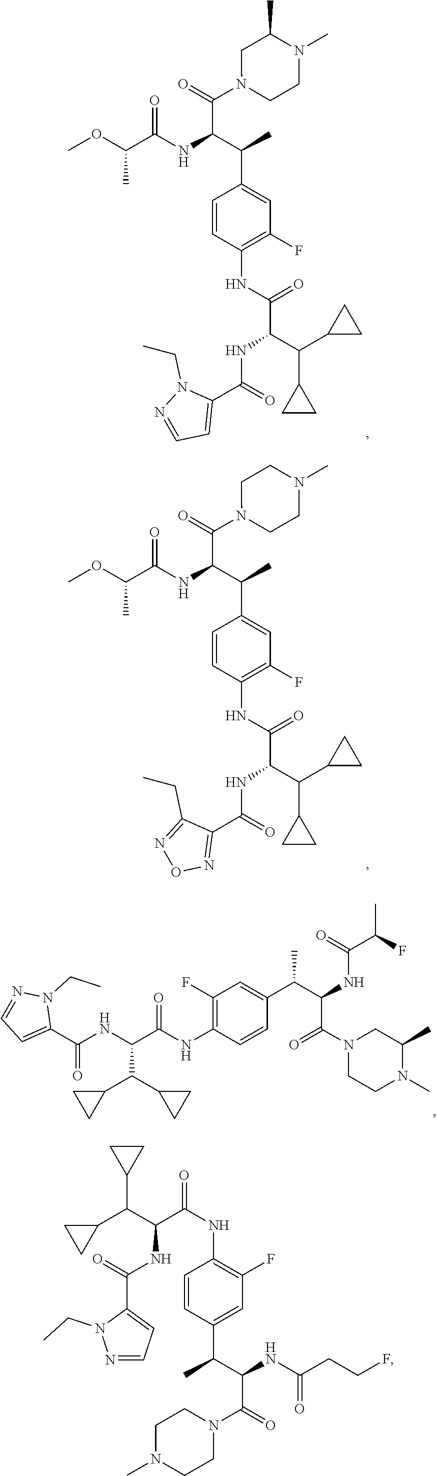 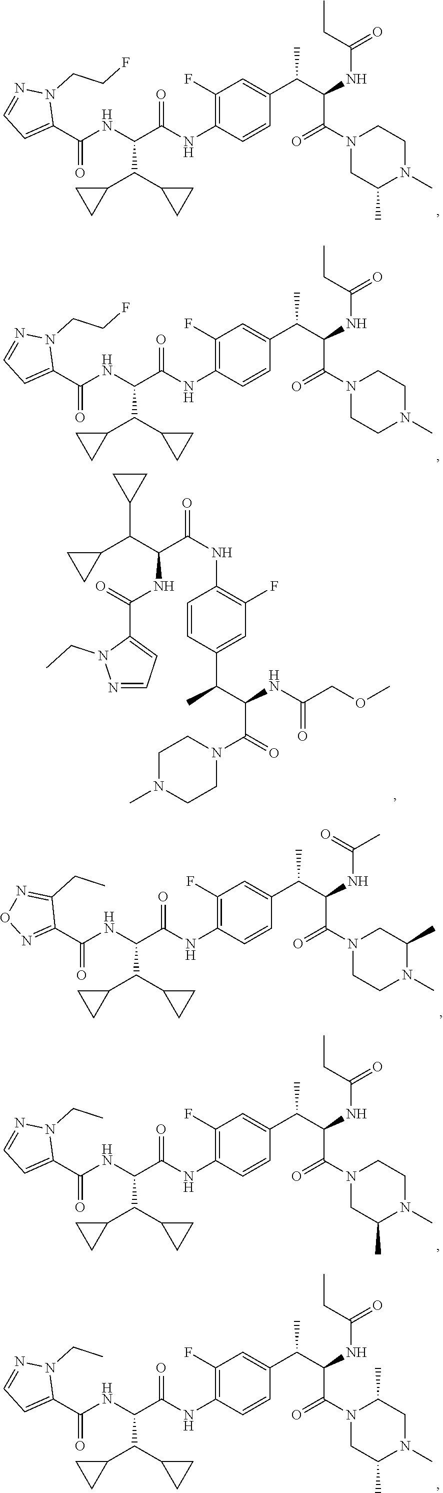 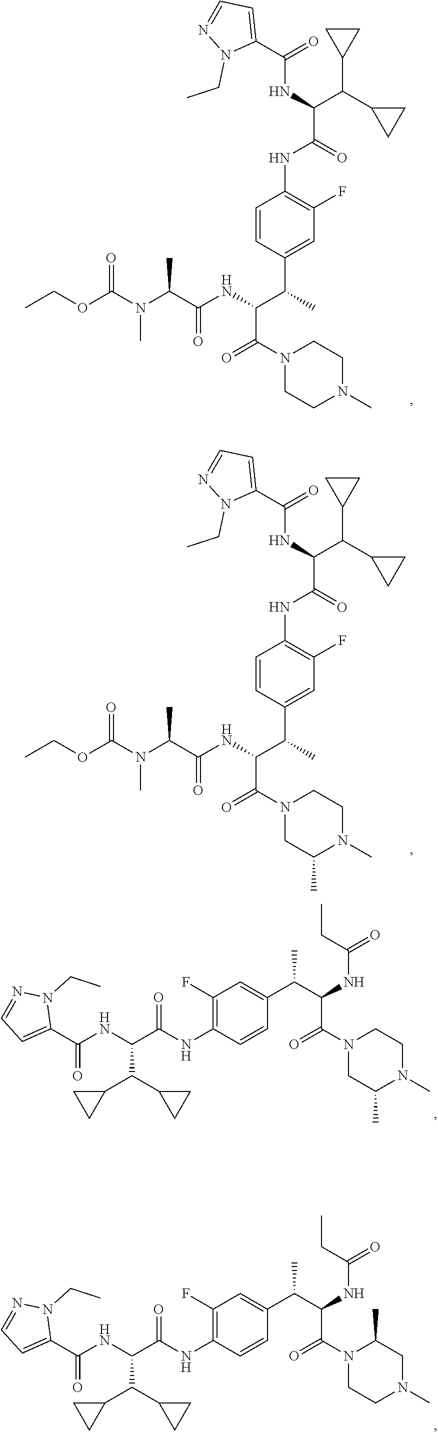 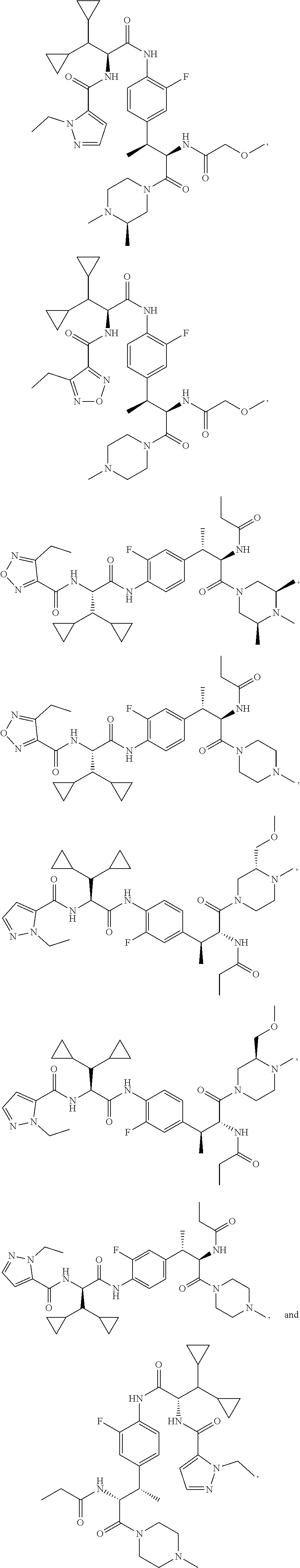
or a pharmaceutically acceptable salt thereof.

62. A pharmaceutical composition comprising a compound or salt of claim 1, and a pharmaceutically acceptable excipient.

63. A method of modulating IL-17A in a subject in need thereof, comprising administering to the subject a compound or salt of claim 1.

64. A method of treating an inflammatory disease or condition comprising administering to a subject in need thereof a compound or salt of claim 1.

65. The method of claim 64, wherein the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis.

66. The compound or salt of claim 5 of the formula: 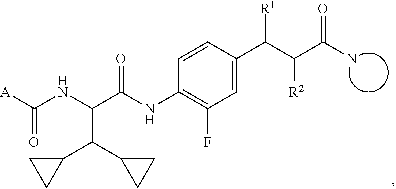
wherein:
R¹ is —CH₃ or —CF₃;
R² is selected from: 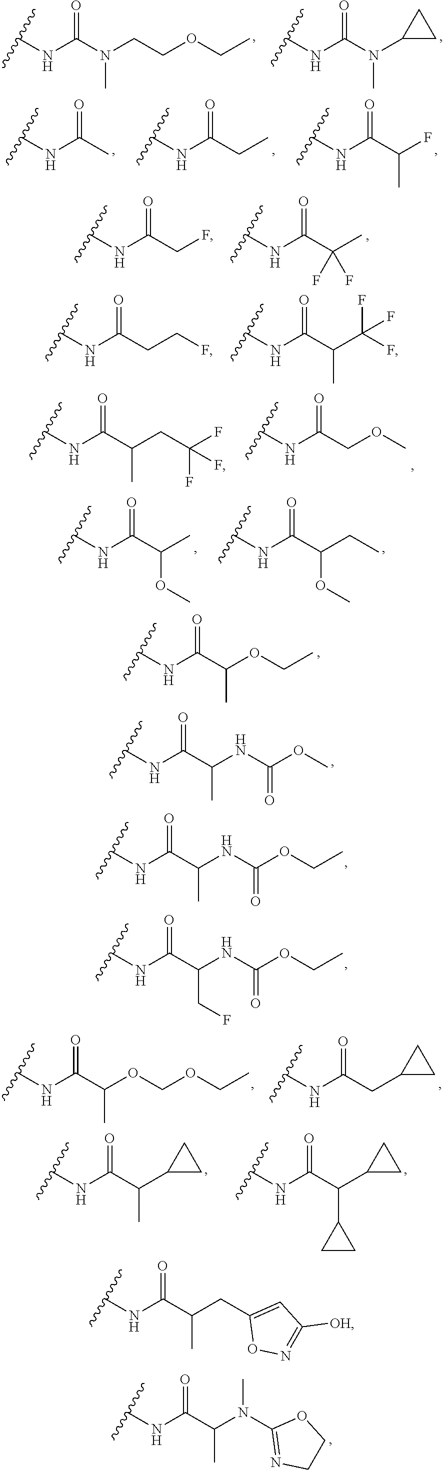 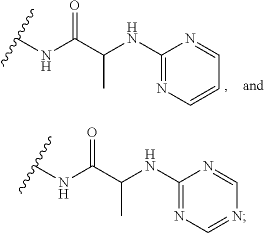
A is selected from: 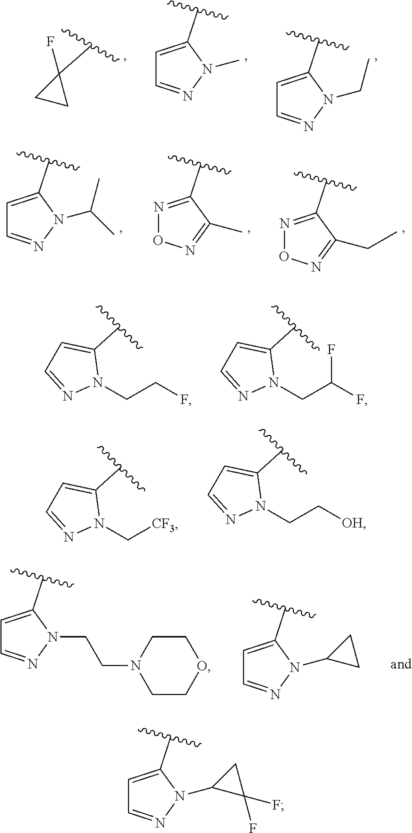
and 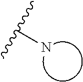 [SMILES: *N]
is selected from: 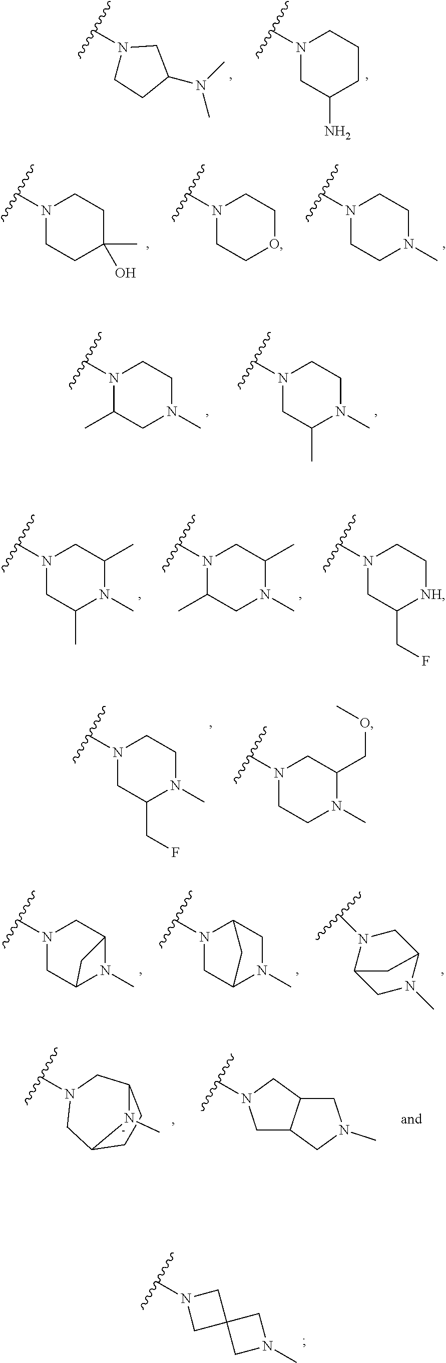
or a pharmaceutically acceptable salt thereof.

67. The compound or salt of claim 5 of the formula: 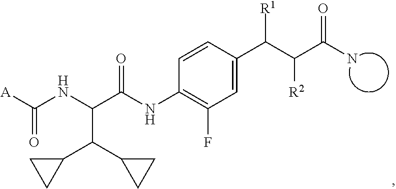
wherein:
R¹ is methyl;
R² is selected from: 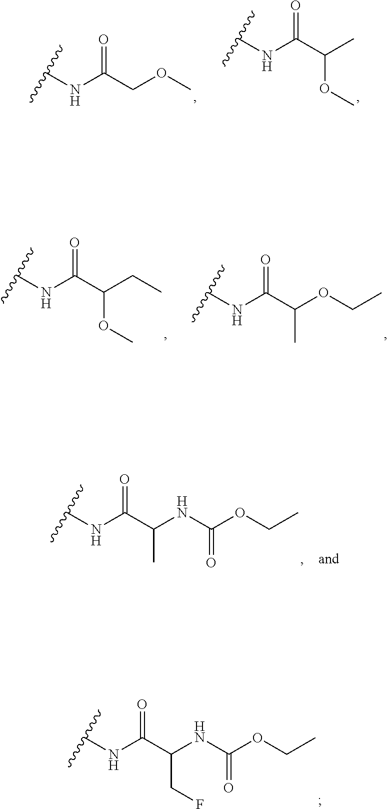
A is selected from: 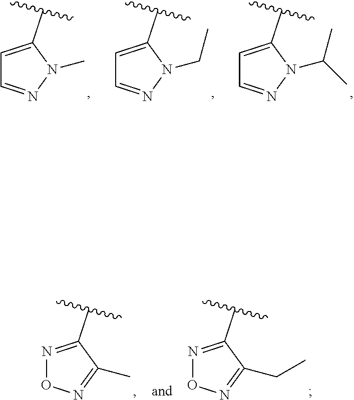
and 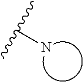 [SMILES: *N]
is selected from: 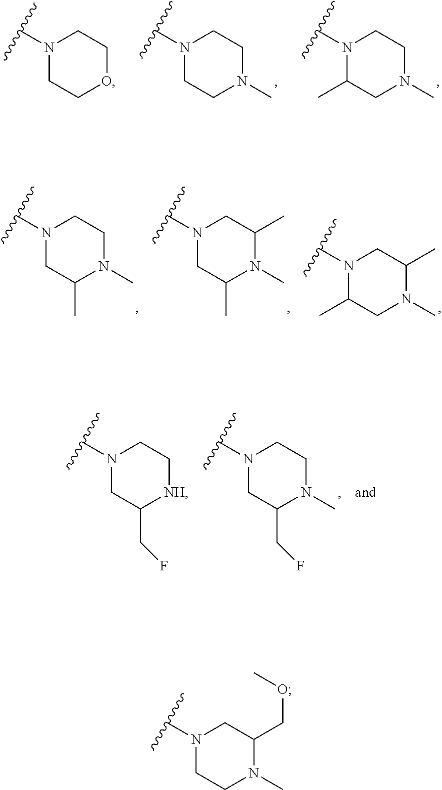
or a pharmaceutically acceptable salt thereof.

68. The compound or salt of claim 67 of the formula: 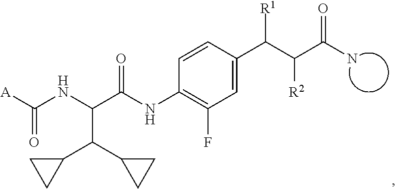
wherein:
R¹ is methyl;
R² is; 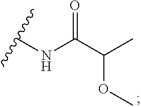 [SMILES: *NC(=O)C(C)OC]
A is 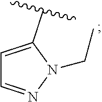 [SMILES: *c1ccnn1CC]
and 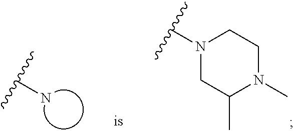
or a pharmaceutically acceptable salt thereof.

69. The compound or salt of claim 68 of the formula: 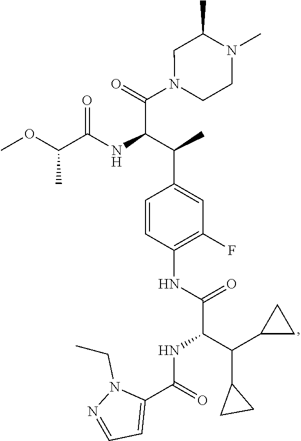 [SMILES: CCn1nccc1C(=O)N[C@H](C(=O)Nc1ccc([C@H](C)[C@@H](NC(=O)[C@H](C)OC)C(=O)N2CCN(C)[C@H](C)C2)cc1F)C(C1CC1)C1CC1]
or a pharmaceutically acceptable salt thereof.

70. The compound of claim 69 which is:

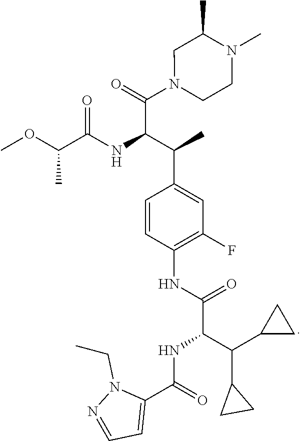 [SMILES: CCn1nccc1C(=O)N[C@H](C(=O)Nc1ccc([C@H](C)[C@@H](NC(=O)[C@H](C)OC)C(=O)N2CCN(C)[C@H](C)C2)cc1F)C(C1CC1)C1CC1]

71. A pharmaceutical composition comprising a compound or salt of claim 68, and a pharmaceutically acceptable excipient.

72. A pharmaceutical composition comprising a compound or salt of claim 69, and a pharmaceutically acceptable excipient.

73. A method of treating an inflammatory disease or condition comprising administering to a subject in need thereof a compound or salt of claim 69, wherein the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis.

74. The method of claim 73 wherein the compound is: 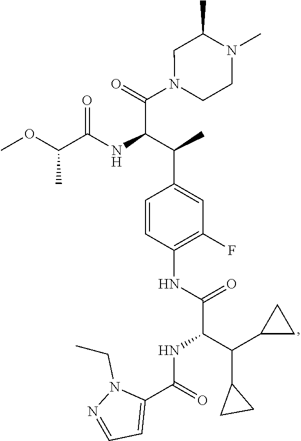 [SMILES: CCn1nccc1C(=O)N[C@H](C(=O)Nc1ccc([C@H](C)[C@@H](NC(=O)[C@H](C)OC)C(=O)N2CCN(C)[C@H](C)C2)cc1F)C(C1CC1)C1CC1]
and wherein the inflammatory disease or condition is plaque psoriasis.

75. The method of claim 73 wherein the compound is: 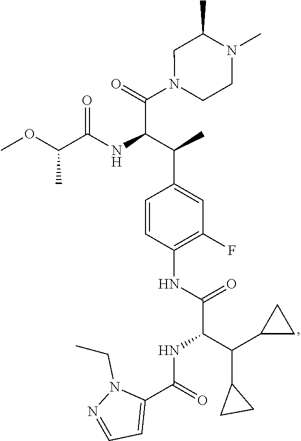 [SMILES: CCn1nccc1C(=O)N[C@H](C(=O)Nc1ccc([C@H](C)[C@@H](NC(=O)[C@H](C)OC)C(=O)N2CCN(C)[C@H](C)C2)cc1F)C(C1CC1)C1CC1]
and wherein the inflammatory disease or condition is selected from psoriatic arthritis, ankylosing spondylitis, or spondyloarthritis.

76. The compound or salt of claim 67 of the formula: 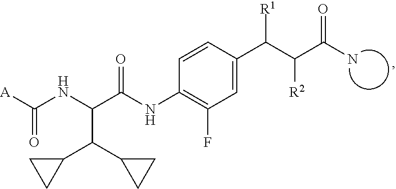
wherein:
R¹ is methyl;
R² is 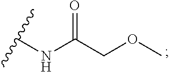 [SMILES: *NC(=O)COC]
A is 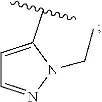 [SMILES: *c1ccnn1CC]
and 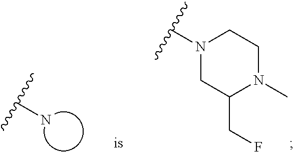
or a pharmaceutically acceptable salt thereof.

77. The compound or salt of claim 76 of the formula: 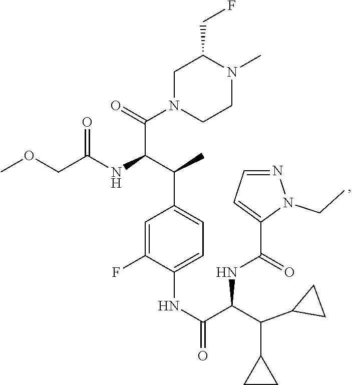 [SMILES: CCn1nccc1C(=O)N[C@H](C(=O)Nc1ccc([C@H](C)[C@@H](NC(=O)COC)C(=O)N2CCN(C)[C@@H](CF)C2)cc1F)C(C1CC1)C1CC1]
or a pharmaceutically acceptable salt thereof.

78. The compound of claim 77 which is:

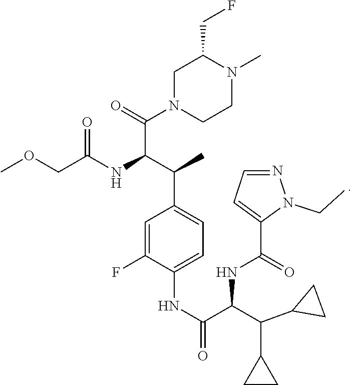 [SMILES: CCn1nccc1C(=O)N[C@H](C(=O)Nc1ccc([C@H](C)[C@@H](NC(=O)COC)C(=O)N2CCN(C)[C@@H](CF)C2)cc1F)C(C1CC1)C1CC1]

79. A pharmaceutical composition comprising a compound or salt of claim 77, and a pharmaceutically acceptable excipient.

80. A method of treating an inflammatory disease or condition comprising administering to a subject in need thereof a compound or salt of claim 77, wherein the inflammatory disease or condition is selected from plaque psoriasis, guttate psoriasis, inverse psoriasis, pustular psoriasis, erythrodermic psoriasis, psoriatic arthritis, ankylosing spondylitis, hidradenitis suppurativa, rheumatoid arthritis, Palmoplantar Psoriasis, Spondyloarthritis, and Non-infectious Uveitis.

81. The method of claim 80 wherein the compound is: 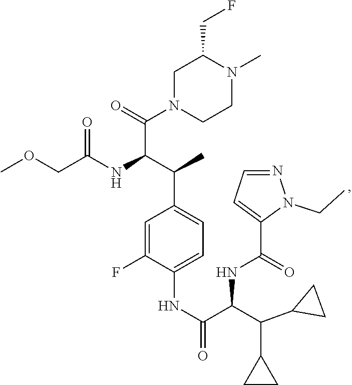 [SMILES: CCn1nccc1C(=O)N[C@H](C(=O)Nc1ccc([C@H](C)[C@@H](NC(=O)COC)C(=O)N2CCN(C)[C@@H](CF)C2)cc1F)C(C1CC1)C1CC1]
and wherein the inflammatory disease or condition is plaque psoriasis.

82. The method of claim 80 wherein the compound is: 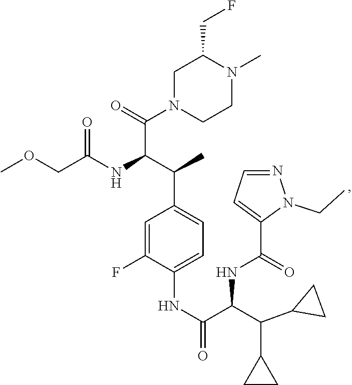 [SMILES: CCn1nccc1C(=O)N[C@H](C(=O)Nc1ccc([C@H](C)[C@@H](NC(=O)COC)C(=O)N2CCN(C)[C@@H](CF)C2)cc1F)C(C1CC1)C1CC1]
and wherein the inflammatory disease or condition is selected from psoriatic arthritis, ankylosing spondylitis, or spondyloarthritis.
